(19) United States
(12) Patent Application Publication
(10) Pub. No.: US 2026/0200896 A1
(43) Pub. Date: Jul. 16, 2026

(54) INHIBITORS OF SPNS2 AND USES THEREOF

(71) Applicant: NEW YORK UNIVERSITY, New York, NY (US)

(72) Inventors: John Kenneth DICKSON, JR., New York, NY (US); Susan SCHWAB, New York, NY (US); Xinyan HUANG, New York, NY (US)

(73) Assignee: NEW YORK UNIVERSITY, New York, NY (US)

(21) Appl. No.: 19/118,582

(22) PCT Filed: Oct. 4, 2023
(86) PCT No.: PCT/US2023/034433

§ 371 Date: Apr. 4, 2025

Related U.S. Application Data
(60) Provisional application No. 63/413,443, filed on Oct. 5, 2022.

(51) Int. Cl. C07D 403/04; A61K 31/4709; A61K 31/498; A61K 31/5377; C07D 241/44; C07D 401/04; C07D 401/14; C07D 403/14; C07D 405/14; C07D 413/04; C07D 491/056

(52) U.S. Cl. CPC C07D 403/04; A61K 31/4709; A61K 31/498; A61K 31/5377; C07D 241/44; C07D 401/04; C07D 401/14; C07D 403/14; C07D 405/14; C07D 413/04; C07D 491/056

(57) ABSTRACT

Compounds are disclosed that have a formula (I) and wherein R₁, R₂, R₃, R₄, X, and Y are as described herein. The compounds may be prepared as compositions, e.g., pharmaceutical compositions, or as dosage forms, e.g., pharmaceutical dosage forms, and may be used for the prevention and treatment of a variety of conditions in mammals including humans, including by way of non-limiting example, autoimmune diseases, such as multiple sclerosis (MS) and inflammatory bowel disease (IBD), fibrosis, muscle wasting, metastases, acute lung injury, rheumatoid arthritis, colitis, Alzheimer's disease, and other diseases related to Sphingolipid Transporter 2 (SP-NS2) activity.

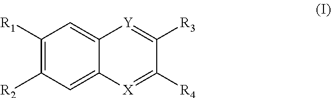

### STATEMENT REGARDING FEDERALLY SPONSORED RESEARCH OR DEVELOPMENT

This invention was made with government support under Grant #636528 awarded by the Crohn's & Colitis Foundation and FAIN #R01AI148178 awarded by the National Institutes of Health. The government has certain rights in the invention.

### FIELD OF INVENTION

The present invention relates to compounds capable of modulating the activity of Sphingolipid Transporter 2 (SPNS2). Specifically, this invention relates to compounds capable of inhibiting SPNS2, and uses of such compounds to treat diseases or conditions related to SPNS2 activity. More particularly, the compounds may be used to treat autoimmune diseases, such as, but not limited to, multiple sclerosis (MS) and inflammatory bowel disease (IBD), fibrosis, muscle wasting, metastases, acute lung injury, rheumatoid arthritis, colitis, Alzheimer's disease, and other diseases related to Sphingolipid Transporter 2 (SPNS2) activity.

### BACKGROUND OF THE INVENTION

The ability of T cells to exit secondary lymphoid organs, and subsequently migrate to sites of inflammation, depends on their ability to respond to and migrate towards a gradient of the bioactive lipid sphingosine 1-phosphate (S1P). The concentration of S1P is higher in lymph than in lymph nodes. This gradient guides lymphocytes out of lymph nodes into the lymph and back into circulation.

The major facilitator superfamily transporter SPNS2 supplies S1P into lymph but not blood, and SPNS2 remains the only known requirement for lymph but not blood S1P.

SPNS2 plays a critical role in maintaining the S1P gradient. When SPNS2 is disrupted, T cells remain trapped within the lymph nodes and are unable to migrate to sites of inflammation.

Disrupting the S1P pathway is a well-validated modality that has been demonstrated to be protective in multiple sclerosis (MS) and inflammatory bowel disease (IBD) in both mouse studies and human clinical trials. FTY720 (Gilenya®, fingolimod) modulates S1P receptors, and its approval for MS marked the first time this pathway had been successfully drugged. Unfortunately, the S1P receptors targeted by Gilenya® also play important roles in a wide range of cell types including endothelial cells, cardiomyocytes, and hematopoietic stem cells. Gilenya® thus has numerous, sometimes life threatening, side effects including dyspnea, hypertension, brachycardia and leukopenia.

This adverse event profile has limited the use of Gilenya® to patients where the risks associated with disease progression outweigh the risks associated with taking the drug. SPNS2 is thus a potential immunosuppressive drug target that could trap T cells in the lymph nodes without substantial cardiac and vascular side effects.

Thus, there exists an unmet need for an SPNS2-targeting pharmaceutical with immunosuppressive properties with reduced cardiac and vascular side effects.

### SUMMARY OF THE INVENTION

Various non-limiting aspects and embodiments of the invention are described below.

The present invention provides compounds capable of inhibiting SPNS2 and demonstrated to have structure activity relationships (SAR) encompassing multiple positions around a core heterocyclic scaffold. This SAR is observed with cellular potency against the target SPNS2 transporters' actions and their druggability properties such as solubility, permeability, and metabolic stability. At least one compound of this series has been shown to have good oral bioavailability. Good selectivity at the target level has been demonstrated for multiple members of this chemotype against related cellular proteins.

Various non-limiting aspects and embodiments of the invention are described below.

In one aspect, the present application is directed to a compound having the structure according to Formula (I):

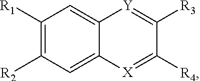

wherein:
X is —C(H)═ or —N═;
Y is —C(H)═ or —N═;
R₁ is a hydrogen, —Cl, —F, or a C₁-C₆ alkyl;
R₂ is a hydrogen, —Cl, —F, or a C₁-C₆ alkyl;
R₃ is —Cl, R₅, —OR₅, —NHR₅, or —N(R₅)₂;
R₄ is —OR₅, —NHR₅, —N(R₅)₂,

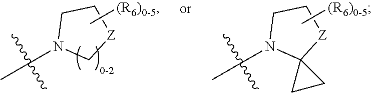

R₅ is independently at each occurrence an optionally substituted C₁-C₁₂ alkyl, an optionally substituted C₁-C₁₂ alkenyl, an optionally substituted C₁-C₁₂ alkoxy, an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 5-6 membered heteroaryl having 1-4 heteroatoms independently selected from N, O, and S, a fused bicyclic ring system having 1-4 heteroatoms independently selected from N, O, and S, or an optionally substituted combination of any two of a C₁-C₁₂ alkyl, a C₁-C₂ alkenyl and a ring;
Z is —C(H)—, —CH₂—, or —O—;
R₆ is independently at each occurrence an optionally substituted C₁-C₂ alkyl; an optionally substituted C₁-C₂ alkenyl; an optionally substituted phenyl, —N(R*)₂, —OR*, —N(R*)C(O)R*, —NH—SO₂—R*, —C(O)N(R*)₂, or

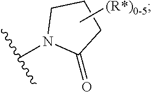

R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S,
or a pharmaceutically acceptable salt thereof,
with a proviso that wherein (i) R₁ and R₂ are both hydrogens, X and Y are both —N═, and R₃ is a 6 membered saturated heterocyclyl having 2 nitrogen atoms, R₄ is not

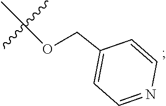 [SMILES: *OCc1ccncc1]

(ii) R₁ and R₂ are both hydrogens, X and Y are both —N═, and R₃ is phenyl substituted with —OCH₃ or —F,
R₄ is not

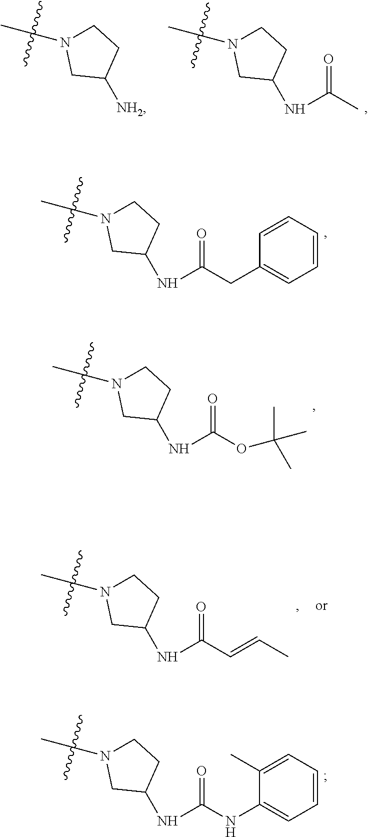

or
(iii) R₁ is a hydrogen, —Cl, —F, or a C₁-C₆ alkyl; R₂ is a hydrogen, —Cl, —F, or a C₁-C₆ alkyl; X is —N═; Y is —C(H)═ or —N═; and R₃ is —CH₃ or a combination of C₁-C₄ alkylene and a fused bicyclic ring system consisting of two fused six-membered aromatic rings containing at least one nitrogen atom, R₄ is not

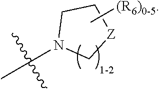

In another aspect, the present application is directed to a compound having the structure according to Formula (II):

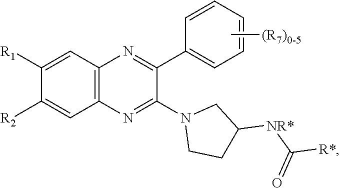

wherein
R₁ is a hydrogen, —Cl, —F, or a C₁-C₆ alkyl;
R₂ is a hydrogen, —Cl, —F, or a C₁-C₆ alkyl;
R₇ is independently at each occurrence —CN, —F, —Cl, —CH₃, —CH₂CH₃, —CF₃, —OH, —OCH₃, —OCH₂CH₃, —OCF₃, —CH₂OCH₃, —O—CH₂CH₂OCH₃, CONH₂, —CO₂CH₃,

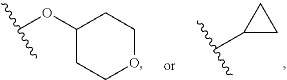

and
R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S,
or a pharmaceutically acceptable salt thereof.

In another aspect, the present application is directed to a compound having the structure according to Formula (III):

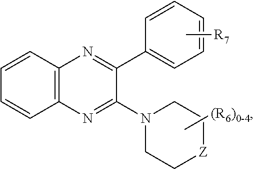

wherein
R₆ is —CH₃, —NH—C(O)—CH₃, —NH—C(O)—CH(CH₃)₂, or —NH—C(O)—C(CH₃)₃;
R₇ is —CN; and
Z is —CH₂— or —O—,
or a pharmaceutically acceptable salt thereof.

In another aspect, the present application is directed to a compound having the structure according to Formula (IV):

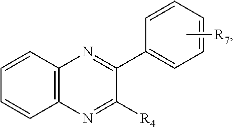

wherein
R₄ is —OR₅, —NHR₅, or —N(R₅)₂;
R₅ is independently at each occurrence —CH₃ or —(CH₂)₂—N(CH₃)—C(O)—CH(CH₃)₂; and
R₇ is —CN;
or a pharmaceutically acceptable salt thereof.

In another aspect, the present application is directed to a compound having the structure according to Formula (V):

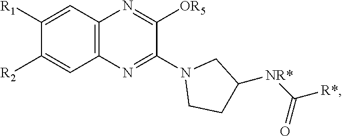

wherein
R₁ is a hydrogen, —Cl, or —F;
R₂ is a hydrogen, —Cl, or —F;
—OR₅ is —O—CH₂CH₂—N(CH₃)—C(O)—CH(CH₃)₂, —O—(CH₂)₃—CH₃, —O—CH₂—CH(CH₃)₂, —O—(CH₂)₂OH, —O—(CH₂)₂NH₂, —O(CH₂)₂OCH₃, —O—(CH₂)₂—N(CH₂CH₃)₂, —O—(CH₂)₂—NH—CH₂—CH₃, or selected from the group consisting of:

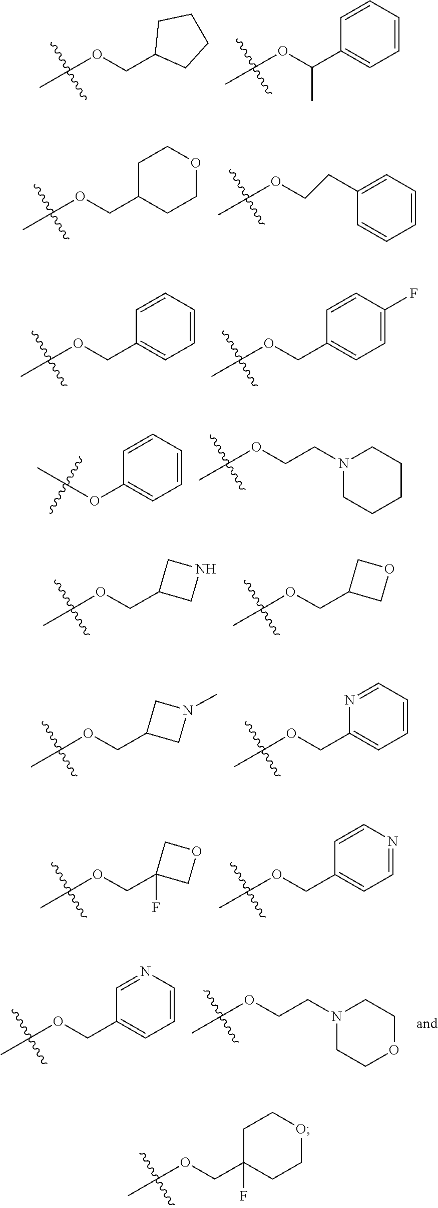

and
R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S;
or a pharmaceutically acceptable salt thereof.

In another aspect, the present application is directed to a compound having the structure according to Formula (VI):

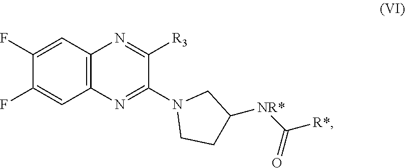

wherein
R₃ is

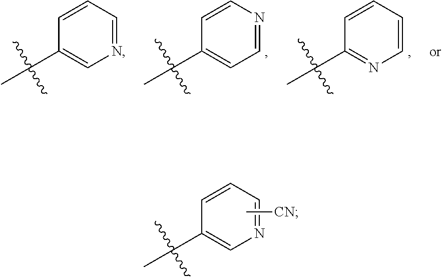

and
   R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S,
or a pharmaceutically acceptable salt thereof.

In another aspect, the present application is directed to a compound having the structure according to Formula (VII):

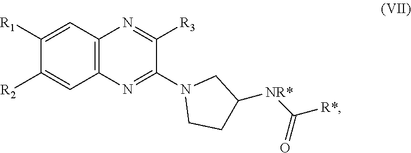

wherein
   R₁ is a hydrogen, —Cl, or —F;
   R₂ is a hydrogen, —Cl, or —F;
R₃ is —NHR₅, or —N(R₅)₂;
R₅ is —CH₃, —CH₂CH₂OCH₃, -phenyl, or —CH₂-phenyl,
   R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S,
or a pharmaceutically acceptable salt thereof.

In another aspect, the present application is directed to a compound having the structure according to Formula (VIII):

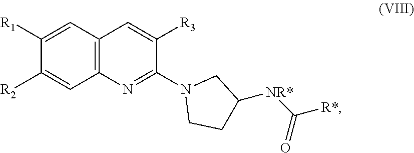

wherein
R₁ is a hydrogen, —Cl, or —F;
R₂ is a hydrogen, —Cl, or —F;

R₃ is

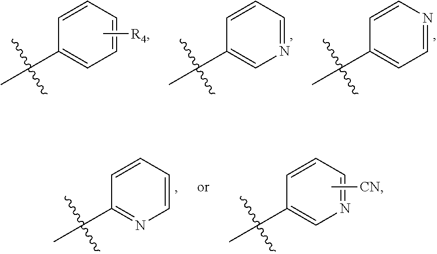

wherein
R₄ is a hydrogen, —C(O)NH₂, or —CN;
R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S, or a pharmaceutically acceptable salt thereof.

In another aspect, the present application is directed to a compound having the structure according to Formula (IX):

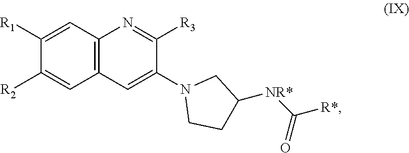

wherein
R₁ is a hydrogen, —Cl, or —F;
R₂ is a hydrogen, —Cl, or —F;
R₃ is

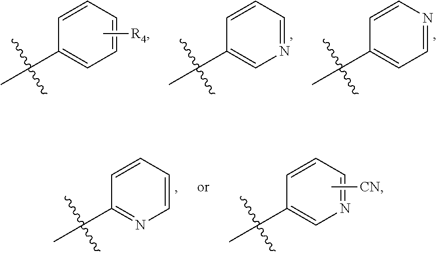

wherein
R₄ is a hydrogen, —C(O)NH₂, or —CN;
R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S, or a pharmaceutically acceptable salt thereof.

In another aspect, the present application is directed to a pharmaceutical composition comprising any of the herein described compounds or a pharmaceutically acceptable salt thereof and a pharmaceutically acceptable carrier.

In another aspect, the present application is directed to a pharmaceutical dosage form comprising any of the herein-described compounds or pharmaceutical compositions or a pharmaceutically acceptable salt thereof and a pharmaceutically acceptable carrier.

In another aspect, the present application is directed to a method of inhibiting Sphingolipid Transporter 2 (SPNS2) in a subject in need thereof comprising administering to the subject a therapeutically effective amount of any of the herein-described compounds or pharmaceutical compositions or a pharmaceutically acceptable salt thereof and a pharmaceutically acceptable carrier.

In another aspect, the present application is directed to a method of treating a disease or condition in a subject in need thereof comprising administering to the subject a therapeutically effective amount of any of the herein-described compounds or pharmaceutical compositions or a pharmaceutically acceptable salt thereof and a pharmaceutically acceptable carrier.

These and other aspects of the present invention will become apparent to those skilled in the art after a reading of the following detailed description of the invention, including the appended claims.

### BRIEF DESCRIPTION OF THE DRAWINGS

FIG. 1A is a plot of lymphocytes per microliter of blood for mice treated with a control and with compound 510 according to the disclosure. FIG. 1B is a plot of red blood cells per microliter of blood for mice treated with a control and with compound 510 according to the disclosure.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

Unless defined otherwise, all technical and scientific terms used herein have the same meaning as commonly understood by one of ordinary skill in the art to which this invention belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof, or (3) relieving the disease, i.e., causing regression of the state, disorder or condition or at least one of its clinical or sub-clinical symptoms. The benefit to a subject to be treated is either statistically significant or at least perceptible to the patient or to the physician.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon, bicyclic hydrocarbon, or tricyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-30 aliphatic carbon atoms. In some embodiments, aliphatic groups contain 1-20 aliphatic carbon atoms. In other embodiments, aliphatic groups contain 1-10 aliphatic carbon atoms. In still other embodiments, aliphatic groups contain 1-6 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “cycloaliphatic,” as used herein, refers to saturated or partially unsaturated cyclic aliphatic monocyclic, bicyclic, or polycyclic ring systems, as described herein, having from 3 to 14 members, wherein the aliphatic ring system is optionally substituted as defined above and described herein. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, the cycloalkyl has 3-6 carbons. The terms “cycloaliphatic,” may also include aliphatic rings that are fused to one or more aromatic or nonaromatic rings, such as decahydronaphthyl or tetrahydronaphthyl, where the radical or point of attachment is on the aliphatic ring. In some embodiments, a carbocyclic group is bicyclic. In some embodiments, a ‘carbocyclic group is tricyclic. In some embodiments, a carbocyclic group is polycyclic. In some embodiments, “cycloaliphatic” (or “carbocycle” or “cycloalkyl”) refers to a monocyclic C₃-C₆ hydrocarbon, or a C₈-C₁₀ bicyclic hydrocarbon that is completely saturated or that contains one or more units of unsaturation, but which is not aromatic, that has a single point of attachment to the rest of the molecule, or a C₉-C₁₆ tricyclic hydrocarbon that is completely saturated or that contains one or more units of unsaturation, but which is not aromatic, that has a single point of attachment to the rest of the molecule.

As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 1-20 carbon atoms in its backbone (e.g., C₁-C₂₀ for straight chain, C₂-C₂₀ for branched chain), and alternatively, about 1-10 carbon atoms, or about 1 to 6 carbon atoms. In some embodiments, a cycloalkyl ring has from about 3-10 carbon atoms in their ring structure where such rings are monocyclic or bicyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

As used herein, the term “alkenyl” refers to an alkyl group, as defined herein, having one or more double bonds.

As used herein, the term “alkynyl” refers to an alkyl group, as defined herein, having one or more triple bonds.

The term “heteroalkyl” is given its ordinary meaning in the art and refers to alkyl groups as described herein in which one or more carbon atoms is replaced with a heteroatom (e.g., oxygen, nitrogen, sulfur, and the like). Examples of heteroalkyl groups include, but are not limited to, alkoxy, poly(ethylene glycol)-, alkyl-substituted amino, tetrahydrofuranyl, piperidinyl, morpholinyl, etc.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, binaphthyl, anthracyi and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone of as part of a larger moiety, e.g., “heteroaralkyl,” or “heteroaralkoxy,” refer to groups having 5 to 10 ring atoms (i.e., monocyclic or bicyclic), in some embodiments 5, 6, 9, or 10 ring atoms. In some embodiments, such rings have 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic, tricyclic, tetracyclic, and/or otherwise polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic, tricyclic, tetracyclic, and/or otherwise polycyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring.

The term “unsaturated,” as used herein, means that a moiety has one or more units of unsaturation.

The term “halogen” means F, Cl, Br, or I; the term “halide” refers to a halogen radical or substituent, namely —F, —Cl, —Br, or —I.

As described herein, compounds of the invention may contain “optionally substituted” moieties. In general, the term “substituted,” whether preceded by the term “optionally” or not, means that one or more hydrogens of the designated moiety are replaced with a suitable substituent.

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C- or ¹³C- or ¹⁴C-enriched carbon are within the scope of this invention.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Unless otherwise stated, all crystalline forms of the compounds of the invention and salts thereof are also within the scope of the invention. The compounds of the invention may be isolated in various amorphous and crystalline forms, including without limitation forms which are anhydrous, hydrated, non-solvated, or solvated. Example hydrates include hemihydrates, monohydrates, dihydrates, and the like. In some embodiments, the compounds of the invention are anhydrous and non-solvated. By “anhydrous” is meant that the crystalline form of the compound contains essentially no bound water in the crystal lattice structure, i.e., the compound does not form a crystalline hydrate.

As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. The different crystalline lattices can be identified by solid state characterization methods such as by X-ray powder diffraction (PXRD). Other characterization methods such as differential scanning calorimetry (DSC), thermogravimetric analysis (TGA), dynamic vapor sorption (DVS), solid state NMR, and the like further help identify the crystalline form as well as help determine stability and solvent/water content.

Crystalline forms of a substance include both solvated (e.g., hydrated) and non-solvated (e.g., anhydrous) forms. A hydrated form is a crystalline form that includes water in the crystalline lattice. Hydrated forms can be stoichiometric hydrates, where the water is present in the lattice in a certain water/molecule ratio such as for hemihydrates, monohydrates, dihydrates, etc. Hydrated forms can also be non-stoichiometric, where the water content is variable and dependent on external conditions such as humidity.

In some embodiments, the compounds of the invention are substantially isolated. By “substantially isolated” is meant that a particular compound is at least partially isolated from impurities. For example, in some embodiments a compound of the invention comprises less than about 50%, less than about 40%, less than about 30%, less than about 20%, less than about 15%, less than about 10%, less than about 5%, less than about 2.5%, less than about 1%, or less than about 0.5% of impurities. Impurities generally include anything that is not the substantially isolated compound including, for example, other crystalline forms and other substances.

The term “polyfluoroalkyl” refers to alkyl groups in which multiple carbon-bonded hydrogen atoms have been replaced by fluorine.

### The Compounds

In one aspect, the present invention is directed to a compound according to Formula (I):

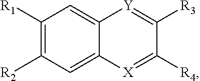

wherein:
X is —C(H)═ or —N═;
Y is —C(H)═ or —N═;
R₁ is a hydrogen, —Cl, —F, or a C₁-C₆ alkyl;
R₂ is a hydrogen, —Cl, —F, or a C₁-C₆ alkyl;
R₃ is —Cl, R₅, —OR₅, —NHR₅, or —N(R₅)₂;
R₄ is —OR₅, —NHR₅, —N(R₅)₂,

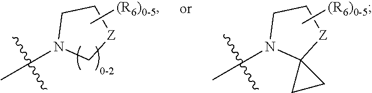

R₅ is independently at each occurrence an optionally substituted C₁-C₁₂ alkyl, an optionally substituted C₁-C₁₂ alkenyl, an optionally substituted C₁-C₁₂ alkoxy, an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 5-6 membered heteroaryl having 1-4 heteroatoms independently selected from N, O, and S, a fused bicyclic ring system having 1-4 heteroatoms independently selected from N, O, and S, or an optionally substituted combination of any two of a C₁-C₁₂ alkyl, a C₁-C₁₂ alkenyl and a ring;
Z is —C(H)—, —CH₂—, or —O—;
R₆ is independently at each occurrence an optionally substituted C₁-C₂ alkyl; an optionally substituted C₁-C₂ alkenyl; an optionally substituted phenyl, —N(R*)₂, —OR*, —N(R*)C(O)R*, —NH—SO₂—R*, —C(O)N(R*)₂, or

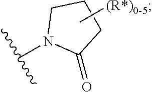

R* is independently at each occurrence hydrogen, an optionally substituted C₁-C₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S,
or a pharmaceutically acceptable salt thereof,
with a proviso that wherein (i) R₁ and R₂ are both hydrogens, X and Y are both —N═, and R₃ is a 6 membered saturated heterocyclyl having 2 nitrogen atoms, R₄ is not

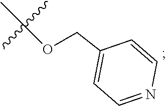 [SMILES: *OCc1ccncc1]

(ii) R₁ and R₂ are both hydrogens, X and Y are both —N═, and R₃ is phenyl substituted with —OCH₃ or —F,
R₄ is not

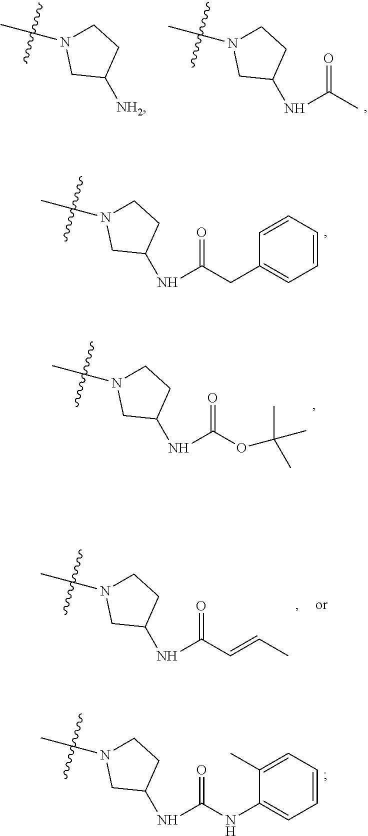

or
(iii) R₁ is a hydrogen, —Cl, —F, or a C₁-C₆ alkyl; R₂ is a hydrogen, —Cl, —F, or a C₁-C₆ alkyl; X is —N═; Y is —C(H)═ or —N═; and R₃ is —CH₃ or a combination of C₁-C₄ alkylene and a fused bicyclic ring system consisting of two fused six-membered aromatic rings containing at least one nitrogen atom, R₄ is not

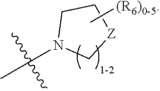

In one embodiment of the compound of Formula (I), Y is —N═. In another embodiment, X is —N═. In yet another embodiment, Y is —N═ and X is —N═.

In one embodiment of the compound of Formula (I), R₁ is a hydrogen. In another embodiment, R₂ is a hydrogen. In yet another embodiment, R₁ and R₂ are hydrogens.

In one embodiment of the compound of Formula (I), R₁ is —Cl. In another embodiment, R₂ is —Cl. In yet another embodiment, R₁ and R₂ are —Cl.

In one embodiment of the compound of Formula (I), R₁ is —F. In another embodiment, R₂ is —F. In yet another embodiment, R₁ and R₂ are —F.

In one embodiment of the compound of Formula (I), R₁ is a C₁-C₆ alkyl. In another embodiment, R₂ is a C₁-C₆ alkyl. In yet another embodiment, R₁ and R₂ are C₁-C₆ alkyl.

In one embodiment of the compound of Formula (I), R₁ is methyl. In another embodiment, R₂ is methyl. In another yet embodiment, R₁ and R₂ are methyl.

In one embodiment of the compound of Formula (I), R₃ is —Cl.

In one embodiment of the compound of Formula (I), R₃ is R₅. In another embodiment, R₅ is a phenyl. In yet another embodiment, R₅ is a phenyl substituted with one or more of —CN, —F, —Cl, —CH₃, —CH₂CH₃, —CF₃, —OH, —OCH₃, —OCH₂CH₃, —OCF₃, —CH₂OCH₃, —O—CH₂CH₂OCH₃, CONH₂, —CO₂CH₃,

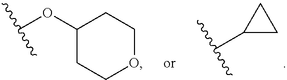

In one embodiment of the compound of Formula (I), R₃ is —OR₅. In another embodiment, OR₅ is —O—(CH₂)₂OH, —O—(CH₂)₃CH₃, —O—CH₂—CH(CH₃)₂, —O—(CH₂)₂NH₂, —O(CH₂)₂OCH₃, —O—(CH₂)₂—N(CH₂CH₃)₂, —O—(CH₂)₂—NH—CH₂—CH₃, —O—CH₂CH₂—N(CH₃)—C(O)—CH(CH₃)₂, or selected from the group consisting of:

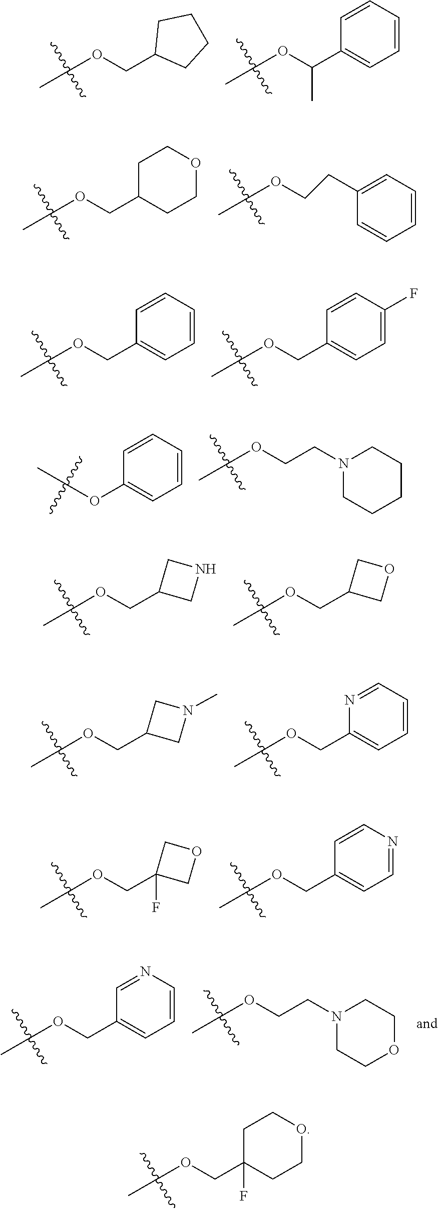

In one embodiment of the compound of Formula (I), R₃ is —NHR₅. In another embodiment, —NHR₅ is

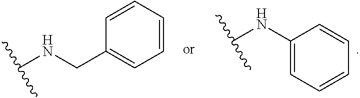 [SMILES: *NCc1ccccc1 ; *Nc1ccccc1]

In one embodiment of the compound of Formula (I), R₃ is —N(R₅)₂. In another embodiment, —N(R₅)₂ is

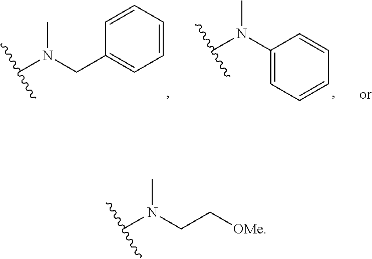

In one embodiment of the compound of Formula (I), the compound has the structure of Formula (II):

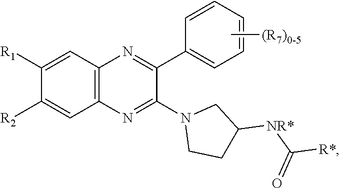

wherein
R₁ is a hydrogen, —Cl, —F, or a C₁-C₆ alkyl;
R₂ is a hydrogen, —Cl, —F, or a C₁-C₆ alkyl;
R₇ is independently at each occurrence —CN, —F, —Cl, —CH₃, —CH₂CH₃, —CF₃, —OH, —OCH₃, —OCH₂CH₃, —OCF₃, —CH₂OCH₃, —O—CH₂CH₂OCH₃, CONH₂, —CO₂CH₃,

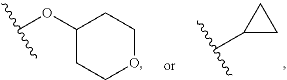

and
R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S,
or a pharmaceutically acceptable salt thereof.

In one embodiment of the compound of Formula (II), the compound has the structure according to Formula (II-1):

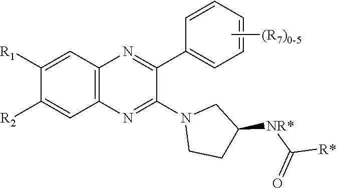

In one embodiment of the compound of Formula (I), the compound has the structure of Formula (III):

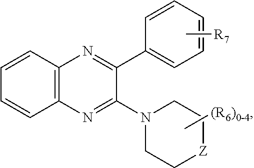

wherein
R₆ is —CH₃, —NH—C(O)—CH₃, —NH—C(O)—CH(CH₃)₂, or —NH—C(O)—C(CH₃)₃;
R₇ is —CN; and
Z is —CH₂— or —O—,
or a pharmaceutically acceptable salt thereof.

In one embodiment of the compound of Formula (I), the compound has the structure of Formula (IV):

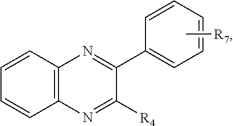

wherein
R₄ is —OR₅, —NHR₅, or —N(R₅)₂;
R₅ is independently at each occurrence —CH₃ or —(CH₂)₂—N(CH₃)—C(O)—CH(CH₃)₂; and
R₇ is —CN;
or a pharmaceutically acceptable salt thereof.

In one embodiment of the compound of Formula (I), the compound has the structure of Formula (V):

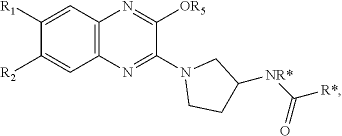

wherein
R₁ is a hydrogen, —Cl, or —F;
R₂ is a hydrogen, —Cl, or —F;
—OR₅ is —O—CH₂CH₂—N(CH₃)—C(O)—CH(CH₃)₂, —O—(CH₂)₃—CH₃, —O—CH₂—CH(CH₃)₂, —O—(CH₂)₂OH, —O—(CH₂)₂NH₂, —O(CH₂)₂OCH₃, —O—(CH₂)₂—N(CH₂CH₃)₂, —O—(CH₂)₂—NH—CH₂—CH₃, or selected from the group consisting of:

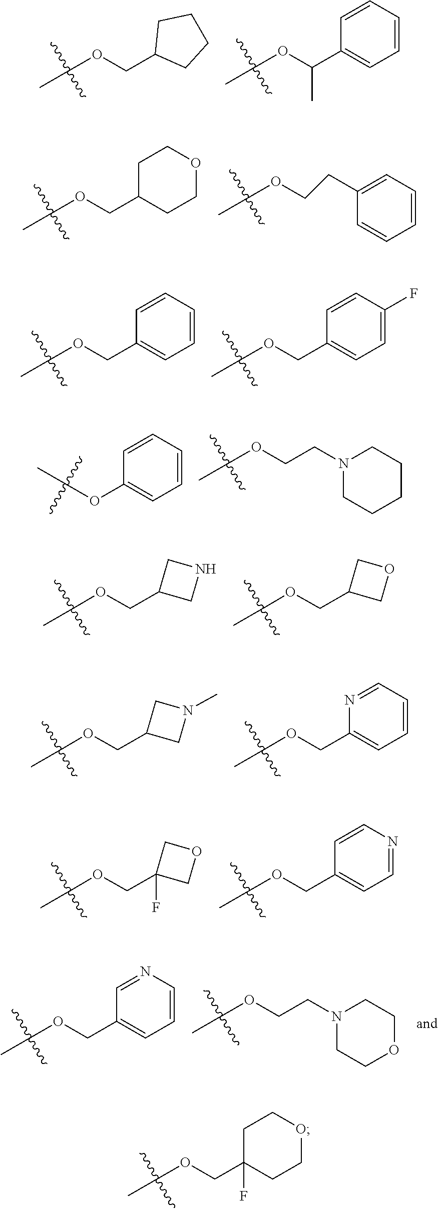

and
R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S;
or a pharmaceutically acceptable salt thereof.

In one embodiment of the compound of Formula (V), R* is independently at each occurrence is —CH₂CH₃ or —CH(CH₃)₂.

In one embodiment of the compound of Formula (V), the compound has the structure of Formula (V-1):

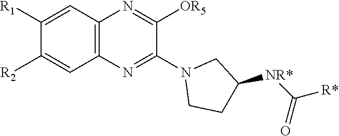

In one embodiment of the compound of Formula (I), the compound has the structure of Formula (VI):

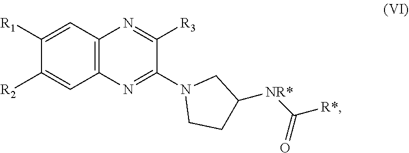

wherein
R₃ is

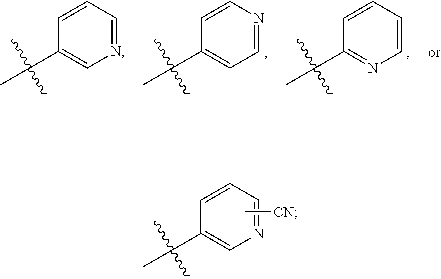

and
R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S,
or a pharmaceutically acceptable salt thereof.

In one embodiment of the compound of Formula (VI), R* is independently at each occurrence —CH₂CH₃, —CH₂CF₃, or —CH(CH₃)₂.

In one embodiment of the compound of Formula (I), the compound has the structure of Formula (VII):

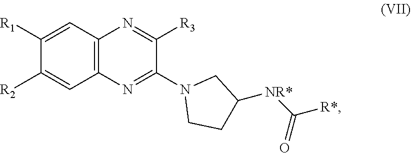

wherein
   R₁ is a hydrogen, —Cl, or —F;
   R₂ is a hydrogen, —Cl, or —F;
R₃ is —NHR₅, or —N(R₅)₂;
R₅ is —CH₃, —CH₂CH₂OCH₃, -phenyl, or —CH₂-phenyl,
R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S,
or a pharmaceutically acceptable salt thereof.

In one embodiment of the compound of Formula (VII), R* is independently at each occurrence —CH₂CH₃ or —CH(CH₃)₂.

In one embodiment of the compound of Formula (I), the compound has the structure of Formula (VIII):

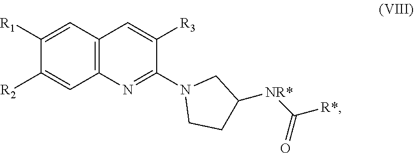

wherein
R₁ is a hydrogen, —Cl, or —F;
R₂ is a hydrogen, —Cl, or —F;
R₃ is

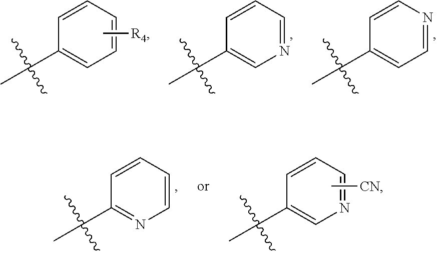

wherein
R₄ is a hydrogen, —C(O)NH₂, or —CN;
R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S, or a pharmaceutically acceptable salt thereof.

In one embodiment of the compound of Formula (VIII), R* is independently at each occurrence —H, —CH₃, —CH₂CH₃, —CH₂CF₃, —CH₂CH₂OH, —CH(CH₃)₂, or —C(CH₃)₃.

In one embodiment of the compound of Formula (I), the compound has the structure of Formula (IX):

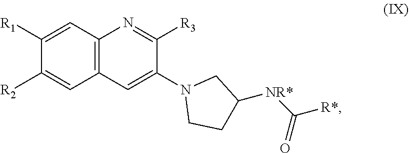

wherein
R₁ is a hydrogen, —Cl, or —F;
R₂ is a hydrogen, —Cl, or —F;
R₃ is

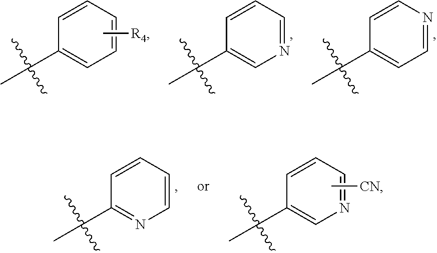

wherein
R₄ is a hydrogen, —C(O)NH₂, or —CN;
R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S, or a pharmaceutically acceptable salt thereof.

In one embodiment of the compound of Formula (IX), R* is independently at each occurrence —H, —CH₃, —CH₂CH₃, —CH₂CF₃, —CH₂CH₂OH, —CH(CH₃)₂, or —C(CH₃)₃.

In one embodiment, the compound has a structure selected from the group consisting of:

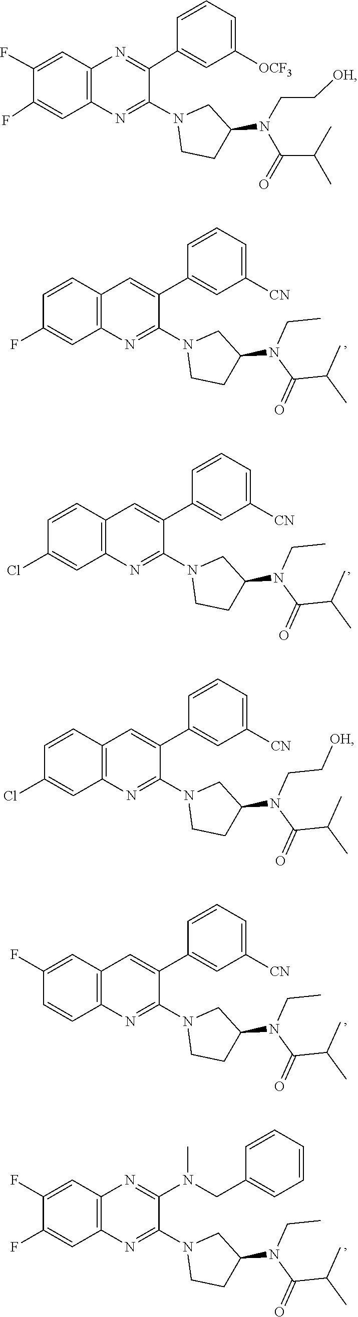

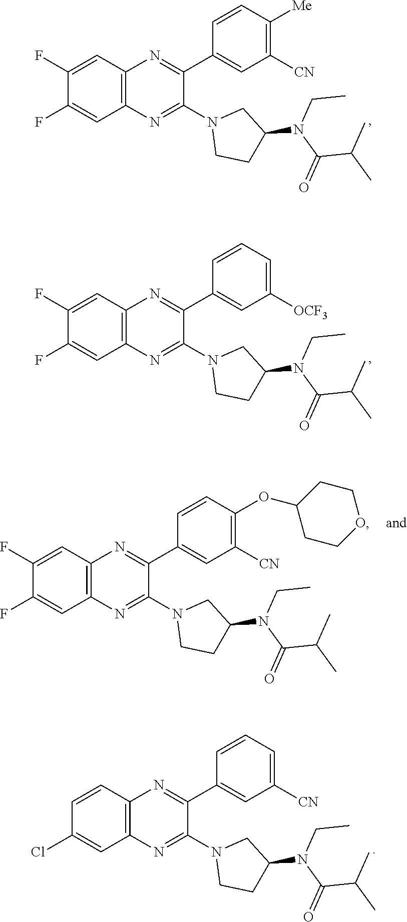 [SMILES: CCN(C(=O)C(C)C)[C@H]1CCN(c2nc3cc(F)c(F)cc3nc2-c2ccc(C)c(C#N)c2)C1 ; CCN(C(=O)C(C)C)[C@H]1CCN(c2nc3cc(F)c(F)cc3nc2-c2cccc(OC(F)(F)F)c2)C1 ; CCN(C(=O)C(C)C)[C@H]1CCN(c2nc3cc(F)c(F)cc3nc2-c2ccc(OC3CCOCC3)c(C#N)c2)C1 ; CCN(C(=O)C(C)C)[C@H]1CCN(c2nc3cc(Cl)ccc3nc2-c2cccc(C#N)c2)C1]

The present invention also includes salts of the compounds described herein. As used herein, “salts” refers to derivatives of the disclosed compounds wherein the parent compound is modified by converting an existing acid or base moiety to its salt form. Examples of salts include, but are not limited to, mineral acid (such as HCl, HBr, H₂SO₄) or organic acid (such as acetic acid, benzoic acid, trifluoroacetic acid salts of basic residues such as amines; alkali (such as Li, Na, K, Mg, Ca) or organic (such as trialkylammonium) salts of acidic residues such as carboxylic acids; and the like. The salts of the present application can be synthesized from the parent compound which contains a basic or acidic moiety conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile (ACN) are preferred.

The present application also includes pharmaceutically acceptable salts of the compounds described herein. The “pharmaceutically acceptable salts” include a subset of the “salts” described above which are conventional non-toxic salts of the parent compound formed, for example, from non-toxic inorganic or organic acids. Lists of suitable salts are found in Remington's Pharmaceutical Sciences, 17th ed., Mack Publishing Company, Easton, Pa., 1985, p. 1418 and Journal of Pharmaceutical Science, 66, 2 (1977). The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Preparation of compounds can involve the protection and deprotection of various chemical groups. The need for protection and deprotection, and the selection of appropriate protecting groups can be readily determined by one skilled in the art. The chemistry of protecting groups can be found, for example, in Wuts and Greene, Greene Protective Groups in Organic Synthesis, 4th Ed., John Wiley & Sons: New York, 2006.

### Pharmaceutical Compositions

When employed as pharmaceuticals, the compounds of this invention are typically administered in the form of a pharmaceutical composition. Such compositions can be prepared in a manner well known in the pharmaceutical art and comprise at least one active compound.

Generally, the compounds of this invention are administered in a therapeutically effective amount. The amount of the compound actually administered will typically be determined by a physician, in the light of the relevant circumstances, including the condition to be treated, the chosen route of administration, the actual compound-administered, the age, weight, and response of the individual patient, the severity of the patient's symptoms, and the like.

The pharmaceutical compositions of this invention can be administered by a variety of routes including oral, rectal, intraocular, transdermal, subcutaneous, intravenous, intramuscular, intraperitoneal, intradermal, directly into cerebrospinal fluid, intratracheal, and intranasal.

Depending on the intended route of delivery, the compounds of this invention are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the active compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10%) by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10%) by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

The compounds of this invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of this invention can also be administered in sustained release forms or from sustained release drug delivery systems. A description of representative sustained release materials can be found in Remington's Pharmaceutical Sciences.

Pharmaceutical compositions containing the compounds of the invention can be prepared in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In some embodiments, the pharmaceutical composition of the invention is in liquid form. Liquid forms include, by way of non-limiting example, emulsions, solutions, suspensions, syrups, slurries, dispersions, colloids and the like. In some embodiments, a pharmaceutical composition described herein is in liquid, semi-solid or solid (e.g., powder) form. In specific embodiments, a pharmaceutical composition described herein is in semi-solid form, e.g., a gel, a gel matrix, a cream, a paste, or the like. In some embodiments, semi-solid forms comprise a liquid vehicle. In some embodiments, the pharmaceutical composition of the invention is a solid dosage form, such a tablet, a granule, a sachet, or a powder. Also provided are pharmaceutical compositions comprising a compound of the invention or a pharmaceutically acceptable salt thereof in the form of a dissolving tablet, a dissolving wafer, a capsule, or a gel capsule. In certain embodiments, solid dosage forms described herein comprise a solid vehicle (e.g., as used in a tablet), and/or a gaseous vehicle (e.g., as used in DPI).

In some embodiments, a composition is in a unit dose formulation for oral, intranasal, or other administration to a patient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

In some embodiments, the compounds or compositions described herein are administered intranasally. As used herein, “nasal delivery-enhancing agents” include agents which enhance the release or solubility (e.g., from a formulation delivery vehicle), diffusion rate, penetration capacity and timing, uptake, residence time, stability, effective half-life, peak or sustained concentration levels, clearance and other desired nasal delivery characteristics (e.g., as measured at the site of delivery, or at a selected target site of activity such as the brain) of the compounds or compositions of the invention. Enhancement of mucosal delivery can thus occur by any of a variety of mechanisms, for example by increasing the diffusion, transport, persistence or stability of the compounds or compositions of the invention, enzyme inhibition, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, increasing nasal blood flow and other mechanisms. Suitable mucosal delivery enhancing agents will be clear to a person skilled in the art of pharmacology and are further described hereafter.

Compositions of the invention can be simple aqueous (e.g., saline) solutions. Alternatively, they can contain various additional ingredients which enhance stability and/or nasal delivery of the compounds of the invention. Such additional ingredients are well known in the art. Non-limiting examples of useful additional ingredients for enhancing nasal delivery include, e.g., (a) aggregation inhibitory agents (e.g., polyethylene glycol, dextran, diethylaminoethyl dextran, and carboxymethyl cellulose), (b) charge modifying agents, (c) pH control agents, (d) degradative enzyme inhibitors (e.g., amastatin and bestatin [see, e.g., O'Hagan et al., Pharm. Res. 1990, 7: 772-776 and WO 05/120551]; (e) mucolytic or mucus clearing agents (e.g., n-acetyl-cysteine, propyl gallate and cysteine methionine dimers, chaotropes [see, e.g., WO 04/093917]), (f) ciliostatic agents; (g) membrane penetration enhancing agents, (h) modulatory agents of epithelial junction physiology, such as nitric oxide (NO) stimulators, chitosan, and chitosan derivatives; (i) vasodilator agents, (j) selective transport-enhancing agents, and (k) stabilizing delivery vehicles, carriers, supports or complex-forming agents. See, e.g., EP 037943, EP 094157, EP 173990, EP 214898, EP 215697, EP 327756, EP 490806, U.S. Pat. Nos. 4,476,116, 5,759,565, WO 04/093917 and WO 05/120551.

Non-limiting examples of membrane penetration-enhancing agents useful in the compositions of the invention include, e.g., (i) a surfactant (e.g., Tween 80, Poloxamer 188, polysorbates; see also EP 490806, U.S. Pat. No. 5,759,565, and WO04/093917), (ii) a bile salt or bile salt derivative (e.g., unsaturated cyclic ureas and Transcutol), (iii) a phospholipid or fatty acid additive, mixed micelle, liposome, or carrier, (iv) an alcohol, (v) an enamine, (vi) a nitric oxide donor compound (e.g., S-nitroso-N-acetyl-DL-penicillamine, NOR1, NOR4, which are preferably co-administered with an NO scavenger such as carboxy-PITO or doclofenac sodium), (vii) a long-chain amphipathic molecule (e.g., deacylmethyl sulfoxide, azone, sodium lauryl sulfate, oleic acid) (viii) a small hydrophobic penetration enhancer, (ix) sodium salicylate or a salicylic acid derivative (e.g., acetyl salicylate, choline salicylate, salicylamide, etc.), (x) a glycerol ester of acetoacetic acid, (xi) a cyclodextrin or betacyclodextrin derivative, (xii) a medium-chain fatty acid including mono- and diglycerides (e.g., sodium caprate—extracts of coconut oil, Capmul), (xiii) a chelating agent (e.g., citric acid, salicylates), (xiv) an amino acid or salt thereof (e.g. monoaminocarboxlic acids such as glycine, alanine, phenylalanine, proline, hydroxyproline, etc.; hydroxyamino acids such as serine; acidic amino acids such as aspartic acid, glutamic acid, etc; and basic amino acids such as lysine etc., inclusive of their alkali metal or alkaline earth metal salts), (xv) an N-acetylamino acid or salt thereof, (xvi) an enzyme degradative to a selected membrane component, (xvii) an inhibitor of fatty acid synthesis, (xviii) an inhibitor of cholesterol synthesis, (xix) cationic polymers, or any combination thereof. The membrane penetration-enhancing agent can be also selected from small hydrophilic molecules, including but not limited to, dimethyl sulfoxide (DMSO), dimethylformamide, ethanol, propylene glycol, and the 2-pyrrolidones. Additional membrane penetration enhancers include emulsifiers (e.g. sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like; mixed micelles; glycerol esters of acetoacetic acid (e.g., glyceryl-1,3-diacetoacetate or 1,2-isopropylideneglycerine-3-acetoacetate), and triglycerides (e.g., amylodextrin, Estaram 299, Miglyol 810); cyclodextrins and β-cyclodextrin derivatives (e.g., 2-hydroxypropyl-p-cyclodextrin and heptakis (2,6-di-O-methyl-β-cyclodextrin) which can be optionally conjugated with Peptide and further optionally formulated in an oleaginous base; and N-acetylamino acids (N-acetylalanine, N-acetylphenylalanine, Nacetylserine, N-acetylglycine, N-acetyllysine, N-acetylglutamic acid, N-acetylproline, Nacetylhydroxyproline, etc.) and their salts (alkali metal salts and alkaline earth metal salts), as well as other penetration-promoting agents that are physiologically compatible for intranasal delivery. See, e.g., WO 04/093917, WO 05/120551 and Davis and Ilium (Clin. Pharmacokinet 2003, 42: 1107-1128).

Non-limiting examples of useful absorption enhancers include, e.g., surfactants, glycosides, cyclodextrin and glycols. Non-limiting examples of useful bioadhesive agents include, e.g., carbopol, cellulose agents, starch, dextran, and chitosan.

In various embodiments of the invention, a compound of the invention is combined with one or more of the nasal delivery-enhancing agents recited above. These nasal delivery-enhancing agents may be admixed, alone or together, with the nasal carrier and with the compound of the invention, or otherwise combined therewith in a pharmaceutically acceptable formulation or delivery vehicle. For nasal delivery-enhancing agents to be of value within the invention, it is generally desired that any significant changes in permeability of the mucosa be reversible within a time frame appropriate to the desired duration of drug delivery.

Furthermore, there should be no substantial, cumulative toxicity, nor any permanent deleterious changes induced in the barrier properties of the nasal mucosa with long term use.

In addition to the compound of the invention, the nasal carrier and, optionally, one or more further additives and/or agents, the composition of the invention may further comprise one or more additional therapeutic ingredients (or active substances). These therapeutic ingredients can be any compound that elicits a desired activity or therapeutic or biological response in the subject.

The proportion of each further component in the nasal composition of the invention may vary depending on the components used. For example, but without being limiting, the amount of nasal carrier may be in the range of from 0.1 to 99.9% by weight of the total weight or volume of the composition. When present, the amount surfactant may be in the range from about 0.01 to about 10% or higher and preferably about 0.05 to about 1.0% by weight of the total volume or weight of the composition, the amount depending on the specific surfactant used. The amount is generally kept as low as possible since above a certain level no further enhancement of absorption can be achieved and also too high of a surfactant level may cause irritation of the nasal mucosa. The amount of delivery enhancing agents may be at least 0.1%, suitably in the range from about 0.5 to 10% of the total weight of the composition. Where the composition is liquid, the enhancing agent may suitably be present in an amount of from 0.1 to 5% w/v of the total composition. Preserving agents may be present in an amount of from about 0.002 to 0.02% by weight of the total weight or volume of the composition.

The useful delivery volume of the pharmaceutical compositions of the invention is limited by the size of the nasal cavity. Suitable delivery volumes will be clear to a person skilled in the art of pharmacology. Preferably, the total composition quantity administered at each nasal application comprises from about 0.02 to 0.5 ml, preferably about 0.07 to 0.3 ml, typically about 0.09-0.1 ml.

The liquid compositions of the invention may be prepared by bringing into intimate admixture a compound the invention in the liquid carrier optionally together with the further ingredients, additives and/or agents. The solid nasal composition of the invention may be prepared in conventional manner. A compound of the invention may be admixed with the carrier particles, e.g. a polymer base or cellulose product in conventional manner, optionally with further ingredients, additives and/or agents as indicated above e.g. a mucosal delivery enhancing agent or surfactant such as disclosed. A compound of the invention may be in solution e.g. an aqueous or alcoholic solution when being mixed with the carrier particles and the solvent evaporated, e.g. under freeze-drying or spray drying. Such drying may be effected under the conventional conditions. Alternatively, the mixture may be compacted or granulated and then be pulverized and/or sieved. If desired the particles may be coated. In one embodiment of the invention, the nasal composition is prepared by lyophilisation. A homogeneous solution, preferably aqueous, containing a compound of the invention and optionally containing further ingredients, additives and/or agents as discussed above, is prepared and then submitted to lyophilisation in analogy with known lyophilisation procedures, and to subsequent drying. The resulting powder may then be dissolved in a liquid excipient or nasal carrier before administration, e.g. to reconstitute nasal drops, gel or spray. Alternatively, it may be administered as such in the form of lyophilized powder or it may be mixed with further ingredients, additives and/or agents as discussed above. For example, a lyophilized powder comprising a compound of the invention but free of any nasal carrier may be prepared and then admixed with the desired nasal carrier or mixture of nasal carriers.

The present invention encompasses any delivery device that is suitable for nasal administration of the compositions of the invention. Preferably, such means administers a metered dosage of the composition. The composition of the present invention may be packed in any appropriate form or container as long as a means is provided to deliver the composition to the nasal mucosa. Non-limiting examples of useful intranasal delivery devices include, e.g., instillation catheters, droppers, unit-dose containers, squeeze bottles pump sprays, airless and preservative-fee sprays, compressed air nebulizers, metered-dose inhalers, insufflators and pressurized metered dose inhalers.

For administration of a liquid in drop form, compositions of the invention can be placed in a container provided with a conventional dropper/closure device, e.g. comprising a pipette or the like, preferably delivering a substantially fixed volume of composition/drop.

For administration of an aqueous solution as a nasal spray, the aqueous solution may be dispensed in spray form by a variety of methods known to those skilled in the art. For example, such compositions will be put up in an appropriate atomising device, e.g. in a pump-atomiser, or the like. The atomising device will be provided with appropriate means, such as a spray adaptor for delivery of the aqueous spray to the naris. Preferably it will be provided with means ensuring delivery of a substantially fixed volume of composition/actuation (i.e. per spray-unit). Examples of nasal sprays include nasal actuators produced by Ing. Erich Pfeiffer GmbH, Radolfzell, Germany (see U.S. Pat. Nos. 4,511,069, 4,778,810, 5,203,840, 5,860,567, 5,893,484, 6,227,415, and 6,364,166. Additional aerosol delivery forms may include, e.g., compressed air-, jet-, ultrasonic-, and piezoelectric nebulizers.

Alternatively, the spray may be bottled under pressure in an aerosol device. The propellant may be a gas or a liquid (e.g. a fluorinated and/or chlorinated hydrocarbon). The spray composition may be suspended or dissolved in a liquid propellant. Stabilizing and/or suspending agents and/or co-solvents may be present.

A dry powder may be readily dispersed in an inhalation device as described in U.S. Pat. No. 6,514,496 and Garcia-Arieta et al., Biol. Pharm. Bull. 2001; 24: 1411-1416.

If desired a powder or liquid may be filled into a soft or hard capsule or in a single dose device adapted for nasal administration. The powder may be sieved before filled into the capsules such as gelatine capsules. The delivery device may have means to break open the capsule. The powdery nasal composition can be directly used as a powder for a unit dosage form. The contents of the capsule or single dose device may be administered using e.g. an insufflator. Preferably it will be provided with means ensuring dosing of a substantially fixed amount of composition.

In another embodiment, the composition of the invention can be provided as a nasal insert having the compound of the invention dispersed therein. The insert may be retained in the naris, but flushed by the nasal mucus, and may be designed to release the compound of the invention at the same place in the naris. Suitable nasal insert types include nasal plugs, tampons and the like. Further examples of nasal inserts, their characteristics and preparation are described in EP 490806.

In one aspect, a composition or unit dosage form according to the invention is formulated for sublingual administration, wherein the unit dosage form is a film including one or more disintegrants (e.g., materials that favor disintegration or fast dissolution by virtue of their solubility in water, such as hydrolyzed starches, sugars, and glycerin, which may play a dual role as a plasticizer and disintegrant) and a plasticizing agent, the film having a first portion including apomorphine hydrochloride, and a second portion including pH neutralizing agent, wherein the unit dosage form includes from 0.5 to 5 mg, from 4 to 10 mg, or from 8 to 20 mg of apomorphine hydrochloride and the pH neutralizing agent is present in an amount sufficient to produce a solution having a pH of between 3.0 and 6.0, preferably between 4.5 and 6.5, (e.g., a pH of between 2.5 and 4.5, 3.0 and 6.0, 3.5 and 6.5, 4.5 and 6.5, or 5.0 and 6.0) when the unit dosage form is placed in unbuffered water at pH 7 (e.g., the pH observed within 5 minutes of placing the unit dosage form in 1, 5, or 10 mL of unbuffered water). The film can include from 1 to 50% (w/w) (e.g., 1±0.75%, 2±1.5%, 3±0.5%, 5±2%, 7±2.5%, 10±2%, 14±3%, 18±4%, 22±5%, 25±5%, 30±5%, 35±5%, 40±5%, 45±5%, or 50±5% (w/w)) of the one or more disintegrants. In certain embodiments, the unit dosage form further includes a high molecular weight polymer having a weight average molecular weight of greater than 60 KDa selected from hydroxypropyl cellulose, hydroxypropyl methyl cellulose, hydroxyethyl cellulose, carboxymethyl cellulose, and methyl cellulose. In other embodiments, the unit dosage form further includes a low molecular weight polymer having a weight average molecular weight of from 5 KDa to 50 KDa selected from hydroxypropyl cellulose, hydroxypropyl methyl cellulose, hydroxyethyl cellulose, carboxymethyl cellulose, and methyl cellulose. The pH neutralizing agent can be an organic base (e.g., pyridoxine, meglumine, or any organic base described herein) or an inorganic base (e.g., magnesium hydroxide, sodium bicarbonate, or an inorganic base described herein). In particular embodiments, the unit dosage form includes 35±5% (w/w) disintegrant, from 0.5 to 5 mg, from 4 to 10 mg, or from 8 to 20 mg of apomorphine hydrochloride and pyridoxine present in an amount sufficient to produce a solution having a pH of between 4.5 and 6.5 when the unit dosage form is placed in unbuffered water at pH 7. Suitable film for oral administration of the compositions according to the invention is disclosed in, e.g., U.S. Pat. No. 8,846,074.

In some embodiments, a composition or unit dosage form described herein is administered as an emulsion, a solution, a suspension, a syrup, a slurry, a dispersion, a colloid, a dissolving tablet, a dissolving wafer, a capsule, a gel capsule, a semi-solid, a solid forma gel, a gel matrix, a cream, a paste, a tablet, a granule, a sachet, a powder, or the like. In certain aspects, about 0.000001 mg to about 2000 mg, about 0.00001 mg to about 1000 mg, or about 0.0001 mg to about 750 mg, about 0.001 mg to about 500 mg, about 0.01 mg to about 250 mg, about 0.1 mg to about 100 mg, about 0.5 mg to about 75 mg, about 1 mg to about 50 mg, about 2 mg to about 40 mg, about 5 mg to about 20 mg, or about 7.5 mg to about 15 mg of compound of formula (I) per day or per dose is administered to an individual.

In some embodiments, the compound of the invention is present in a composition or a unit dose of a composition described herein in an amount of from about 0.01 mg to about 10 mg (e.g., about 0.1-10 mg, about 0.25-5 mg, about 0.25-2.5 mg, about 1-2 mg or about 2-3 mg, about 0.5 mg to about 2 mg, about 1 to about 2 mg, about 1 mg, or about 2 mg). In some embodiments, the amount of corticosteroid administered daily or in a unit dose is between about 0.5 mg and about 3 mg, between about 0.5 mg and about 4 mg, or between about 0.35 mg and about 4 mg. In other embodiments, the amount of the compound present in a unit dose or administered daily is between about 1 and about 3 mg, or between about 1 and about 2 mg, or between about 2 and about 3 mg.

In certain aspects, about 0.05 mg to about 50 mg, about 0.25 mg to about 20 mg, about 0.25 mg to about 15 mg, about 0.25 mg to about 10 mg, or about 0.25 mg to about 5 mg (e.g., about 0.1 to about 5 mg, about 0.25 to about 2.5 mg, about 0.3 mg to about 2 mg, about 0.5 mg to about 1 mg, about 0.7 mg to about 1.5 mg, about 0.375 mg, about 0.75 mg, about 1 mg, about 1.25 mg, about 1.5 mg or about 2 mg) of the compound per day or per dose is administered to a patient.

In some embodiments, the compound is present in a unit dose in an amount of between about 5 mg and about 500 mg. In some embodiments, the amount of the compound administered daily or in a unit dose is between about 5 mg and about 300 mg. In other embodiments, the amount of the compound present in a unit dose or administered daily is between about 5 and about 250 mg, or between about 5 and about 200 mg, between about 5 mg and about 150 mg, between about 5 mg and about 100 mg, or between about 5 and about 50 mg.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

For preparing solid compositions such as tablets, the principal active ingredient is mixed with a pharmaceutical excipient to form a solid pre-formulation composition containing a homogeneous mixture of the compound of Formula I. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above containing from, for example, 0.000001 to about 2000 mg of the active ingredient of the present application.

The tablets or pills containing the compound of Formula I can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present application can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9. It will be understood that use of certain of the foregoing excipients, carriers, or stabilizers will result in the formation of pharmaceutical salts.

The therapeutic dosage of the compounds of the invention can vary according to, for example, the particular use for which the treatment is made, the manner of administration of the compound, the health and condition of the patient, and the judgment of the prescribing physician. The proportion or concentration of the compounds of the invention in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The present application also includes pharmaceutical kits useful, for example, in the treatment or prevention of diseases which include one or more containers containing a pharmaceutical composition comprising a therapeutically effective amount of the compounds of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

Delivery devices are important not only for delivering the compounds of the invention, but also for providing an appropriate environment for storage. This would include protection from microbial contamination and chemical degradation. The device and formulation should be compatible so as to avoid potential leaching or adsorption. The delivery device (or its packaging) can be optionally provided with a label and/or with instructions for use indicating that the composition should be used intranasally.

### Methods of Use

The human immune system is a multi-faceted network of functionally diverse cells expressing a broad array of receptors that collectively function to respond to infection, eliminate pre-cancerous cells, and maintain metabolic health. Breakdown of this delicately poised immune response is typically life limiting; however, even subtle changes in its ability to distinguish an invading pathogen from the host can give rise to a spectrum of autoimmune diseases, of which more than 80 have been described. Indeed, autoimmune diseases affect approximately 5%-8% of the world population and cause tremendous suffering to patients while also representing a major global socioeconomic issue.

Despite significant advances in the treatment of autoimmune disorders, definitive means to prevent the complications arising from these treatments are not yet on the immediate horizon. For example, rigorous control of multiple sclerosis with immunomodulatory therapeutics may be fraught with significant sequelae, such as dyspnea, hypertension, brachycardia, and leukopenia. Thus, there is a pressing need for development of new drugs based on a molecular and clinical understanding of the specific autoimmune diseases in individual patients.

The ability of T cells to exit secondary lymphoid organs, and subsequently migrate to sites of inflammation, depends on their ability to respond to and migrate towards a gradient of the bioactive lipid sphingosine 1-phosphate (S1P). The concentration of S1P is higher in lymph than in lymph nodes. This gradient guides lymphocytes out of lymph nodes into the lymph and back into circulation. The major facilitator superfamily transporter SPNS2 supplies S1P into lymph but not blood, and SPNS2 remains the only known requirement for lymph but not blood S1P.

SPNS2 plays a critical role in maintaining the S1P gradient. When SPNS2 is disrupted, T cells remain trapped within the lymph nodes and are unable to migrate to sites of inflammation.

In light of that which is understood in the art and described herein regarding the prominent role of SPNS2 in diseases/conditions characterized by inflammation and autoimmunity, methods are presented herein for treating such diseases/conditions, including but not limited to multiple sclerosis (MS) and inflammatory bowel disease (IBD), and other diseases related to SPNS2 activity, including but not limited to fibrosis, muscle wasting, metastases, acute lung injury, rheumatoid arthritis, colitis, and Alzheimer's disease, which methods comprise administering to a subject in need thereof a compound described herein in a therapeutically effective amount. In a particular embodiment, at least one compound described herein is utilized, either alone or in combination with one or more known therapeutic agents. In a further particular embodiment, the present invention provides a method for treating SPNS2 mediated human diseases, wherein treatment alleviates one or more symptoms resulting from that disorder, the method comprising administration to a human in need thereof a therapeutically effective amount of a compound described herein.

Further to the above, the present compounds are inhibitors of SPNS2 and are used as therapeutic agents for the treatment of conditions in mammals that are causally related or attributable to SPNS2 activity. Accordingly, the compounds and pharmaceutical compositions of this invention find use as therapeutics for preventing and/or treating a variety of conditions related to, for example, rheumatoid arthritis in mammals, including humans.

In a method of treatment aspect, this invention provides a method of treating a mammal susceptible to or afflicted with a condition associated with autoimmune disorders, such as multiple sclerosis (MS) and inflammatory bowel disease (IBD), fibrosis, muscle wasting, metastases, acute lung injury, rheumatoid arthritis, colitis, and Alzheimer's disease, which method comprises administering an effective amount of one or more of the pharmaceutical compositions just described.

As a further aspect of the invention there is provided the present compounds for use as a pharmaceutical especially in the treatment or prevention of the aforementioned conditions and diseases. Also provided herein is the use of the present compounds in the manufacture of a medicament for the treatment or prevention of one of the aforementioned conditions and diseases.

Transdermal doses are generally selected to provide similar or lower blood levels than are achieved using injection doses. Modes of administration suitable for mucosal sites are also envisioned herein and include without limitation: intra-anal swabs, enemas, intranasal sprays, and aerosolized or vaporized compounds and/or compositions for delivery to the lung mucosa. One of skill in the art would choose an appropriate delivery mode/s based on a variety of parameters, including the organ or tissue site in a patient with a disease or condition that is most severely affected by the disease or condition.

When used to prevent the onset of an inflammatory condition or autoimmune disorder, the compounds of this invention will be administered to a patient at risk for developing the condition or disorder, typically on the advice and under the supervision of a physician, in the dosage forms described above. Patients at risk for developing a particular condition generally include those that have a family history of the condition, or those who have been identified by genetic testing or screening to be particularly susceptible to developing the condition.

The compounds of this invention can be administered as the sole active agent or they can be administered in combination with other agents, including other compounds that demonstrate the same or a similar therapeutic activity and are determined to safe and efficacious for such combined administration.

### EXAMPLES

The following examples illustrate specific aspects of the instant description. The examples should not be construed as limiting, as the examples merely provide specific understanding and practice of the embodiments and their various aspects.

### Example 1: Synthesis of Analogs 002A and 001A, 001B, 002B, 003, 004, 009, 110, 111, 211, 240, 241 According to the Invention

Compound 002A according to the present disclosure was prepared as shown in Scheme 1, and described below.

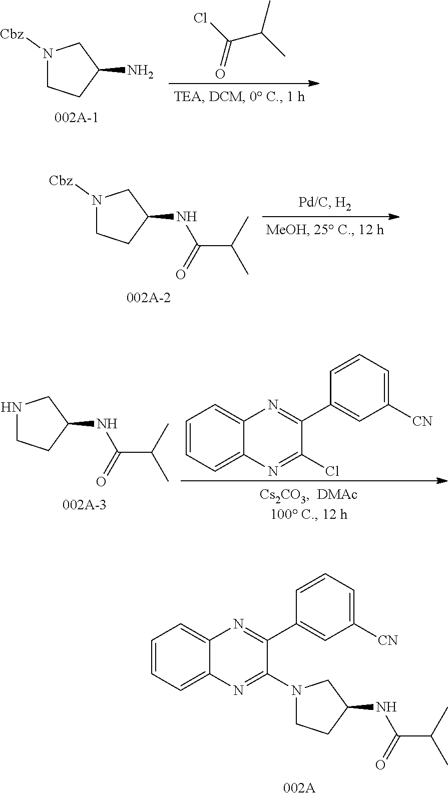

### Example 1A: Preparation of Compound 002A-2

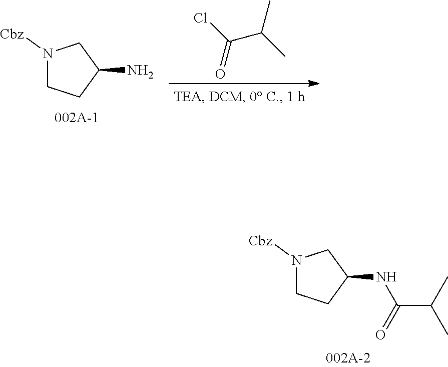

To a 50 mL three-necked round-bottom flask equipped with a magnetic stir bar was added benzyl (3S)-3-aminopyrrolidine-1-carboxylate 002A-1 (500 mg, 2.27 mmol, 1 eq) followed by the addition of DCM (5 mL). Then TEA (344 mg, 3.41 mmol, 1.5 eq) and 2-methylpropanoyl chloride (290 mg, 2.72 mmol, 1.2 eq) was added into the mixture at 0° C. The mixture was stirred at 0° C. for 1 h. The mixture was quenched by slow addition of H₂O (10 mL). The resulting mixture was transferred to a separatory funnel, and the aqueous layer mixture was extracted with ethyl acetate (15 mL×2). The combined organic layers were washed with brine (10 mL), dried over anhydrous sodium sulfate, filtered, and concentrated under reduced pressure affording the residue as a light yellow oil. The crude product was purified by silica gel column chromatography (petroleum ether/ethyl acetate, from 10/1 to 1/1) to give the benzyl (3S)-3-(2-methylpropanoylamino)pyrrolidine-1-carboxylate 002A-2 (608 mg, 92% yield) as a colorless oil. ¹H NMR of 002A-2: (400 MHz, DMSO-d₆) δ 8.05-7.89 (m, 1H), 7.45-7.24 (m, 5H), 5.17-4.99 (m, 2H), 4.28-4.08 (m, 1H), 3.35 (br s, 3H), 3.17-3.08 (m, 1H), 2.40-2.27 (m, 1H), 2.08-1.94 (m, 1H), 1.80-1.67 (m, 1H), 1.04-0.93 (m, 6H).

### Example 2A: Preparation of Compound 002A-3

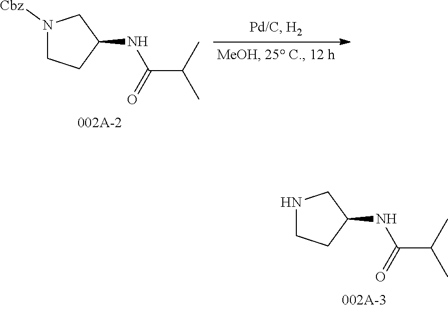 [SMILES: CC(C)C(=O)N[C@H]1CCN(C(=O)OCc2ccccc2)C1 ; CC(C)C(=O)N[C@H]1CCNC1]

To a 50 mL round-bottom flask equipped with a magnetic stir bar and a reflux condenser was added benzyl (3S)-3-(2-methylpropanoylamino)pyrrolidine-1-carboxylate 002A-2 (580 mg, 2.00 mmol, 1 eq) followed by the addition of MeOH (10 mL). Then Pd/C (100 mg, 2.00 mmol, 10% purity on carbon, 1 eq) was added into the mixture at 25° C. and placed under an atmosphere of H₂ (3.98 mg, 1.97 mmol) (15 Psi). The mixture was stirred at 25° C. for 12 h. The TLC (petroleum ether/ethyl acetate: 2/1) showed the reaction had completed. The suspension was filtered through a pad of Celite. The Celite pad was washed with methanol (20 mL) and concentrated under reduced pressure to afford 2-methyl-N-[(3S)-pyrrolidin-3-yl]propanamide 002A-3 (340 mg, crude) as a colorless solid. ¹H NMR of 002A-3: (400 MHz, DMSO-d₆) δ 7.84-7.67 (m, 1H), 4.14-3.98 (m, 1H), 2.94-2.77 (m, 2H), 2.75-2.64 (m, 1H), 2.48 (br s, 1H), 2.38-2.24 (m, 1H), 1.92-1.78 (m, 1H), 1.51-1.38 (m, 1H), 0.98-0.95 (m, 6H).

### Example 1C: Preparation of 002A

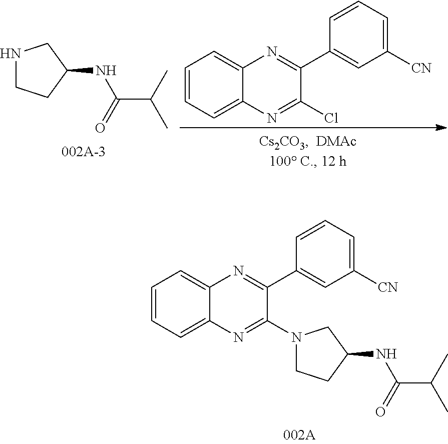

To a 10 mL round-bottom flask equipped with a magnetic stir bar and a reflux condenser was added 2-methyl-N-[(3S)-pyrrolidin-3-yl]propanamide 002A-3 (80 mg, 512 umol, 1.2 eq) followed by the addition of DMAc (3 mL). Then Cs₂CO₃ (278 mg, 853 umol, 2 eq) and 3-(3-chloroquinoxalin-2-yl)benzonitrile (117 mg, 426 umol, 96.6% purity, 1 eq) were added into the mixture dropwise at 25° C. The mixture was heated to 100° C. and stirred for 12 h. The suspension was cooled to room temperature and filtered through a pad of filter paper. The crude product was purified by preparative HPLC: (Waters Xbridge C18 column (150×25 mm, 5 um); flow rate: 25 mL/min; gradient: 40%-70% B over 8 min; mobile phase A: 10 mM NH₄HCO₃, mobile phase B: acetonitrile). After lyophilization, N-[(3S)-1-[3-(3-cyanophenyl)quinoxalin-2-yl]pyrrolidin-3-yl]-2-methyl-propanamide 002A (88 mg, 53% yield, 99.98% purity) was obtained as a yellow solid. SFC of 002A: Ret. Time: 1.714 min, 98.4% e.e. LCMS of 002A: Ret. Time: 0.869 min, MS (ESI) m/z: 386.2, M+H⁺. ¹H NMR of 002A: (400 MHz, DMSO-d₆) δ 8.13 (s, 1H), 8.06-7.99 (m, 1H), 7.98-7.93 (m, 1H), 7.91-7.84 (m, 2H), 7.79-7.69 (m, 2H), 7.65 (dt, J=1.4, 7.6 Hz, 1H), 7.45 (t, J=7.2 Hz, 1H), 4.26-4.13 (m, 1H), 3.45-3.39 (m, 2H), 3.28-3.20 (m, 1H), 3.04 (dd, J=4.8, 11.2 Hz, 1H), 2.36-2.21 (m, 1H), 2.14-1.89 (m, 1H), 1.75 (qd, J=6.2, 12.2 Hz, 1H), 0.95 (dd, J=6.8, 11.6 Hz, 6H).

### Example 1D: Preparation of Analogous Compounds 001A, 001B, 002B, 003, 004, 009, 110, 111, 211, 240, and 241

Table 1, below, depicts exemplary compounds synthesized according to an analogous procedure to the one described for 002A.

TABLE 1
Compounds synthesized analogously to 002A.

| Compound Structure | Compound No. | Yield/Purity | Characterization |
|---|---|---|---|
| 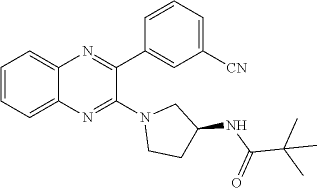 [SMILES: CC(C)(C)C(=O)N[C@H]1CCN(c2nc3ccccc3nc2-c2cccc(C#N)c2)C1] | 001A | 25% yield, 99.5% purity | SFC of 001A: Ret. Time: 1.714 min, 100% e. e. LCMS of 001A: (Ret. Time: 0.940 min, MS (ESI) m/z: 400.4, M + H⁺;. ¹H NMR of 001A: (400 MHz, DMSO-d₆) δ 8.15-8.11 (m, 1H), 8.05-8.00 (m, 1H), 7.98-7.93 (m, 1H), 7.91-7.85 (m, 1H), 7.76-7.69 (m, 2H), 7.68-7.62 (m, 1H). 7.48-7.40 (m, 2H), 4.25-4.16 (m, 1H), 3.49-3.40 (m, 1H), 3.24 (br s, 3H), 2.00- 1.90 (m. 1H), 1.87-1.75 (m, 1H), 1.06 (s, 9H) |
| 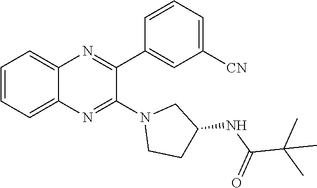 [SMILES: CC(C)(C)C(=O)N[C@@H]1CCN(c2nc3ccccc3nc2-c2cccc(C#N)c2)C1] | 001B | 41% yield, 99.4% purity | SFC of 001B: Ret. Time: 2.015 min, 100% e.e. LCMS of 001B: Ret. Time: 1.022 min, MS (ESI) m/z: 400.4, M + H⁺; ¹H NMR of 001B: (400 MHz, DMSO-d₆) δ 8.13 (s, 1H), 8.03 (d, J = 7.8 Hz, 1H), 7.98-7.93 (m, 1H), 7.88 (dd, J = 1.0, 8.2 Hz, 1H). 7.76-7.69 (m, 2H), 7.65 (dt, J = 1.4, 7.6 Hz, 1H), 7.49-7.39 (m, 2H), 4.27- 4.16 (m, 1H), 3.45 (br d, J = 4.8 Hz, 1H), 3.25-3.15 (m, 3H), 1.95 (qd, J = 6.4, 12.3 Hz, 1H), 1.87-1.76 (m, 1H), 1.06 (s, 9H) |
| 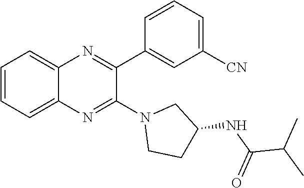 [SMILES: CC(C)C(=O)N[C@@H]1CCN(c2nc3ccccc3nc2-c2cccc(C#N)c2)C1] | 002B | 21% yield, 98.9% purity | LCMS of 002B: Ret. Time: 0.974 min, MS (ESI) m/z: 386.4, M + H⁺; ¹H NMR of 002B: (400 MHz, DMSO-d₆) δ 8.17-8.09 (m, 1H), 8.01 (dd, J = 1.6, 8.0 Hz, 1H), 7.96 (dd, J = 1.2, 7.8 Hz, 1H), 7.91-7.83 (m, 2H), 7.77-7.69 (m, 2H), 7.65 (dt, J = 1.4, 7.6 Hz, 1H), 7.48- 7.41 (m, 1H), 7.06 (br s, 1H), 4.23-4.13 (m, 1H), 3.41 (dd, J = 6.2, 11.2 Hz, 1H), 3.37-3.32 (m, 1H), 3.28-3.21 (m, 1H), 3.04 (dd, J = 4.8, 11.2 Hz, 1H), 2.29 (td, J = 6.8, 13.6 Hz, 1H), 2.05-1.94 (m, 1H), |
| | | | 1.75 (qd. J = 6.2, 12.4 Hz. 1H), 0.95 (dd, |
| | | | J = 6.8, 11.6 Hz, 6H). |
| 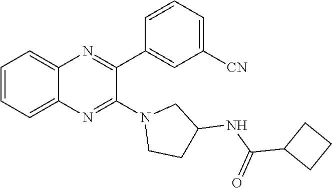 [SMILES: N#Cc1cccc(-c2nc3ccccc3nc2N2CCC(NC(=O)C3CCC3)C2)c1] | 003 | 14% yield, 91.9% purity | LCMS of 003: (Ret. Time: 0.939 min, MS (ESI) m/z: 398.2, M + H⁺; ¹H NMR of 003: (400 MHz, DMSO-d₆) δ 8.16-8.09 (m, 1H), 8.06-7.94 (m, 2H), 7.94-7.83 (m, 2H), 7.78-7.69 (m, 2H), 7.65 (dt, J = 1.4, 7.6 Hz, 1H), 7.45 (ddd, J = 1.4, 6.8, 8.2 Hz, 1H), 4.25-4.11 (m, 1H), 3.41 (br dd, J = 6.2, 11.2 Hz, 2H), 3.28-3.22 (m, 1H), 3.04 (dd, J = 4.8, 11.2 Hz, 1H), 2.37-2.22 (m, 1H), 1.99 (br dd, J = 5.6, 12.6 Hz, 1H), 1.83-1.69 (m, 1H), 0.95 (dd, J = 6.8, 11.6 Hz, 6H). |
| 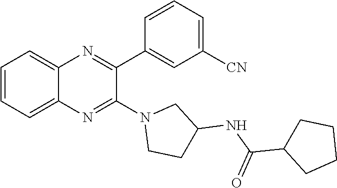 [SMILES: N#Cc1cccc(-c2nc3ccccc3nc2N2CCC(NC(=O)C3CCCC3)C2)c1] | 004 | 16% yield, 99.2% purity | LCMS of 004: Ret. Time: 0.973 min, MS (ESI) m/z: 412.2, M + H⁺; ¹H NMR of 004: (400 MHz, DMSO-d₆) δ 8.13 (s, 1H), 8.01 (brd, J = 7.8 Hz, 1H), 7.96 (d, J = 7.8 Hz, 1H), 7.93-7.86 (m, 2H), 7.76-7.70 (m, 2H), 7.65 (dt, J = 1.4, 7.6 Hz, 1H), 7.48-7.41 (m, 1H), 4.21- 4.15 (m, 1H), 3.42-3.38 (m, 1H), 3.30- 3.20 (m, 2H), 3.03 (dd, J = 4.8, 10.8 Hz, 1H), 2.04-1.94 (m, 1H), 1.78-1.64 (m. 3H), 1.63-1.50 (m, 5H), 1.49-1.39 (m, 2H). |
| 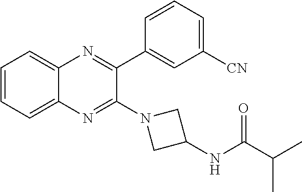 [SMILES: CC(C)C(=O)NC1CN(c2nc3ccccc3nc2-c2cccc(C#N)c2)C1] | 009 | 46% yield. 99.5% purity | LCMS of 009: Ret. Time: 0.900 min, MS (ESI) m/z: 372.2, M + H⁺; ¹H NMR of 009: (400 MHz, DMSO-d₆) δ 8.28 (br d, J = 6.6 Hz, 1H), 8.13 (s, 1H), 8.01 (t, J = 8.8 Hz, 2H), 7.92 (d, J = 7.6 Hz, 1H), 7.79-7.72 (m, 2H), 7.69 (dt, J = 1.4, 7.6 Hz, 1H), 7.54-7.48 (m, 1H), 4.45- 4.35 (m, 1H), 3.99 (t, J = 8.4 Hz, 2H), 3.62 (br dd, J = 5.8, 8.8 Hz, 2H), 2.31- 2.24 (m, 1H), 0.95 (d, J = 6.8 Hz, 6H). |
| 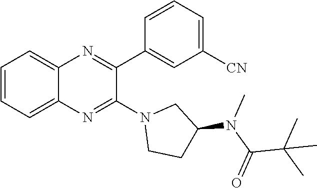 [SMILES: CN(C(=O)C(C)(C)C)[C@H]1CCN(c2nc3ccccc3nc2-c2cccc(C#N)c2)C1] | 110 | 24% yield, 97.7% purity | LC MS of 110: Ret. Time: 0.979 min, MS (ESI) m/z: 414.1, M + H⁺; ¹H NMR of 110: (400 MHz, CDCl₃) δ 8.10 (t, J = 1.6 Hz, 1H), 8.02 (td, J = 1.6, 8.0 Hz, 1H), 7.96 (dd, J = 0.8, 8.4 Hz, 1H), 7.83 (dd, J = 0.8, 8.4 Hz, 1H), 7.75 (td, J = 1.6, 8.0 Hz, 1H), 7.68-7.61 (m, 2H), 7.49 (ddd, J = 2.0, 6.8, 8.4 Hz, 1H), 5.04 (quin, J = 8.0 Hz. 1H), 3.47 (dd, J = 7.6, 11.6 Hz, 1H), 3.42-3.27 (m, 3H), 2.93 (s, 3H), 2.12-1.96 (m, 2H), 1.30 (s, 9H). |
| 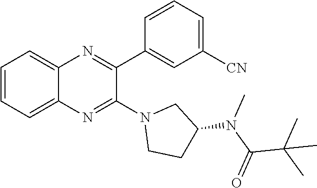 [SMILES: CN(C(=O)C(C)(C)C)[C@@H]1CCN(c2nc3ccccc3nc2-c2cccc(C#N)c2)C1] | 111 | 23% yield, 96.6% purity, TFA salt | LC MS of 111: Ret. Time: 0.987 min, MS (ESI) m/z: 414.1, M + H⁺; ¹H NMR of 111: (400 MHz, CDCl₃) δ 8.10 (s, 1H), 7.99 (dd, J = 8.0, 17.6 Hz, 2H), 7.84 (d, J = 8.4 Hz, 1H), 7.76 (d, J = 7.6 Hz. 1H), 7.70-7.59 (m, 2H), 7.55-7.46 (m, 1H), 5.03 (q, J = 7.6 Hz, 1H), 3.50 (dd, J = 8.0, 11.4 Hz, 1H), 3.43-3.26 (m, 3H). 2.94 (s, 3H). 2.12-1.98 (m, 2H), 1.30 (s, 9H). |
| 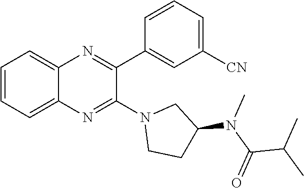 [SMILES: CC(C)C(=O)N(C)[C@H]1CCN(c2nc3ccccc3nc2-c2cccc(C#N)c2)C1] | 211 | 38.2% yield, 99.1% purity | LC MS of 211: (Ret. Time: 1.207 min, MS (ESI) m/z: 400.1, M + H+). 1H NMR of 211: 1H NMR (400 MHz, DMSO) 6 8.16 (s, 1H), 8.03 (d, J = 7.6 Hz, 1H), 7.96 (d, J = 7.6 Hz, 1H), 7.90 (d, J = 8.0 Hz, 1H), 7.73 (t, J = 8.0 Hz, 2H), 7.66 (1, J = 7.6 Hz, 1H), 7.47 (1, J = 7.6 Hz, 1H), 5.01 (t, J = 6.4 Hz. 1H), 4.63 (s. 1H), 3.42-3.34 (m, 1H), 3.32-3.27 (m, 1H), 3.20 (dd, J = 17.2, 9.2 Hz, 2H), 2.98- 2.77 (m, 3H), 2.68 (s, 1H), 1.96 (dd, J = |
| | | | 39.6, 7.2 Hz, 2H), 0.97 (d, J = 6.4 Hz, |
| | | | 6H). |
| 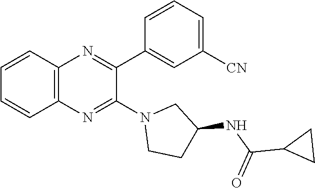 [SMILES: N#Cc1cccc(-c2nc3ccccc3nc2N2CC[C@H](NC(=O)C3CC3)C2)c1] | 240 | 9.08% yield, >99.9% purity | LC MS of 240: Ret. Time: 1.035 min, MS (ESI) m/z: 384.2, M + H⁺). ¹H NMR of 240: (400 MHz, DMSO) δ 8.24 (d, J = 6.8 Hz, 1H), 8.13 (1, J = 1.6 Hz. 1H), 8.04-7.99 (m, 1H), 7.99-7.94 (m, 1H), 7.89 (dd, J = 8.4, 1.2 Hz, 1H), 7.76-7.70 (m, 2H), 7.65 (m, J = 8.4, 6.8, 1.6 Hz, 1H), 7.45 (m. J = 8.4, 6.8, 1.6 Hz, 1H), 4.21 (dd, J = 11.6, 6.0 Hz, 1H), 3.39 (dd, J = 11.6, 6.8 Hz, 3H), 3.29-3.26 (m, 1H), 3.02 (dd, J = 11.2, 4.8 Hz, 1H), 2.02 (m, J = 12.8, 6.4 Hz., 1H), 1.84-1.69 (m, |
| | | | 1H), 1.54-1.41 (m, 1H), 0.67-0.57 (m, |
| | | | 4H). |
| 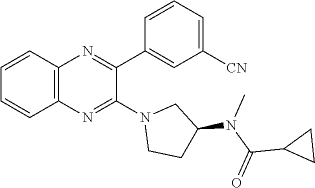 [SMILES: CN(C(=O)C1CC1)[C@H]1CCN(c2nc3ccccc3nc2-c2cccc(C#N)c2)C1] | 241 | 16.6% yield, 98.7% purity | LC MS of 241: Ret. Time: 1.131 min, MS (ESI) m/z: 398.2, M + H⁺. ¹H NMR of 241: ¹H NMR (400 MHz, DMSO) δ 8.16 (s, 1H), 8.06-8.00 (m, 1H), 7.99-7.93 (m, 1H), 7.92-7.87 (m, 1H), 7.77-7.70 (m, 2H), 7.70-7.63 (m, 1H), 7.47 (m, J = 8.4, 6.8, 1.2 Hz, 1H), 4.99 (s, 1H), 3.38 (s, 1H), 3.23 (s, 3H), 2.86 (d, J = 115.6 Hz, 3H), 2.11-1.84 (m, 3H), 0.70 (s, 4H). |

Compound 0011A according to the present disclosure was prepared as shown in Scheme 2 by an analogous procedure to the one described in Examples 1A-1C.

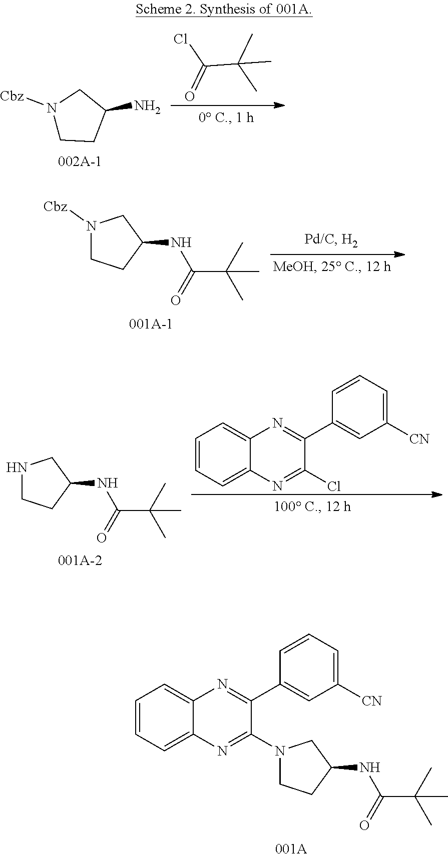

Compound 001B according to the present disclosure was prepared as shown in Scheme 3 by an analogous procedure to the one described in Examples 1A-1C.

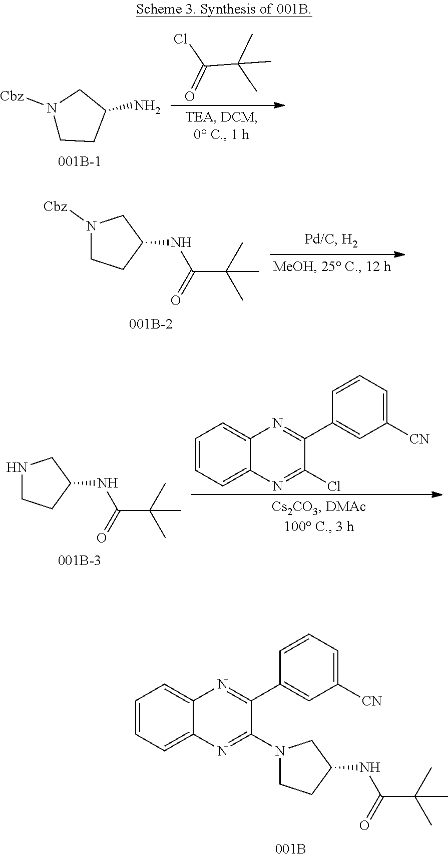

Compound 002B according to the present disclosure was prepared as shown in Scheme 4 by an analogous procedure to the one described in Exampes 1A-1C.

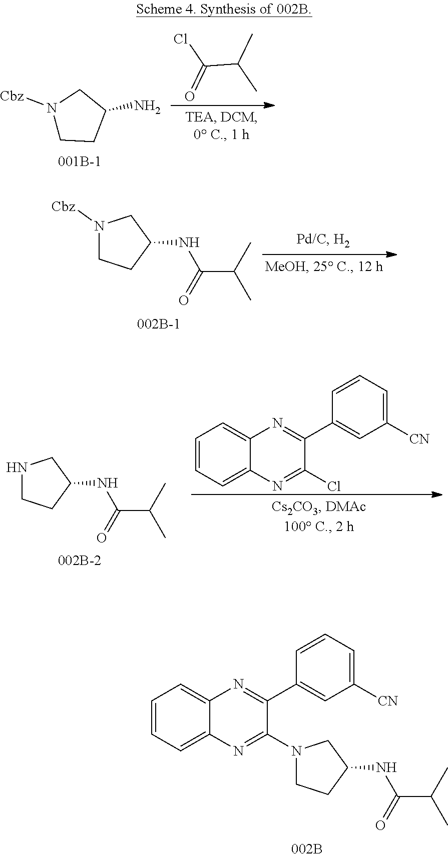

Compound 003 according to the present disclosure was prepared as shown in Scheme 5 by an analogous procedure to the one described in Examples 1A-1C.

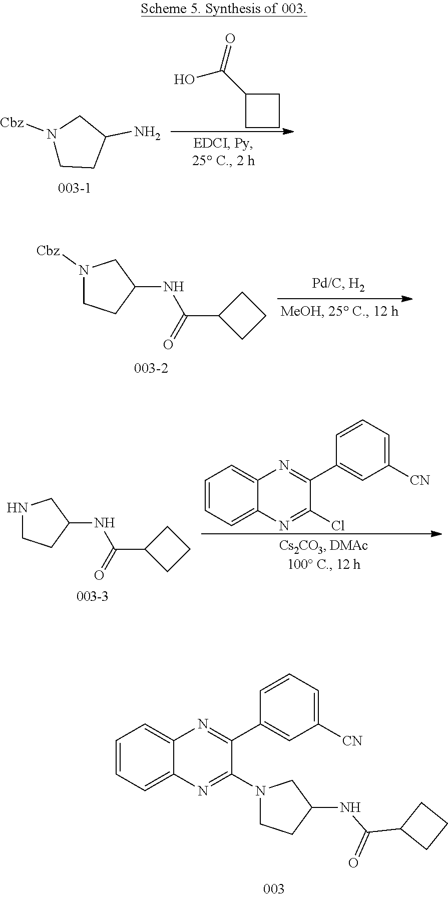

Compound 004 according to the present disclosure was prepared as shown in Scheme 6 by an analogous procedure to the one described in Examples 1A-1C.

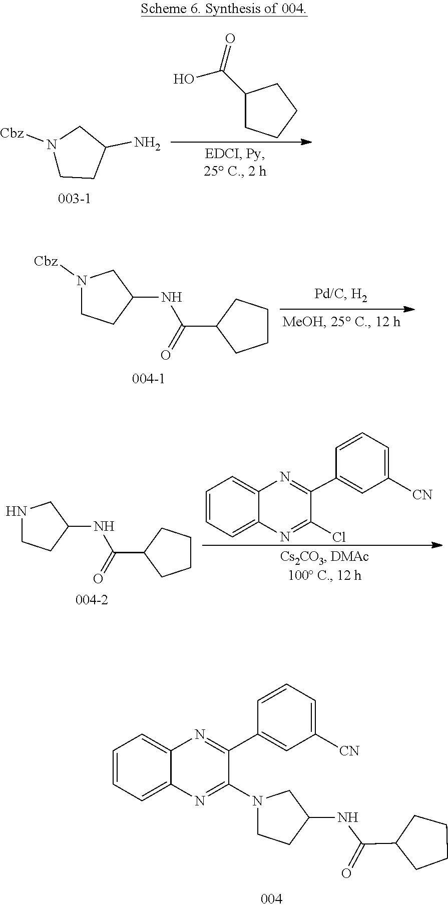

Compound 009 according to the present disclosure was prepared as shown in Scheme 7 by an analogous procedure to the one described in Examples 1A-1C.

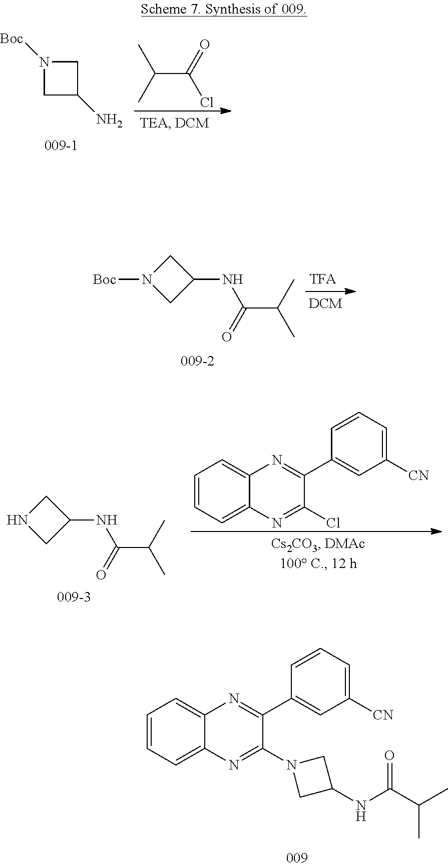

Compound 110 according to the present disclosure was prepared as shown in Scheme 8 by an analogous procedure to the one described in Examples 1A-1C.

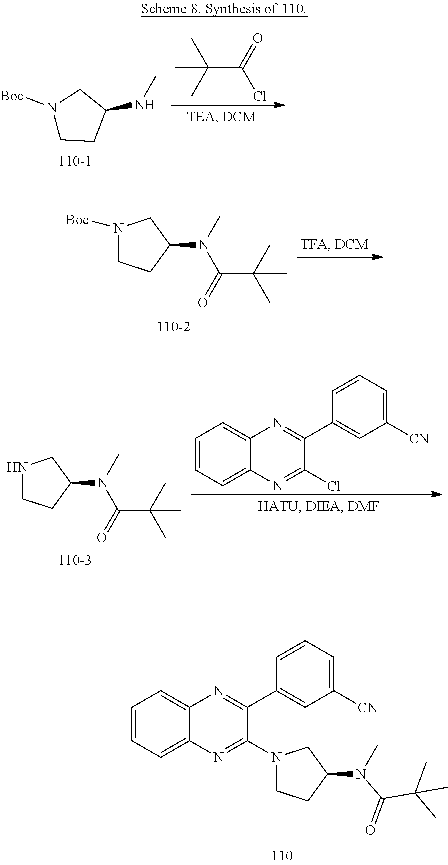

Compound 111 according to the present disclosure was prepared as shown in Scheme 9 by an analogous procedure to the one described in Examples 1A-1C.

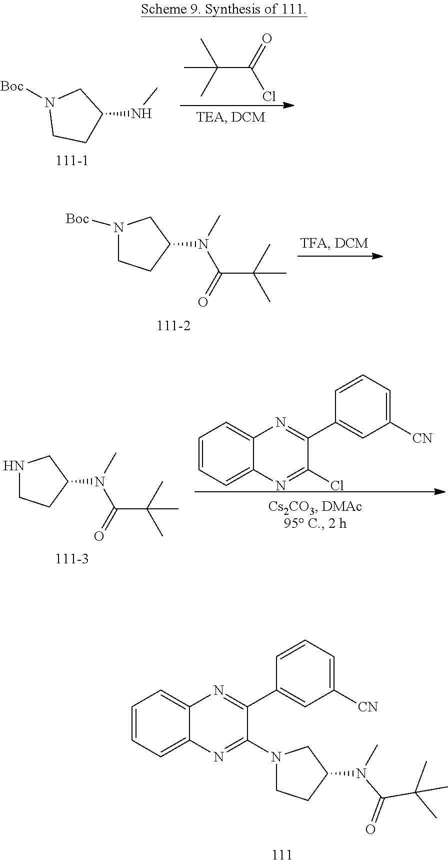

Compound 211 according to the present disclosure was prepared as shown in Scheme 10 by an analogous procedure to the one described in Examples 1A-1C.

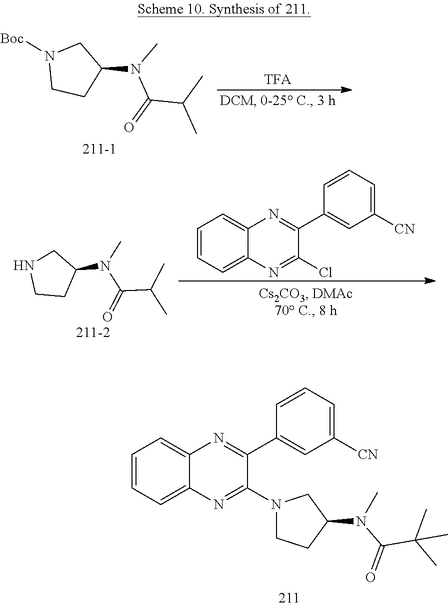

Compound 240 according to the present disclosure was prepared as shown in Scheme 11 by an analogous procedure to the one described in Examples 1A-1C.

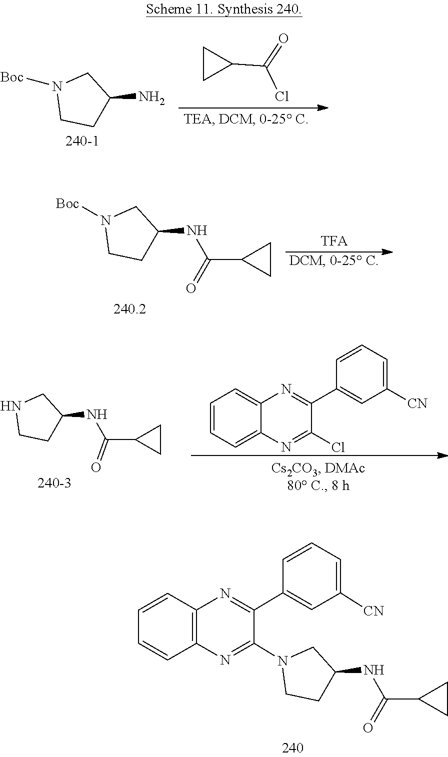 [SMILES: CC(C)(C)OC(=O)N1CC[C@H](N)C1 ; O=C(Cl)C1CC1 ; CC(C)(C)OC(=O)N1CC[C@H](NC(=O)C2CC2)C1 ; O=C(N[C@H]1CCNC1)C1CC1 ; N#Cc1cccc(-c2nc3ccccc3nc2Cl)c1 ; N#Cc1cccc(-c2nc3ccccc3nc2N2CC[C@H](NC(=O)C3CC3)C2)c1]

Compound 241 according to the present disclosure was prepared as shown in Scheme 12 by an analogous procedure to the one described in Examples 1A-1C.

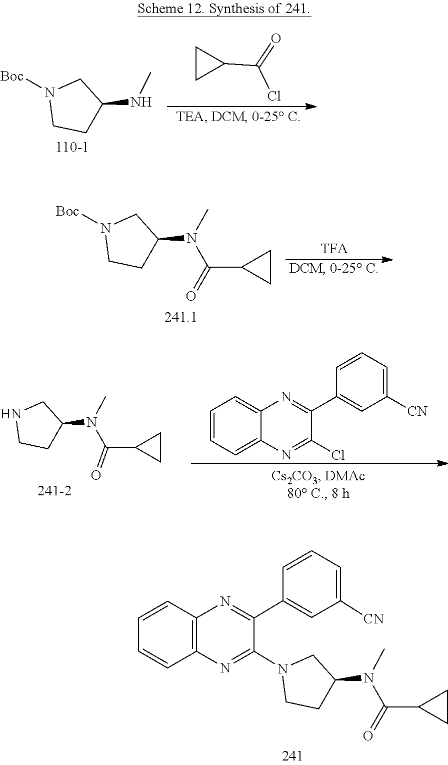

### Example 2: Synthesis of Analogs 006 and 005, 011, 219, 220, 221, 281 According to the Invention

Compound 006 according to the present disclosure was prepared as shown in Scheme 13 and described below.

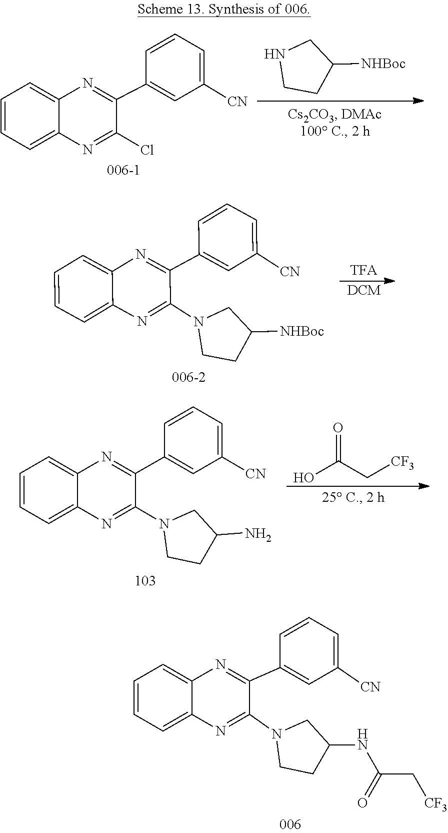 [SMILES: N#Cc1cccc(-c2nc3ccccc3nc2Cl)c1 ; CC(C)(C)OC(=O)NC1CCNC1 ; CC(C)(C)OC(=O)NC1CCN(c2nc3ccccc3nc2-c2cccc(C#N)c2)C1 ; N#Cc1cccc(-c2nc3ccccc3nc2N2CCC(N)C2)c1 ; O=C(O)CC(F)(F)F ; N#Cc1cccc(-c2nc3ccccc3nc2N2CCC(NC(=O)CC(F)(F)F)C2)c1]

### Example 2A Preparation of Compound 006-2

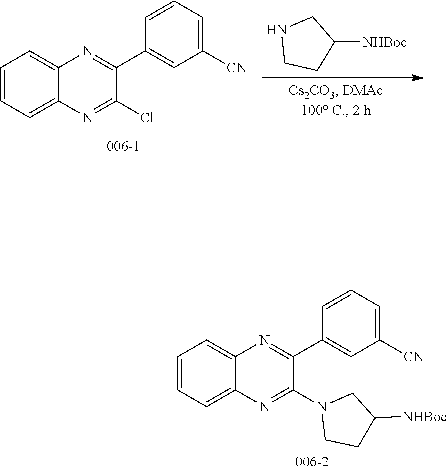 [SMILES: N#Cc1cccc(-c2nc3ccccc3nc2Cl)c1 ; CC(C)(C)OC(=O)NC1CCNC1 ; CC(C)(C)OC(=O)NC1CCN(c2nc3ccccc3nc2-c2cccc(C#N)c2)C1]

To a 50 mL round-bottom flask equipped with a magnetic stir bar and a reflux condenser was added 3-(3-chloroquinoxalin-2-yl)benzonitrile 006-1 (1 g, 3.64 mmol, 96.6% purity, 1 eq) followed by the addition of DMAc (10 mL). Then Cs₂CO₃ (2.37 g, 7.28 mmol, 2 eq) and tert-butyl N-pyrrolidin-3-ylcarbamate 2 (813 mg, 4.37 mmol, 1.2 eq) was added into the mixture dropwise at 25° C. The mixture was heated to 100° C. and stirred for 2 h. The mixture was quenched by slow addition of H₂O (30 mL). The resulting mixture was transferred to a separatory funnel, and the aqueous layer mixture was extracted with ethyl acetate (20 mL×3). The combined organic layers were washed with brine (20 mL), dried over anhydrous sodium sulfate, filtered, and concentrated under reduced pressure affording the residue as a yellow oil. The crude product was purified by silica gel column chromatography (petroleum ether/ethyl acetate, from 10/1 to 5/1) to give the desired product as a yellow solid. tert-Butyl N-[1-[3-(3-cyanophenyl)quinoxalin-2-yl]pyrrolidin-3-yl]carbamate 006-2 (1.5 g, 96% yield, 96.8% purity) was obtained as a yellow solid. LCMS of 006-2: Ret. Time: 0.940 min, MS (ESI) m/z: 416.2, M+H⁺. ¹H NMR of 006-2: (400 MHz, CDCl₃) δ 8.07 (d, J=1.4 Hz, 1H), 8.00-7.92 (m, 2H), 7.80 (dd, J=0.8, 8.4 Hz, 1H), 7.74 (td, J=1.4, 7.8 Hz, 1H), 7.66-7.59 (m, 2H), 7.47 (ddd, J=1.4, 7.0, 8.4 Hz, 1H), 4.13 (q, J=7.2 Hz, 2H), 3.53-3.48 (m, 2H), 3.46 (br d, J=3.0 Hz, 2H), 3.11 (dd, J=4.6, 11.4 Hz, 1H), 1.42 (s, 9H).

### Example 2B: Preparation of Compound 103

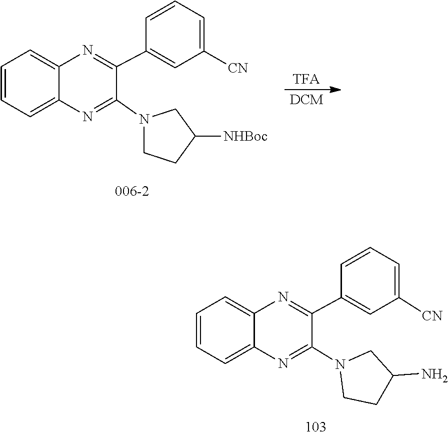 [SMILES: CC(C)(C)OC(=O)NC1CCN(c2nc3ccccc3nc2-c2cccc(C#N)c2)C1 ; N#Cc1cccc(-c2nc3ccccc3nc2N2CCC(N)C2)c1]

To a 50 mL round-bottom flask equipped with a magnetic stir bar was added tert-butyl N-[1-[3-(3-cyanophenyl)quinoxalin-2-yl]pyrrolidin-3-yl]carbamate 006-2 (700 mg, 1.63 mmol, 96.86% purity, 1 eq) followed by the addition of DCM (6 mL). Then TFA (1.54 g, 13.5 mmol, 8.28 eq) was added into the mixture at 25° C. The mixture was stirred at 25° C. for 12 h. The mixture was quenched by slow addition of H₂O (15 mL). The resulting mixture was transferred to a separatory funnel, and the aqueous layer mixture was extracted with ethyl acetate (15 mL×2). The combined organic layers were washed with brine (10 mL), dried over anhydrous sodium sulfate, filtered, and concentrated under reduced pressure affording 3-[3-(3-aminopyrrolidin-1-yl)quinoxalin-2-yl]benzonitrile 103 (465 mg, 90% yield, 100% purity, TFA salt) as a yellow solid. LCMS of 103: Ret. Time: 0.892 min, MS (ESI) m/z: 305.2, M+H⁺. ¹H NMR of 103: (400 MHz, CDC₃) δ 8.08 (t, J=1.6 Hz, 1H), 7.98 (td, J=1.4, 7.8 Hz, 1H), 7.93 (dd, J=1.4, 8.4 Hz, 1H), 7.79 (dd, J=1.4, 8.4 Hz, 1H), 7.72 (td, J=1.4, 7.8 Hz, 1H), 7.65-7.58 (m, 2H), 7.44 (ddd, J=1.4, 6.8, 8.4 Hz, 1H), 3.63 (q, J=5.6 Hz, 1H), 3.54-3.43 (m, 2H), 3.39-3.32 (m, 1H), 3.08 (dd, J=4.8, 11.2 Hz, 1H), 2.15-2.11 (m, 1H), 1.76 (qd, J=6.4, 12.8 Hz, 1H)

### Example 2C: Preparation of Compound 006

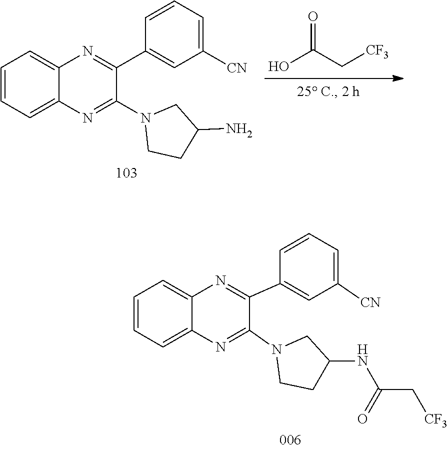 [SMILES: N#Cc1cccc(-c2nc3ccccc3nc2N2CCC(N)C2)c1 ; O=C(O)CC(F)(F)F ; N#Cc1cccc(-c2nc3ccccc3nc2N2CCC(NC(=O)CC(F)(F)F)C2)c1]

To a 50 mL round-bottom flask equipped with a magnetic stir bar was added 3,3,3-trifluoropropanoic acid (35.7 mg, 279 umol, 1.1 eq) followed by the addition of DMF (3 mL). Then DIPEA (98.3 mg, 761 umol, 3 eq) and HATU (192 mg, 507 umol, 2 eq) were added into the mixture at 25° C. The mixture was stirred at 25° C. for 5 min. Then 3-[3-(3-aminopyrrolidin-1-yl)quinoxalin-2-yl]benzonitrile 103 (80 mg, 253 umol, 100% purity, TFA salt, 1 eq) was added into the mixture. The mixture was stirred at 25° C. for 2 h. The mixture was quenched by slow addition of H₂O (15 mL). The resulting mixture was transferred to a separatory funnel, and the aqueous layer mixture was extracted with ethyl acetate (15 mL×2). The combined organic layers were washed with brine (10 mL), dried over anhydrous sodium sulfate, filtered, and concentrated under reduced pressure affording the residue as yellow oil. The crude product was purified by preparative HPLC: (3_Phenomenex Luna C18 column (75×30 mm, 3 um); flow rate: 25 mL/min; gradient: 37%-67% B over 7 min; mobile phase A: 0.1% aqueous TFA, mobile phase B: acetonitrile). After lyophilization, N-[1-[3-(3-cyanophenyl)quinoxalin-2-yl]pyrrolidin-3-yl]-3,3,3-trifluoro-propanamide 006 (30 mg, 26% yield, 94.3% purity, TFA salt) was obtained as a yellow solid. LCMS of 006: Ret. Time: 0.934 min, MS (ESI) m/z: 426.1, M+H⁺. ¹H NMR of 006: (400 MHz, DMSO-d₆) δ 8.45 (br d, J=6.0 Hz, 1H), 8.13 (s, 1H), 8.00 (dd, J=7.8, 17.8 Hz, 2H), 7.90 (d, J=7.4 Hz, 1H), 7.80-7.71 (m, 2H), 7.70-7.61 (m, 1H), 7.47 (t, J=6.8 Hz, 1H), 4.23 (br d, J=5.8 Hz, 1H), 3.45 (br dd, J=6.2, 11.4 Hz, 1H), 3.39-3.26 (m, 2H), 3.26-3.12 (m, 2H), 3.03 (br dd, J=4.8, 11.4 Hz, 1H), 2.13-1.99 (m, 1H), 1.85-1.69 (m, 1H), 1.41 (s, 1H).

### Example 2D: Preparation of Analogous Compounds 005, 011, 219, 220, 221, and 281

Table 2, below, depicts exemplary compounds synthesized according to an analogous procedure to the one described for 006.

TABLE 2
Compounds synthesized analogously to 006.

| Compound Structure | Compound No. | Yield/Purity | Characterization |
|---|---|---|---|
| 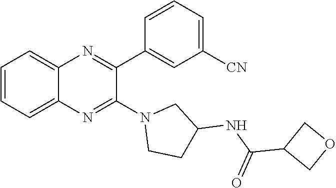 [SMILES: N#Cc1cccc(-c2nc3ccccc3nc2N2CCC(NC(=O)C3COC3)C2)c1] | 005 | 6% yield, 99.6% purity | LC MS of 005: Ret. Time: 0.868 min, MS (ESI) m/z: 400.3. ¹H NMR of 005: (400 MHz, DMSO-d₆) δ 8.12 (t, J = 1.4 Hz, 1H), 8.07 (d, J = 6.6 Hz, 1H), 8.00 (dd, J = 1.4, 7.8 Hz, 1H), 7.98-7.94 (m, 1H), 7.88 (dd, J = 1.2, 8.2 Hz, 1H), 7.73 (ddd, J = 3.2, 4.4, 8.0 Hz, 2H), 7.65 (dt, J = 1.4, 7.6 Hz, 1H), 7.45 (dt, J = 1.4, 7.6 Hz, 1H), 4.63-4.50 (m, 4H), 4.29-4.19 (m, 1H), 3.71-3.61 (m. 1H), 3.44-3.40 (m, 1H), 3.32-3.22 (m, 2H), 3.02 (dd, J = 4.8, 11.2 Hz, 1H), 2.09- 1.96 (m, 1H), 1.80-1.70 (m, 1H) |
| 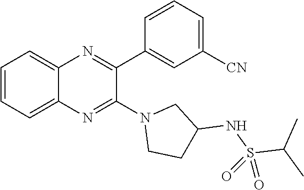 [SMILES: CC(C)S(=O)(=O)NC1CCN(c2nc3ccccc3nc2-c2cccc(C#N)c2)C1] | 011 | 24% yield, 94.6% purity, TFA salt | LC MS of 011: Ret. Time: 0.857 min, MS (ESI) m/z: 422.2, N + H⁺; ¹H NMR of 011: (400 MHz, DMSO-d₆) δ 8.11 (t, J = 1.4 Hz, 1H), 8.01 (td, J = 1.6. 7.8 Hz, 1H), 7.96 (td, J = 1.4, 7.8 Hz. 1H), 7.88 (dd, J = 0.8, 8.2 Hz, 1H), 7.78-7.70 (m, 2H), 7.65 (ddd, J = 1.6, 6.8, 8.4 Hz, 1H), 7.45 (ddd, J = 1.4. 6.8, 8.2 Hz, 1H), 7.34 (d, J = 7.2 Hz, 1H), 3.89-3.83 (m, 1H), 3.35-3.22 (m, 3H), 3.15-3.04 (m, 2H), 2.13-1.99 (m, 1H), 1.88-1.74 (m. 1H), 1.18 (d, J = 6.8 H7, 3H), 1.14 (d, J = |
| | | | 6.8 Hz, 3H). |
| 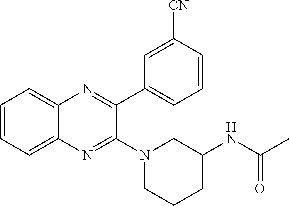 [SMILES: CC(=O)NC1CCCN(c2nc3ccccc3nc2-c2cccc(C#N)c2)C1] | 219 | 30.1% yield | LCMS of 219: Ret. Time: 1.060 min, MS (ESI) m/z: 372.2, M + H⁺. ¹H NMR of 219: ¹H NMR (400 MHz, CDCl₃) δ 8.35-8.17 (m, 2H), 8.05-7.88 (m, 2H), 7.70-7.67 (m, 4H), 6.68 (s, 1H), 4.08 (s, 1H), 3.60 (t, J = 27.2 Hz. 2H), 3.11 (d, J = 16.0 Hz, 2H), 1.99 (s, 3H), 1.80 (d, J = 10.4 Hz, 2H), 1.56 (d, J = 18.0 Hz, 2H). |
| 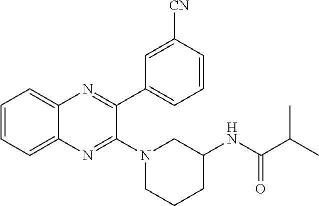 [SMILES: CC(C)C(=O)NC1CCCN(c2nc3ccccc3nc2-c2cccc(C#N)c2)C1] | 220 | 15.1% yield | LC MS of 220: Ret. Time: 1.222 min, MS (ESI) m/z: 400.3, M + H⁺. ¹H NMR of 220: ¹H NMR (400 MHz, CDCl₃) δ 8.24 (d, J = 18.4 Hz, 2H), 7.99 (d, J = 22.0 Hz, 2H), 7.81-7.53 (m, 4H), 6.51 (s, 1H), 4.11 (s, 1H), 3.60 (s, 2H), 3.14 (s, 2H), 2.33 (s, 1H), 1.82 (s, 2H), 1.64 (s, 1H), 1.52 (s, 1H), 1.13 (s, 6H). |
| 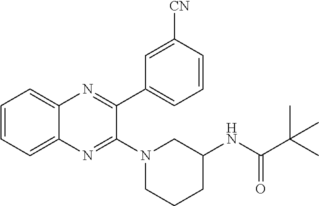 [SMILES: CC(C)(C)C(=O)NC1CCCN(c2nc3ccccc3nc2-c2cccc(C#N)c2)C1] | 221 | 29.2% yield | LC MS of 221: Ret. Time: 1.280 min, MS (ESI) m/z: 414.3, M + H⁺. ¹H NMR of 221: ¹H NMR (400 MHz, CDCl₃) δ 8.31-8.15 (m, 2H), 7.99 (d, J = 8.4 Hz, 1H), 7.88 (d, J = 8.0 Hz, 1H), 7.80-7.51 (m, 4H), 6.43 (s, 1H), 4.10 (s, 1H), 3.57 (t, J = 15.2 Hz, 2H), 3.20- 2.98 (m, 2H), 1.79 (s, 2H), 1.59 (s, 1H), 1.49 (s, 1H), 1.15 (s, 9H). |
| 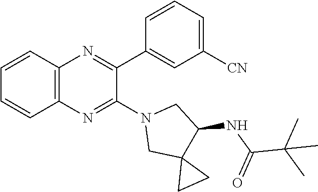 [SMILES: CC(C)(C)C(=O)N[C@@H]1CN(c2nc3ccccc3nc2-c2cccc(C#N)c2)CC12CC2] | 281 | 11.1% yield | LC MS of 281: Ret. Time: 1.294 min, MS (ESI) m/z: 426.1, M + H⁺. ¹H NMR of 281: ¹H NMR (400 MHz, DMSO) 8 8.16 (s, 1H), 8.03 (d, J = 7.6 Hz, 1H), 7.96 (d. J = 7.6 Hz, 1H), 7.89 (d, J = 7.2 Hz, 1H), 7.78-7.70 (m, 2H), 7.69- 7.62 (m, 1H), 7.51-7.43 (m, 1H), 7.32 (d, J = 8.0 Hz, 1H), 4.20 (d, J = 7.2 Hz, 1H), 3.60-3.56 (m, 1H), 3.39 (dd, J = 11.2, 5.6 Hz, 1H), 3.27 (d, J = 10.8 Hz, 1H), 3.17 (d, J = 10.8 Hz, 1H), 1.06 (s, 9H), 0.75- 0.60 (m, 1H). 0.46 (dd, J = 9.2. 4.8 Hz, |
| | | | 2H), 0.40-0.33 (m, 1H). |

Compound 005 according to the present disclosure was prepared as shown in Scheme 14 by an analogous procedure to the one described in Examples 2A-2C.

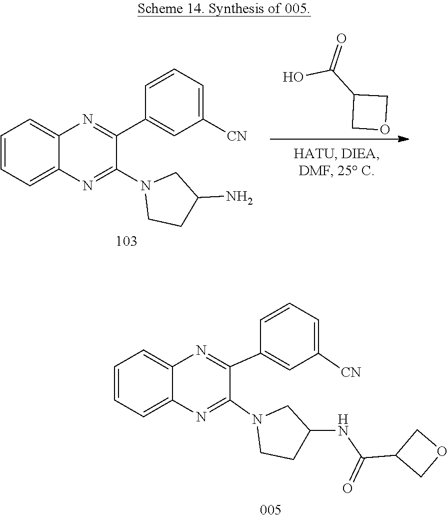 [SMILES: N#Cc1cccc(-c2nc3ccccc3nc2N2CCC(N)C2)c1 ; O=C(O)C1COC1 ; N#Cc1cccc(-c2nc3ccccc3nc2N2CCC(NC(=O)C3COC3)C2)c1]

Compound 011 according to the present disclosure was prepared as shown in Scheme 15 by an analogous procedure to the one described in Examples 2A-2C.

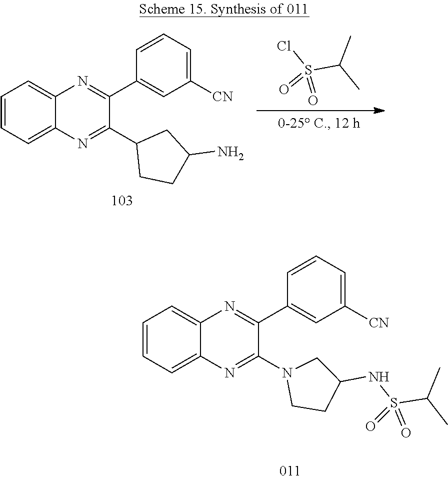

Compound 219 according to the present disclosure was prepared as shown in Scheme 16 by an analogous procedure to the one described in Examples 2A-2C.

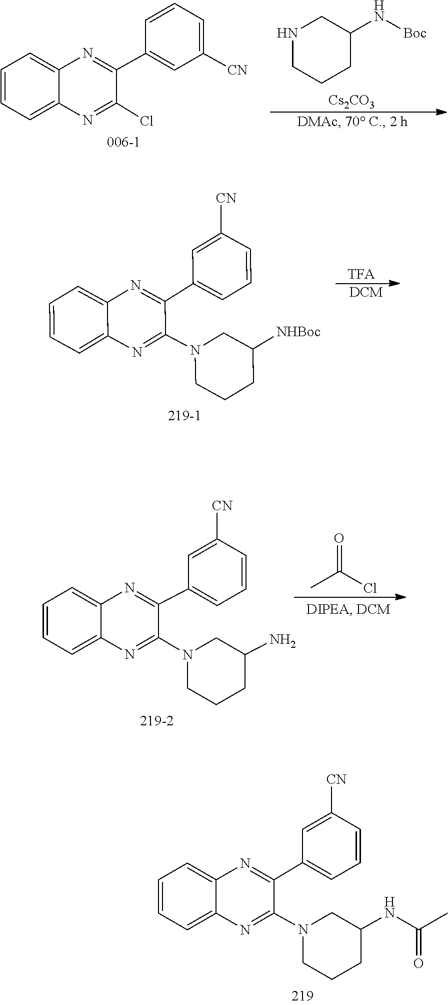

Compound 220 according to the present disclosure was prepared as shown in Scheme 17 by an analogous procedure to the one described in Examples 2A-2C.

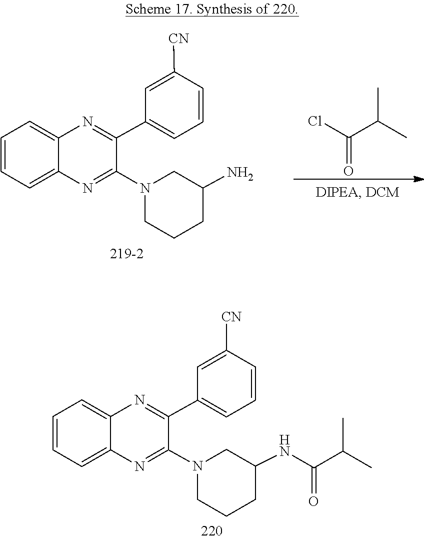

Compound 221 according to the present disclosure was prepared as shown in Scheme 18 by an analogous procedure to the one described in Examples 2A-2C.

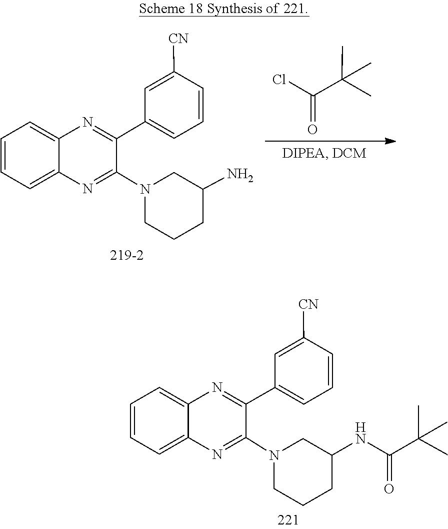

Compound 281 according to the present disclosure was prepared as shown in Scheme 19 by an analogous procedure to the one described in Examples 2A-2C.

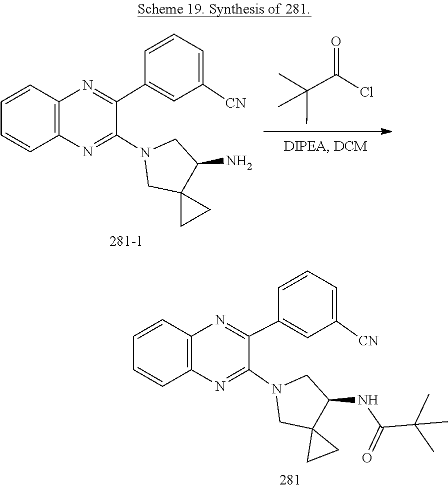

### Example 3: Synthesis of Analogs 010 and 214, 222, 223 According to the Invention

Compound 010 according to the present disclosure was prepared as shown in Scheme 20 and described below.

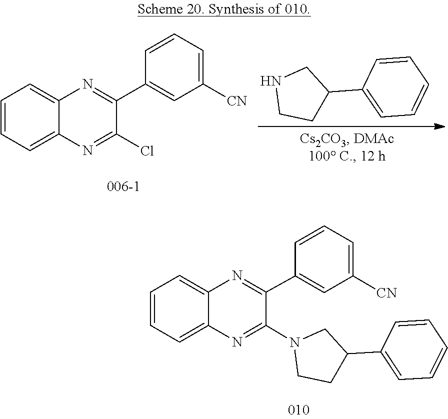 [SMILES: N#Cc1cccc(-c2nc3ccccc3nc2Cl)c1 ; c1ccc(C2CCNC2)cc1 ; N#Cc1cccc(-c2nc3ccccc3nc2N2CCC(c3ccccc3)C2)c1]

### Example 3A: Preparation of Compound 010

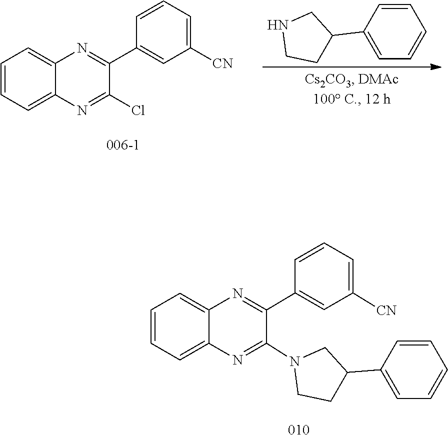 [SMILES: N#Cc1cccc(-c2nc3ccccc3nc2Cl)c1 ; c1ccc(C2CCNC2)cc1 ; N#Cc1cccc(-c2nc3ccccc3nc2N2CCC(c3ccccc3)C2)c1]

To a 50 mL round-bottom flask equipped with a magnetic stir bar and a reflux condenser was added 3-phenylpyrrolidine (80 mg, 543 umol, 1.2 eq) followed by the addition of DMAc (10 mL). Then Cs₂CO₃ (295 mg, 905 umol, 2 eq) and 3-(3-chloroquinoxalin-2-yl)benzonitrile 006-1 (124 mg, 452 umol, 96.6% purity, 1 eq) were added into the mixture dropwise at 25° C. The mixture was heated to 100° C. and stirred for 12 h. The suspension was filtered through a pad of filter paper. The crude product was purified by preparative HPLC: (Phenomenex Synergi C18 column (150×25 mm, 10 um); flow rate: 25 mL/min; gradient: 61%-91% B over 10 min; mobile phase A: 0.1% aqueous trifluoroacetic acid, mobile phase B: acetonitrile). After lyophilization, 3-[3-(3-phenylpyrrolidin-1-yl)quinoxalin-2-yl]benzonitrile 010 (65 mg, 37% yield, 97.8% purity, TFA salt) was obtained as a yellow solid. LCMS of 010: Ret. Time: 1.081 min, MS (ESI) m/z: 377.2, M+H⁺. ¹H NMR of 010: (400 MHz, DMSO-d₆) δ 8.16 (s, 1H), 8.03 (d, J=7.8 Hz, 1H), 7.95-7.87 (m, 2H), 7.72 (q, J=7.6 Hz, 2H), 7.68-7.62 (m, 1H), 7.49-7.42 (m, 1H), 7.34-7.28 (m, 2H), 7.27-7.21 (m, 3H), 3.65 (br dd, J=6.8, 10.2 Hz, 1H), 3.40-3.26 (m, 4H), 2.21 (dt, J=2.8, 5.8 Hz, 1H), 2.01-1.86 (m, 1H).

### Example 3B: Preparation of Analogous Compounds 214, 222, and 223

Table 3, below, depicts exemplary compounds synthesized according to an analogous procedure to the one described for 010.

TABLE 3
Compounds synthesized analogously to 010.

| Compound Structure | Compound No. | Yield | Characterization |
|---|---|---|---|
| 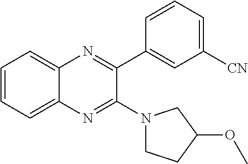 [SMILES: COC1CCN(c2nc3ccccc3nc2-c2cccc(C#N)c2)C1] | 214 | 22.5% yield | LCMS of 214: Ret. Time: 1.245 min, MS (ESI) m/z: 331.1, M + H⁺. ¹H NMR of 214: ¹H NMR (400 MHz, CDCl₃) δ 8.08 (s, 1H), 7.94 (dd, J = 15.6, 8.0 Hz, 2H), 7.81 (d, J = 8.0 Hz, 1H), 7.72 (d, J = 7.6 Hz, 1H), 7.66-7.52 (m. 2H), 7.44 (t. J = 7.6 Hz, 1H), 3.95 (s, 1H), 3.47 (dd, J = 14.4, 9.2 Hz, 2H), 3.32-3.28 (m, 5H), 2.04 (s, 1H), 1.93- 1.91 (m, 1H). |
| 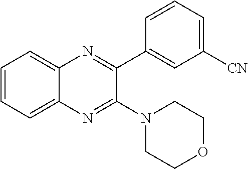 [SMILES: N#Cc1cccc(-c2nc3ccccc3nc2N2CCOCC2)c1] | 222 | 3.2% yield | LCMS of 222: Ret. Time: 1.211 min, MS (ESI) m/z: 317.1, M + H⁺. ¹H NMR of 222: ¹H NMR (400 MHz, CDCl₃) δ 8.40 (s, 1H), 8.33 (d, J = 8.0 Hz, 1H), 7.99 (d. J = 8.4 Hz, 1H), 7.87 (d, J = 8.4 Hz, 1H), 7.75 (d, J = 7.6 Hz, 1H), 7.72-7.50 (m, 3H), 3.74 (s, 4H), 3.27 (s, 4H). |
| 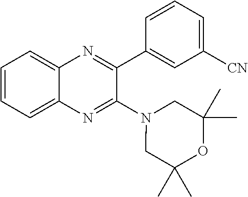 [SMILES: CC1(C)CN(c2nc3ccccc3nc2-c2cccc(C#N)c2)CC(C)(C)O1] | 223 | 13.7% yield | LC MS of 223: Ret. Time: 1.471 min, MS (ESI) m/z: 373.3, M + H⁺. ¹H NMR of 223: ¹H NMR (400 MHz, DMSO) δ 8.28 (s, 1H), 8.14 (d, J = 8.0 Hz, 1H), 7.98 (dd, J = 7.6, 4.0 Hz, 2H), 7.89 (d, J = 7.6 Hz, 1H), 7.77 (s, 2H), 7.70-7.59 (m, 1H), 2.97 (s, 4H), 1.07 (s, 12H). |

Compound 214 according to the present disclosure was prepared as shown in Scheme 21 by an analogous procedure to the one described in Example 3A.

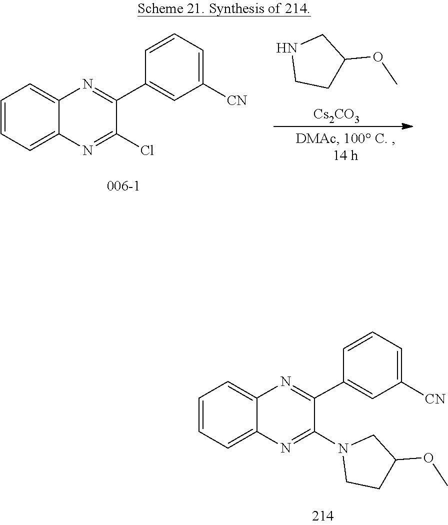

Compound 222 according to the present disclosure was prepared as shown in Scheme 22 by an analogous procedure to the one described in Example 3A.

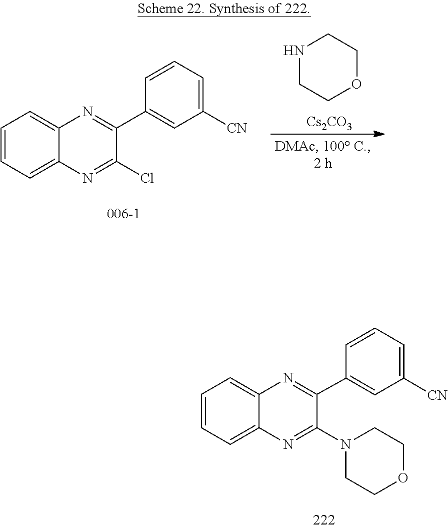

Compound 223 according to the present disclosure was prepared as shown in Scheme 23 by an analogous procedure to the one described in Example 3A.

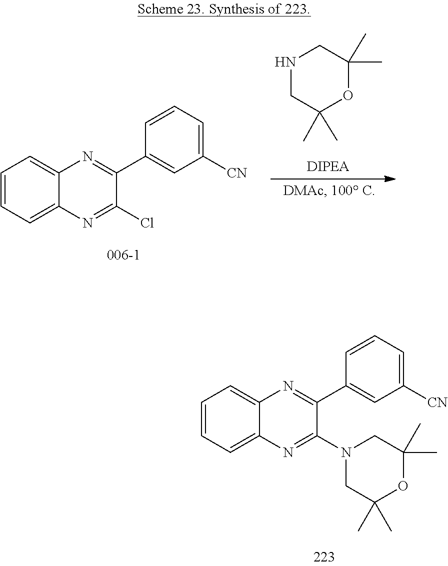

### Example 4: Synthesis of Analogs 012 and 014, 015, 016, 017, 018, 107, 126, 127, 226, 227, 229, 239, 260, 261, 262, 263, 264, 265, 266, 267, 268, 269, 270, 271, 272, 273, 274, 275, 276, 277, 278, 279, 280, 298, 406

Compounds 107 and 012 according to the present disclosure were prepared as shown in Scheme 24 and described below.

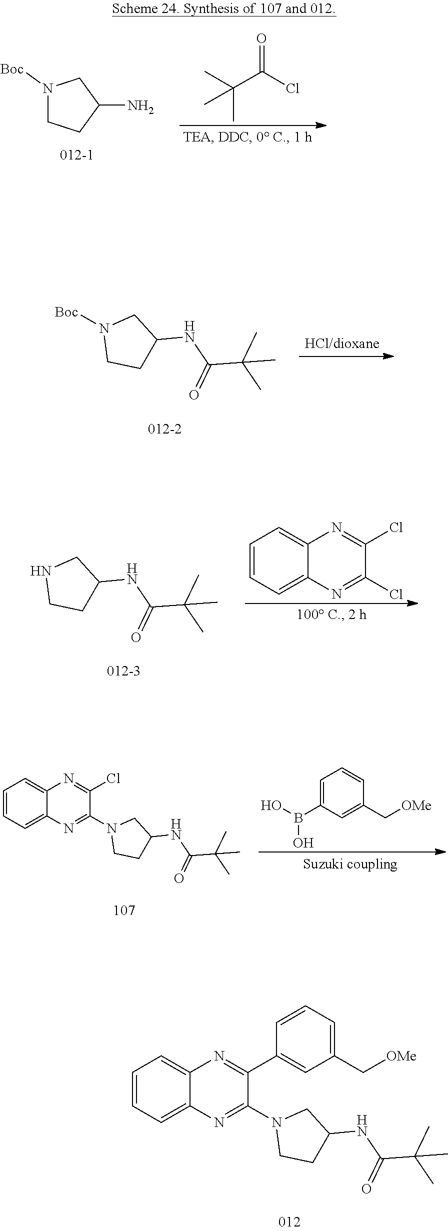

### Example 4A: Preparation of Compound 012-2

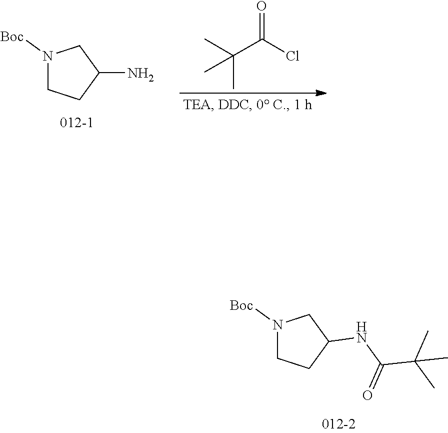

To a 250 mL round-bottom flask equipped with a magnetic stir bar was added tert-butyl 3-aminopyrrolidine-1-carboxylate 012-1 (10 g, 53.69 mmol, 1 eq) followed by the addition of DCM (70 mL). Then TEA (8.15 g, 80.5 mmol, 1.5 eq) and 2,2-dimethylpropanoyl chloride (7.77 g, 64.4 mmol, 1.2 eq) was added into the mixture at 0° C. The mixture was stirred at 0° C. for 1 h. The TLC (petroleum ether/ethyl acetate: 0/1) showed the reaction had completed. The mixture was quenched by slow addition of H₂O (100 mL). The resulting mixture was transferred to a separatory funnel, and the aqueous layer mixture was extracted with ethyl acetate (40 mL×2). The combined organic layers were concentrated under reduced pressure to afford a yellow oil. The crude product was purified by silica gel column chromatography (petroleum ether/ethyl acetate, from I/O to 2/1) to give tert-butyl 3-(2,2-dimethylpropanoylamino)pyrrolidine-1-carboxylate 012-2 (13.4 g, 92% yield, 100% purity) as a colorless oil. LC MS of 012-2: Ret. Time: 0.800 min, MS (ESI) m/z: 171.2, M+H⁺;

### Example 4B: Preparation of Compound 012-3

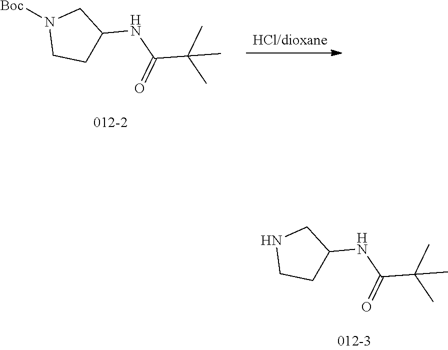 [SMILES: CC(C)(C)OC(=O)N1CCC(NC(=O)C(C)(C)C)C1 ; CC(C)(C)C(=O)NC1CCNC1]

To a 50 mL round-bottom flask equipped with a magnetic stir bar was added tert-butyl 3-(2,2-dimethylpropanoylamino)pyrrolidine-1-carboxylate 012-2 (3 g, 11.1 mmol, 100% purity, 1 eq) followed by the addition of dioxane (20 mL). Then HCl/dioxane (4 M, 10 mL, 3.60 eq) was added into the mixture at 25° C. The mixture was stirred at 25° C. for 3 h. The TLC (petroleum ether/ethyl acetate: 0/1) showed the reaction had completed. The pH of mixture was adjusted to pH 9 using saturated aqueous sodium bicarbonate. The resulting mixture was transferred to a separatory funnel, and the aqueous layer was extracted with ethyl acetate (15 mL×2). The combined organic layers were concentrated under reduced pressure to afford a residue as a white solid. The crude product was triturated from ethyl acetate (100 mL) at 25° C. by stirring for 1 h. After filtration and drying under vacuum, 2,2-dimethyl-N-pyrrolidin-3-yl-propanamide 012-3 (310 mg, 16.41% yield) was obtained as a white solid.

### Example 4C: Preparation of Compound 107

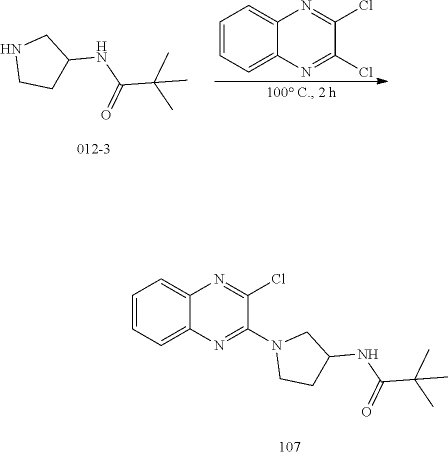

To a 50 mL round-bottom flask equipped with a magnetic stir bar and a reflux condenser was added 2,2-dimethyl-N-pyrrolidin-3-yl-propanamide 012-3 (180 mg, 1.06 mmol, 1 eq) followed by the addition of dioxane (4 mL). Then Cs₂CO₃ (688 mg, 2.11 mmol, 2 eq) was added followed by dropwise addition of 2,3-dichloroquinoxaline (210 mg, 1.06 mmol, 1 eq) at 25° C. The mixture was heated to 100° C. and stirred for 2 h. The TLC (petroleum ether/ethyl acetate: 3/1) showed the reaction was completed. The mixture was cooled to room temperature and quenched by slow addition of H₂O (30 mL). The resulting mixture was transferred to a separatory funnel, and the aqueous layer mixture was extracted with ethyl acetate (20 mL×3). The combined organic layers were washed with brine (20 mL), dried over anhydrous sodium sulfate, filtered, and concentrated under reduced pressure to afford a residue as a yellow oil. The crude product was purified by silica gel column chromatography (petroleum ether/ethyl acetate, from 10/1 to 5/1) to give N-[1-(3-chloroquinoxalin-2-yl)pyrrolidin-3-yl]-2,2-dimethyl-propanamide 107 (351 mg, 97% yield, 97.5% purity) as a yellow solid. LC MS of 107: Ret. Time: 0.958 min, MS (ESI) m/z: 333.3, M+H⁺. ¹H NMR of 107: (400 MHz, DMSO-d₆) δ 7.80-7.75 (m, 1H), 7.69-7.61 (m, 2H), 7.56 (br d, J=6.4 Hz, 1H), 7.44 (ddd, J=1.8, 6.8, 8.4 Hz, 1H), 4.36-4.32 (m, 1H), 3.98 (dd, J=6.6, 11.4 Hz, 1H), 3.88-3.78 (m, 2H), 3.70 (dd, J=5.4, 11.2 Hz, 1H), 2.11 (qd, J=6.4, 12.5 Hz, 1H), 1.98-1.89 (m, 1H), 1.10 (s, 9H).

### Example 4D: Preparation of Compound 012

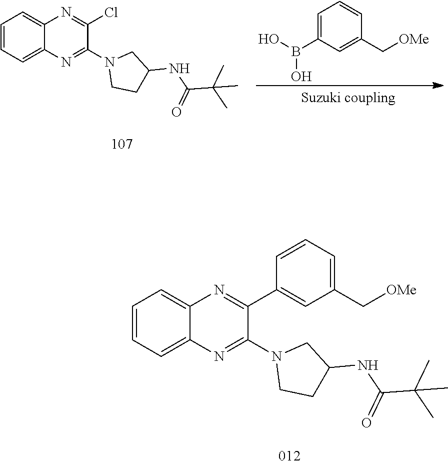 [SMILES: CC(C)(C)C(=O)NC1CCN(c2nc3ccccc3nc2Cl)C1 ; COCc1cccc(B(O)O)c1 ; COCc1cccc(-c2nc3ccccc3nc2N2CCC(NC(=O)C(C)(C)C)C2)c1]

To a 10 mL round-bottom flask equipped with a magnetic stir bar and a reflux condenser was added N-[1-(3-chloroquinoxalin-2-yl)pyrrolidin-3-yl]-2,2-dimethyl-propanamide 107 (70 mg, 206 μmol, 98% purity, 1 eq) followed by the addition of dioxane (3 mL). Then [3-(methoxymethyl)phenyl]boronic acid (41 mg, 247 umol, 1.2 eq), Pd(dppf)Cl₂ (15 mg, 20.6 umol, 0.1 eq) and KOAc (40.46 mg, 412 umol, 2 eq) were added into the mixture at 25° C. The flask was then evacuated and backfilled with nitrogen three times. The mixture was heated to 70° C. and stirred for 2 h. The mixture was cooled to room temperature and quenched by slow addition of H₂O (30 mL). The resulting mixture was transferred to a separatory funnel, and the aqueous layer mixture was extracted with ethyl acetate (20 mL×3). The combined organic layers were washed with brine (20 mL), dried over anhydrous sodium sulfate, filtered, and concentrated under reduced pressure to afford a residue as a yellow oil. The crude product was purified by preparative HPLC: (Phenomenex Synergi C18 column (150×25 mm, 10 um); flow rate: 25 mL/min; gradient: 40% -70% B over 7 min; mobile phase A: 0.1% aqueous TFA, mobile phase B: acetonitrile). N-(1-(3-(3-(Methoxymethyl)phenyl)quinoxalin-2-yl)pyrrolidin-3-yl)pivalamide 012 (81.9 mg, 72% yield, 96.50 purity, TFA salt) was obtained as a yellow solid. LC MS of 012: Ret. Time: 0.923 min, MS (ESI) m/z: 419.2, M+H⁺. ¹H NMR of 012: (400 MHz, DMSO-d₆) δ 7.86 (dd, J=1.0, 8.4 Hz, 1H), 7.71 (dd, J=1.0, 8.4 Hz, 1H), 7.66-7.57 (i, 3H), 7.48 (t, J=7.6 Hz, 1H), 7.45-7.38 (, 3H), 4.51 (s, 2H), 4.20-4.17 (m, 1H), 3.44 (dd, J=6.6, 11.4 Hz, 15H), 3.32 (s, 3H), 3.29-3.23 (, 1H), 3.23-3.13 (m, 2H), 2.01-1.88 (m, 1H), 1.86-1.74 (m, 3H), 1.05 (s, 9H)

### Example 4E: Preparation of Analogous Compounds 014, 015, 016, 017, 018, 107, 126, 127, 226, 227, 229, 239, 260, 261, 262, 263, 264, 265, 266, 267, 268, 269, 270, 271, 272, 273, 274, 275, 276, 277, 278, 279, 280, 298, and 406

Table 4, below, depicts exemplary compounds synthesized according to an analogous procedure to the one described for 012.

TABLE 4
Compounds synthesized analogously to 012.

| Compound Structure | Compound No. | Yield/Purity | Characterization |
|---|---|---|---|
| 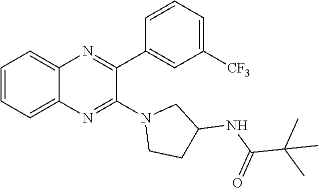 [SMILES: CC(C)(C)C(=O)NC1CCN(c2nc3ccccc3nc2-c2cccc(C(F)(F)F)c2)C1] | 014 | 31% yield, 95.5% purity | LC MS of 014: Ret. Time: 1.023 min, MS (ESI) m/z: 443.2, M + H⁺; ¹H NMR of 014: (400 MHz, DMSO-d₆) δ 8.05 (s, 1H), 7.99 (d, J = 7.6 Hz. 1H), 7.92-7.88 (m, 1H), 7.85 (br d, J = 7.8 Hz, 1H), 7.79-7.71 (m, 2H), 7.64 (dt, J = 1.4, 7.6 Hz, 1H), 7.49-7.39 (m. 2H), 4.24- 4.15 (m, 1H), 3.48 (br s, 1H), 3.25-3.13 (m, 3H), 2.02-1.88 (m, 1H), 1.87-1.75 (m, 1H), 1.05 (s, 9H) |
| 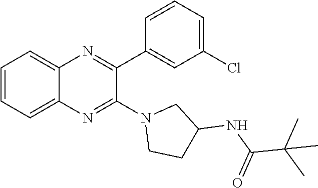 [SMILES: CC(C)(C)C(=O)NC1CCN(c2nc3ccccc3nc2-c2cccc(Cl)c2)C1] | 015 | 50% yield, 100% purity, TFA salt | LC MS of 015: Ret. Time: 0.996 min, MS (ESI) m/z: 409.2, M + H⁺; ¹H NMR of 015: (400 MHz, DMSO-d₆) δ 7.88 (dd, J = 1.0, 8.2 Hz, 1H), 7.78-7.70 (m. 2H), 7.67-7.60 (m, 2H), 7.57-7.51 (m, 2H), 7.47-7.40 (m, 2H), 4.20 (br d, J = 6.0 Hz, 1H), 3.46 (dd, J = 6.4, 11.2 Hz, 1H), 3.33-3.24 (m, 1H), 3.24-3.16 (m, 2H), 1.95 (qd, J = 6.4, 12.6 Hz, 1H), 1.88-1.77 (m, 1H), 1.06 (s, 9H) |
| 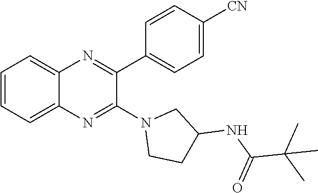 [SMILES: CC(C)(C)C(=O)NC1CCN(c2nc3ccccc3nc2-c2ccc(C#N)cc2)C1] | 016 | 92% yield, 97.8% purity, TFA salt | LC MS of 016: Ret. Time: 0.862 min, MS (ESI) m/z: 400.2, M + H⁺; ¹H NMR of 016: (400 MHz, DMSO-d₆) δ 8.01-7.96 (m, 3H), 7.88 (d, J = 8.4 Hz, 3H), 7.77-7.70 (m, 1H), 7.65 (dt, J = 1.6, 7.6 Hz, 1H), 7.49-7.36 (m, 2H), 4.24- 4.13 (m, 1H), 3.42 (dd, J = 6.4, 11.2 Hz. 1H), 3.32-3.18 (m, 2H), 3.18-3.10 (m, 1H), 1.96 (qd, J = 6.4, 12.4 Hz, 1H), 1.88- 1.72 (m. 1H), 1.05 (s. 9H) |
| 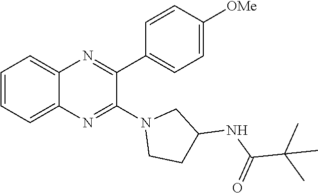 | 017 | 43% yield, 98.7% purity, TFA salt | LC MS of 017: Ret. Time: 1.072 min, MS (ESI) m/z: 372.2, M + H⁺; ¹H NMR of 017: (400 MHz, DMSO-d₆) δ 7.84 (dd, J = 1.0, 8.2 Hz, 1H), 7.70 (dd, J = 1.0, 8.3 Hz, 1H), 7.68-7.63 (m, 2H), 7.59 (ddd, J = 1.4, 6.9, 8.3 Hz, 1H), 7.46- 7.37 (m, 2H), 7.11-7.00 (m. 2H), 4.23- 4.15 (m, 1H), 3.83 (s, 3H), 3.44 (dd, J = 6.5, 11.2 Hz, 1H), 3.34-3.16 (m, 3H), 1.96 (qd, J = 6.3, 12.6 Hz, 1H), 1.87- 1.74 (m, 1H), 1.05 (s, 9H) |
| 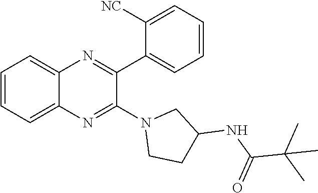 [SMILES: CC(C)(C)C(=O)NC1CCN(c2nc3ccccc3nc2-c2ccccc2C#N)C1] | 018 | 12% yield, 99.0% purity, TFA salt | LC MS of 018: Ret. Time: 0.933 min, MS (ESI) m/z: 400.2, M + H⁺; ¹H NMR of 018: (400 MHz, DMSO-d₆) δ 78.03-7.99 (m, 1H), 7.90-7.82 (m. 2H), 7.82-7.78 (m, 1H), 7.77-7.73 (m, 1H), 7.72-7.65 (m, 2H), 7.46 (ddd, J = 1.6, 6.8, 8.4 Hz, 1H), 7.40 (br d, J = 6.4 Hz, 1H), 4.24-4.15 (m, 1H), 3.31-3.24 (m, 2H), 3.21-3.16 (m, 1H), 3.13-3.05 (m, 1H), 1.96 (td, J = 6.0. 12.4 Hz, 1H), 1.89- 1.78 (m, 1H), 1.04 (s, 9H) |
| 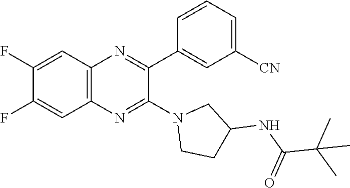 [SMILES: CC(C)(C)C(=O)NC1CCN(c2nc3cc(F)c(F)cc3nc2-c2cccc(C#N)c2)C1] | 126 | 64% yield, 99.5% purity, TFA salt | LC MS of 126: Ret. Time: 1.002 min, MS (ESI) m/z: 436.1, M + H⁺; ¹H NMR of 126: (400 MHz, DMSO-d₆) δ 8.11 (s, 1H), 8.02-7.89 (m, 3H), 7.76- 7.67 (m, 2H), 7.43 (d, J = 6.4 Hz, 1H), 4.26-4.15 (m, 1H), 3.44 (br d, J = 4.8 Hz, 1H), 3.27-3.04 (m, 3H), 1.95 (qd, J = 6.4, 12.4 Hz, 1H), 1.87-1.76 (m, 1H), 1.06 (s, 9H). |
| 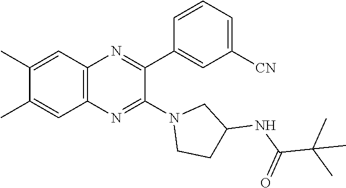 [SMILES: Cc1cc2nc(-c3cccc(C#N)c3)c(N3CCC(NC(=O)C(C)(C)C)C3)nc2cc1C] | 127 | 63.5% yield, 96.2% purity, TFA salt | LC MS of 127: Ret. Time: 0.979 min, MS (ESI) m/z: 428.2. M + H⁺; ¹H NMR of 127: (400 MHz, DMSO-d₆) δ 8.10 (s, 1H), 8.01 (d, J = 8.0 Hz, 1H), 7.93 (d, J = 7.6 Hz, 1H), 7.71 (t, J = 7.6 Hz, 1H), 7.65 (s, 1H), 7.54 (s, 1H), 7.42 (br d, J = 6.0 Hz, 1H), 4.20 (br dd, J = 6.0. 12.4 Hz, 1H), 3.42 (dd, J = 6.4, 10.8 Hz, 1H), 3.25-3.09 (m, 3H), 2.39 (d, J = 13.2 Hz, 6H), 2.00-1.88 (m, 1H), 1.86-1.75 (m, 1H), 1.06 (s, 9H). |
| 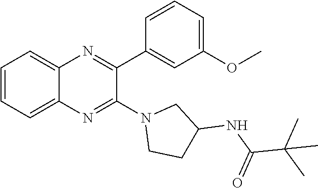 [SMILES: COc1cccc(-c2nc3ccccc3nc2N2CCC(NC(=O)C(C)(C)C)C2)c1] | 226 | 36.8% yield, 99.1% purity | LC MS of 226: Ret. Time: 1.408 min, MS (ESI) m/z: 405.3. M + H⁺. ¹H NMR of 226: ¹H NMR (400 MHz, DMSO) δ 7.85 (d, J = 8.4 Hz, 1H), 7.70 (d, J = 8.4 Hz, 1H), 7.61 (t, J = 7.6 Hz, 1H), 7.41 (t, J = 7.6 Hz, 3H), 7.23 (d, J = 7.2 Hz, 2H), 7.04 (d, J = 6.0 Hz, 1H), 4.23 -4.14 (m, 1H), 3.82 (s, 3H), 3.50-3.41 (m, 1H), 3.25-3.17 (m, 2H), 1.98-1.74 (m, 2H), 1.05 (s, 9H). |
| 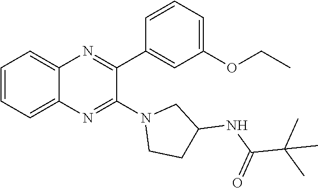 [SMILES: CCOc1cccc(-c2nc3ccccc3nc2N2CCC(NC(=O)C(C)(C)C)C2)c1] | 227 | 33.0% yield | LC MS of 227: MS (ESI) m/z: 419.3, M + H⁺. ¹H NMR of 227: ¹H NMR (400 MHz, DMSO) δ 7.85 (d, J = 8.4 Hz, 1H), 7.70 (d, J = 8.4 Hz, IH), 7.60 (t, J = 8.4 Hz, 1H), 7.41 (dt, J = 12.8, 5.6 Hz, 3H), 7.23- 7.18 (m, 2H), 7.03 (dd, J = 8.0, 2.4 Hz, 1H), 4.18 (dd, J = 12.0, 6.0 Hz, IH), 4.09 (m, .J = 7.2 H7., 2H), 3.46 (dd, J = 11.2, 6.4 Hz, 1H), 3.21 (dd, J = 11.2, 5.6 Hz, 2H), 1.87 (m, J = 52.0, 12.4, 6.0 Hz, 2H), 1.35 (t, J = 7.1 Hz, 3H), 1.05 (s, 9H). |
| 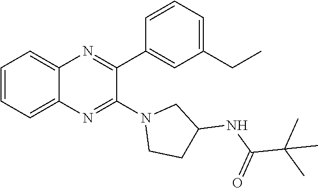 [SMILES: CCc1cccc(-c2nc3ccccc3nc2N2CCC(NC(=O)C(C)(C)C)C2)c1] | 229 | 19.3% yield | LC MS of 229: MS (ESI) m/z: 403.3, M + H⁺. ¹H NMR of 229: ¹H NMR (400 MHz, DMSO) δ 7.85 (d. J = 9.2 Hz, 1H), 7.70 (d, J = 8.4 Hz, 1H), 7.63-7.57 (m, 1H), 7.49 (d, J = 8.8 Hz, 2H), 7.41 (t, J = 7.2 Hz, 3H), 7.32 (d, J = 7.6 Hz. 1H), 4.18 (dd, J = 12.0, 6.0 Hz, 1H), 3.45 (dd, J = 11.2, 6.4 Hz, 1H), 3.19 (dd, J = 11.6, 5.6 Hz, 2H), 2.69 (m, J = 7.6 Hz, 2H), 1.96- 1.75 (m, 2H), 1.23 (t, J = 7.6 Hz, 3H), 1.05 (s, 9H). |
| 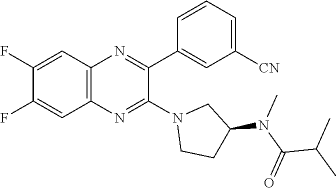 [SMILES: CC(C)C(=O)N(C)[C@H]1CCN(c2nc3cc(F)c(F)cc3nc2-c2cccc(C#N)c2)C1] | 239 | 46.1% yield, 99.9% purity | LC MS of 239: Ret. Time: 1.290 min, MS (ESI) m/z: 436.2. M + H⁺. ¹H NMR of 239: ¹H NMR (400 MHz, DMSO) δ 8.13 (s, 1H), 7.97 (m, J = 16.8, 8.4 Hz, 3H), 7.74 (dd, J = 14.0, 6.4 Hz, 2H), 4.91 (dd, J = 8.8, 1.2 Hz, 1H), 3.42- 3.35 (m. 1H), 3.19 (d. J = 8.8 Hz, 3H), 2.98-2.74 (m, 3H), 2.67 (d, J = 1.6 Hz, 1H), 1.96 (d, J = 1.2 Hz, 2H), 0.97 (d, J = 6.8 Hz, 6H). |
| 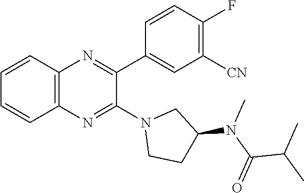 [SMILES: CC(C)C(=O)N(C)[C@H]1CCN(c2nc3ccccc3nc2-c2ccc(F)c(C#N)c2)C1] | 260 | 49.4% yield | LC MS of 260: Ret. Time: 1.140 min, MS (ESI) m/z: 418.2. M + H⁺. ¹H NMR of 260: ¹H NMR (400 MHz, DMSO) δ 8.29-8.22 (m, 1H), 8.15- 8.07 (m, 1H), 7.89 (d, J = 8.0 Hz, 1H), 7.75 (d, J = 8.0 Hz, 1H), 7.71-7.63 (m. 2H), 7.47 (t. J = 7.6 Hz, 1H). 5.02-4.65 (m, 1H), 3.38 (s, 2H), 3.24 (s, 2H), 2.90 (s, 2H), 2.73 (t, J = 12.0 Hz, 2H), 2.07- 1.93 (m, 2H), 0.98 (d. J = 6.4 Hz, 6H). |
| 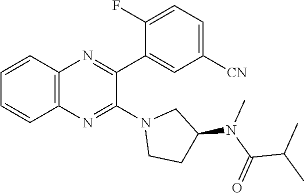 [SMILES: CC(C)C(=O)N(C)[C@H]1CCN(c2nc3ccccc3nc2-c2cc(C#N)ccc2F)C1] | 261 | 29.8% yield | LC MS of 261: MS (ESI) m/z: 418.3, M + H⁺. ¹H NMR of 261: ¹H NMR (400 MHz, DMSO) δ 8.25 (s, 1H), 8.11 (dd, J = 4.8, 2.0 Hz. 1H), 7.97-7.84 (m, 1H), 7.81- 7.59 (m, 3H), 7.47 (dd, J = 12.8, 2.8 Hz, 1H), 5.11-4.60 (m,1H), 4.02-3.34 (m, 2H), 3.25-3.20 (m, 2H), 3.02-2.78 (m, 3H), 2.70 (d, J = 21.6 Hz, 1H), 2.00 (d, J = 10.0 Hz, 2H), 1.00 (dd, J = 17.6, 6.8 Hz, 6H) |
| 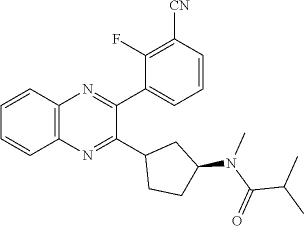 [SMILES: CC(C)C(=O)N(C)[C@H]1CCC(c2nc3ccccc3nc2-c2cccc(C#N)c2F)C1] | 262 | 24.3% yield | LC MS of 262: MS (ESI) m/z: 418.3, M + H⁺. ¹H NMR of 262: ¹H NMR (400 MHz, DMSO) δ 8.18-7.98 (m, 2H), 7.89 (d, J = 8.0 Hz, 1H), 7.80-7.65 (m, 2H), 7.59 (t, J = 7.6 Hz, 1H), 7.52-7.41 (m, 1H), 4.83 (s, 1H), 3.47 (s, 1H), 3.23 (s, 3H), 3.05-2.74 (m, 3H), 2.67 (s, 1H), 2.14- 1.85 (m, 2H), 1.00 (dd, J = 18.0, 6.8 Hz, 6H). |
| 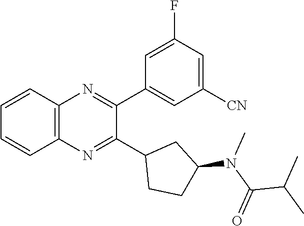 [SMILES: CC(C)C(=O)N(C)[C@H]1CCC(c2nc3ccccc3nc2-c2cc(F)cc(C#N)c2)C1] | 263 | 38.5% yield | LC MS of 263: MS (ESI) m/z: 418.2, M + H⁺. ¹H NMR of 263: ¹H NMR (400 MHz, DMSO) δ 8.06-7.97 (m, 2H), 7.92 (dd, J = 15.2, 8.8 Hz, 2H), 7.75 (d, J = 8.0 Hz, 1H), 7.68 (t, J = 7.2 Hz, 1H), 7.48 (t, J = 7.2 Hz, 1H), 5.11-4.60 (m, 1H), 3.49- 3.36 (m, 1H), 3.25 (s, J = 6.0 Hz, 3H), 3.03-2.74 (m, 3H), 2.70 (s, 1H), 2.10- 1.84 (m. 2H), 0.98 (d. J = 6.4 Hz, 6H). |
| 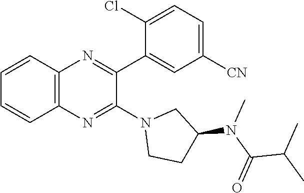 [SMILES: CC(C)C(=O)N(C)[C@H]1CCN(c2nc3ccccc3nc2-c2cc(C#N)ccc2Cl)C1] | 264 | 35.0% yield | LC MS of 264: MS (ESI) m/z: 434.1, M + H⁺. ¹H NMR of 264: ¹H NMR (400 MHz, DMSO) δ 8.24 (d, J = 23.6 Hz, 1H), 8.02 (t, J = 6.8 Hz. 1H), 7.93-7.82 (m, 2H). 7.78-7.64 (m, 2H), 7.46 (t, J = 7.2 Hz, 1H), 4.85 (s, 1H), 3.50 (d, J = 27.2 Hz, 2H), 3.26-3.08 (m, 2H), 2.88 (d, J = 11.2 Hz, 3H), 2.67 (s, 1H), 1.93 (s, 2H), 1.03 (s, 6H). |
| 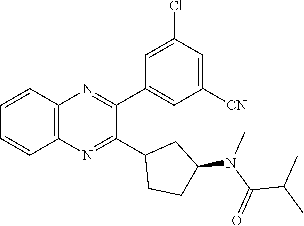 [SMILES: CC(C)C(=O)N(C)[C@H]1CCC(c2nc3ccccc3nc2-c2cc(Cl)cc(C#N)c2)C1] | 265 | 54.9% yield | LC MS of 265: MS (ESI) m/z: 434.2, M + H⁺. ¹H NMR of 265: ¹H NMR (400 MHz, DMSO) δ 8.18 (d, J = 1.6 Hz, 1H), 8.13 (d, J = 1.6 Hz, 2H), 7.91 (d, J = 8.0 Hz, 1H), 7.75 (d, J = 8.0 Hz, IH), 7.68 (t, J = 7.2 Hz, 1H), 7.48 (t, J = 7.2 Hz, 1H), 5.10-4.61 (m, 1H), 3.42 (d, J = 8.8 Hz, 1H), 3.25 (s. 3H), 3.02-2.75 (m, 3H), 2.74-2.66 (m, 1H), 2.10-1.89 (m, 2H), 0.99 (d, J = 6.4 Hz, 6H). |
| 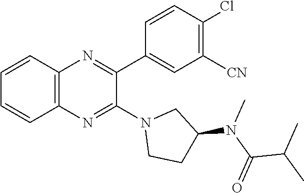 [SMILES: CC(C)C(=O)N(C)[C@H]1CCN(c2nc3ccccc3nc2-c2ccc(Cl)c(C#N)c2)C1] | 266 | 60.2% yield | LC MS of 266: MS (ESI) m/z: 434.2, M + H⁺. ¹H NMR of 266: ¹H NMR (400 MHz, DMSO) δ 8.30 (d, J = 1.6 Hz, 1H), 8.06 (dd, J = 8.4, 2.0 Hz, 1H), 7.90 (dd, J = 8.0, 2.8 Hz, 2H), 7.75 (d, J = 8.0 Hz. 1H), 7.67 (t, J = 7.2 Hz. 1H), 7.48 (t, J = 7.2 Hz, 1H), 4.93 (s, 1H), 3.40 (s, 1H), 3.24 (d, J = 6.4 Hz, 3H), 3.01-2.77 (m, 3H), 2.70 (s, 1H), 1.98 (d, 6.0 Hz, 2H), 0.99 (d, J = 6.4 Hz, 6H). |
| 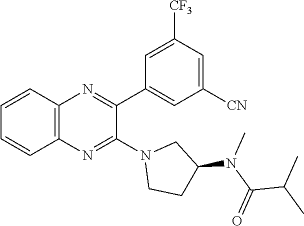 [SMILES: CC(C)C(=O)N(C)[C@H]1CCN(c2nc3ccccc3nc2-c2cc(C#N)cc(C(F)(F)F)c2)C1] | 267 | 14.4% yield | LC MS of 267: MS (ESI) m/z: 468.1, M + H⁺. ¹H NMR of 267: ¹H NMR (400 MHz, DMSO) δ 8.48 (s, 2H), 8.40 (s, 1H), 7.93 (d, J = 8.0 Hz, 1H), 7.77 (d, J = 8.4 Hz, 1H), 7.69 (1, J = 7.6 Hz, 1H), 7.49 (t, J = 7.2 Hz, 1H), 4.85 (s, 1H), 3.41 (dd. J = 11.2, 7.6 Hz, 2H), 3.21 (dd, J = 11.2, 7.2 Hz, 2H), 2.86 (d, J = 26.4 Hz, 3H), 2.69 (s, 1H), 1.98 (s, 2H), 0.98 (d. J = 6.4 Hz, 6H). |
| 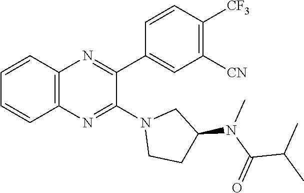 [SMILES: CC(C)C(=O)N(C)[C@H]1CCN(c2nc3ccccc3nc2-c2ccc(C(F)(F)F)c(C#N)c2)C1] | 268 | 13.8% yield | LC MS of 268: MS (ESI) m/z: 468.1, M + H⁺. ¹H NMR of 268: ¹H NMR (400 MHz, DMSO) δ 8.49 (s, 1H), 8.20 (d, J = 8.2 Hz. 2H), 7.92 (d, J = 8.0 Hz, 1H). 7.83- 7.65 (m, 2H), 7.49 (t, J = 7.6 Hz, 1H), 4.84 (s, 1H), 3.40 (s, 2H), 3.24 (s, 1H), 2.86 (d, J = 28.0 Hz, 3H), 2.68 (d, J = 10.8 Hz, 2H), 1.99 (s, 2H), 0.98 (d, J = 5.6 Hz, 6H). |
| 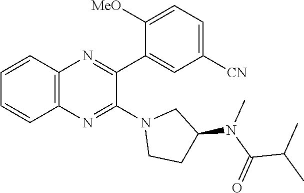 [SMILES: COc1ccc(C#N)cc1-c1nc2ccccc2nc1N1CC[C@H](N(C)C(=O)C(C)C)C1] | 269 | 28.9% yield | LC MS of 269: MS (ESI) m/z: 430.1, M + H⁺. ¹H NMR of 269: ¹H NMR (400 MHz, DMSO) δ 7.96 (dd, J = 20.4, 10.0 Hz, 2H), 7.83 (d, J = 7.6 Hz, 1H), 7.74-7.56 (m. 2H), 7.42 (t, J = 7.6 Hz, IH), 7.33 (d, J = 8.8 Hz, 1H), 5.05-4.51 (m, 1H), 3.83 (d, J = 8.4 Hz, 3H), 3.59 (s, 2H), 3.12 (d, J = 7.2 Hz, 2H), 3.01-2.74 (m, 3H), 2.67 (dd, J = 10.4, 8.4 Hz, 1H), 2.12-1.79 (m, 2H), 0.98 (d, J = 6.4 Hz, 6H). |
| 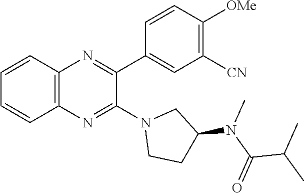 [SMILES: COc1ccc(-c2nc3ccccc3nc2N2CC[C@H](N(C)C(=O)C(C)C)C2)cc1C#N] | 270 | 62.7% yield, 99.9 % purity | LCMS of 270: Ret. Time: 1.197 min, MS (ESI) m/z: 430.1. M + H⁺. ¹H NMR of 270: ¹H NMR (400 MHz, DMSO) δ 8.07 (d, J = 2.4 Hz, 1H), 8.02 (d, J = 8.8 Hz, 1H), 7.88 (d, J = 8.0 Hz, 1H), 7.73 (d, J = 8.4 Hz, 1H), 7.64 (t, J = 7.2 Hz, 1H), 7.46 (t. J = 7.2 Hz, 1H), 7.39 (d, J = 8.8 Hz, 1H), 4.84 (d, J = 18.0 Hz, 1H), 3.98 (d, J = 17.2 Hz, 3H), 3.39 (s, 1H), 3.24 (dd, J = 16.4, 8.0 Hz, 2H), 2.98-2.75 (m, 3H), 2.70 (s, 1H), 2.08-1.87 (m, 2H), 0.98 (d, J = 6.8 Hz, 6H). |
| 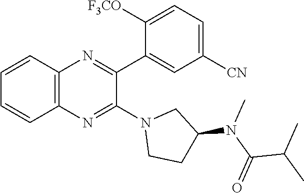 [SMILES: CC(C)C(=O)N(C)[C@H]1CCN(c2nc3ccccc3nc2-c2cc(C#N)ccc2OC(F)(F)F)C1] | 271 | 48.5% yield | LC MS of 271: MS (ESI) m/z: 484.1, M + H⁺. ¹H NMR of 271: ¹H NMR (400 MHz, DMSO) δ 8.39 (s, 1H), 8.16 (dd, J = 8.8, 2.4 Hz, 1H), 7.88 (d, J = 8.0 Hz, 1H), 7.79-7.64 (m, 3H), 7.47 (t, J = 6.8 Hz, 1H). 4.99 (s, 1H), 4.63 (s, 1H), 3.45 (s, 1H), 3.12 (s, 2H), 2.84 (d, J = 16.4 Hz, 3H), 2.66 (d. J = 1.6 Hz, 1H), 1.97 (d, J = 43.2 Hz, 2H), 0.97 (d, J = 6.8 Hz, 6H). |
| 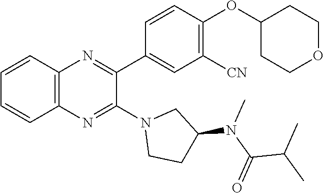 [SMILES: CC(C)C(=O)N(C)[C@H]1CCN(c2nc3ccccc3nc2-c2ccc(OC3CCOCC3)c(C#N)c2)C1] | 272 | 38.5 mg, 25.6% yield | LC MS of 272: MS (ESI) m/z: 500.4, M + H⁺. ¹H NMR of 272: ¹H NMR (400 MHz, DMSO) δ 8.07 (d, J = 2.2 Hz, 1H), 7.98 (d. J = 8.8 Hz, 1H), 7.88 (d, J = 7.6 Hz. 1H), 7.73 (d, J = 8.0 Hz, 1H), 7.67-7.61 (m, 1H), 7.52-7.43 (m, 2H), 5.01 (d, J = 7.6 Hz. 1H), 4.93-4.88 (m, 1H), 3.94- 3.82 (m, 2H), 3.55 (m, 2H), 3.45-3.19 (m, 4H), 2.99-2.77 (m, 3H), 2.70 (s, 1H), 2.12-1.88 (m, 4H), 1.76-1.63 (m, 2H), 0.98 (d, J = 5.6 H7., 6H). |
| 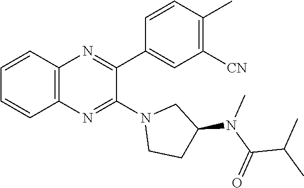 [SMILES: Cc1ccc(-c2nc3ccccc3nc2N2CC[C@H](N(C)C(=O)C(C)C)C2)cc1C#N] | 273 | 52.6% yield | LC MS of 273: MS (ESI) m/z: 414.2, M + H⁺. ¹H NMR of 273: ¹H NMR (400 MHz, DMSO) δ 7.98 (s, 1H), 7.88-7.81 (m, 2H), 7.74 (d, J = 8.0 Hz, 1H), 7.70-7.63 (m, 1H), 7.57 (s, 1H), 7.45 (dd, J = 11.2, 4.0 Hz, 1H), 4.81 (d, J = 13.2 Hz, 1H), 3.39 (s, 1H), 3.16 (d, J = 18.8 Hz, 3H), 2.83 (d, J = 15.6 Hz, 2H), 2.65 (s, 1H), 2.37-2.12 (m, 3H), 1.95 (d, J = 14.0 Hz, 2H), 0.97 (d, J = 6.8 Hz, 6H). |
| 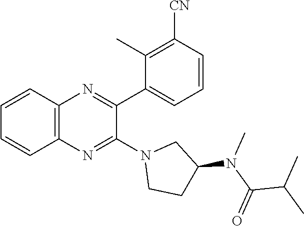 [SMILES: Cc1c(C#N)cccc1-c1nc2ccccc2nc1N1CC[C@H](N(C)C(=O)C(C)C)C1] | 274 | 22.8% yield | LC MS of 274: MS (ESI) m/z: 414.3, M + H⁺. ¹H NMR of 274: ¹H NMR (400 MHz, DMSO) δ 7.93 (d. J = 7.6 Hz, 1H), 7.80 (m, 3H), 7.70-7.63 (m, 1H), 7.54 (d, J = 7.8 Hz, 1H), 7.46 (dd, J = 11.2, 4.0 Hz, 1H), 4.96-4.64 (m, 1H), 3.37 (s, 1H), 3.16 (m, 3H), 3.00-2.73 (m, 3H), 2.65 (s, 1H), 2.35 (m, 3H), 2.05-1.81 (m, 2H), 0.97 (d, J = 6.6 Hz. 6H). |
| 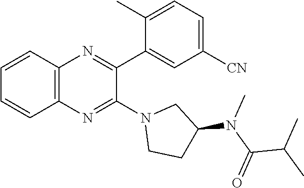 [SMILES: Cc1ccc(C#N)cc1-c1nc2ccccc2nc1N1CC[C@H](N(C)C(=O)C(C)C)C1] | 275 | 45.9% yield | LC MS of 275: MS (ESI) m/z: 414.2, M + H⁺. ¹H NMR of 275: ¹H NMR (400 MHz, DMSO) δ 8.09 (d, J = 1.6 Hz, 1H), 7.90 (dd, J = 14.8, 8.0 Hz, 2H), 7.74 (d, J = 8.4 Hz. 1H), 7.67-7.59 (m, 2H), 7.46 (t, J = 7.2 Hz, 1H), 4.92 (dd, J = 20.8, 13.2 Hz, 1H), 3.39 (d, J = 17.6 Hz, 2H), 3.23 (d, J = 7.2 Hz. 2H), 2.99-2.78 (m, 3H), 2.69 (s, 1H), 2.57 (s, 3H), 1.96 (dd, J = 2.4, 6.4 Hz, 2H), 0.98 (d, J = 6.8 Hz, 6H). |
| 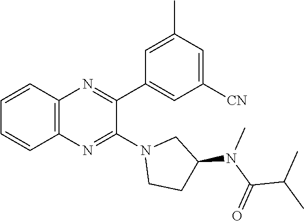 [SMILES: Cc1cc(C#N)cc(-c2nc3ccccc3nc2N2CC[C@H](N(C)C(=O)C(C)C)C2)c1] | 276 | 13.3% yield | LC MS of 276: MS (ESI) m/z: 414.1, M + H⁺. ¹H NMR of 276: ¹H NMR (400 MHz, DMSO) δ 7.94 (s, 1H), 7.89 (d, J = 7.6 Hz, 1H), 7.85 (s, 1H), 7.79 (s, 1H), 7.74 (d, J = 8.4 Hz, 1H), 7.66 (t, J = 7.2 Hz, 1H), 7.47 (t, J = 7.2 Hz, 1H), 4.84 (s, 1H), 3.35 (s, 2H), 3.23 (d, J = 6.4 Hz. 2H), 2.86 (d, J = 26.8 Hz, 3H), 2.68 (d, J = 7.2 Hz, 1H), 2.45 (s, 3H), 1.99 (d, J = 11.2 Hz, 2H), 0.98 (d, J = 6.8 Hz, 6H). |
| 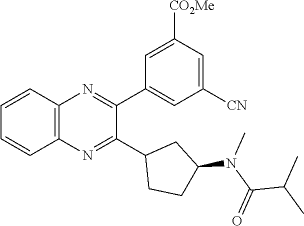 [SMILES: COC(=O)c1cc(C#N)cc(-c2nc3ccccc3nc2C2CC[C@H](N(C)C(=O)C(C)C)C2)c1] | 277 | 34.2% yield | LC MS of 277: MS (ESI) m/z: 458.1, M + H⁺. ¹H NMR of 277: ¹H NMR (400 MHz, DMSO) 8 8.55 (s, 1H), 8.43 (s, 2H), 7.93 (d. J = 8.4 Hz, 1H), 7.77 (d, J = 8.4 Hz. 1H), 7.69 (t, J = 7.2 Hz, 1H), 7.49 (t, J = 7.6 Hz, 1H), 4.85 (s, 1H), 3.93 (s, 3H), 3.42 (s, 1H), 3.25-3.15 (m, 2H), 2.86 (s, 3H), 2.68 (dd, J = 6.4, 4.4 Hz, 2H), 1.97 (s, 2H), 0.98 (d, J = 6.8 Hz, 6H). |
| 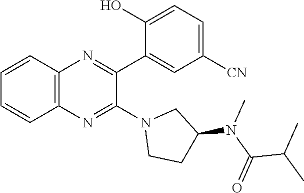 [SMILES: CC(C)C(=O)N(C)[C@H]1CCN(c2nc3ccccc3nc2-c2cc(C#N)ccc2O)C1] | 278 | 32.5% yield | LC MS of 278: MS (ESI) m/z: 416.2, M + H⁺. ¹H NMR of 278: ¹H NMR (400 MHz, DMSO) δ 11.00 (s, 1H), 7.90 (d, J = 7.2 Hz, 1H), 7.83 (d, J = 7.2 Hz, 1H), 7.77 (dd, J = 8.4, 2.0 Hz, 1H), 7.69 (d, J = 8.0 Hz, 1H), 7.62 (dd, J = 14.4, 7.2 Hz, 1H), 7.41 (dd, J = 14.8, 7.6 Hz, 1H), 7.08 (t, J = 9.6 Hz, 1H), 4.81 (s. 1H), 3.49 (d, J = 10.8 Hz, 2H), 3.24 (d, J = 6.4 Hz, 2H), 2.85 (d, J = 20.4 Hz, 3H), 2.67 (s, 1H), 1.99 (s, 2H), 0.98 (d, J = 6.8 Hz, 6H). |
| 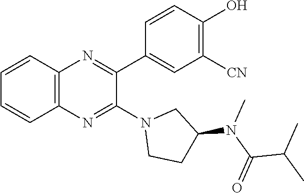 [SMILES: CC(C)C(=O)N(C)[C@H]1CCN(c2nc3ccccc3nc2-c2ccc(O)c(C#N)c2)C1] | 279 | 26.6% yield | LC MS of 279: MS (ESI) m/z: 416.1, M + H⁺. ¹H NMR of 279: ¹H NMR (400 MHz, DMSO) δ 8.25 (s, 1H), 8.11 (dd, J = 8.4, 4.8 Hz, 1H), 7.97-7.84 (m, 1H), 7.81- 7.59 (m, 3H). 7.47 (dd, J = 12.8, 2.8 Hz. 1H), 4.92 (s, 1H), 4.02-3.34 (m, 2H), 3.20 (d, J = 25.2 Hz, 2H), 3.02-2.78 (m, 3H), 2.70 (d, J = 21.6 Hz, 1H), 2.00 (d, J = 10.0 Hz, 2H), 1.00 (dd, J = 18.0, 6.8 Hz, 6H). |
| 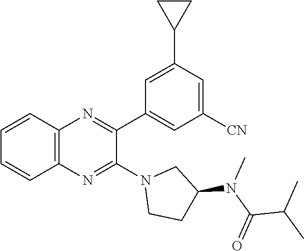 [SMILES: CC(C)C(=O)N(C)[C@H]1CCN(c2nc3ccccc3nc2-c2cc(C#N)cc(C3CC3)c2)C1] | 280 | 36.1% yield | LC MS of 280: MS (ESI) m/z: 440.2, M + H⁺. ¹H NMR of 280: ¹H NMR (400 MHz, DMSO) δ 7.89 (d, J = 7.2 Hz, 2H), 7.74 (d. J = 7.6 Hz, 2H), 7.69-7.59 (m. 2H), 7.47 (t, J = 7.2 Hz, 1H), 4.84 (s, 1H), 3.46-3.33 (m, 2H), 3.20 (dd, J = 11.2, 7.2 Hz, 2H). 3.02-2.76 (m, 3H), 2.69 (s, 1H), 2.17-1.87 (m, 3H), 1.05 (dd, J = 8.4, 2.0 Hz, 2H), 0.98 (d, J = 6.4 Hz, 6H), 0.88-0.78 (m, 2H). |
| 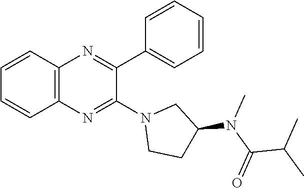 [SMILES: CC(C)C(=O)N(C)[C@H]1CCN(c2nc3ccccc3nc2-c2ccccc2)C1] | 298 | 26.4% yield | LC MS of 298: MS (ESI) m/z: 375.3, M + H⁺. ¹H NMR of 298: ¹H NMR (400 MHz, DMSO) δ 7.87 (d, J = 7.6 Hz, 1H), 7.71 (dd. J = 13.2, 7.6 Hz, 3H), 7.66-7.58 (m, 1H), 7.55-7.42 (m, 4H), 5.13-4.51 (m. 1H), 3.33 (s, 2H), 3.20 (s, 2H), 3.01- 2.72 (m. 3H), 2.66 (s, 1H), 2.07-1.83 (m, 2H), 0.97-0.95 (m, 6H). |
| 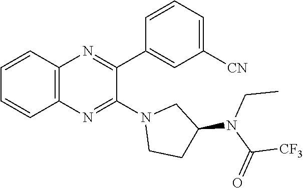 [SMILES: CCN(C(=O)C(F)(F)F)[C@H]1CCN(c2nc3ccccc3nc2-c2cccc(C#N)c2)C1] | 406 | 31.8% yield | LC MS of 406: Ret. Time: 1.436 min, MS (ESI) m/z: 440.2, [M + H]⁺. ¹H NMR of 406: ¹H NMR (400 MHz, CDCl₃) δ 8.10 (d, J = 5.2 Hz, 1H), 8.06- 7.83 (m. 3H), 7.82-7.74 (m, 1H), 7.71- 7.61 (m, 2H), 7.52 (td, J = 7.2, 2.4 Hz, 1H), 4.64-4.36 (m, 1H), 3.87-3.21 (m, 6H), 2.35-2.03 (m, 2H), 1.24 (dt, J = 25.0, 7.0 Hz, 3H). |

Compound 014 according to the present disclosure was prepared as shown in Scheme 25 by an analogous procedure to the one described in Examples 4A-4D.

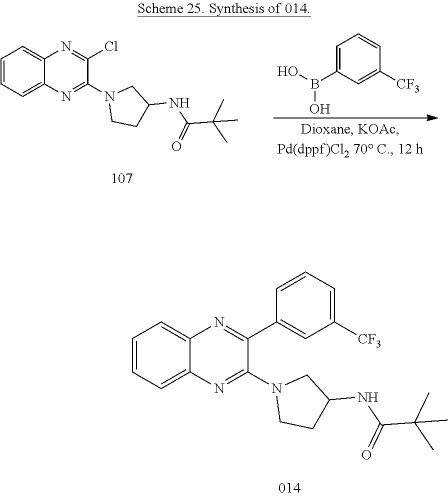 [SMILES: CC(C)(C)C(=O)NC1CCN(c2nc3ccccc3nc2Cl)C1 ; OB(O)c1cccc(C(F)(F)F)c1 ; CC(C)(C)C(=O)NC1CCN(c2nc3ccccc3nc2-c2cccc(C(F)(F)F)c2)C1]

Compound 015 according to the present disclosure was prepared as shown in Scheme 26 by an analogous procedure to the one described in Examples 4A-4D.

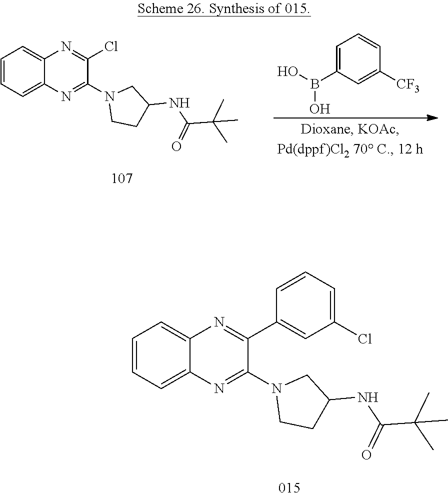 [SMILES: CC(C)(C)C(=O)NC1CCN(c2nc3ccccc3nc2Cl)C1 ; OB(O)c1cccc(C(F)(F)F)c1 ; CC(C)(C)C(=O)NC1CCN(c2nc3ccccc3nc2-c2cccc(Cl)c2)C1]

Compound 016 according to the present disclosure was prepared as shown in Scheme 27 by an analogous procedure to the one described in Examples 4A-4D.

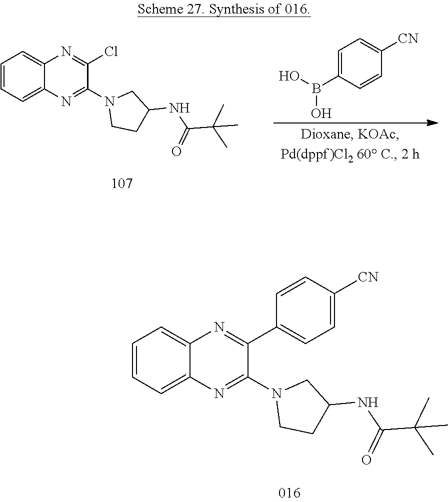 [SMILES: CC(C)(C)C(=O)NC1CCN(c2nc3ccccc3nc2Cl)C1 ; N#Cc1ccc(B(O)O)cc1 ; CC(C)(C)C(=O)NC1CCN(c2nc3ccccc3nc2-c2ccc(C#N)cc2)C1]

Compound 017 according to the present disclosure was prepared as shown in Scheme 28 by an analogous procedure to the one described in Examples 4A-4D.

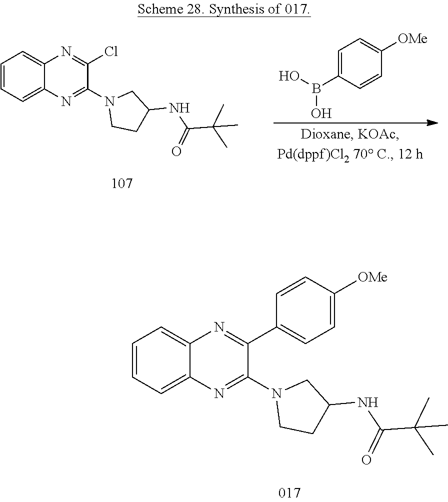 [SMILES: CC(C)(C)C(=O)NC1CCN(c2nc3ccccc3nc2Cl)C1 ; COc1ccc(B(O)O)cc1 ; COc1ccc(-c2nc3ccccc3nc2N2CCC(NC(=O)C(C)(C)C)C2)cc1]

Compound 018 according to the present disclosure was prepared as shown in Scheme 29 by an analogous procedure to the one described in Examples 4A-4D.

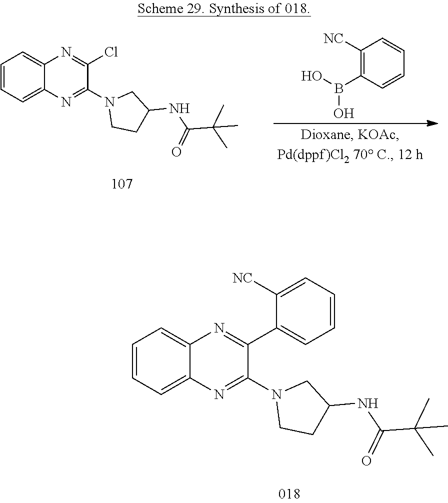 [SMILES: CC(C)(C)C(=O)NC1CCN(c2nc3ccccc3nc2Cl)C1 ; N#Cc1ccccc1B(O)O ; CC(C)(C)C(=O)NC1CCN(c2nc3ccccc3nc2-c2ccccc2C#N)C1]

Compound 126 according to the present disclosure was prepared as shown in Scheme 30 by an analogous procedure to the one described in Examples 4A-4D.

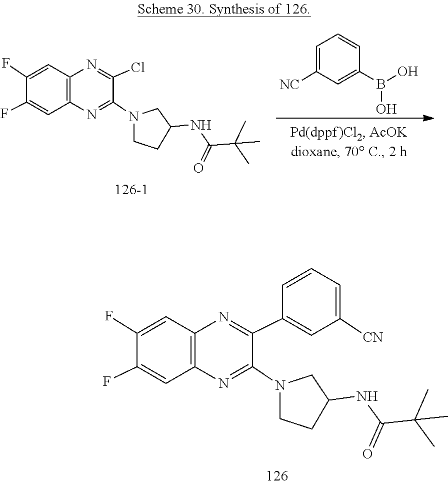 [SMILES: CC(C)(C)C(=O)NC1CCN(c2nc3cc(F)c(F)cc3nc2Cl)C1 ; N#Cc1cccc(B(O)O)c1 ; CC(C)(C)C(=O)NC1CCN(c2nc3cc(F)c(F)cc3nc2-c2cccc(C#N)c2)C1]

Compound 127 according to the present disclosure was prepared as shown in Scheme 31 by an analogous procedure to the one described in Examples 4A-4D.

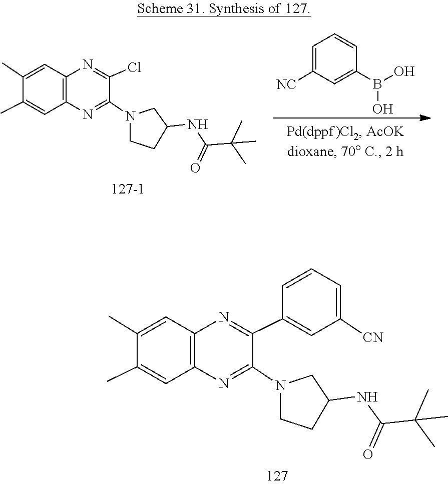 [SMILES: Cc1cc2nc(Cl)c(N3CCC(NC(=O)C(C)(C)C)C3)nc2cc1C ; N#Cc1cccc(B(O)O)c1 ; Cc1cc2nc(-c3cccc(C#N)c3)c(N3CCC(NC(=O)C(C)(C)C)C3)nc2cc1C]

Compound 226 according to the present disclosure was prepared as shown in Scheme 32 by an analogous procedure to the one described in Examples 4A-4D.

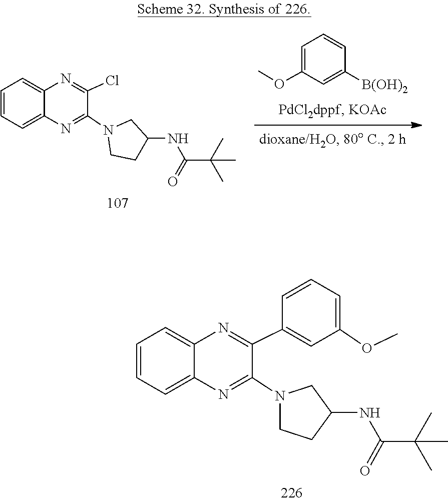 [SMILES: CC(C)(C)C(=O)NC1CCN(c2nc3ccccc3nc2Cl)C1 ; COc1cccc(B(O)O)c1 ; COc1cccc(-c2nc3ccccc3nc2N2CCC(NC(=O)C(C)(C)C)C2)c1]

Compound 227 according to the present disclosure was prepared as shown in Scheme 33 by an analogous procedure to the one described in Examples 4A-4D.

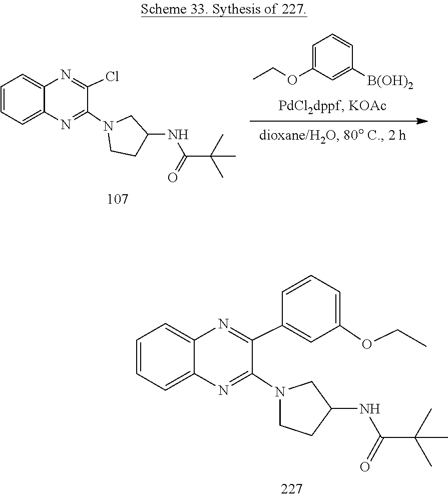 [SMILES: CC(C)(C)C(=O)NC1CCN(c2nc3ccccc3nc2Cl)C1 ; CCOc1cccc(B(O)O)c1 ; CCOc1cccc(-c2nc3ccccc3nc2N2CCC(NC(=O)C(C)(C)C)C2)c1]

Compound 229 according to the present disclosure was prepared as shown in Scheme 34 by an analogous procedure to the one described in Examples 4A-4D.

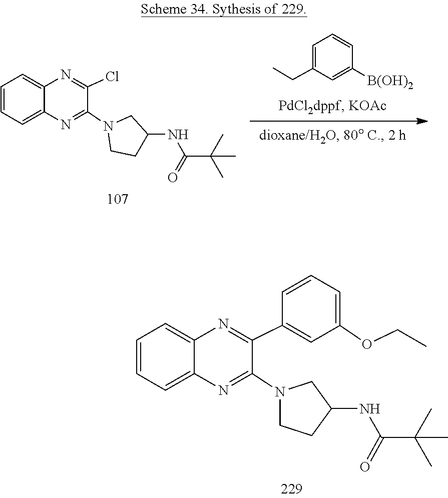 [SMILES: CC(C)(C)C(=O)NC1CCN(c2nc3ccccc3nc2Cl)C1 ; CCc1cccc(B(O)O)c1 ; CCOc1cccc(-c2nc3ccccc3nc2N2CCC(NC(=O)C(C)(C)C)C2)c1]

Compound 239 according to the present disclosure was prepared as shown in Scheme 35 by an analogous procedure to the one described in Examples 4A-4D.

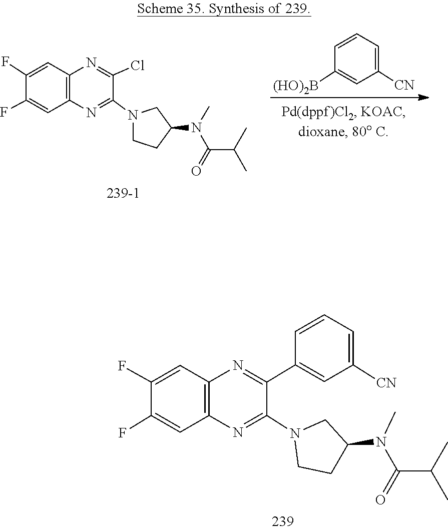 [SMILES: CC(C)C(=O)N(C)[C@H]1CCN(c2nc3cc(F)c(F)cc3nc2Cl)C1 ; N#Cc1cccc(B(O)O)c1 ; CC(C)C(=O)N(C)[C@H]1CCN(c2nc3cc(F)c(F)cc3nc2-c2cccc(C#N)c2)C1]

Compound 260 according to the present disclosure was prepared as shown in Scheme 36 by an analogous procedure to the one described in Examples 4A-4D.

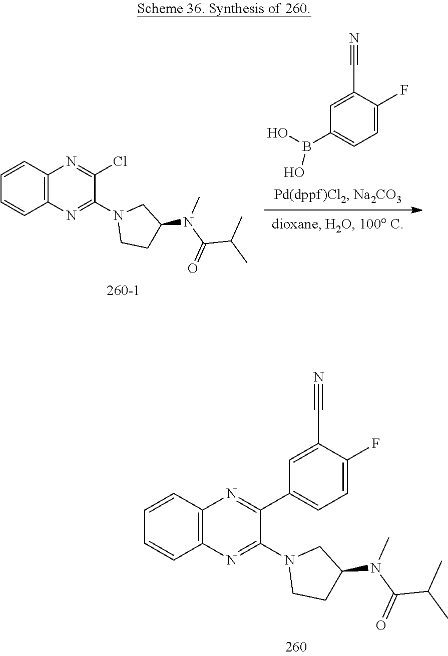 [SMILES: CC(C)C(=O)N(C)[C@H]1CCN(c2nc3ccccc3nc2Cl)C1 ; N#Cc1cc(B(O)O)ccc1F ; CC(C)C(=O)N(C)[C@H]1CCN(c2nc3ccccc3nc2-c2ccc(F)c(C#N)c2)C1]

Compound 261 according to the present disclosure was prepared as shown in Scheme 37 by an analogous procedure to the one described in Examples 4A-4D.

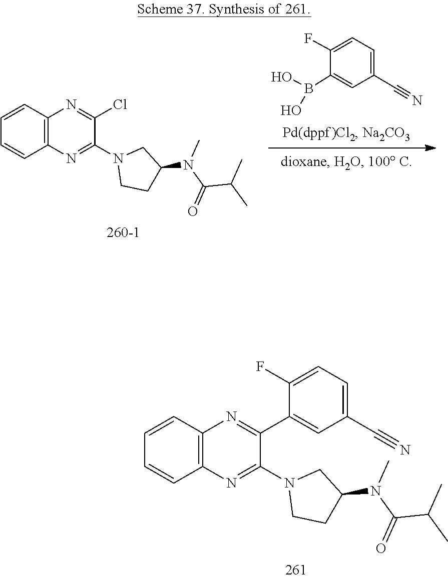 [SMILES: CC(C)C(=O)N(C)[C@H]1CCN(c2nc3ccccc3nc2Cl)C1 ; N#Cc1ccc(F)c(B(O)O)c1 ; CC(C)C(=O)N(C)[C@H]1CCN(c2nc3ccccc3nc2-c2cc(C#N)ccc2F)C1]

Compound 262 according to the present disclosure was prepared as shown in Scheme 38 by an analogous procedure to the one described in Examples 4A-4D.

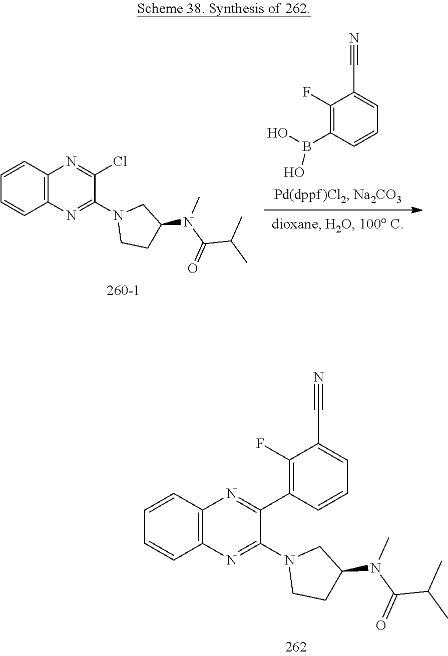 [SMILES: CC(C)C(=O)N(C)[C@H]1CCN(c2nc3ccccc3nc2Cl)C1 ; N#Cc1cccc(B(O)O)c1F ; CC(C)C(=O)N(C)[C@H]1CCN(c2nc3ccccc3nc2-c2cccc(C#N)c2F)C1]

Compound 263 according to the present disclosure was prepared as shown in Scheme 39 by an analogous procedure to the one described in Examples 4A-4D.

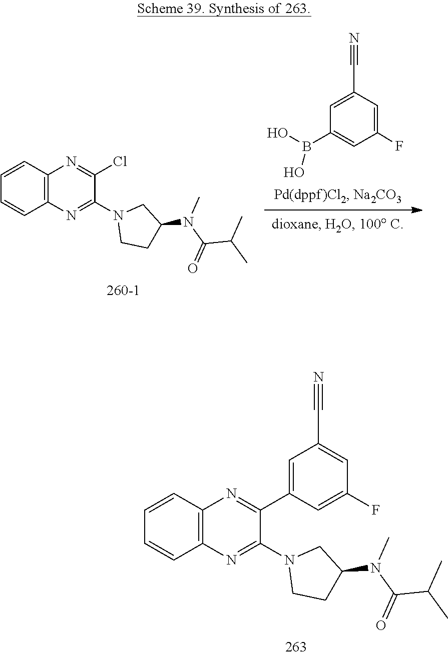 [SMILES: CC(C)C(=O)N(C)[C@H]1CCN(c2nc3ccccc3nc2Cl)C1 ; N#Cc1cc(F)cc(B(O)O)c1 ; CC(C)C(=O)N(C)[C@H]1CCN(c2nc3ccccc3nc2-c2cc(F)cc(C#N)c2)C1]

Compound 264 according to the present disclosure was prepared as shown in Scheme 40 by an analogous procedure to the one described in Examples 4A-4D.

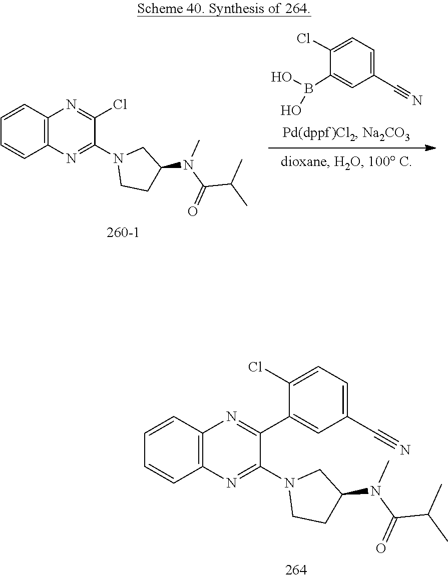 [SMILES: CC(C)C(=O)N(C)[C@H]1CCN(c2nc3ccccc3nc2Cl)C1 ; N#Cc1ccc(Cl)c(B(O)O)c1 ; CC(C)C(=O)N(C)[C@H]1CCN(c2nc3ccccc3nc2-c2cc(C#N)ccc2Cl)C1]

Compound 265 according to the present disclosure was prepared as shown in Scheme 41 by an analogous procedure to the one described in Examples 4A-4D.

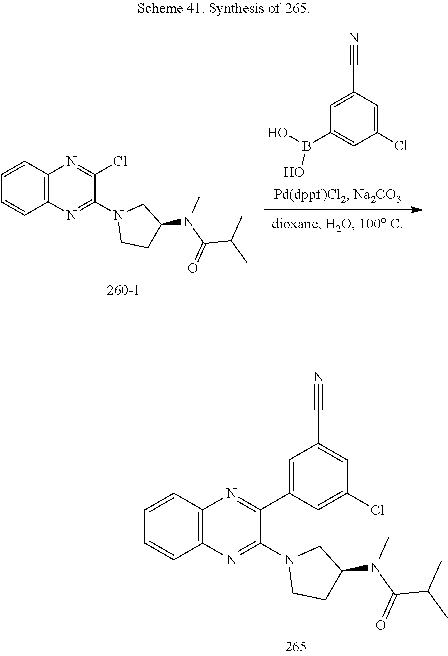 [SMILES: CC(C)C(=O)N(C)[C@H]1CCN(c2nc3ccccc3nc2Cl)C1 ; N#Cc1cc(Cl)cc(B(O)O)c1 ; CC(C)C(=O)N(C)[C@H]1CCN(c2nc3ccccc3nc2-c2cc(Cl)cc(C#N)c2)C1]

Compound 266 according to the present disclosure was prepared as shown in Scheme 42 by an analogous procedure to the one described in Examples 4A-4D.

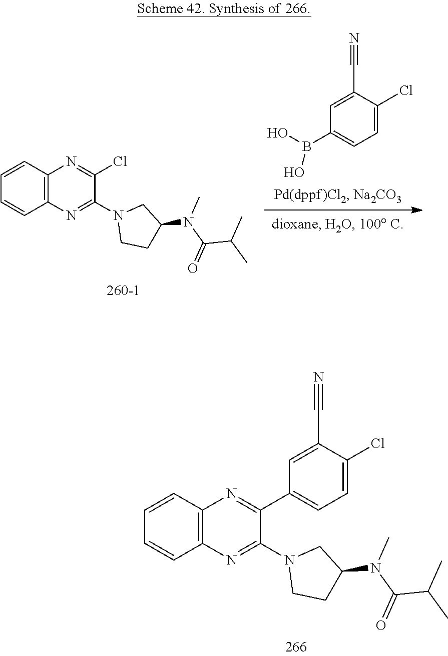 [SMILES: CC(C)C(=O)N(C)[C@H]1CCN(c2nc3ccccc3nc2Cl)C1 ; N#Cc1cc(B(O)O)ccc1Cl ; CC(C)C(=O)N(C)[C@H]1CCN(c2nc3ccccc3nc2-c2ccc(Cl)c(C#N)c2)C1]

Compound 267 according to the present disclosure was prepared as shown in Scheme 43 by an analogous procedure to the one described in Examples 4A-4D.

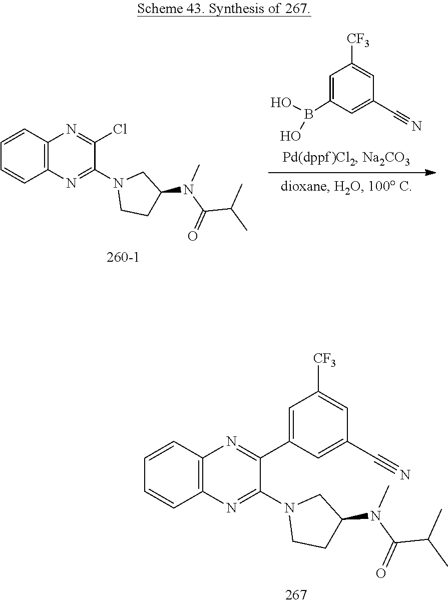 [SMILES: CC(C)C(=O)N(C)[C@H]1CCN(c2nc3ccccc3nc2Cl)C1 ; N#Cc1cc(B(O)O)cc(C(F)(F)F)c1 ; CC(C)C(=O)N(C)[C@H]1CCN(c2nc3ccccc3nc2-c2cc(C#N)cc(C(F)(F)F)c2)C1]

Compound 268 according to the present disclosure was prepared as shown in Scheme 44 by an analogous procedure to the one described in Examples 4A-4D.

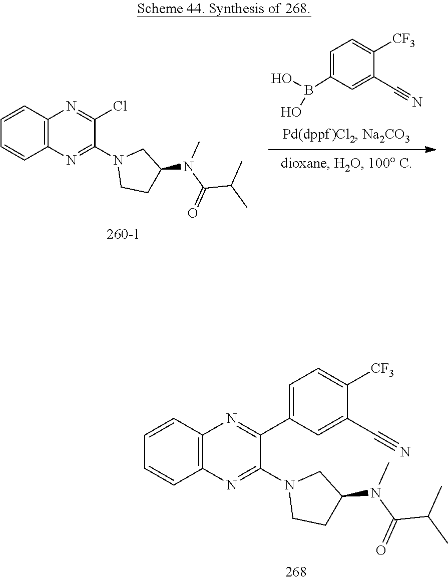 [SMILES: CC(C)C(=O)N(C)[C@H]1CCN(c2nc3ccccc3nc2Cl)C1 ; N#Cc1cc(B(O)O)ccc1C(F)(F)F ; CC(C)C(=O)N(C)[C@H]1CCN(c2nc3ccccc3nc2-c2ccc(C(F)(F)F)c(C#N)c2)C1]

Compound 269 according to the present disclosure was prepared as shown in Scheme 45 by an analogous procedure to the one described in Examples 4A-4D.

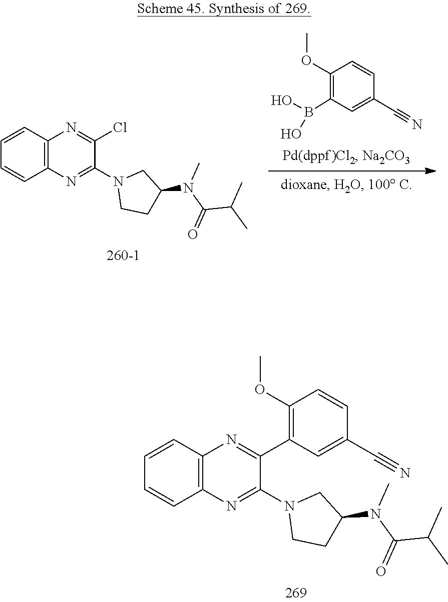 [SMILES: CC(C)C(=O)N(C)[C@H]1CCN(c2nc3ccccc3nc2Cl)C1 ; COc1ccc(C#N)cc1B(O)O ; COc1ccc(C#N)cc1-c1nc2ccccc2nc1N1CC[C@H](N(C)C(=O)C(C)C)C1]

Compound 270 according to the present disclosure was prepared as shown in Scheme 46 by an analogous procedure to the one described in Examples 4A-4D.

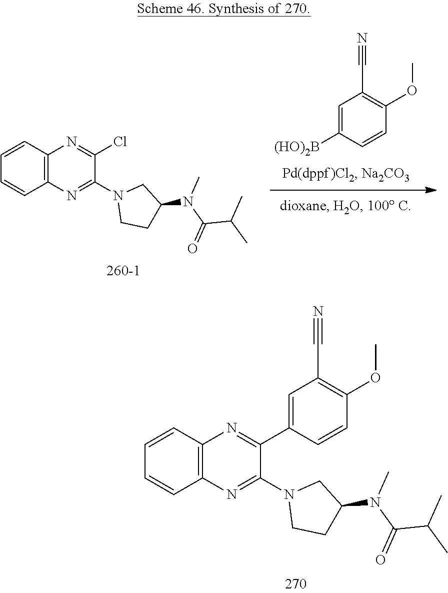 [SMILES: CC(C)C(=O)N(C)[C@H]1CCN(c2nc3ccccc3nc2Cl)C1 ; COc1ccc(B(O)O)cc1C#N ; COc1ccc(-c2nc3ccccc3nc2N2CC[C@H](N(C)C(=O)C(C)C)C2)cc1C#N]

Compound 271 according to the present disclosure was prepared as shown in Scheme 47 by an analogous procedure to the one described in Examples 4A-4D.

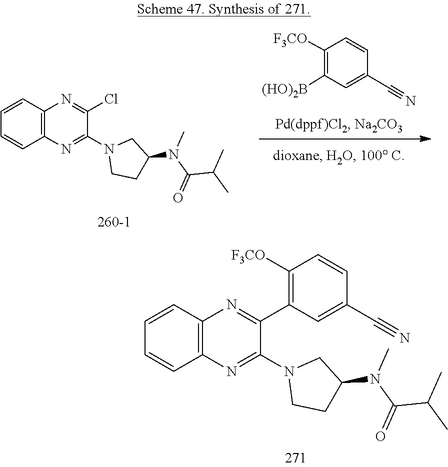 [SMILES: CC(C)C(=O)N(C)[C@H]1CCN(c2nc3ccccc3nc2Cl)C1 ; N#Cc1ccc(OC(F)(F)F)c(B(O)O)c1 ; CC(C)C(=O)N(C)[C@H]1CCN(c2nc3ccccc3nc2-c2cc(C#N)ccc2OC(F)(F)F)C1]

Compound 272 according to the present disclosure was prepared as shown in Scheme 48 by an analogous procedure to the one described in Examples 4A-4D.

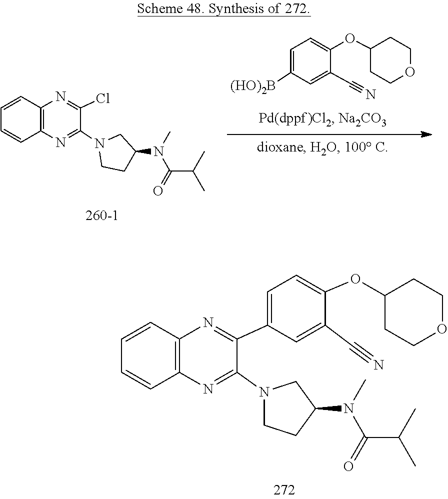 [SMILES: N#Cc1cc(B(O)O)ccc1OC1CCOCC1 ; CC(C)C(=O)N(C)[C@H]1CCN(c2nc3ccccc3nc2Cl)C1 ; CC(C)C(=O)N(C)[C@H]1CCN(c2nc3ccccc3nc2-c2ccc(OC3CCOCC3)c(C#N)c2)C1]

Compound 273 according to the present disclosure was prepared as shown in Scheme 49 by an analogous procedure to the one described in Examples 4A-4D.

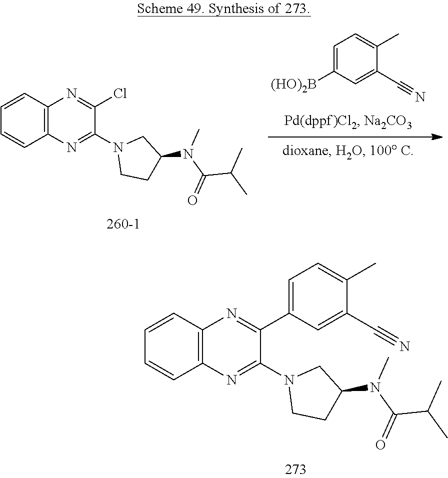 [SMILES: CC(C)C(=O)N(C)[C@H]1CCN(c2nc3ccccc3nc2Cl)C1 ; Cc1ccc(B(O)O)cc1C#N ; Cc1ccc(-c2nc3ccccc3nc2N2CC[C@H](N(C)C(=O)C(C)C)C2)cc1C#N]

Compound 274 according to the present disclosure was prepared as shown in Scheme 50 by an analogous procedure to the one described in Examples 4A-4D.

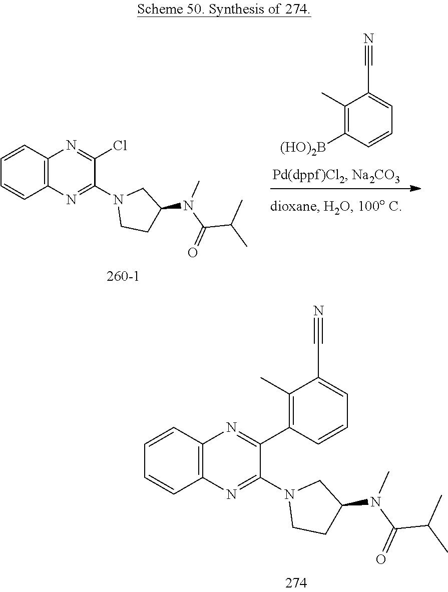 [SMILES: CC(C)C(=O)N(C)[C@H]1CCN(c2nc3ccccc3nc2Cl)C1 ; Cc1c(C#N)cccc1B(O)O ; Cc1c(C#N)cccc1-c1nc2ccccc2nc1N1CC[C@H](N(C)C(=O)C(C)C)C1]

Compound 275 according to the present disclosure was prepared as shown in Scheme 51 by an analogous procedure to the one described in Examples 4A-4D.

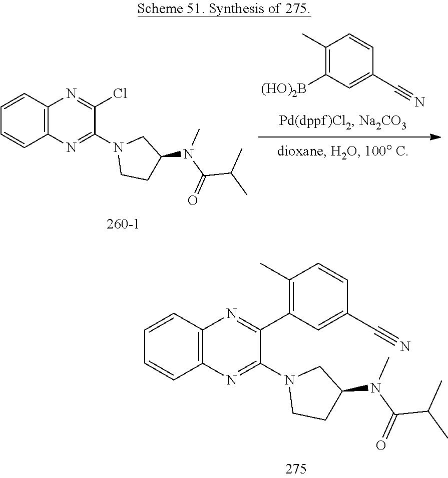 [SMILES: CC(C)C(=O)N(C)[C@H]1CCN(c2nc3ccccc3nc2Cl)C1 ; Cc1ccc(C#N)cc1B(O)O ; Cc1ccc(C#N)cc1-c1nc2ccccc2nc1N1CC[C@H](N(C)C(=O)C(C)C)C1]

Compound 276 according to the present disclosure was prepared as shown in Scheme 52 by an analogous procedure to the one described in Examples 4A-4D.

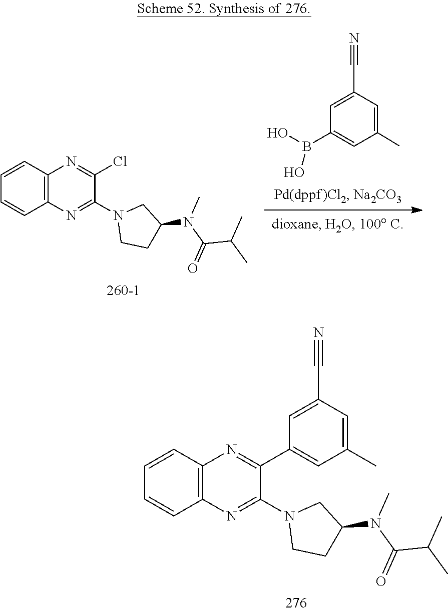 [SMILES: CC(C)C(=O)N(C)[C@H]1CCN(c2nc3ccccc3nc2Cl)C1 ; Cc1cc(C#N)cc(B(O)O)c1 ; Cc1cc(C#N)cc(-c2nc3ccccc3nc2N2CC[C@H](N(C)C(=O)C(C)C)C2)c1]

Compound 277 according to the present disclosure was prepared as shown in Scheme 53 by an analogous procedure to the one described in Examples 4A-4D.

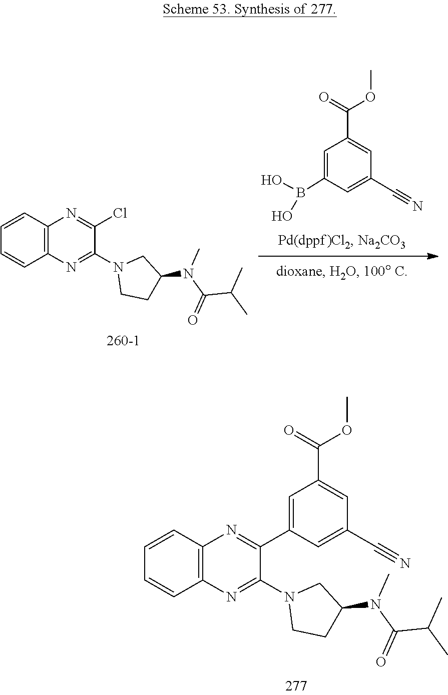 [SMILES: CC(C)C(=O)N(C)[C@H]1CCN(c2nc3ccccc3nc2Cl)C1 ; COC(=O)c1cc(C#N)cc(B(O)O)c1 ; COC(=O)c1cc(C#N)cc(-c2nc3ccccc3nc2N2CC[C@H](N(C)C(=O)C(C)C)C2)c1]

Compound 278 according to the present disclosure was prepared as shown in Scheme 54 by an analogous procedure to the one described in Examples 4A-4D.

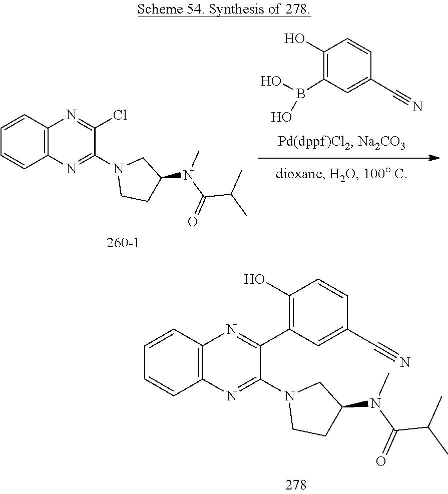 [SMILES: CC(C)C(=O)N(C)[C@H]1CCN(c2nc3ccccc3nc2Cl)C1 ; N#Cc1ccc(O)c(B(O)O)c1 ; CC(C)C(=O)N(C)[C@H]1CCN(c2nc3ccccc3nc2-c2cc(C#N)ccc2O)C1]

Compound 279 according to the present disclosure was prepared as shown in Scheme 55 by an analogous procedure to the one described in Examples 4A-4D.

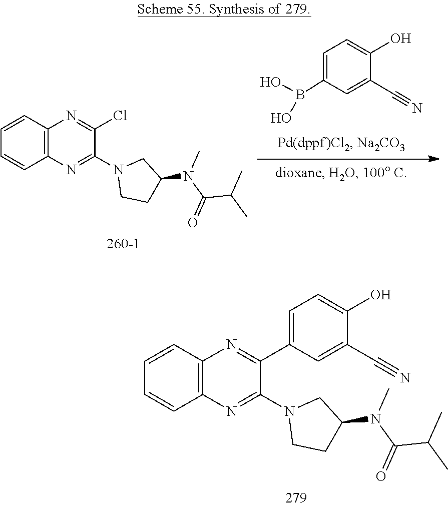 [SMILES: CC(C)C(=O)N(C)[C@H]1CCN(c2nc3ccccc3nc2Cl)C1 ; N#Cc1cc(B(O)O)ccc1O ; CC(C)C(=O)N(C)[C@H]1CCN(c2nc3ccccc3nc2-c2ccc(O)c(C#N)c2)C1]

Compound 280 according to the present disclosure was prepared as shown in Scheme 56 by an analogous procedure to the one described in Examples 4A-4D.

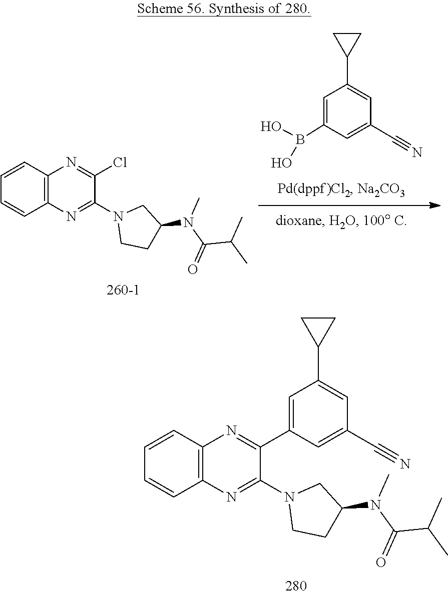 [SMILES: CC(C)C(=O)N(C)[C@H]1CCN(c2nc3ccccc3nc2Cl)C1 ; N#Cc1cc(B(O)O)cc(C2CC2)c1 ; CC(C)C(=O)N(C)[C@H]1CCN(c2nc3ccccc3nc2-c2cc(C#N)cc(C3CC3)c2)C1]

Compound 298 according to the present disclosure was prepared as shown in Scheme 57 by an analogous procedure to the one described in Examples 4A-4D.

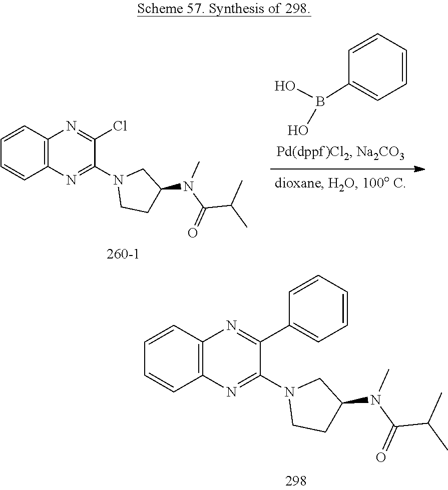 [SMILES: CC(C)C(=O)N(C)[C@H]1CCN(c2nc3ccccc3nc2Cl)C1 ; OB(O)c1ccccc1 ; CC(C)C(=O)N(C)[C@H]1CCN(c2nc3ccccc3nc2-c2ccccc2)C1]

Compound 406 according to the present disclosure was prepared as shown in Scheme 58 by an analogous procedure to the one described in Examples 4A-4D.

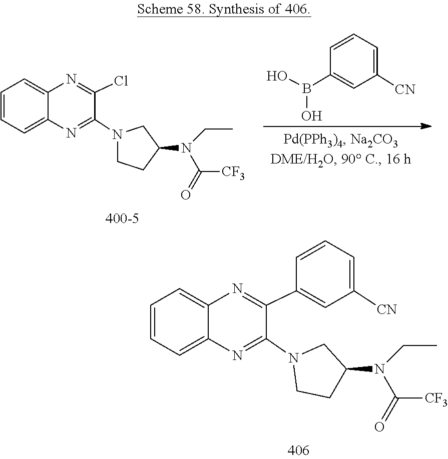 [SMILES: CCN(C(=O)C(F)(F)F)[C@H]1CCN(c2nc3ccccc3nc2Cl)C1 ; N#Cc1cccc(B(O)O)c1 ; CCN(C(=O)C(F)(F)F)[C@H]1CCN(c2nc3ccccc3nc2-c2cccc(C#N)c2)C1]

### Example 5: Synthesis of Analogs 103 and 104

Compounds 103 and 104 according to the present disclosure were prepared as shown in Scheme 59 and described below.

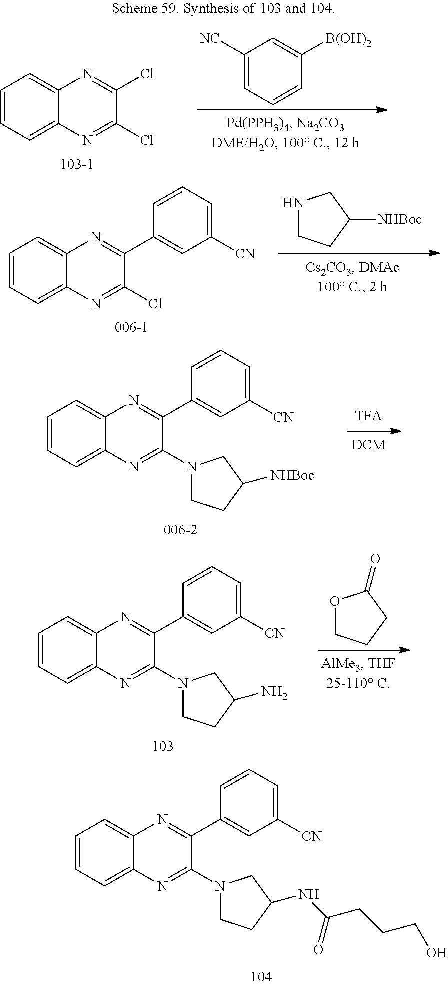

### Example 5A: Preparation of Compound 006-1

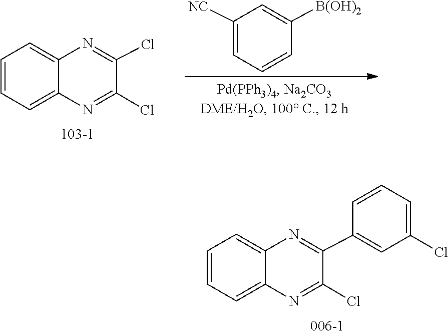 [SMILES: Clc1nc2ccccc2nc1Cl ; N#Cc1cccc(B(O)O)c1 ; Clc1cccc(-c2nc3ccccc3nc2Cl)c1]

To a 250 mL round-bottom flask equipped with a magnetic stir bar and a reflux condenser was added 2,3-dichloroquinoxaline 103-1 (5 g, 25.1 mmol, 1 eq) followed by the addition of DME (50 mL) and H₂O (10 mL). Then (3-cyanophenyl)boronic acid (3.69 g, 25.1 mmol, 1 eq), Na₂CO₃ (5.33 g, 50.2 mmol, 2 eq) and Pd(PPh₃)₄ (2.90 g, 2.51 mmol, 0.1 eq) were added into the mixture at 25° C. The flask was evacuated and backfilled with nitrogen three times. The mixture was heated to 100° C. and stirred for 12 h. The suspension was filtered through filter paper. The mixture was added H₂O (30 mL). Then water phase was extracted by ethyl acetate (40 mL×3) and the combined organic phases were washed with brine (20 mL×3), dried over anhydrous sodium sulfate, filtered, and concentrated under reduced pressure to afford the crude product as a yellow oil. The crude product was purified by silica gel column chromatography (petroleum ether/ethyl acetate, from 1/0 to 2/1) to give 3-(3-chloroquinoxalin-2-yl)benzonitrile 006-1 (2.45 g, 35% yield, 96.6% purity) as a white solid. LC MS of 006-1: Ret. Time: 0.978 min, MS (ESI) m/z: 266.1, M+H⁺. ¹H NMR of 006-1: (400 MHz, DMSO-d₆) δ 8.33-8.27 (m, 1H), 8.24-8.16 (m, 2H), 8.16-8.10 (m, 1H), 8.05 (td, J=1.4, 7.8 Hz, 1H), 8.01-7.92 (m, 2H), 7.83-7.77 (m, 1H).

### Example 5B: Preparation of Compound 006-2

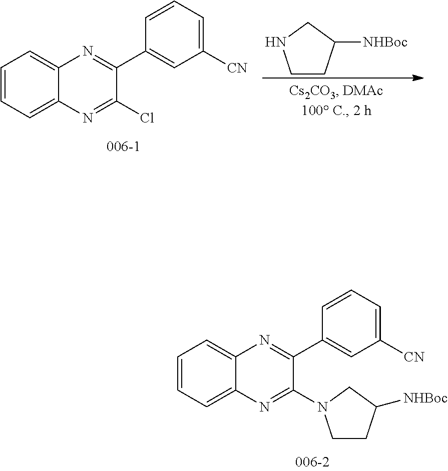 [SMILES: N#Cc1cccc(-c2nc3ccccc3nc2Cl)c1 ; CC(C)(C)OC(=O)NC1CCNC1 ; CC(C)(C)OC(=O)NC1CCN(c2nc3ccccc3nc2-c2cccc(C#N)c2)C1]

Compound 006-2 according to the present disclosure was prepared by the same procedure described in Example 2A.

### Example 5C: Preparation of Compound 103

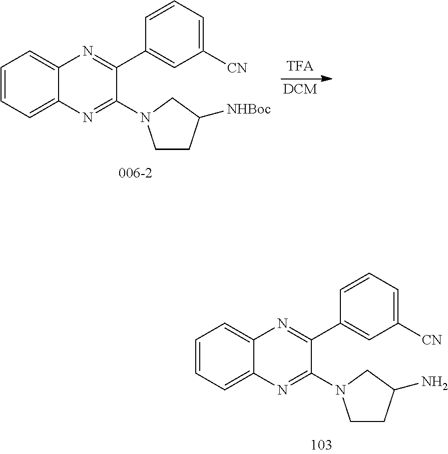

To a 50 mL round-bottom flask equipped with a magnetic stir bar was added tert-butyl N-[1-[3-(3-cyanophenyl)quinoxalin-2-yl]pyrrolidin-3-yl]carbamate 006-2 (700 mg, 1.63 mmol, 96.86% purity, 1 eq) followed by the addition of DCM (7 mL). Then TFA (1.54 g, 13.5 mmol, 8.28 eq) was added into the mixture at 25° C. The mixture was stirred at 25° C. for 3 h. The TLC (petroleum ether/ethyl acetate: 1/1) showed the reaction had completed. The mixture was quenched by slow addition of H₂O (15 mL). The resulting mixture was transferred to a separatory funnel, and the aqueous layer mixture was extracted with ethyl acetate (15 mL×3). The combined organic layers were washed with brine (15 mL), dried over anhydrous sodium sulfate, filtered, and concentrated under reduced pressure affording the residue as light yellow oil. The crude product (45 mg) was purified by preparative HPLC: (Phenomenex Gemini C18 column (75×30 mm, 3 um); flow rate: 25 mL/min; gradient: 22%-42% B over 7 min; mobile phase A: 0.1% aqueous trifluoroacetic acid, mobile phase B: acetonitrile). After lyophilization, 3-[3-(3-aminopyrrolidin-1-yl)quinoxalin-2-yl]benzonitrile 103 (31 mg, 49% yield, 96.6% purity, TFA salt) was obtained as a yellow gum. LC MS of 103: Ret. Time: 0.778 min, MS (ESI) m/z: 316.0, M+H⁺. ¹H NMR of 103: (400 MHz, DMSO-d₆+D₂O) δ 8.15-8.09 (m, 1H), 8.04-8.00 (m, 1H), 8.00-7.93 (m, 1H), 7.90 (dd, J=0.8, 8.0 Hz, 1H), 7.78-7.71 (m, 2H), 7.68 (dt, J=1.2, 7.6 Hz, 1H), 7.49 (ddd, J=1.4, 6.8, 8.2 Hz, 1H), 3.84-3.74 (m, 1H), 3.54 (br dd, J=6.4, 12.0 Hz, 1H), 3.38 (td, J=7.2, 10.8 Hz, 1H), 3.33-3.22 (m, 2H), 2.16 (qd, J=6.8, 13.4 Hz, 1H), 1.97-1.84 (m, 1H).

### Example 5D: Preparation of Compound 104

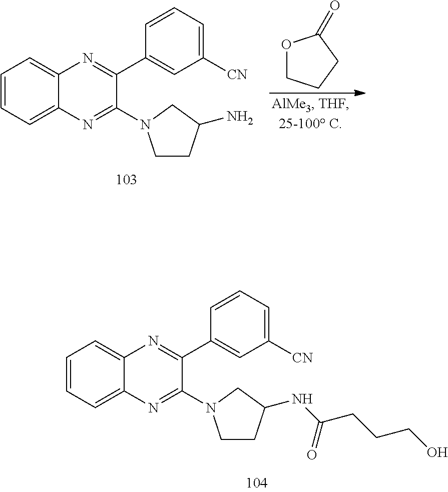 [SMILES: N#Cc1cccc(-c2nc3ccccc3nc2N2CCC(N)C2)c1 ; O=C1CCCO1 ; N#Cc1cccc(-c2nc3ccccc3nc2N2CCC(NC(=O)CCCO)C2)c1]

To a 10 mL round-bottom flask equipped with a magnetic stir bar was added 3-[3-(3-aminopyrrolidin-1-yl)quinoxalin-2-yl]benzonitrile 103 (150 mg, 472 umol, 99.3% purity, 1 eq) followed by the addition of THF (2 mL). Then tetrahydrofuran-2-one (81.3 mg, 944 umol, 2 eq), AlMe₃ (2 M, 708.45 uL, 3 eq) and THF (2 mL) were added into the mixture at 25° C. The mixture was stirred at 110° C. for 2 h. The reaction was quenched by addition of saturated aqueous NH₄Cl (10 mL), then extracted with ethyl acetate (10 mL×3). The organic layer was concentrated to give a crude product. The crude product was purified by preparative HPLC: (Phenomenex Luna C18 column (75×30 mm, 3 um); flow rate: 25 mL/min; gradient: 34%-54% B over 7 min; mobile phase A: 0.1% aqueous TFA, mobile phase B: acetonitrile). After lyophilization, N-[1-[3-(3-cyanophenyl)quinoxalin-2-yl]pyrrolidin-3-yl]-4-hydroxy-butanamide 104 (17 mg, 6.58% yield, 94.2% purity, TFA salt) was obtained as a yellow gum. LC MS of 104: Ret. Time: 0.838 min, MS (ESI) m/z: 402.2. ¹H NMR of 104: (400 MHz, DMSO-d₆) δ 8.12 (t, J=1.4 Hz, 1H), 8.00 (td, J=1.4, 7.8 Hz, 1H), 7.98-7.93 (m, 2H), 7.88 (dd, J=0.8, 8.4 Hz, 1H), 7.76-7.70 (m, 2H), 7.65 (ddd, J=1.4, 6.8, 8.4 Hz, 1H), 7.45 (ddd, J=1.4, 6.8, 8.2 Hz, 1H), 4.23-4.13 (m, 1H), 3.46-3.35 (m, 2H), 3.35-3.22 (m, 4H), 3.01 (dd, J=4.8, 11.2 Hz, 1H), 2.10-1.95 (m, 3H), 1.81-1.70 (m, 1H), 1.64-1.53 (m, 2H).

### Example 6: Synthesis of Analogs 113 and 212, 213, 249, 250, 405

Compound 113 according to the present disclosure was prepared as shown in Scheme 60 and described below.

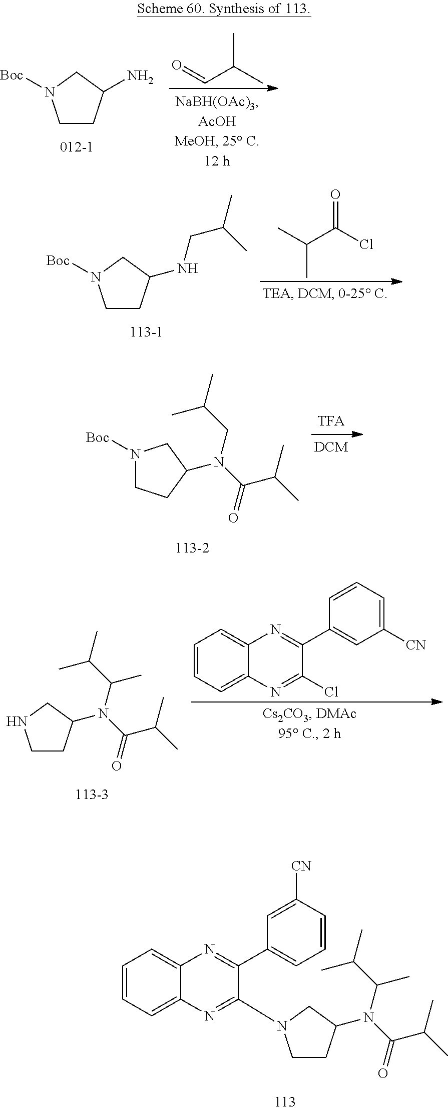

### Example 6A: Preparation of Compound 113-1

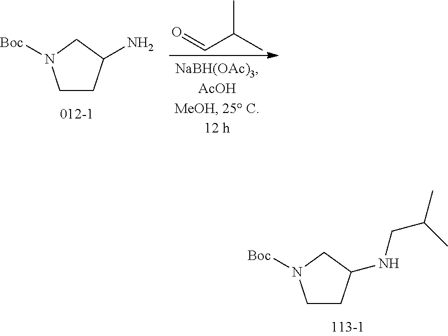

To a 50 mL round-bottom flask equipped with a magnetic stir bar was added 2-methylpropanal (387 mg, 5.4 mmol, 490 uL, 1 eq) followed by the addition of MeOH (15 mL), AcOH (161 mg, 2.7 mmol, 153 uL, 0.5 eq) and NaBH(OAc)₃ (2.28 g, 10.7 mmol, 2 eq). Then tert-butyl 3-aminopyrrolidine-1-carboxylate 012-1 (1 g, 5.4 mmol, 1 eq) was added into the mixture at 25° C. The mixture was 25° C. and stirred for 12 h. The LC-MS showed that the starting material 012-1 was consumed and the desired mass was detected. The reaction mixture was concentrated under reduced pressure to remove MeOH. Then water (50 mL) was added. The mixture was acidified with aqueous HCl (1 M) to pH 2-3. The resulting mixture was transferred to a separatory funnel, and the aqueous layer mixture was washed with ethyl acetate (50 mL×2). The water layer was saved and the organic layer was discarded. The water layer was basified with a saturated aqueous solution of NaHCO₃ to pH 8. The resulting mixture was transferred to a separatory funnel, and the aqueous mixture was extracted with ethyl acetate (50 mL×2). The organic layers were combined and washed with brine (20 mL), dried over anhydrous sodium sulfate, filtered, and concentrated under reduced pressure to afford tert-butyl 3-(isobutylamino)pyrrolidine-1-carboxylate 113-1 (1.1 g, crude) as a colorless oil. LCMS of 113-1: Ret. Time: 0.931 min, MS (ESI) m/z: 243.2, M+H⁺.

### Example 6B: Preparation of Compound 113-2

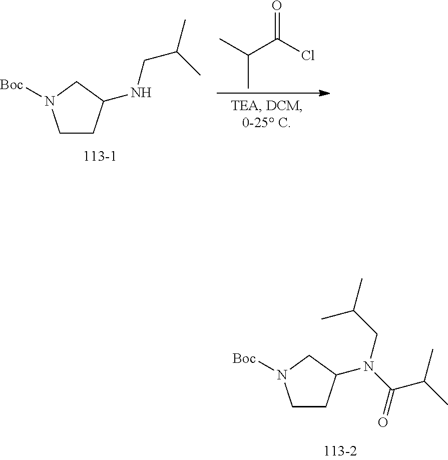

To a 40 mL vial equipped with a magnetic stir bar was added tert-butyl 3-(isobutylamino)pyrrolidine-1-carboxylate (1 g, 4.13 mmol, 1 eq) followed by the addition of DCM (10 mL). Then TEA (835 mg, 8.25 mmol, 1.15 mL, 2 eq) and isobutyryl chloride (527 mg, 4.95 mmol, 517 uL, 1.2 eq) were added into the mixture at 25° C. The mixture was stirred at 25° C. for 2 h. The TLC (petroleum ether/ethyl acetate: 2/1) showed the starting material 113-1 was consumed. The mixture was quenched by slow addition of H₂O (40 mL). The resulting mixture was transferred to a separatory funnel, and the aqueous layer mixture was extracted with ethyl acetate (50 mL×2). The combined organic layers were washed with brine (30 mL), dried over anhydrous sodium sulfate, filtered, and concentrated under reduced pressure to afford a residue as a yellow oil. The crude product was purified by silica gel column chromatography (petroleum ether/ethyl acetate, from 10/1 to 2/1) to give tert-butyl 3-[isobutyl(2-methylpropanoyl)amino]pyrrolidine-1-carboxylate 113-2 (1.1 g, 3.33 mmol, 80.72% yield, 94.6% purity) as a colorless oil. LC MS of 113-2: Ret. Time: 0.959 min, MS (ESI) m/z: 257.1, M+H⁺. ¹H NMR of 113-2: (400 MHz, CDCl₃) δ 4.53-4.29 (m, 1H), 3.70-3.42 (m, 2H), 3.28 (td, J=8.4, 10.4 Hz, 2H), 3.20-3.06 (m, 2H), 2.94-2.70 (m, 1H), 2.04-1.98 (m, 1H), 1.91-1.77 (m, 2H), 1.45 (s, 9H), 1.17-1.07 (m, 6H), 0.96-0.84 (m, 6H).

### Example 6C: Preparation of Compound 113-3

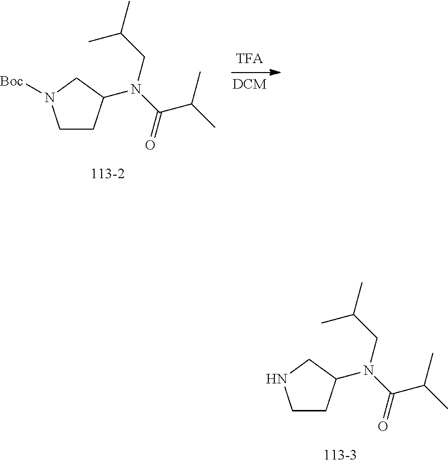 [SMILES: CC(C)CN(C(=O)C(C)C)C1CCN(C(=O)OC(C)(C)C)C1 ; CC(C)CN(C(=O)C(C)C)C1CCNC1]

To a 50 mL round-bottom flask equipped with a magnetic stir bar was added tert-butyl 3-[isobutyl(2-methylpropanoyl)amino]pyrrolidine-1-carboxylate 113-2 (1.1 g, 3.33 mmol, 94.6% purity, 1 eq) followed by the addition of DCM (10 mL). Then TFA (3.1 g, 27 mmol, 2 mL, 8.11 eq) was added into the mixture at 0° C. The mixture was stirred at 25° C. for 1 h. The LC-MS showed the starting material 113-2 was consumed and the desired mass was detected. The mixture was concentrated under reduced pressure to afford N-isobutyl-2-methyl-N-pyrrolidin-3-yl-propanamide 113-3 (2 g, crude, TFA salt) as a brown oil which was used directly in the next step. LC MS of 113-3: Ret. Time: 0.701 min, MS (ESI) m/z: 213.1, M+H⁺;

### Example 6D: Preparation of Compound 113

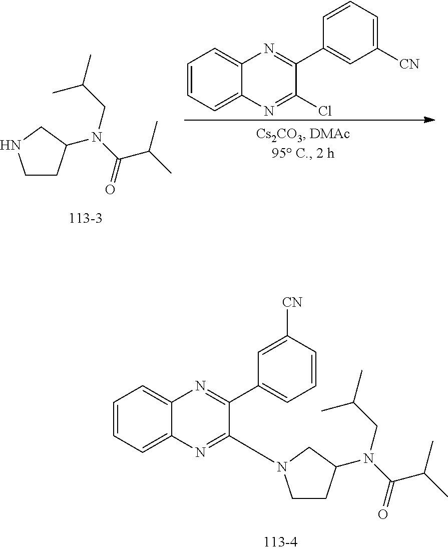

To a 10 mL round-bottom flask equipped with a magnetic stir bar was added N-isobutyl-2-methyl-N-pyrrolidin-3-yl-propanamide 113-3 (121.9 mg, 373 umol, 2 eq, TFA salt) followed by the addition of DMAc (3 mL). Then Cs₂CO₃ (304 mg, 933 umol, 5 eq) and 3-(3-chloroquinoxalin-2-yl)benzonitrile (50 mg, 186 umol, 99.257% purity, 1 eq) were added into the mixture at 25° C. The mixture was heated to 70° C. and stirred for 2 h. The LC-MS showed the starting material was consumed and the desired mass was detected. The suspension was filtrated and the filtrate was concentrated to give a crude product. The crude product was purified by preparative HPLC: (Phenomenex Luna C18 75×30 mm, 3 um); flow rate: 25 mL/min; gradient: 60%-90% B over 7 min; mobile phase A: water 0.1% aqueous TFA, mobile phase B: acetonitrile). After lyophilization, N-[1-[3-(3-cyanophenyl)quinoxalin-2-yl]pyrrolidin-3-yl]-N-isobutyl-2-methyl-propanamide 113 (76 mg, 136.38 umol, 36.53% yield, 99.7% purity, TFA salt) was obtained as a yellow solid. LCMS of 113: Ret. Time: 1.034 min, m/z: 442.2, M+H⁺. ¹H NMR of 113: (400 MHz, CDCl₃) δ 8.09 (s, 1H), 8.03-7.92 (m, 2H), 7.85 (br d, J=8.0 Hz, 1H), 7.73 (br d, J=7.6 Hz, 1H), 7.64 (td, J=7.2, 14.4 Hz, 2H), 7.53-7.45 (m, 1H), 4.55-4.31 (m, 1H), 3.57-3.50 (m, 1H), 3.50-3.29 (m, 3H), 3.11 (br d, J=7.6 Hz, 2H), 2.89-2.72 (m, 1H), 2.30-2.24 (m, 1H), 2.11-2.06 (m, 1H), 1.81 (td, J=6.4, 13.4 Hz, 1H), 1.16-1.06 (m, 6H), 0.94-0.81 (m, 6H).

### Example 6E: Preparation of Analogous Compounds 212, 213, 249, 250, and 405

Table 5, below, depicts exemplary compounds synthesized according to an analogous procedure to the one described for 113.

TABLE 5
Compounds synthesized analogously to 113.

| Compound Structure | Compound No. | Yield/Purity | Characterization |
|---|---|---|---|
| 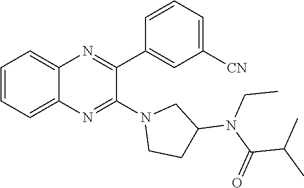 [SMILES: CCN(C(=O)C(C)C)C1CCN(c2nc3ccccc3nc2-c2cccc(C#N)c2)C1] | 212 | 4.61% yield, 99.6% purity | LC MS of 212: Ret. Time: 1.210 min, MS (ESI) m/z: 414.3, M + H⁺. ¹H NMR of 212: ¹H NMR (400 MHz, DMSO) δ 8.14 (s, 1H), 8.03 (t, J = 6.0 Hz. 1H), 7.96 (d, J = 7.6 Hz, 1H), 7.89 (d, J = 8.0 Hz, 1H), 7.74 (d, J = 7.2 Hz, 2H), 7.66 (t, J = 7.6 Hz, 1H), 7.46 (t, J = 7.2 Hz, 1H), 4.74-4.50 (m, 1H), 3.27-3.04 (m, 4H), 2.79 (m, J = 19.6, 13.2, 6.4 Hz, 1H), 2.07- 1.92 (m, 2H), 1.07-0.95 (m, 8H), 0.92 (d, J = 6.8 Hz, 3H). |
| 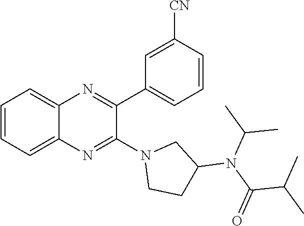 [SMILES: CC(C)C(=O)N(C(C)C)C1CCN(c2nc3ccccc3nc2-c2cccc(C#N)c2)C1] | 213 | 18.9% yield | LC MS of 213: MS (ESI) m/z: 428.1, M + H⁺. ¹H NMR of 213: ¹H NMR (400 MHz, CDCl₃) δ 8.17 (s, 1H), 8.05 (s, 1H), 7.94 (d, J = 16.0 Hz. 1H), 7.84 (s. 1H), 7.70 (s. 1H), 7.61 (s, 2H), 7.45 (s, 1H), 4.10 (s, 1H), 3.80 (s, 2H), 3.54 (s, 1H), 3.37 (s, 2H), 2.75 (s, 1H), 2.64 (s, 1H), 1.88 (s, 1H), 1.23 (t, J = 14.4 Hz, 6H), 1.08 (s, 6H). |
| 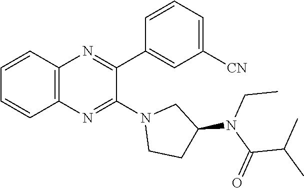 [SMILES: CCN(C(=O)C(C)C)[C@H]1CCN(c2nc3ccccc3nc2-c2cccc(C#N)c2)C1] | 249 | 4.4% yield | LC MS of 249: MS (ESI) m/z: 414.2, M + H⁺. ¹H NMR of 249: ¹H NMR (400 MHz, DMSO) δ 8.14 (s, 1H), 8.02 (d, J = 6.4 Hz, 1H), 7.96 (d, J = 7.6 Hz, 1H), 7.89 (d, J = 8.4 Hz, 1H), 7.73 (t, J = 9.2 Hz, 2H), 7.66 (t, J = 7.6 Hz. 1H), 7.46 (t. J = 7.6 Hz, 1H), 4.80-4.48 (m, 1H), 3.37 (s, 1H), 3.28 (d, J = 7.2 Hz, 2H), 3.21-3.06 (m, 2H), 2.74 (s, 1H), 2.00 (m, J = 20.4, 9.3 Hz. 2H), 0.98 (m, J = 29.6, 6.9 Hz, 9H). |
| 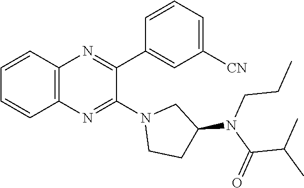 [SMILES: CCCN(C(=O)C(C)C)[C@H]1CCN(c2nc3ccccc3nc2-c2cccc(C#N)c2)C1] | 250 | 6.54% yield | LC MS of 250: Ret. Time: 1.339 min, MS (ESI) m/z: 428.2, M + H⁺. ¹H NMR of 250: ¹H NMR (400 MHz, DMSO) δ 8.13 (s, 1H), 8.07-7.85 (m. 3H), 7.73 (t, J = 9.6 Hz, 2H), 7.66 (t, J = 7.6 Hz. 1H), 7.47 (d, J = 6.4 Hz, 1H), 4.65-4.52 (m. 1H), 3.38 (s. 1H), 3.28-3.21 (m, 1H). 3.10-2.89 (m, 3H), 2.68-2.67 (m, 1H), 2.56 (s, 1H), 2.03 (d, J = 8.0 Hz, 2H), 2.02-1.99 (m, 2H), 1.05-0.89 (m, 6H), 0.83-0.75 (m, 3H). |
| 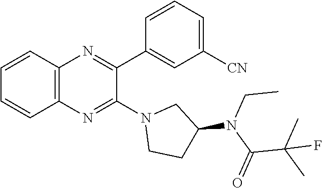 [SMILES: CCN(C(=O)C(C)(C)F)[C@H]1CCN(c2nc3ccccc3nc2-c2cccc(C#N)c2)C1] | 405 | 28.8% yield | LC MS of 405: Ret. Time: 1.433 min, MS (ESI) m/z: 432.0, [M + H]⁺. ¹H NMR of 405: ¹H NMR (400 MHz, DMSO-d6) δ 8.12 (t, J = 1.6 Hz, 1H), 8.03 (dt, J = 7.8, 1.3 Hz, 1H), 7.96-7.83 (m, 2H), 7.73 (ddd, J = 12.9, 8.7, 4.5 Hz, 2H), 7.64 (ddd, J = 8.3, 6.9, 1.4 Hz, 1H), 7.46 (ddd. J = 8.2, 7.0, 1.4 Hz, 1H), 4.88-4.39 (m, 1H), 3.49-3.20 (m, 6H), 2.21-1.87 (m, 2H), 1.52 (dd, J = 21.7, 5.6 Hz, 6H), 1.04 (t. J = 6.9 Hz, 3H). |

Compound 212 according to the present disclosure was prepared as shown in Scheme 61 by an analogous procedure to the one described in Examples 6A-6D.

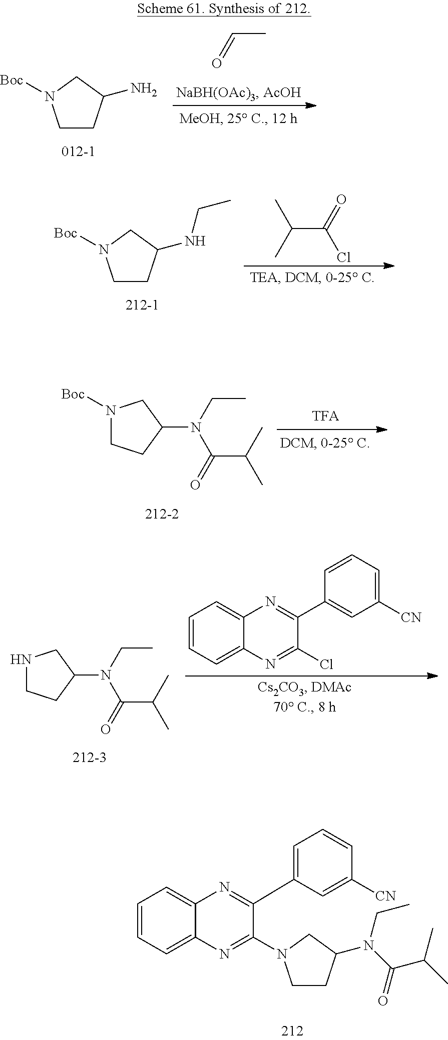

Compound 213 according to the present disclosure was prepared as shown in Scheme 61 by an analogous procedure to the one described in Examples 6A-6D.

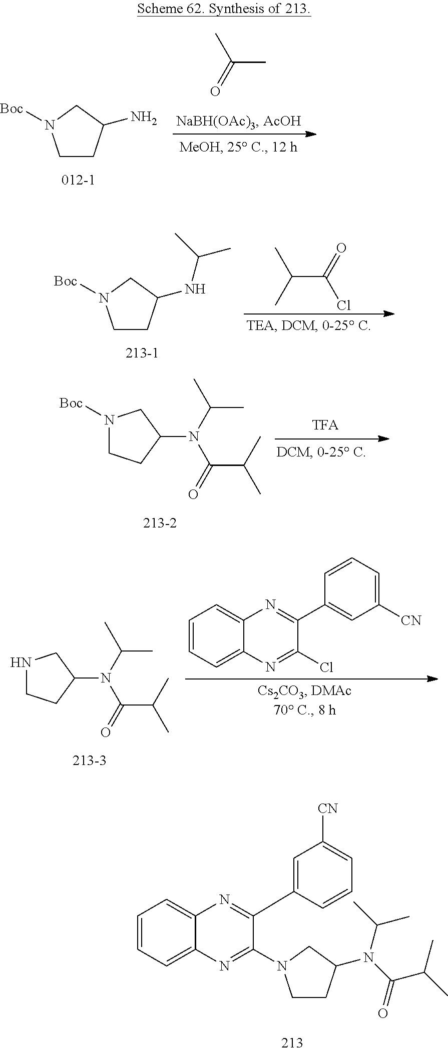

Compound 249 according to the present disclosure was prepared as shown in Scheme 63 by an analogous procedure to the one described in Examples 6A-6D.

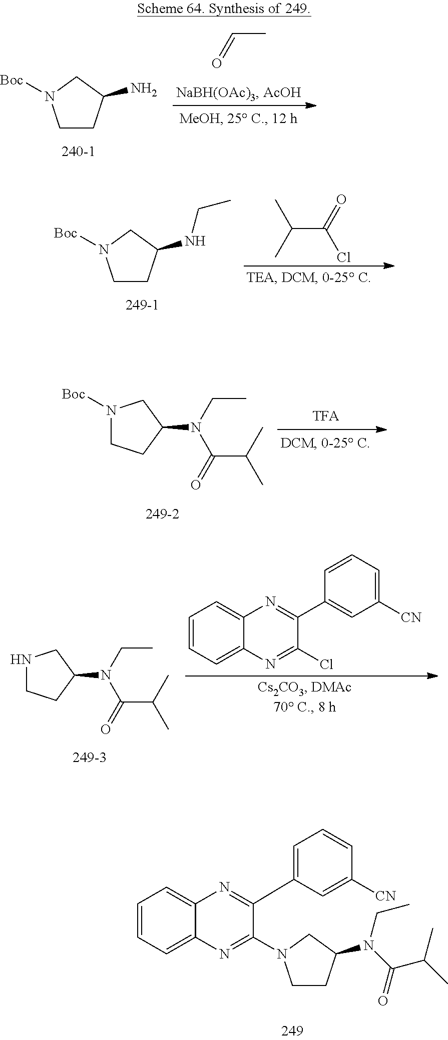

Compound 250 according to the present disclosure was prepared as shown in Scheme 64 by an analogous procedure to the one described in Examples 6A-6D.

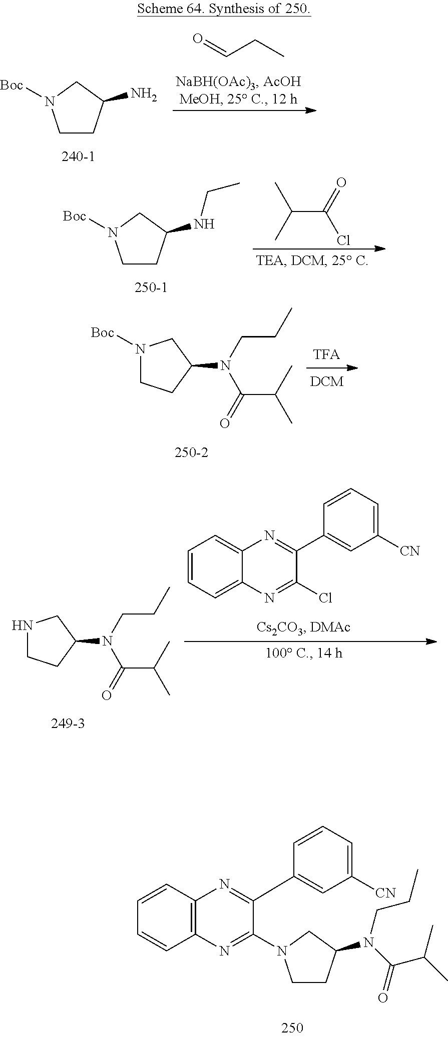

Compound 405 according to the present disclosure was prepared as shown in Scheme 65 by an analogous procedure to the one described in Examples 6A-6D.

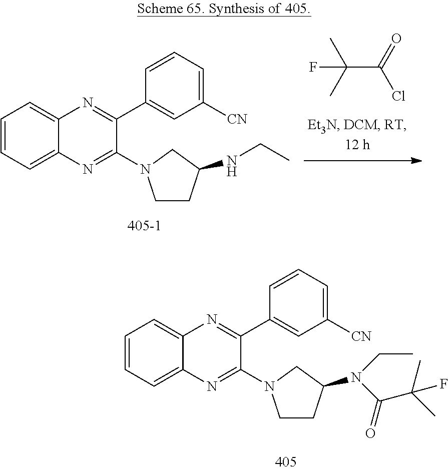

### Example 7: Synthesis of Analog 205

Compound 205 according to the present disclosure was prepared as shown in Scheme 66 and described below.

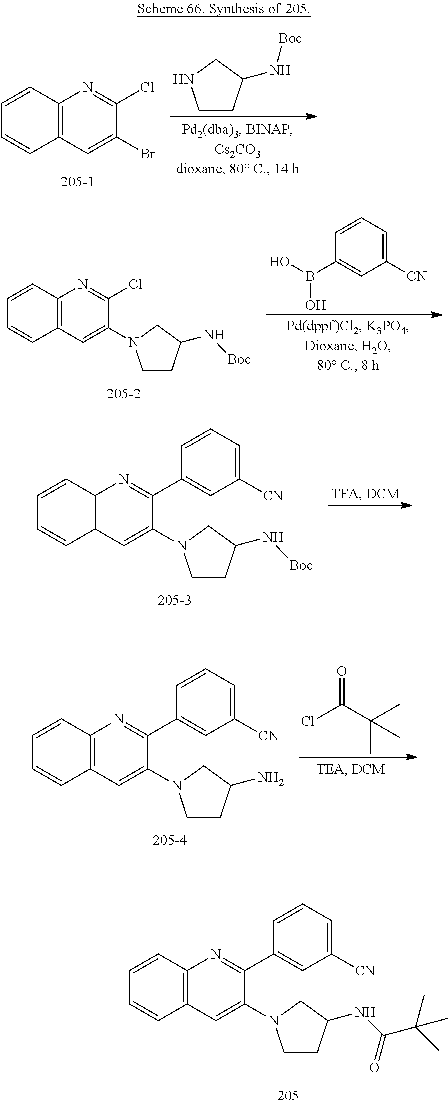

### Example 7A: Preparation of Compound 205-2

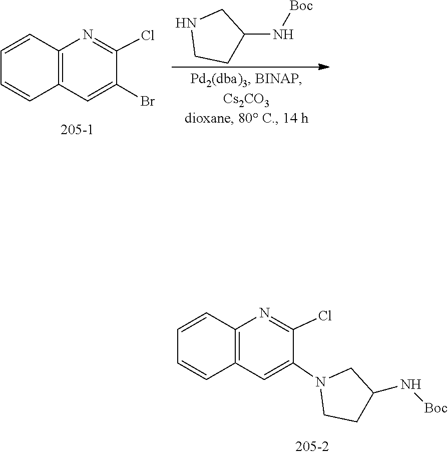 [SMILES: Clc1nc2ccccc2cc1Br ; CC(C)(C)OC(=O)NC1CCNC1 ; CC(C)(C)OC(=O)NC1CCN(c2cc3ccccc3nc2Cl)C1]

To a solution of 3-bromo-2-chloroquinoline (2.0 g, 8.2 mmol), tert-butyl pyrrolidin-3-ylamino formate (1.5 mg, 8.2 mmol) and Cs₂CO₃ (8.0 g, 24.6 mmol) in 1,4-dioxane (40 mL) stirred under nitrogen at 25° C. was added tris(dibenzylideneacetone) dipalladium (0.8 g, 0.8 mmol) and 2,2′-bis(diphenylphosphino)-1,1′-binaphthyl (1.0 g, 1.6 mmol). The reaction mixture was stirred at 80° C. for 14 h. After completion, the mixture was cooled to room temperature and quenched with H₂O (40 mL), then extracted with EtOAc (40 mL×3). The combined organic layers were washed with brine three times and dried over sodium sulphate, filtered, and concentrated. The residue was purified by a flash chromatography to afford compound 205-2 (150 mg, yield: 4.72%) as a yellow solid. ¹H NMR of 205-2: ¹H NMR (400 MHz, CDCl₃) δ 7.92 (d, J=8.2 Hz, 1H), 7.68 (dd, J=8.0, 1.2 Hz, 1H), 7.56-7.45 (m, 3H), 7.15-7.05 (m, 1H), 4.91 (s, 1H), 4.40 (s, 1H), 3.61 (dd, J=10.0, 6.0 Hz, 1H), 3.38 (dd, J=24.8, 6.8 Hz, 2H), 2.45-2.29 (m, 1H), 1.96 (d, J=6.0 Hz, 1H), 1.47 (s, 9H).

### Example 7B: Preparation of Compound 205-3

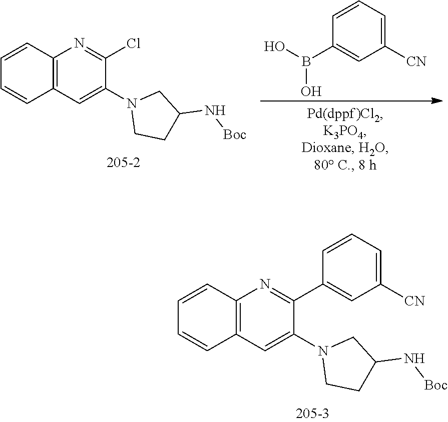 [SMILES: CC(C)(C)OC(=O)NC1CCN(c2cc3ccccc3nc2Cl)C1 ; N#Cc1cccc(B(O)O)c1 ; CC(C)(C)OC(=O)NC1CCN(c2cc3ccccc3nc2-c2cccc(C#N)c2)C1]

To a solution of compound 205-2 (35 mg, 0.10 mmol), 3-(dihydroxyboranyl)benzonitrile (14.7 mg, 0.10 mmol) and Na₂CO₃ (31.9 mg, 0.30 mmol) in a 1,4-dioxane:H₂O 5:1 solution (3 mL) stirred under nitrogen at 25° C. was added 1,1′-bis(diphenylphosphino)ferrocenepalladium dichloride (7.3 mg, 0.01 mmol). The reaction mixture was stirred at 80° C. for 8 h. After completion, the mixture was cooled to room temperature and quenched with H₂O (5 mL), then extracted with EtOAc (5 mL×3). The combined organic layers were washed with brine three times and dried over sodium sulphate, filtered, and concentrated. The residue was purified by flash chromatography to afford compound 205-3 (15 mg, yield: 32.40%) as a yellow solid. LC MS of 205-3: Ret. Time: 1.336 min, MS (ESI) m/z: 415.3, M+H⁺.

### Example 7C: Preparation of Compound 205-4

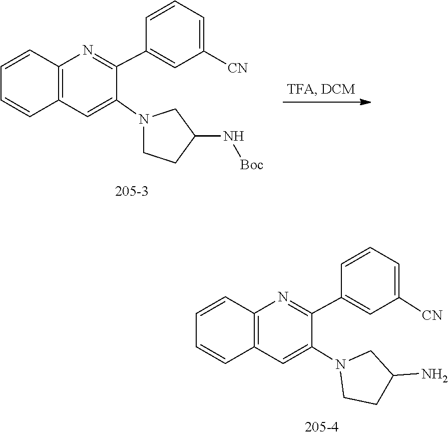 [SMILES: CC(C)(C)OC(=O)NC1CCN(c2cc3ccccc3nc2-c2cccc(C#N)c2)C1 ; N#Cc1cccc(-c2nc3ccccc3cc2N2CCC(N)C2)c1]

To a solution of compound 205-3 (30 mg, 0.07 mmol) in DCM (1 mL) stirred under nitrogen at 0° C. was added trifluoroacetic acid (0.3 mL). The reaction mixture was stirred at 25° C. for 1 h. After completion, the mixture was adjusted to pH 10 with saturated aqueous NaHCO₃ solution and extracted with DCM (3 mL×3). The combined organic layers were washed with brine three times and dried over sodium sulphate, filtered, and concentrated. The residue was purified by flash chromatography eluting with petroleum ether:EtOAc (3:1) to afford compound 205-4 (15 mg, yield: 59.42%) as a yellow solid. LC MS of 205-4: Ret. Time: 0.740 min, MS (ESI) m/z: 315.2, M+H⁺.

### Example 7D: Preparation of Compound 205

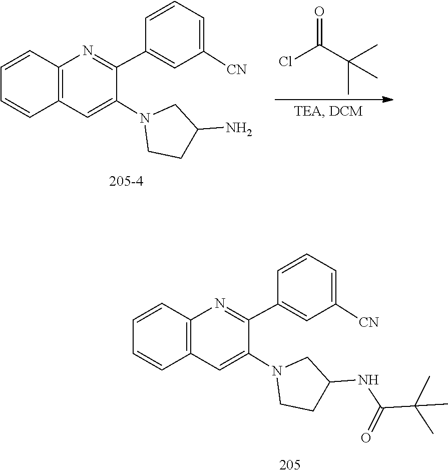

To a solution of compound 205-4 (30 mg, 0.10 mmol) and N,N-diisopropylethylamine (37.0 mg, 0.29 mmol) in DCM (2 mL) stirred under nitrogen at 0° C. was added 2,2-dimethylpropanoyl chloride (12.7 mg, 0.10 mmol). The reaction mixture was stirred at 25° C. for 1 h. After completion, the mixture was quenched with H₂O (3 mL) and extracted with DCM (3 mL×3). The combined organic layers were washed with brine three times and dried over sodium sulphate, filtered, and concentrated. The residue was purified by a preparative HPLC to afford compound 205 (6.2 mg, yield: 15.30%) as a yellow solid. LC MS of 205: Ret. Time: 1.181 min, MS (ESI) m/z: 399.0, M+H⁺. ¹H NMR of 205: ¹H NMR (400 MHz, DMSO) δ 8.12 (dd, J=28.0, 20.4 Hz, 2H), 8.01-7.84 (m, 3H), 7.80-7.62 (m, 2H), 7.60-7.35 (m, 3H), 4.23 (d, J=6.0 Hz, 1H), 3.19 (dd J=9.6, 6.4 Hz, 1H), 3.03 (d, J=7.6 Hz, 1H), 2.96-2.83 (m, 2H), 2.01 (dd, J=12.4, 5.6 Hz, 1H), 1.88-1.77 (m, 1H), 1.14-1.00 (m, 9H).

### Example 8: Synthesis of Analogs 206 and 248

Compound 206 according to the present disclosure was prepared as shown in Scheme 67 and described below.

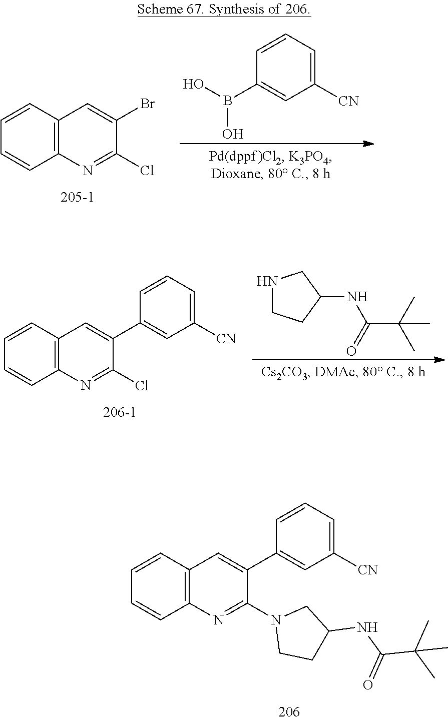 [SMILES: Clc1nc2ccccc2cc1Br ; N#Cc1cccc(B(O)O)c1 ; N#Cc1cccc(-c2cc3ccccc3nc2Cl)c1 ; CC(C)(C)C(=O)NC1CCNC1 ; CC(C)(C)C(=O)NC1CCN(c2nc3ccccc3cc2-c2cccc(C#N)c2)C1]

### Example 8A: Preparation of Compound 206-1

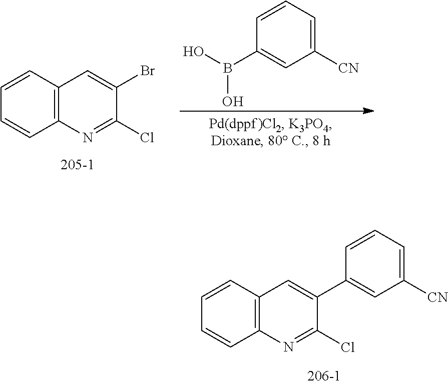 [SMILES: Clc1nc2ccccc2cc1Br ; N#Cc1cccc(B(O)O)c1 ; N#Cc1cccc(-c2cc3ccccc3nc2Cl)c1]

To a solution of 3-bromo-2-chloroquinoline (400 mg, 2.06 mmol), 3-(dihydroxyboranyl)benzonitrile 205-1 (230 mg, 1.57 mmol) and K₃PO₄ (700 mg, 3.30 mmol) in dioxane (5 mL) stirred under nitrogen was added Pd(dppf)Cl₂ (96.6 mg, 0.132 mmol). The reaction mixture was stirred at 80° C. for 8 h. The mixture was cooled to room temperature and quenched with H₂O (15 mL), then extracted with EtOAc (15 mL×3). The combined organic layers were washed with brine three times and dried over anhydrous sodium sulphate, filtered and concentrated. The residue was purified via flash chromatography eluting with petroleum ether/EtOAc (10:1) to give the product 3-(2-chloroquinolin-3-yl)benzonitrile 206-1 as a white solid (340 mg, 77.9% yield). LC MS of 206-1: Ret. Time: 1.203 min, MS (ESI) m/z: 265.2, M+H⁺.

### Example 8B: Preparation of Compound 206

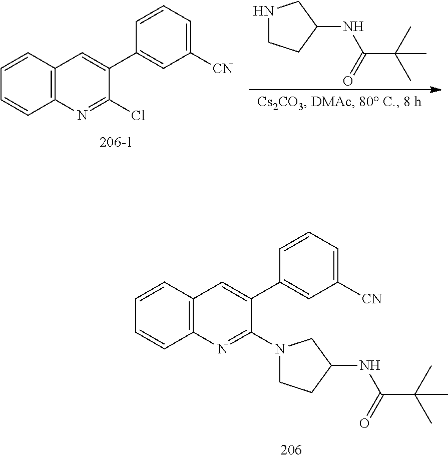 [SMILES: N#Cc1cccc(-c2cc3ccccc3nc2Cl)c1 ; CC(C)(C)C(=O)NC1CCNC1 ; CC(C)(C)C(=O)NC1CCN(c2nc3ccccc3cc2-c2cccc(C#N)c2)C1]

To a solution of 2,2-dimethyl-N-(pyrrolidin-3-yl) propenamide (96.5 mg, 0.567 mmol) in DMAc (5 mL) stirred under nitrogen was added Cs₂CO₃ (923 mg, 2.83 mmol) and 3-(2-chloroquinolin-3-yl) benzonitrile 206-1 (150 mg, 0.567 mmol). The reaction mixture was stirred at 70° C. for 8 h. The mixture was cooled to room temperature and quenched with H₂O (10 mL), then extracted with EtOAc (30 mL×3). The combined organic layers were washed with brine three times and dried over anhydrous sodium sulphate, filtered and concentrated. The residue was purified via preparative HPLC to give the product N-{1-[3-(3-cyanophenyl)quinolin-2-yl]pyrrolidin-3-yl}-2,2-dimethylpropanamide 206 (40.5 mg, 95.6% purity, 17.2% yield) as a yellow solid. LC MS of 206: Ret. Time: 0.840 min, MS (ESI) m/z: 399.1 M+H⁺. ¹H NMR of 206: ¹H NMR (400 MHz, DMSO) δ 8.02 (s, 1H), 7.98 (s, 1H), 7.89-7.81 (m, 2H), 7.77 (d, J=7.2 Hz, 1H), 7.70-7.64 (m, 2H), 7.61-7.55 (m, 1H), 7.41 (d, J=6.4 Hz, 1H), 7.30-7.24 (m, 1H), 4.17 (d, J=6.4 Hz, 1H), 3.42 (dd, J=10.8, 6.4 Hz, 1H), 3.15 (m, J=19.2, 13.9, 7.5 Hz, 3H), 1.84 (m, J=8.8, 12.4, 6.4 Hz, 2H), 1.06 (s, 9H).

### Example 8C: Preparation of Analogous Compound 248

Table 6, below, depicts exemplary compounds synthesized according to an analogous procedure to the one described for 206.

TABLE 6
Compounds synthesized analogously to 206.

| Compound Structure | Compound No. | Yield | Characterization |
|---|---|---|---|
| 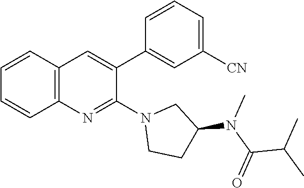 | 248 | 16.1% yield | LC MS of 248: MS (ESI) m/z: 432.0, [M + H]⁺. ¹H NMR of 248: ¹H NMR (400 MHz, DMSO) δ 8.30 (s, 1H), 8.05 (s, 1H), 7.92 (m, J = 14.7, 9.6 Hz, 4H), 7.80-7.69 (m, 2H), 7.46 (s, 1H), 5.01 (s, 1H), 3.50 (s, 1H), 3.26 (dd, J = 20.8, 6.8 Hz, 3H), 2.98-2.79 (m, 3H), 2.70-2.64 (m, 1H), 1.98 (d, J = 30.8 Hz, 2H), 0.98 (d, J = 6.4 Hz, 6H). |

Compound 248 according to the present disclosure was prepared as shown in Scheme 68 by an analogous procedure to the one described in Examples 8A-8B.

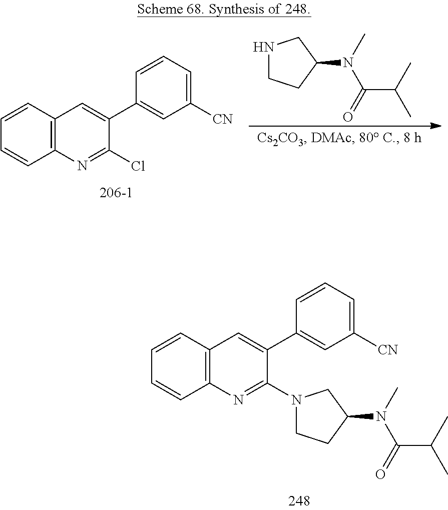 [SMILES: N#Cc1cccc(-c2cc3ccccc3nc2Cl)c1 ; CC(C)C(=O)N(C)[C@H]1CCNC1 ; CC(C)C(=O)N(C)[C@H]1CCN(c2nc3ccccc3cc2-c2cccc(C#N)c2)C1]

### Example 9: Synthesis of Analogs 216, 217, and 218

Compound 217 according to the present disclosure was prepared as shown in Scheme 69 and described below.

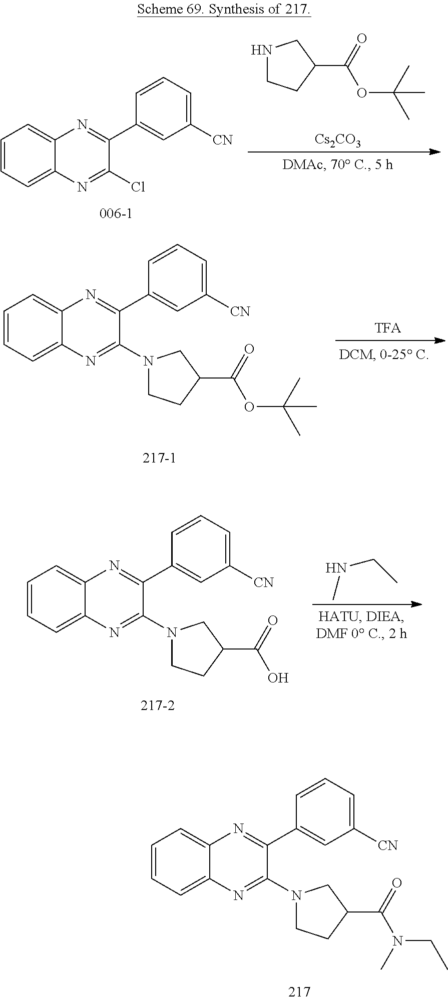

### Example 9A: Preparation of Compound 217-1

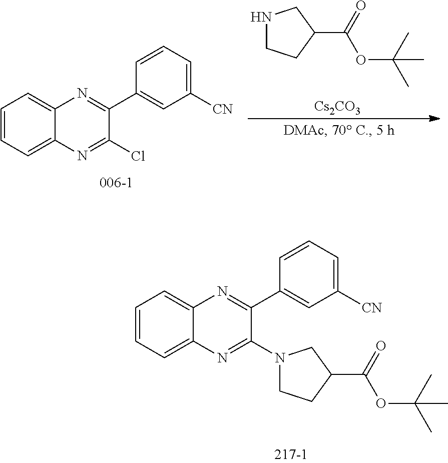

To a solution of 3-(3-chloroquinoxalin-2-yl)benzonitrile (500 mg, 1.88 mmol) 006-1 and tert-butyl pyrrolidine-3-carboxylate (322 mg, 1.88 mmol) in DMAc (10 mL) was added Cs₂CO₃ (3.06 g, 9.41 mmol). The reaction mixture was stirred at 70° C. for 2 h, then cooled to room temperature. The organic phase was washed with water (20 mL). The residue was extracted with EtOAc (3×20 mL). The reaction mixture was concentrated under reduced pressure. The residue was purified via flash chromatography eluting with petroleum ether/EtOAc to give tert-butyl 1-(3-(3-cyanophenyl)quinoxalin-2-yl)pyrrolidine-3-carboxylate 217-1 (480 mg, 57.3% yield) as a brown oil. LC MS of 217-1: Ret. Time: 1.16 min, MS (ESI) m/z: 467, M+H⁺.

### Example 9B: Preparation of Compound 217-2

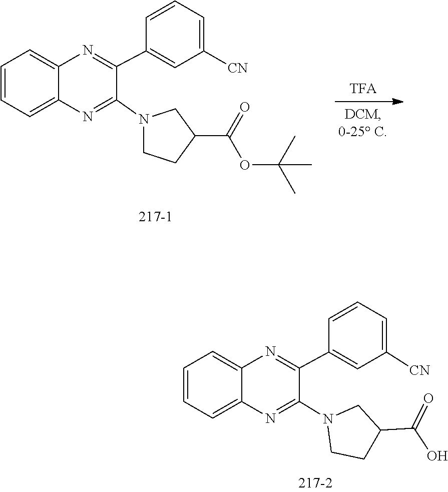 [SMILES: CC(C)(C)OC(=O)C1CCN(c2nc3ccccc3nc2-c2cccc(C#N)c2)C1 ; N#Cc1cccc(-c2nc3ccccc3nc2N2CCC(C(=O)O)C2)c1]

To a solution of tert-butyl 1-[3-(3-cyanophenyl)quinoxalin-2-yl]pyrrolidine-3-carboxylate (430 mg, 1.07 mmol) 217-1 in DCM (10 mL) at 0° C. was added trifluoroacetic acid (367 mg, 3.22 mmol). The reaction mixture was stirred at 25° C. for 3 h. The reaction mixture was concentrated under reduced pressure to give 1-[3-(3-cyanophenyl)quinoxalin-2-yl]pyrrolidine-3-carboxylic acid 217-2 (360 mg, 87.6% yield) as a yellow solid. ¹H NMR of 217-2: ¹H NMR (400 MHz, DMSO) δ 8.13 (s, 1H), 7.98 (dd, J=16.0, 7.2 Hz, 2H), 7.89 (d, J=7.2 Hz, 1H), 7.73 (dd, J=12.8, 5.0 Hz, 2H), 7.66 (t, J=7.6 Hz, 1H), 7.50-7.43 (m, 1H), 3.40 (d, J=6.8 Hz, 2H), 3.27 (t, J=7.2 Hz, 2H), 3.03 (dd, J=14.4, 7.2 Hz, 1H), 2.14-1.91 (m, 2H).

### Example 9C: Preparation of Compound 217

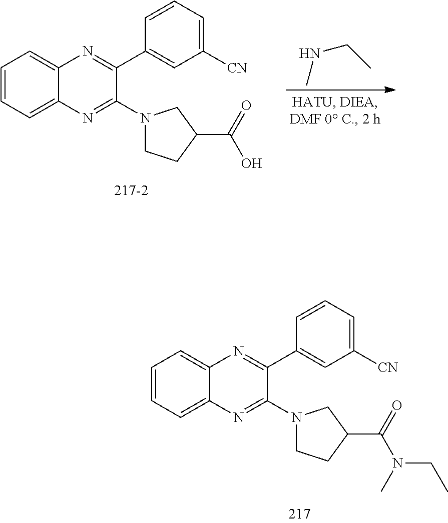

To a solution of 1-[3-(3-cyanophenyl)quinoxalin-2-yl]pyrrolidine-3-carboxylic acid 217-2 (100 mg, 0.290 mmol), DIEA (75.1 mg, 0.581 mmol) and ethyl(methyl)amine (20.6 mg, 0.348 mmol) in DMF (5 mL) at 0° C. was added HATU (133 mg, 0.348 mmol). The reaction mixture was stirred at 0° C. for 2 h. The mixture was quenched with H₂O (5 mL) and extracted with EtOAc (5 mL×3). The combined organic layers were washed with brine and dried over sodium sulphate, filtered, and concentrated. The residue was purified by a preparative HPLC to afford the product 1-[3-(3-cyanophenyl)quinoxalin-2-yl]-N-ethyl-N-methylpyrrolidine-3-carboxamide 217 (25.4 mg, 100% purity, 22.7% yield) as a yellow solid. LC MS of 217: Ret. Time: 1.079 min, MS (ESI) m/z: 386, M+H⁺. ¹H NMR of 217: ¹H NMR (400 MHz, CDCl₃) δ 8.11 (s, 1H), 8.01-7.86 (m, 3H), 7.73 (d, J=7.6 Hz, 1H), 7.63 (dd, J=12.4, 7.2 Hz, 2H), 7.46 (t, J=7.6 Hz, 1H), 3.68-3.52 (m, 2H), 3.46-3.36 (m, 4H), 3.23 (t, J=10.4 Hz, 1H), 2.99 (d, J=51.2 Hz, 3H), 2.25-2.07 (m, 2H), 1.15 (dt, J=39.9, 7.1 Hz, 3H).

### Example 9D: Preparation of Analogous Compounds 216 and 218

Table 7, below, depicts exemplary compounds synthesized according to an analogous procedure to the one described for 217.

TABLE 7
Compounds synthesized analogously to 217.

| Compound Structure | Compound No. | Yield | Characterization |
|---|---|---|---|
| 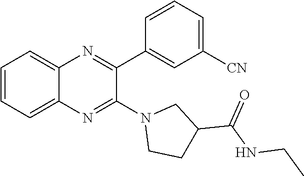 [SMILES: CCNC(=O)C1CCN(c2nc3ccccc3nc2-c2cccc(C#N)c2)C1] | 216 | 18.9% yield | LC MS of 216: MS (ESI) m/z: 372.0, [M + H]⁺. ¹H NMR of 216: ¹H NMR (400 MHz, CDCl₃) δ 8.10 (s, 1H), 7.97 (dd, J = 18.4, 8.8 Hz, 3H), 7.74 (d, J = 7.6 Hz, 1H), 7.64 (q, J = 8.0 Hz, 2H), 7.48 (t, J = 7.6 Hz, 1H), 5.59 (s, 1H), 3.65 (d, J = 33.2 Hz, 2H), 3.51-3.21 (m. 4H), 2.87 (s, 1H), 2.23-2.08 (m, 2H), 1.15 (t, J = 7.3 Hz, 3H). |
| 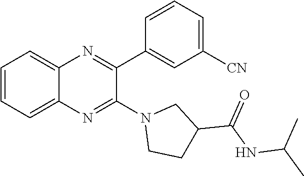 [SMILES: CC(C)NC(=O)C1CCN(c2nc3ccccc3nc2-c2cccc(C#N)c2)C1] | 218 | 32.7% yield | LC MS of 218: MS (ESI) m/z: 386.0, [M + H]⁺. ¹H NMR of 218: ¹H NMR (400 MHz, CDCl₃) δ 8.10 (s, 1H), 8.01-7.89 (m, 3H), 7.74 (d, J = 7.6 Hz. 1H), 7.64 (q, J = 8.0 Hz, 2H), 7.48 (t, J = 7.6 Hz, 1H), 5.40 (s, 1H), 4.07 (m, J = 13.6, 6.8 Hz, 1H), 3.64 (d, J = 28.4 Hz, 2H), 3.38 (d, J = 22.8 Hz, 2H), 2.89-2.78 (m, 1H), 2.19-2.08 (m, 2H), 1.16 (t, J = 6.4 Hz, 6H). |

Compound 216 according to the present disclosure was prepared as shown in Scheme 70 by an analogous procedure to the one described in Examples 9A-9C.

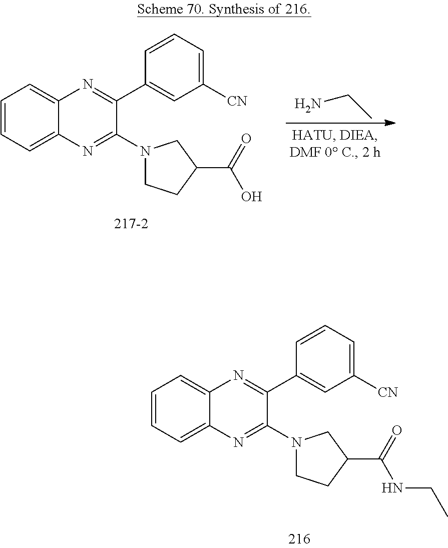

Compound 218 according to the present disclosure was prepared as shown in Scheme 71 by an analogous procedure to the one described in Examples 9A-9C.

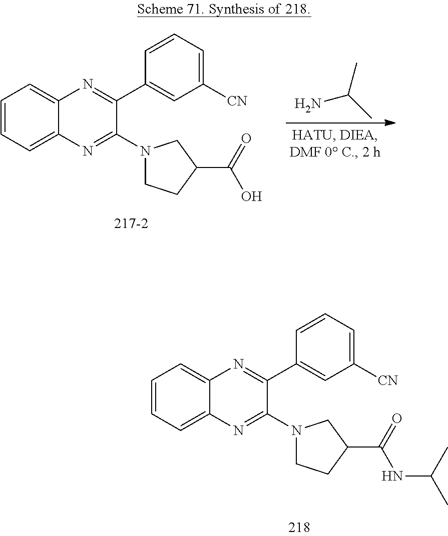

### Example 10: Synthesis of Analogs 225 and 224

Compound 225 according to the present disclosure was prepared as shown in Scheme 72 and described below.

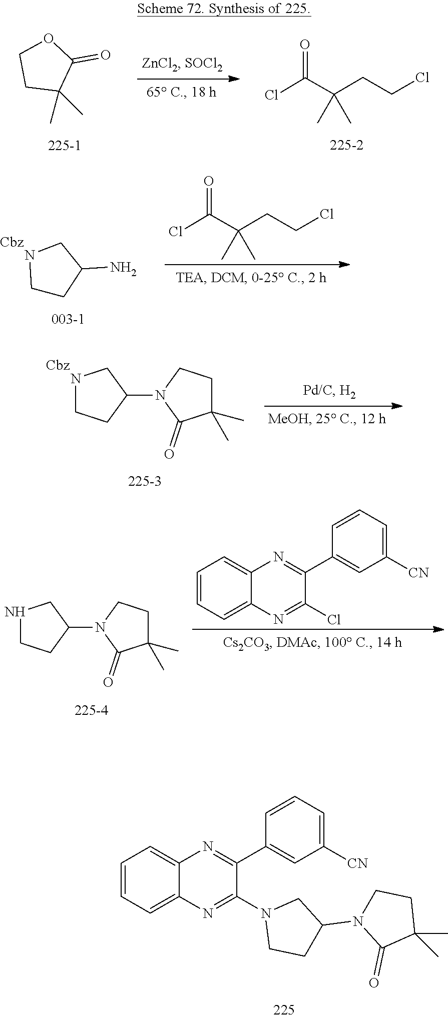

### Example 10A: Preparation of Compound 225-2

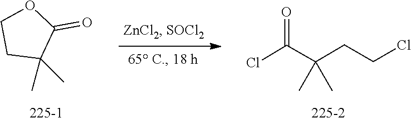

To a solution of 3,3-dimethyldioxolan-2-one 225-1 (800 mg, 7.01 mmol) in thionyl chloride (3.336 g, 28.0 mmol) was added ZnCl₂ (95.5 mg, 0.701 mmol). The reaction mixture was stirred at 65° C. for 16 h then cooled to room temperature. The solution was filtered and the filtrate was concentrated under reduced pressure. 2,2-Dimethylpentanoyl chloride 225-2 (600 mg, crude) was obtained as a liquid and used directly in the next step.

### Example 10B: Preparation of Compound 225-3

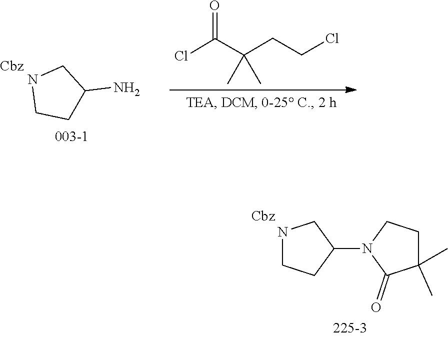

To a solution of benzyl 3-aminopyrrolidine-1-carboxylate 003-1 (1.04 g, 4.73 mmol) in DCM (10 mL) stirred under nitrogen at 0° C. was added triethylamine (1.19 g, 11.8 mmol) and 4-chloro-2,2-dimethylbutanoyl chloride 225-2 (800 mg, 4.73 mmol). The reaction mixture was stirred at 25° C. for 8 h. The mixture was quenched with H₂O (20 mL) and extracted with DCM (3×30 mL), then concentrated under reduced pressure. The residue was purified via flash chromatography eluting with DCM/MeOH (10:1) to give the product 3,3-dimethyl-1-(pyrrolidin-3-yl) pyrrolidin-2-one (868 mg, 52.1% yield) as colorless oil. LC MS of 225-3: Ret. Time: 0.742 min, MS (ESI) m/z: 317.2, M+H⁻.

### Example 10C: Preparation of Compound 225-4

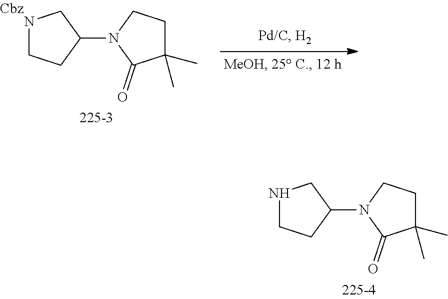

To a solution of 3,3-dimethyl-1-(pyrrolidin-3-yl) pyrrolidin-2-one 225-3 (400 mg, 1.26 mmol) in MeOH (8 mL) was added 10% palladium on carbon (404 mg, 0.379 mmol). The mixture was stirred under an atmosphere of H₂ at 50° C. for 16 h. The solution was cooled to room temperature and filtered. The filtrate was concentrated under reduced pressure to give the product 3,3-dimethyl-1-(pyrrolidin-3-yl)pyrrolidin-2-one (185 mg, 72.2% yield) as a colorless oil. LC MS of 225-4: Ret. Time: 0.177 min, MS (ESI) m/z: 183.3, M+H⁺.

### Example 10D: Preparation of Compound 225

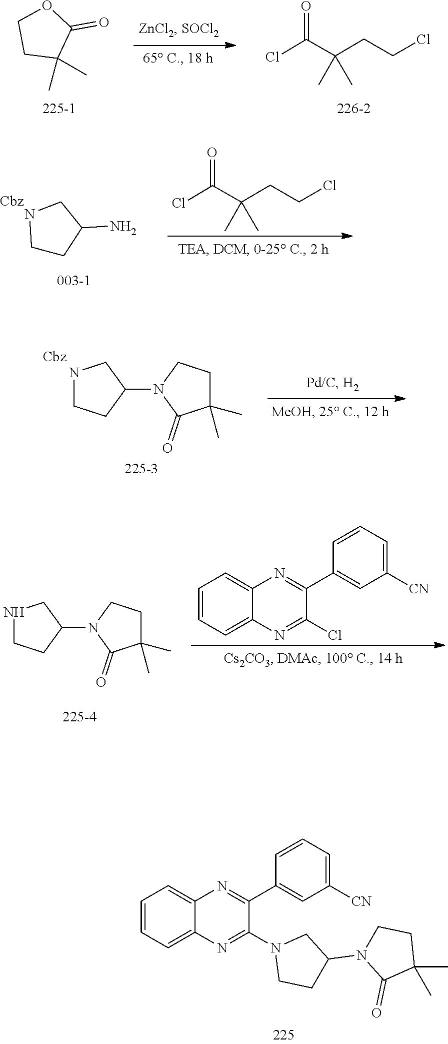

To a solution of 3,3-dimethyl-1-(pyrrolidin-3-yl)pyrrolidin-2-one (100 mg, 0.549 mmol) 225-4 in DMAc (5 mL) stirred under nitrogen was added Cs₂CO₃ (536 mg, 1.65 mmol] and 3-(3-chloroquinoxalin-21-yl)benzonitrile (146 mg, 0.549 mmol). The reaction mixture was stirred at 70° C. for 2 h. The organic phase was washed with water (30 mL). The residue was extracted with EtOAc (2×30 mL). The residue was purified via preparative HPLC to give the product 3-{3-[3-(3,3-dimethyl-2-oxopyrrolidin-1-yl)pyrrolidin-1-yl]quinoxalin-2-yl}benzonitrile 225 (11.3 mg, 99.6% purity, 4.99% yield) as a yellow solid. LC MS of 225: Ret. Time: 1.172 min, MS (ESI) m/z: 412.2, M+H⁺. ¹H NMR of 225: ¹H NMR (400 MHz, DMSO) δ 8.15 (t, J=1.6 Hz, 1H), 8.04-8.00 (m, 1H), 7.97-7.93 (m, 1H), 7.89 (dd, J=8.4, 1.2 Hz, 1H), 7.73 (t, J=8.0 Hz, 2H), 7.66 (m, J=8.4, 6.8, 1.6 Hz, 1H), 7.47 (m, J=8.4, 6.8, 1.6 Hz, 1H), 4.51 (p, J=6.8 Hz, 1H), 3.47 (dd, J=11.2, 7.2 Hz, 1H), 3.31-3.15 (m, 5H), 2.04-1.87 (m, 2H), 1.76 (t, J=6.8 Hz, 2H), 1.00 (t, J=9.2 Hz, 6H).

### Example 10E: Preparation of Analogous Compound 224

Table 8, below, depicts exemplary compounds synthesized according to an analogous procedure to the one described for 225.

TABLE 8
Compounds synthesized analogously to 225.

| Compound Structure | Compound No. | Yield | Characterization |
|---|---|---|---|
| 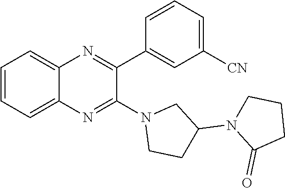 [SMILES: N#Cc1cccc(-c2nc3ccccc3nc2N2CCC(N3CCCC3=O)C2)c1] | 224 | 5.1% yield | [M + H]⁺. ¹H NMR of 224: ¹H NMR (400 MHz, DMSO) δ 8.15 (1, J = 1.6 Hz, 1H). 8.04-8.00 (m, 1H). 7.98-7.94 (m, 1H), 7.90 (dd. J = 8.4, 1.2 Hz, 1H), 7.73 (dd, J = 8.4, 7.6 Hz, 2H), 7.66 (m, J = 8.4, 6.9, 1.4 Hz, 1H), 7.47 (m. J = 8.4, 6.8, 1.6 Hz, 1H), 4.53 (m. J = 6.8 Hz, 1H), 3.44 (dd, J = 11.2, 7.2 Hz, 2H), 3.30-3.23 (m, 4H), 2.20 (dd, J = 10.4, 5.6 Hz, 2H), 2.00-1.85 (m, 4H). |

Compound 224 according to the present disclosure was prepared as shown in

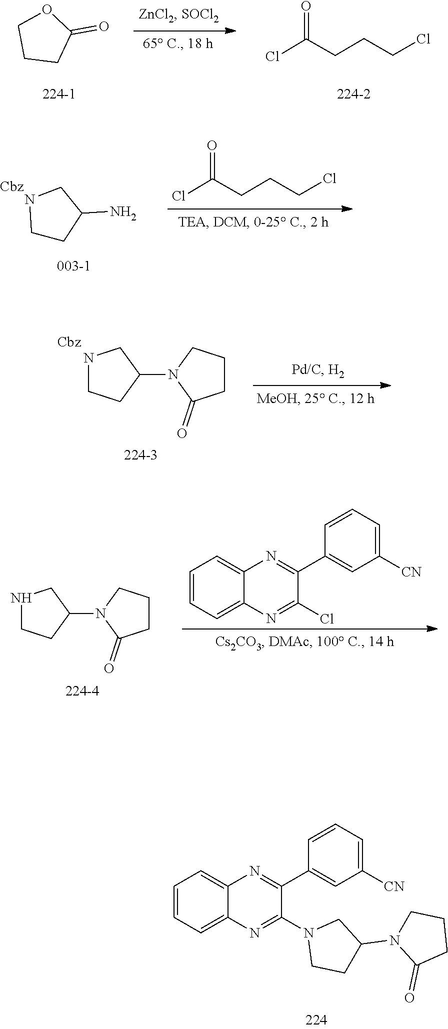

Scheme 73 by an analogous procedure to the one described in Examples 10A-10D.

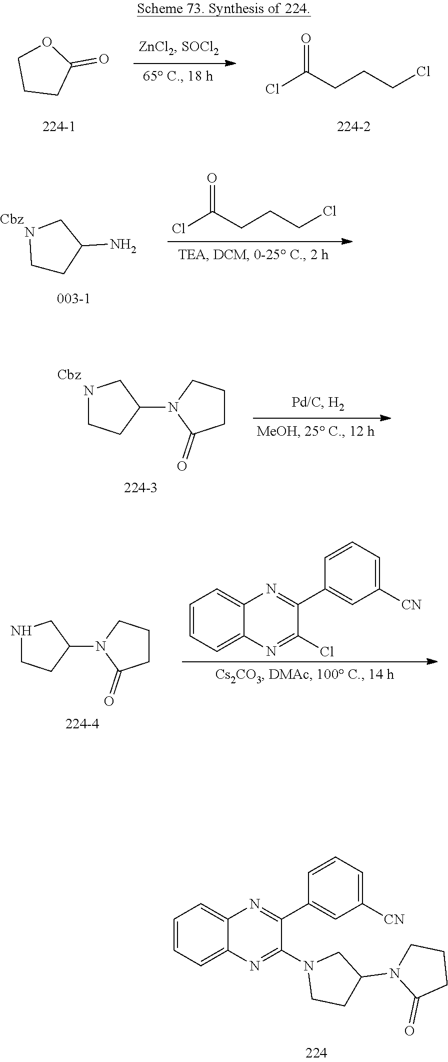

### Example 11: Synthesis of Analogs 304, 299, and 300

Compound 304 according to the present disclosure was prepared as shown in Scheme 74 and described below.

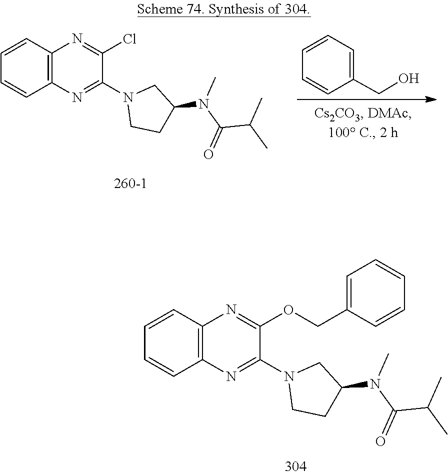 [SMILES: CC(C)C(=O)N(C)[C@H]1CCN(c2nc3ccccc3nc2Cl)C1 ; OCc1ccccc1 ; CC(C)C(=O)N(C)[C@H]1CCN(c2nc3ccccc3nc2OCc2ccccc2)C1]

### Example 11A: Preparation of Compound 304

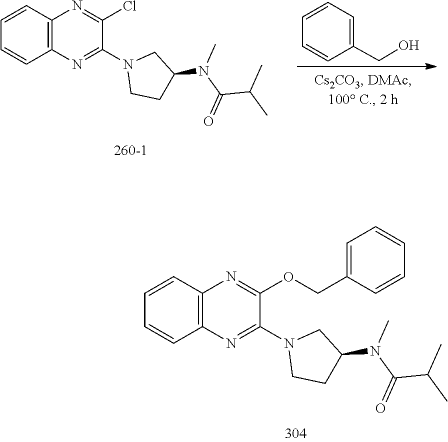 [SMILES: CC(C)C(=O)N(C)[C@H]1CCN(c2nc3ccccc3nc2Cl)C1 ; OCc1ccccc1 ; CC(C)C(=O)N(C)[C@H]1CCN(c2nc3ccccc3nc2OCc2ccccc2)C1]

To a mixture of (S)—N-(1-(3-chloroquinoxalin-2-yl)pyrrolidin-3-yl)-N-methylisobutyramide 260-1 (100 mg, 0.30 mmol, 1.0 eq) in DMAc (5 mL) was added phenylmethanol (39 mg, 0.36 mmol, 1.2 eq) and Cs₂CO₃ (294 mg, 0.90 mmol, 3.0 eq) at 25° C. The mixture was stirred at 100° C. for 2 h under N₂. After completion, the mixture was cooled to room temperature, quenched with H₂O (50 mL) and extracted with EtOAc (30 mL×2). The combined organic layers were washed with H₂O (20 mL×2) and brine (20 mL), then dried over anhydrous Na₂SO₄, filtered and concentrated in vacuo to give a residue. The residue was purified by preparative TLC to afford N-[(3S)-1-[3-(benzyloxy)quinoxalin-2-yl]pyrrolidin-3-yl]-N,2-dimethylpropanamide 304 (17.3 mg, 14.1% yield, 99.5% purity) as a yellow gum. LC MS of 304: Ret. Time: 1.398 min, MS (ESI) m/z: 405.1, M+H⁺. ¹H NMR of 304: ¹H NMR (400 MHz, DMSO) δ 7.61 (d, J=7.6 Hz, 1H), 7.54 (d, J=6.8 Hz, 3H), 7.43-7.27 (m, 5H), 5.51 (s, 2H), 5.15-4.63 (m, 1H), 3.97-3.87 (m, 2H), 3.79-3.55 (m, 2H), 3.04-2.66 (m, 4H), 2.03-1.97 (m, 2H), 1.00 (d, J=4.8 Hz, 6H).

### Example 11B: Preparation of Analogous Compounds 299 and 300

Table 9, below, depicts exemplary compounds synthesized according to an analogous procedure to the one described for 304.

TABLE 9
Compounds synthesized analogously to 304.

| Compound Structure | Compound No. | Yield | Characterization |
|---|---|---|---|
| 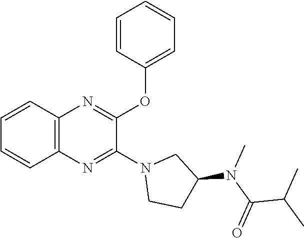 [SMILES: CC(C)C(=O)N(C)[C@H]1CCN(c2nc3ccccc3nc2Oc2ccccc2)C1] | 299 | 23.4% yield | LC MS of 299: Ret. Time: 1.329 min, MS (ESI) m/z: 391.3, [M + H]⁺. ¹H NMR of 299: ¹H NMR (400 MHz, DMSO) δ 7.59 (d, J = 8.0 Hz, 1H), 7.44- 7.38 (m, 4H), 7.34-7.21 (m, 4H), 5.17-4.79 (m. 1H), 4.21-3.95 (m, 2H). 3.94-3.65 (m, 2H). 3.02 (d, J = 23.2 Hz, 2H), 2.95-2.75 (m, 2H), 2.24-2.02 (m, 2H), 1.23-1.01 (d, J = 6.4 Hz, 6H). |
| 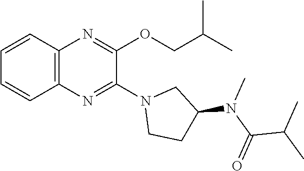 [SMILES: CC(C)COc1nc2ccccc2nc1N1CC[C@H](N(C)C(=O)C(C)C)C1] | 300 | 13.9% yield | LC MS of 300: Ret. Time: 1.421 min, MS (ESI) m/z: 371.3, M + H+. ¹H NMR of 300: ¹H NMR (400 MHz, DMSO) δ 7.54 (t, J = 9.6 Hz, 2H), 7.37 (dd, J = 11.2, 4.0 Hz, 1H), 7.27 (t, J = 6.8 Hz, 1H), 5.08-4.76 (m. 1H), 4.30-4.15 (m, 2H), 4.06- 3.61 (m, 4H), 2.79-2.50 (m, 4H), 2.19- 2.02 (m, 3H), 1.02 (dd. J = 6.8, 3.2 Hz, 12H). |

Compound 299 according to the present disclosure was prepared as shown in Scheme 75 by an analogous procedure to the one described in Example 11A.

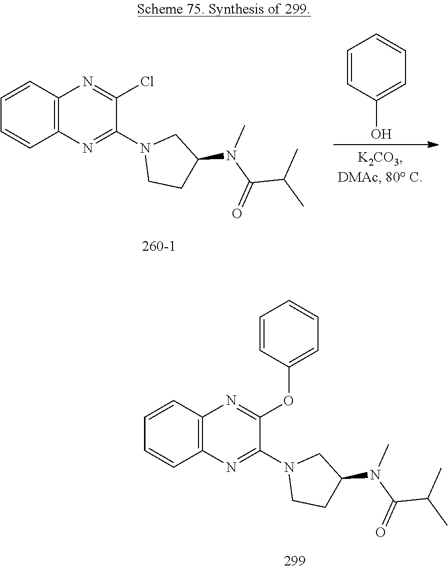 [SMILES: CC(C)C(=O)N(C)[C@H]1CCN(c2nc3ccccc3nc2Cl)C1 ; Oc1ccccc1 ; CC(C)C(=O)N(C)[C@H]1CCN(c2nc3ccccc3nc2Oc2ccccc2)C1]

Compound 300 according to the present disclosure was prepared as shown in Scheme 76 by an analogous procedure to the one described in Example 11A.

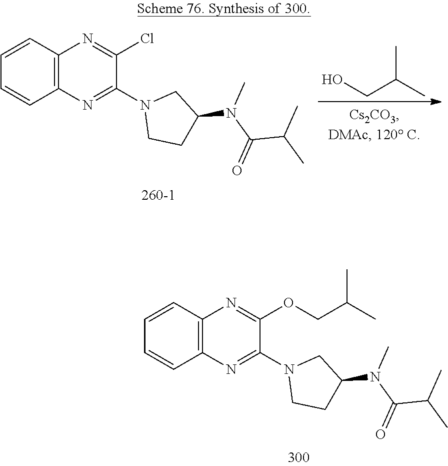

### Example 12: Synthesis of Analogs 400 and 482, 521, 522, 528

Compound 400 according to the present disclosure was prepared as shown in Scheme 77 and described below.

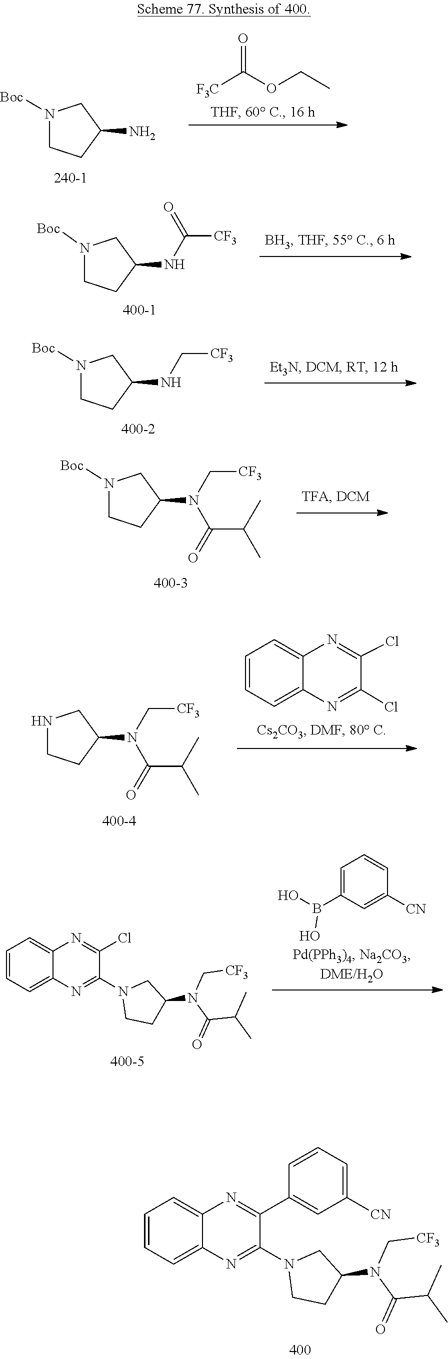

### Example 12A: Preparation of Compound 400-1

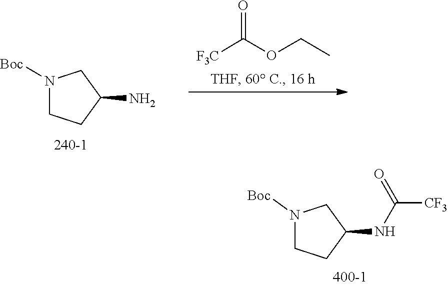

A mixture of tert-butyl (S)-3-aminopyrrolidine-1-carboxylate 240-1 (1 g, 5.37 mmol, 1.0 eq) and ethyl 2,2,2-trifluoroacetate (7.63 g, 53.72 mmol, 10 eq) in THF (50 mL) was stirred at 60° C. under N₂ for 16 h. The reaction was cooled to 25° C. The reaction mixture was concentrated in vacuo to give the product tert-butyl (S)-3-(2,2,2-trifluoroacetamido)pyrrolidine-1-carboxylate 400-1 (1.1 g, yield: 72.6%) as a yellow oil. This residue was used directly in the next step without further purification. LC MS of 400-1: Ret. Time: 1.213 min, MS (ESI) m/z: 227.1, [M−55]⁺; 305.2, [M+23]⁺.

### Example 12B: Preparation of Compound 400-2

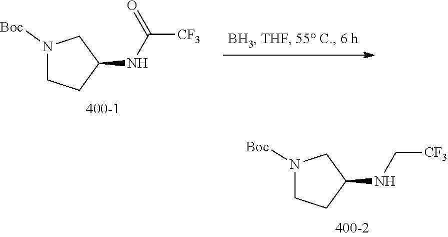 [SMILES: CC(C)(C)OC(=O)N1CC[C@H](NC(=O)C(F)(F)F)C1 ; CC(C)(C)OC(=O)N1CC[C@H](NCC(F)(F)F)C1]

To a mixture of tert-butyl (S)-3-(2,2,2-trifluoroacetamido)pyrrolidine-1-carboxylate 400-1 (1.1 g, 3.90 mmol, 1.0 eq.) in THF (anhydrous, 80 mL) at 0° C. under N₂ a solution of borane in THF (1.0 M, 19.5 ml, 5.0 eq.) was slowly added dropwise. After the addition was complete, the reaction mixture was heated to 55° C. and stirring was continued for 6 h, then the reaction mixture was cooled to 25° C. The reaction was cooled in an ice bath and quenched with MeOH (30 mL). The solution was concentrated and redissolved in MeOH (100 mL). To the solution was added a solution of HCl (4M in dioxane, 7.5 mL) and the mixture was stirred at room temperature for 0.5 h, then concentrated. The residue was taken up in DCM (200 mL) and washed with saturated aqueous Na₂CO₃ (2×50 mL) and brine (50 mL). The organic layer was dried over Na₂SO₄, filtered, and concentrated. The residue was purified via flash chromatography eluting with a gradient of 0%-5% CH₂Cl₂/MeOH to give the product tert-butyl (S)-3-((2,2,2-trifluoroethyl)amino)pyrrolidine-1-carboxylate 400-2 (800 mg, yield: 77%) as a yellow oil. LC MS of 400-2: Ret. Time: 1.215 min, MS (ESI) m/z: 213.1, [M−55]⁺.

### Example 12C: Preparation of Compound 400-4

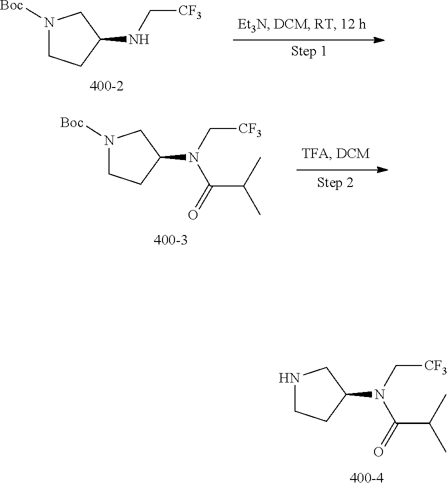 [SMILES: CC(C)(C)OC(=O)N1CC[C@H](NCC(F)(F)F)C1 ; CC(C)C(=O)N(CC(F)(F)F)[C@H]1CCN(C(=O)OC(C)(C)C)C1 ; CC(C)C(=O)N(CC(F)(F)F)[C@H]1CCNC1]

A mixture of tert-butyl (S)-3-((2,2,2-trifluoroethyl)amino)pyrrolidine-1-carboxylate 400-2 (1 g, 3.71 mmol, 1.0 eq) and triethylamine (1.315 g, 13.00 mmol, 3.5 eq) in DCM (30 mL) was stirred at 0° C. under N₂. Isobutyryl chloride (791 mg, 7.43 mmol, 2 eq) in DCM (20 mL) was slowly added dropwise to the reaction system at 0° C. After the addition is complete, the reaction system was warmed to 25° C. and stirring was continued for 12 h. The resulting mixture was quenched with 1N HCl (20 mL) and extracted with EtOAc (30 mL×3). The organic layers were combined and dried over MgSO₄. The filtrate was concentrated in vacuo, and the residue was used directly in the next step without further purification. To a solution of the residue in dichloromethane (50 mL) at 25° C., trifluoroacetic acid (6.35 g, 55.704 mmol) was added. This reaction mixture was stirred at 25° C. for 2 h. The reaction mixture was concentrated under reduced pressure at 40° C. The mixture was adjusted to pH 7~8 with Na₂CO₃. The residue was extracted with DCM (5×50 mL). The reaction mixture was concentrated under reduced pressure at 40° C. The residue was purified via flash chromatography eluting with CH₂Cl₂/MeOH to give the product (S)—N-(pyrrolidin-3-yl)-N-(2,2,2-trifluoroethyl)isobutyramide 400-4 (650 mg, yield: 58.5%) as a yellow oil. LC MS of 400-4: Ret. Time: 0.202 min, MS (ESI) m/z: 239.1, [M+H]⁺.

### Example 12D: Preparation of Compound 400-5

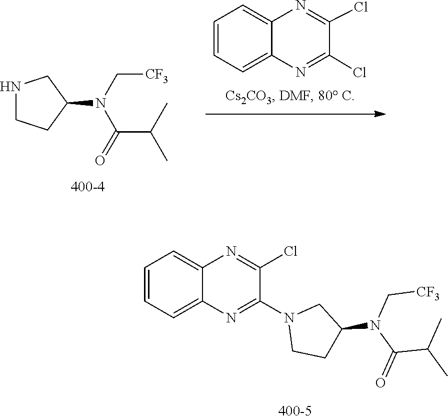 [SMILES: Clc1nc2ccccc2nc1Cl ; CC(C)C(=O)N(CC(F)(F)F)[C@H]1CCNC1 ; CC(C)C(=O)N(CC(F)(F)F)[C@H]1CCN(c2nc3ccccc3nc2Cl)C1]

A round-bottom flask containing a mixture of (S)—N-(pyrrolidin-3-yl)-N-(2,2,2-trifluoroethyl)isobutyramide 400-4 (800 mg, 3.34 mmol), 2,3-dichloroquinoxaline (865 mg, 4.35 mmol) and cesium carbonate (5.447 g, 16.72 mmol) in DMF (20 mL) was placed in an oil bath heated to 80° C. The mixture was continuously stirred at 80° C. for 1 h. The reaction was cooled to 25° C. Water (100 mL) was added. The residue was extracted with EtOAc (3×100 mL). The organic phase was washed with saturated brine (50 mL). The reaction mixture was concentrated under reduced pressure at 40° C. The residue was purified via flash chromatography eluting with petroleum ether/EtOAc (80:20) to give the product (S)—N-(1-(3-chloroquinoxalin-2-yl)pyrrolidin-3-yl)-N-(2,2,2-trifluoroethyl)isobutyramide 400-5 (400 mg, yield: 29.5%) as a pale white solid. LC MS of 400-5: Ret. Time: 1.441 min, MS (ESI) m/z: 401.1, [M+H]⁺.

### Example 12E: Preparation of Compound 400

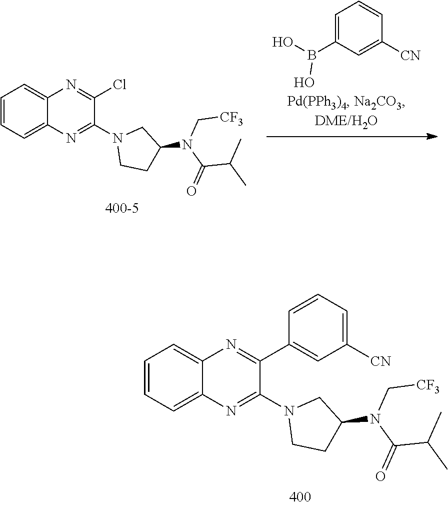 [SMILES: N#Cc1cccc(B(O)O)c1 ; CC(C)C(=O)N(CC(F)(F)F)[C@H]1CCN(c2nc3ccccc3nc2Cl)C1 ; CC(C)C(=O)N(CC(F)(F)F)[C@H]1CCN(c2nc3ccccc3nc2-c2cccc(C#N)c2)C1]

A solution of (S)—N-(1-(3-chloroquinoxalin-2-yl)pyrrolidin-3-yl)-N-(2,2,2-trifluoroethyl)isobutyramide 400-5 (200 mg, 0.50 mmol), 3-cyanophenylboronic acid (146 mg, 1.00 mmol), Pd(PPh₃)₄ (57.5 mg, 0.050 mmol), and Na₂CO₃ (159 mg, 1.50 mmol) in DME/1H₂O (10 mL) was warmed to 90° C. in an oil bath. The flask is equipped with a magnetic stirring bar and a three-way stopcock attached to a balloon filled with nitrogen. The mixture is stirred for 12 h at 90° C. and monitored by LCMS. The reaction was cooled to 25° C. The mixture was diluted with water and was extracted with ethyl acetate (3×30 mL). The reaction mixture was concentrated under reduced pressure at 40° C. The residue was purified via flash chromatography eluting with petroleum ether/EtOAc to give the crude product. The crude product was purified via preparative HPLC to give the product (S)—N-(1-(3-(3-cyanophenyl)quinoxalin-2-yl)pyrrolidin-3-yl)-N-(2,2,2-trifluoroethyl)isobutyramide 400 (151.6 mg, yield: 64.1%) as a yellow solid. LC MS of 400: Ret. Time: 1.424 min, MS (ESI) m/z: 468.2, [M+H]⁺. ¹H NMR of 400: ¹H NMR (400 MHz, DMSO-d₆) δ 8.08 (s, 1H), 8.01 (d, J=7.8, 1H), 7.89 (t, J=8.1 Hz, 2H), 7.71 (dd, J=16.5, 8.3 Hz, 2H), 7.67-7.60 (m, 1H), 7.49-7.42 (m, 1H), 4.51 (s, 1H), 4.28-4.05 (m, 2H), 3.57-3.28 (m, 3H), 3.18 (t, J=9.6 Hz, 1H), 2.99-2.86 (m, 1H), 2.21-1.85 (m, 2H), 0.98 (dd, J=17.8, 8.9 Hz, 6H).

### Example 12F: Preparation of Analogous Compounds 482, 521, 522, and 528

Table 10, below, depicts exemplary compounds synthesized according to an analogous procedure to the one described for 400.

TABLE 10
Compounds synthesized analogously to 400.

| | Compound | | |
|---|---|---|---|
| Compound Structure | No. | Yield | Characterization |
| 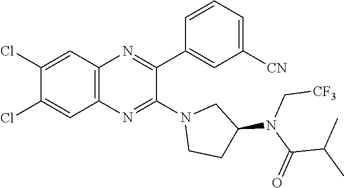 [SMILES: CC(C)C(=O)N(CC(F)(F)F)[C@H]1CCN(c2nc3cc(Cl)c(Cl)cc3nc2-c2cccc(C#N)c2)C1] | 482 | 38.7% yield | LC MS of 482: Ret. Time: 1.555 min. MS (ESI) m/z: 536.0, [M + H]+. ¹H NMR of 482: ¹H NMR (400 MHz, DMSO-d₆) δ 8.10 (s, 1H), 8.06 (s, 1H), 7.98 (d, J = 7.9 Hz, 1H), 7.94-7.89 (m, 2H), 7.70 (t, J = 7.8 Hz, 1H), 4.60-4.41 (s, 1H), 4.30- 4.05 (m, 2H), 3.46-3.38 (m, 2H), 3.38- 3.28 (m, 1H), 3.26-3.12 (m, 1H), 2.98- 2.86 (m, 1H), 2.17-1.93 (m, 2H), 0.98 (dd, J = 12.7, 6.6 Hz, 6H). |
| 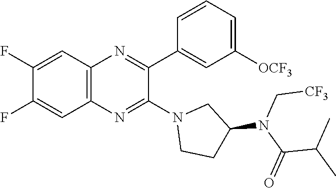 [SMILES: CC(C)C(=O)N(CC(F)(F)F)[C@H]1CCN(c2nc3cc(F)c(F)cc3nc2-c2cccc(OC(F)(F)F)c2)C1] | 521 | 35.2% yield | LC MS of 521: Ret. Time: 1.554 min, MS (ESI) m/z: 563.1, [M + H]+. ¹H NMR of 521: ¹H NMR (400 MHz, DMSO-d₆) δ 7.86 (dd, J = 11.2, 8.6 Hz, 1H), 7.70-7.58 (m, 4H), 7.47-7.40 (m, 1H), 4.48 (s, 1H), 4.27-3.99 (m, 2H), 3.55-3.27 (m, 3H), 3.20-3.10 (m, 1H), 2.91 (dt, J = 12.6, 6.2 Hz, 1H), 2.17-1.96 (m, 2H), 0.97 (dd, J = 14.1, 6.6 Hz, 6H). |
| 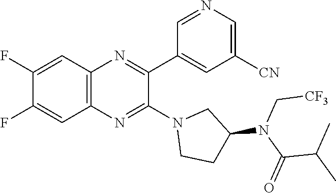 [SMILES: CC(C)C(=O)N(CC(F)(F)F)[C@H]1CCN(c2nc3cc(F)c(F)cc3nc2-c2cncc(C#N)c2)C1] | 522 | 75.0% yield | LC MS of 522: Ret. Time: 1.405 min, MS (ESI) m/z: 505.2, [M + H]+. ¹H NMR of 522: ¹H NMR (400 MHz, DMSO-d₆) δ 9.13 (d, J = 2.1 Hz, 1H), 9.09 (d, J = 2.0 Hz, 1H), 8.52 (t, J = 2.1 Hz, 1H), 7.93 (dd, J = 11.1, 8.6 Hz, 1H), 7.70 (dd, J = 14.6, 9.2 Hz, 1H), 4.51 (s, 1H), 4.37-3.97 (m, 2H), 3.50-3.26 (m, 3H), 3.24-3.14 (m, 1H), 3.04-2.86 (m, 1H), 2.20-1.96 (m, 2H), 0.99 (dd, J = 12.0, 6.6 Hz, 6H). |
| 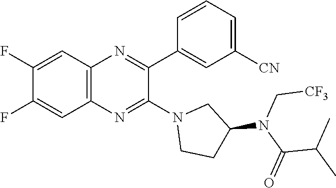 [SMILES: CC(C)C(=O)N(CC(F)(F)F)[C@H]1CCN(c2nc3cc(F)c(F)cc3nc2-c2cccc(C#N)c2)C1] | 528 | 51.3% yield | LC MS of 528: Ret. Time: 1.451 min, MS (ESI) m/z: 504.2, [M + H]+. ¹H NMR of 528: ¹H NMR (400 MHz, DMSO-d₆) δ 8.07 (t, J = 1.5 Hz, 1H), 8.00- 7.96 (m, 1H), 7.95-7.91 (m, 1H), 7.89 (dd, J = 9.0, 6.5 Hz, 1H), 7.74-7.65 (m, 2H), 4.73- 4.36 (m, 1H), 4.36-3.86 (m, 2H), 3.57- 3.20 (m, 4H), 3.05-2.85 (m, 1H), 2.18- 1.95 (m, 2H), 0.98 (dd, J = 13.7, 6.6 Hz, 7H). |

Compound 482 according to the present disclosure was prepared as shown in Scheme 78 by an analogous procedure to the one described in Examples 12A-12E.

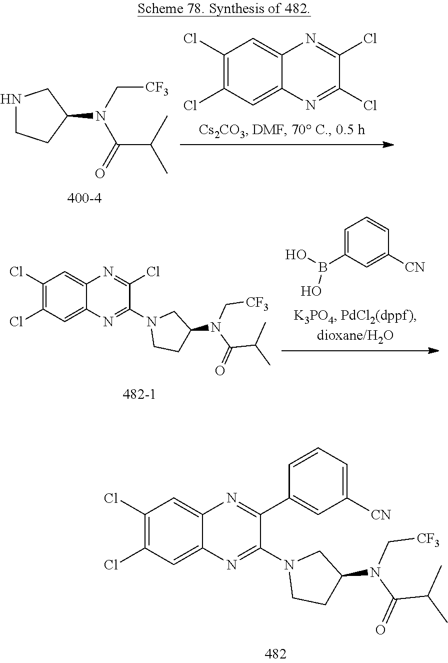

Compound 521 according to the present disclosure was prepared as shown in Scheme 79 by an analogous procedure to the one described in Examples 12A-12E.

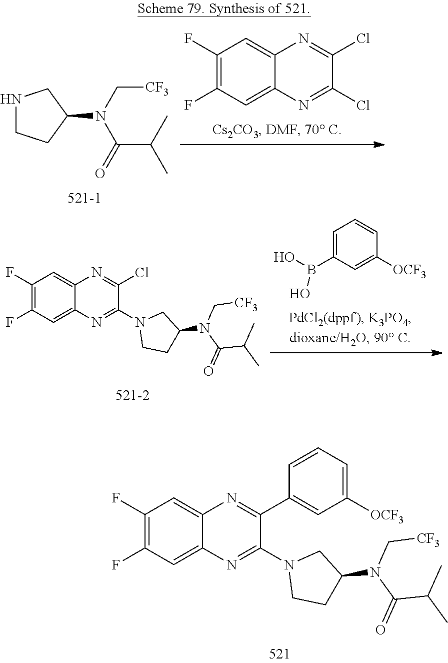

Compound 522 according to the present disclosure was prepared as shown in Scheme 80 by an analogous procedure to the one described in Examples 12A-12E.

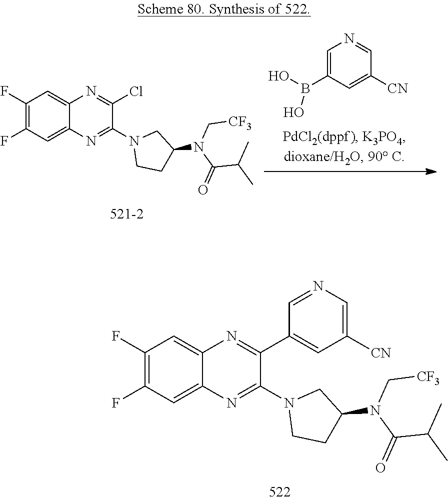 [SMILES: CC(C)C(=O)N(CC(F)(F)F)[C@H]1CCN(c2nc3cc(F)c(F)cc3nc2Cl)C1 ; N#Cc1cncc(B(O)O)c1 ; CC(C)C(=O)N(CC(F)(F)F)[C@H]1CCN(c2nc3cc(F)c(F)cc3nc2-c2cncc(C#N)c2)C1]

Compound 528 according to the present disclosure was prepared as shown in Scheme 81 by an analogous procedure to the one described in Examples 12A-12E.

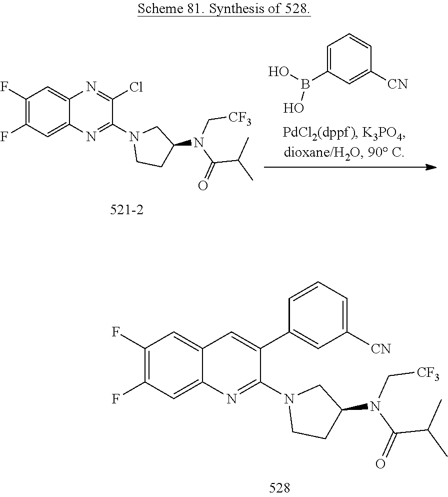 [SMILES: CC(C)C(=O)N(CC(F)(F)F)[C@H]1CCN(c2nc3cc(F)c(F)cc3nc2Cl)C1 ; N#Cc1cccc(B(O)O)c1 ; CC(C)C(=O)N(CC(F)(F)F)[C@H]1CCN(c2nc3cc(F)c(F)cc3cc2-c2cccc(C#N)c2)C1]

### Example 13: Synthesis of Analogs 402 and 401, 495, 496, 510, 511

Compound 402 according to the present disclosure was prepared as shown in Scheme 82 and described below.

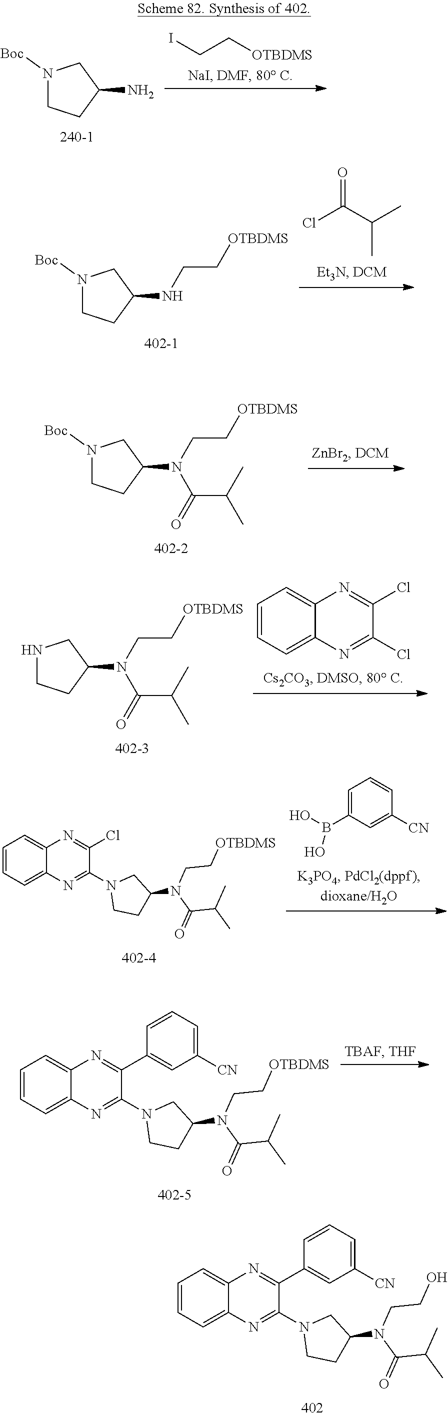

### Example 13A: Preparation of Compound 402-1

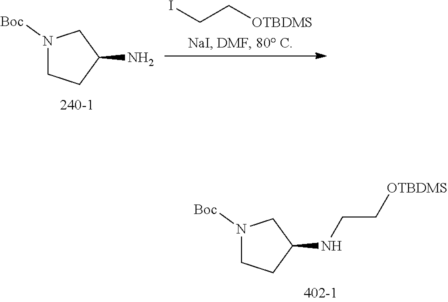 [SMILES: CC(C)(C)OC(=O)N1CC[C@H](N)C1 ; CC(C)(C)[Si](C)(C)OCCI ; CC(C)(C)OC(=O)N1CC[C@H](NCCO[Si](C)(C)C(C)(C)C)C1]

To a solution of tert-butyl (S)-3-aminopyrrolidine-1-carboxylate 240-1 (1.86 g, 10 mmol, 1.0 eq) and tert-butyl(2-iodoethoxy)dimethylsilane (5.72 g, 20 mmol, 2.0 eq) in DMF (30 mL) stirred under nitrogen was added NaI (1.98 g, 12 mmol, 1.2 eq). The reaction mixture was stirred at 85° C. under microwave irradiation for 5 h. The mixture was diluted with saturated brine solution and extracted with ethyl acetate (3×100 mL). The combined organic extracts were washed with brine (5×50 mL), dried (MgSO₄), and evaporated under reduced pressure to give a residue. The residue was purified by flash column chromatography on silica gel (DCM/MeOH=30:1) to give tert-butyl (S)-3-((2-((tert-butyldimethylsilyl)oxy)ethyl)amino)pyrrolidine-1-carboxylate 402-1 (2.5 g, 72.7%) as a yellow oil. LC MS of 402-1: Ret. Time: 1.15~1.17 min, MS (ESI) m/z: 345.1, [M+H]⁺.

### Example 13B: Preparation of Compound 402-2

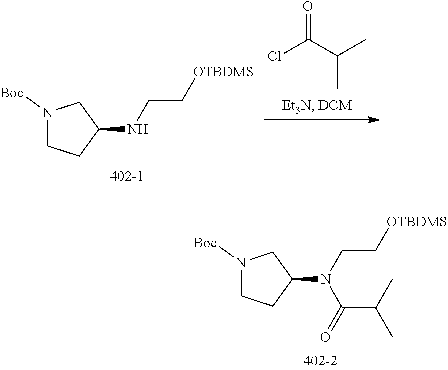

To a round-bottom flask containing a mixture of (S)-3-((2-((tert-butyldimethylsilyl) oxy)ethyl)amino)pyrrolidine-1-carboxylate 402-1 (1.2 g, 3.49 mmol) and TEA (1.06 g, 10.47 mmol) in DCM (30 mL) stirred under nitrogen was added a solution of 2-methylpropanoyl chloride (555 mg, 5.23 mmol) in DCM (10 mL). The mixture was continuously stirred at 25° C. for 2 h.

Saturated aqueous brine (100 mL) was added. The residue was extracted with ethyl acetate (3×100 mL). The organic phases were combined and washed with saturated brine (50 mL), dried (MgSO₄), and evaporated under reduced pressure to give a residue. The residue was purified by flash column chromatography on silica gel (40:1 DCM/MeOH) to give tert-butyl (S)-3-(N-(2-((tert-butyldimethylsilyl)oxy)ethyl)isobutyramido)pyrrolidine-1-carboxylate 402-2 (1.1 g, 76.2%) as a yellow oil. LC MS of 402-2: Ret. Time: 1.606 min, MS (ESI) m/z: 437.2, [M+23]⁺.

### Example 13C: Preparation of Compound 402-3

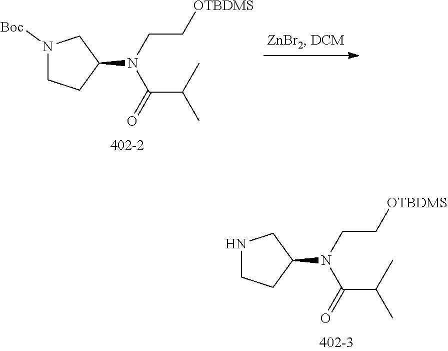 [SMILES: CC(C)C(=O)N(CCO[Si](C)(C)C(C)(C)C)[C@H]1CCN(C(=O)OC(C)(C)C)C1 ; CC(C)C(=O)N(CCO[Si](C)(C)C(C)(C)C)[C@H]1CCNC1]

To a solution of tert-butyl (S)-3-(N-(2-((tert-butyldimethylsilyl)oxy)ethyl)iso-butyramido)-pyrrolidine-1-carboxylate 402-2 (900 mg, 2.17 mmol) in dichloromethane (30 mL) at 25° C., ZnBr₂ (2.93 g, 13.02 mmol) was added. The reaction mixture was stirred at 25° C. for 6 h. The solution was filtered and the filtrate was concentrated under reduced pressure at 40° C. The residue was purified via flash chromatography eluting with DCM/MeOH to give the product (S)—N-(2-((tert-butyldimethylsilyl)oxy)ethyl)-N-(pyrrolidin-3-yl)isobutyramide 402-3 (680 mg, 84.7%) as a yellow oil. LC MS of 402-3: Ret. Time: 1.107 min, MS (ESI) m/z: 315.1, [M+H]⁺.

### Example 13D. Preparation of Compound 402-4

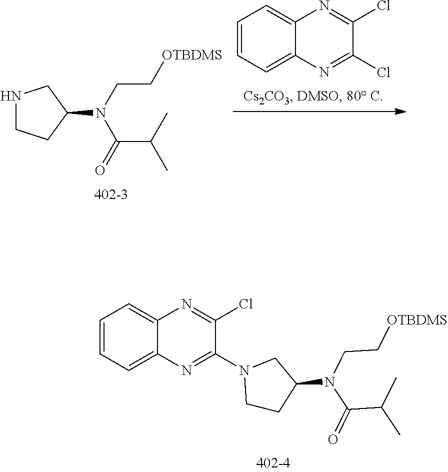 [SMILES: CC(C)C(=O)N(CCO[Si](C)(C)C(C)(C)C)[C@H]1CCNC1 ; Clc1nc2ccccc2nc1Cl ; CC(C)C(=O)N(CCO[Si](C)(C)C(C)(C)C)[C@H]1CCN(c2nc3ccccc3nc2Cl)C1]

A round-bottom flask containing a mixture of (S)—N-(2-((tert-butyldimethylsilyl)oxy) ethyl)-N-(pyrrolidin-3-yl)isobutyramide 402-3 (900 mg, 2.86 mmol), 2,3-dichloroquinoxaline (1.14 g, 5.72 mmol) and cesium carbonate (2.80 g, 8.58 mmol) in DMF (20 mL) was placed in an oil bath heated to 70° C. The mixture was continuously stirred at 70° C. for 1 h. The reaction was cooled to 25° C. Saturated aqueous brine (100 mL) was added. The residue was extracted with ethyl acetate (3×100 mL). The combined organic phases were washed with saturated brine (5×50 mL) and concentrated under reduced pressure at 40° C. The residue was purified via flash chromatography eluting with petroleum ether/EtOAc to give the product (S)—N-(2-((tert-butyldimethylsilyl)oxy)ethyl)-N-(1-(3-chloroquinoxalin-2-yl)pyrrolidin-3-yl)isobutyramide 402-4 (660 mg, yield: 45.9%) as a yellow oil. LC MS of 402-4: Ret. Time: 1.610~1.725 min, MS (ESI) m/z: 477.3, [M+H]⁺.

### Example 13E: Preparation of Compound 402-5

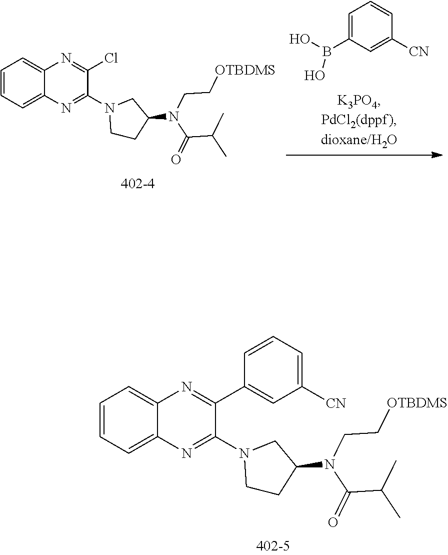 [SMILES: CC(C)C(=O)N(CCO[Si](C)(C)C(C)(C)C)[C@H]1CCN(c2nc3ccccc3nc2Cl)C1 ; N#Cc1cccc(B(O)O)c1 ; CC(C)C(=O)N(CCO[Si](C)(C)C(C)(C)C)[C@H]1CCN(c2nc3ccccc3nc2-c2cccc(C#N)c2)C1]

A solution of(S)—N-(2-((tert-butyldimethylsilyl)oxy)ethyl)-N-(1-(3-chloroquinoxalin-2-yl)pyrrolidin-3-yl)isobutyramide 402-4 (300 mg, 0.63 mmol), 3-cyanophenylboronic acid (185 mg, 1.26 mmol), Pd(dppf)Cl₂ (46 mg, 0.063 mmol), and K₃PO₄ (400 mg, 1.89 mmol) in dioxane/H₂O (20 mL) was warmed to 90° C. in an oil bath under nitrogen. The mixture was stirred for 0.5 h at 90° C. and monitored by LCMS. The reaction was cooled to 25° C. The mixture was diluted with brine and extracted with ethyl acetate (3×50 mL). The combined organic layers were concentrated under reduced pressure at 40° C. The residue was purified via flash chromatography eluting with petroleum ether/EtOAc to give the product (S)—N-(2-((tert-butyldimethylsilyl)oxy)ethyl)-N-(1-(3-(3-cyanophenyl)quinoxalin-2-yl)pyrrolidin-3-yl)isobutyramide 402-5 (330 mg, yield: 91.7%) as a yellow solid.

LC MS of 402-5: Ret. Time: 1.610~1.725 min, MS (ESI) m/z: 544.3, [M+H]⁺.

### Example 13F: Preparation of Compound 402

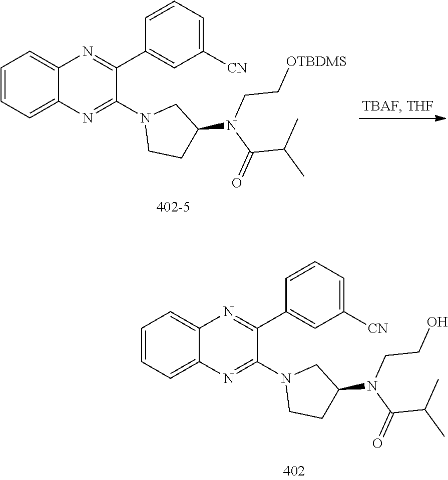 [SMILES: CC(C)C(=O)N(CCO[Si](C)(C)C(C)(C)C)[C@H]1CCN(c2nc3ccccc3nc2-c2cccc(C#N)c2)C1 ; CC(C)C(=O)N(CCO)[C@H]1CCN(c2nc3ccccc3nc2-c2cccc(C#N)c2)C1]

A mixture of (S)—N-(2-((tert-butyldimethylsilyl)oxy)ethyl)-N-(1-(3-(3-cyanophenyl)-quinoxalin-2-yl)pyrrolidin-3-yl)isobutyramide 402-5 (280 mg, 0.52 mmol, 1.0 eq) in THE (50 mL) was stirred at 25° C. under N₂. TBAF (5.2 mL, 1M in THF, 10 eq) was slowly added dropwise to the reaction mixture at 0° C. After the addition was complete, the reaction mixture was warmed to 25° C. and stirring was continued for 0.5 h. The mixture was diluted with ethyl acetate (100 mL). The organic phase was washed with saturated brine (50 mL×6) and concentrated under reduced pressure at 25° C. The residue was purified via preparative HPLC to give the product 402 (200.3 mg, yield: 90.1%) as a yellow solid. LC MS of 402: Ret. Time: 1.225 min, MS (ESI) m/z: 430.3, [M+H]⁺. ¹H NMR of 402: ¹H NMR (400 MHz, DMSO-d₆) δ 8.10 (s, 1H), 8.00 (d, J=7.8 Hz, 1H), 7.88 (t, J=9.1, 2H), 7.71 (dd, J=14.9, 7.4 Hz, 2H), 7.67-7.60 (m, 1H), 7.49-7.41 (m, 1H), 4.68-4.38 (m, 2H), 3.46-3.32 (m, 4H), 3.31-3.15 (m, 4H), 2.96-2.80 (m, 1H), 2.11-1.91 (m, 2H), 0.97 (dd, J=6.6, 4.9 Hz, 6H).

### Example 13G: Preparation of Analogous Compounds 401, 495, 496, 510, and 511

Table 11, below, depicts exemplary compounds synthesized according to an analogous procedure to the one described for 402.

TABLE 11
Compounds synthesized analogously to 402.

| | Compound | | |
|---|---|---|---|
| Compound Structure | No. | Yield | Characterization |
| 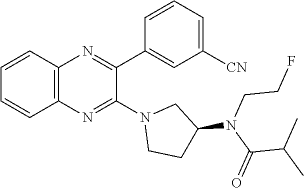 [SMILES: CC(C)C(=O)N(CCF)[C@H]1CCN(c2nc3ccccc3nc2-c2cccc(C#N)c2)C1] | 401 | 20.4% yield | LC MS of 401: Ret. Time: 1.380 min, MS (ESI) m/z: 432.2, [M + H]+. ¹H NMR of 401: ¹H NMR (400 MHz, DMSO-d₆) δ 8.10 (t, J = 1.7 Hz, 1H), 8.04- 7.98 (m, 1H), 7.92-7.84 (m, 2H), 7.71 (q, J = 7.8 Hz, 2H), 7.64 (ddd, J = 8.3, 6.9, 1.4 Hz, 1H), 7.49-7.41 (m, 1H), 4.66-4.51 (m, 1H), 4.51-4.44 (m, 1H), 4.41-4.31 (m, 1H), 3.66-3.39 (m, 2H), 3.47-3.24 (m, 3H), 3.26-3.12 (m, 1H), 2.96-2.80 (m, 1H), 2.10-1.98 (m, 2H), 0.98 (dd, J = 8.4, 6.7 Hz, 6H). |
| 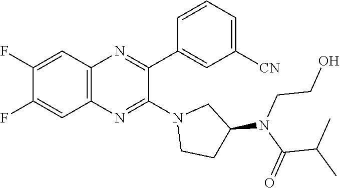 [SMILES: CC(C)C(=O)N(CCO)[C@H]1CCN(c2nc3cc(F)c(F)cc3nc2-c2cccc(C#N)c2)C1] | 495 | 80.5% yield | LC MS of 495: Ret. Time: 1.313 min, MS (ESI) m/z: 466.2, [M + 1]+. ¹H NMR of 495: ¹H NMR (400 MHz, DMSO-d₆) δ 8.08 (t, J = 1.4 Hz, 1H), 8.00- 7.95 (m, 1H), 7.93-7.89 (m, 1H), 7.89- 7.83 (m, 1H), 7.73-7.68 (m, 1H), 7.66 (dd, J = 11.7, 8.1 Hz, 1H), 4.68-4.38 (m, 2H), 3.46-3.32 (m, 4H), 3.30-3.15 (m, 4H), 2.87 (dt, J = 13.3, 6.7 Hz, 1H), 2.11-1.91 (m, 2H), 0.97 (dd, J = 6.6, 5.1 Hz, 6H). |
| 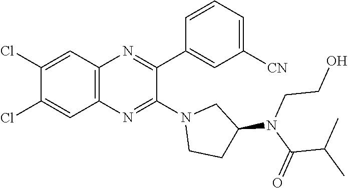 [SMILES: CC(C)C(=O)N(CCO)[C@H]1CCN(c2nc3cc(Cl)c(Cl)cc3nc2-c2cccc(C#N)c2)C1] | 496 | 38.1% yield | LC MS of 496: Ret. Time: 1.416 min. MS (ESI) m/z: 498.0, [M + 1]+. ¹H NMR of 496: ¹H NMR (400 MHz, DMSO-d6) δ 8.11-8.06 (m, 2H), 8.01- 7.95 (m, 1H), 7.94-7.89 (m, 2H), 7.71 (t, J = 7.8 Hz, 1H), 4.66-4.41 (m, 2H), 3.54- 3.32 (m, 4H), 3.32-3.17 (m, 4H), 2.96- 2.77 (m, 1H), 2.13-1.88 (m, 2H), 0.97 (dd, J = 6.6, 5.0 Hz, 6H). |
| 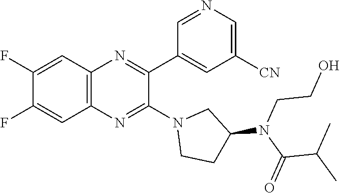 [SMILES: CC(C)C(=O)N(CCO)[C@H]1CCN(c2nc3cc(F)c(F)cc3nc2-c2cncc(C#N)c2)C1] | 511 | 58.1% yield | LC MS of 511: Ret. Time: 1.258 min, MS (ESI) m/z: 466.2, [M + H]+. ¹H NMR of 511: ¹H NMR (400 MHz, CDCl₃) δ 9.17 (s, 1H), 8.95 (d, J = 9.8 Hz, 1H), 8.41 (d, J = 11.8 Hz, 1H), 7.77-7.53 (m, 2H), 4.85-4.55 (m, 1H), 3.71 (t, J = 4.4 Hz, 2H), 3.59-3.15 (m, 6H), 2.92-2.80 (m, 1H), 2.25-2.00 (m, 2H), 1.23-1.05 (m, 6H). |

Compound 401 according to the present disclosure was prepared as shown in Scheme 83 by an analogous procedure to the one described in Examples 13A-13E.

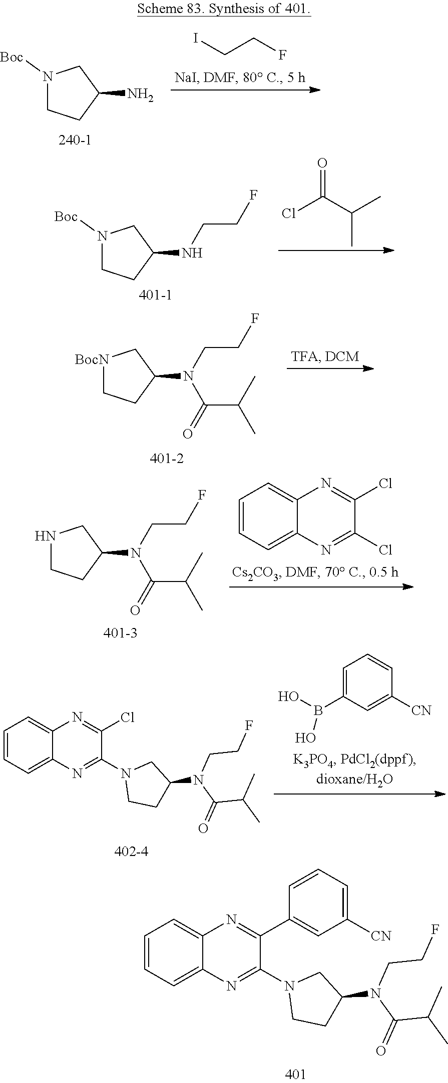

Compound 495 according to the present disclosure was prepared as shown in Scheme 84 by an analogous procedure to the one described in Examples 13A-13E.

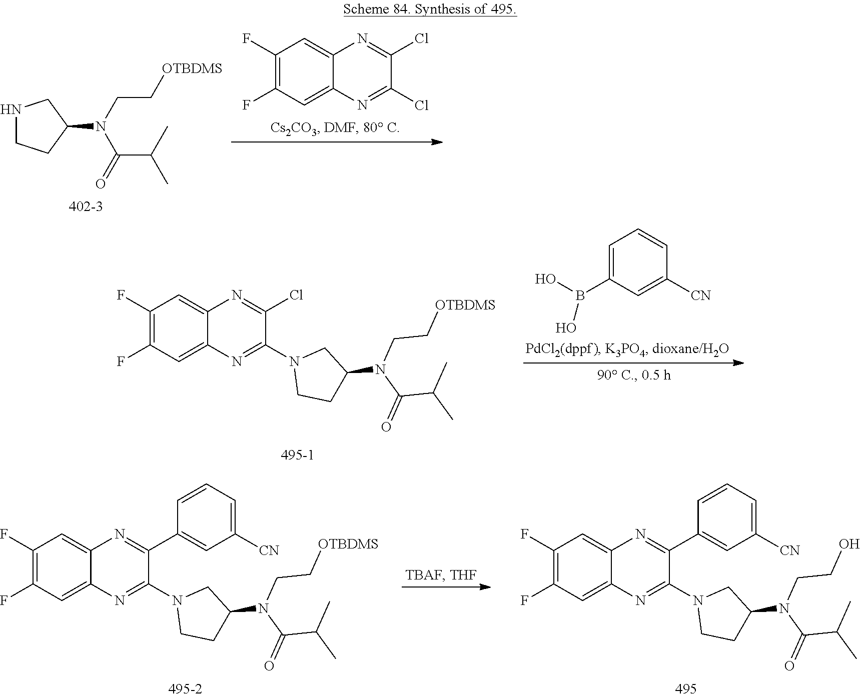 [SMILES: CC(C)C(=O)N(CCO[Si](C)(C)C(C)(C)C)[C@H]1CCNC1 ; Fc1cc2nc(Cl)c(Cl)nc2cc1F ; CC(C)C(=O)N(CCO[Si](C)(C)C(C)(C)C)[C@H]1CCN(c2nc3cc(F)c(F)cc3nc2Cl)C1 ; N#Cc1cccc(B(O)O)c1 ; CC(C)C(=O)N(CCO[Si](C)(C)C(C)(C)C)[C@H]1CCN(c2nc3cc(F)c(F)cc3nc2-c2cccc(C#N)c2)C1 ; CC(C)C(=O)N(CCO)[C@H]1CCN(c2nc3cc(F)c(F)cc3nc2-c2cccc(C#N)c2)C1]

Compound 496 according to the present disclosure was prepared as shown in Scheme 85 by an analogous procedure to the one described in Examples 13A-13E.

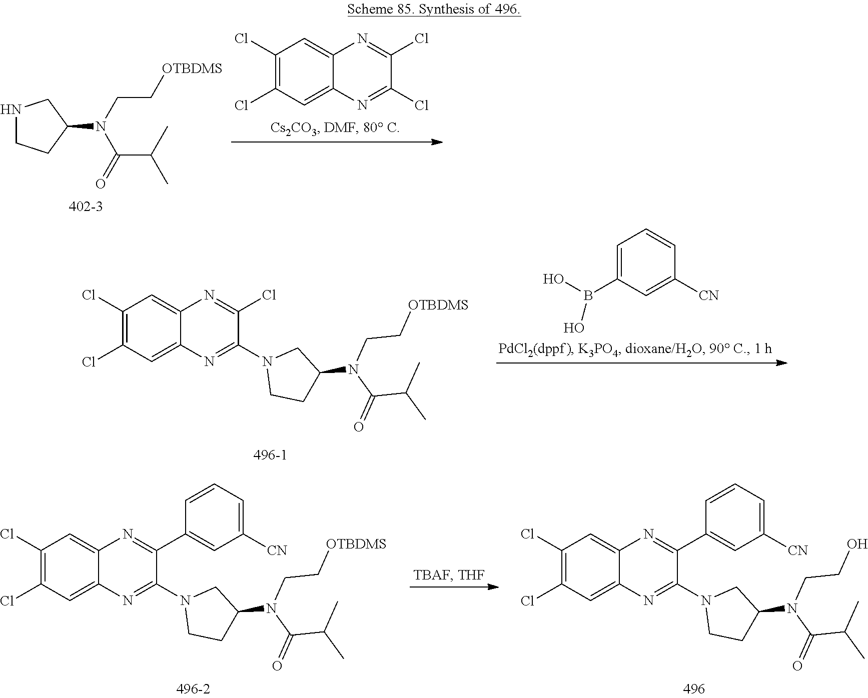 [SMILES: CC(C)C(=O)N(CCO[Si](C)(C)C(C)(C)C)[C@H]1CCNC1 ; Clc1cc2nc(Cl)c(Cl)nc2cc1Cl ; CC(C)C(=O)N(CCO[Si](C)(C)C(C)(C)C)[C@H]1CCN(c2nc3cc(Cl)c(Cl)cc3nc2Cl)C1 ; N#Cc1cccc(B(O)O)c1 ; CC(C)C(=O)N(CCO[Si](C)(C)C(C)(C)C)[C@H]1CCN(c2nc3cc(Cl)c(Cl)cc3nc2-c2cccc(C#N)c2)C1 ; CC(C)C(=O)N(CCO)[C@H]1CCN(c2nc3cc(Cl)c(Cl)cc3nc2-c2cccc(C#N)c2)C1]

Compound 511 according to the present disclosure was prepared as shown in Scheme 86 by an analogous procedure to the one described in Examples 13A-13E.

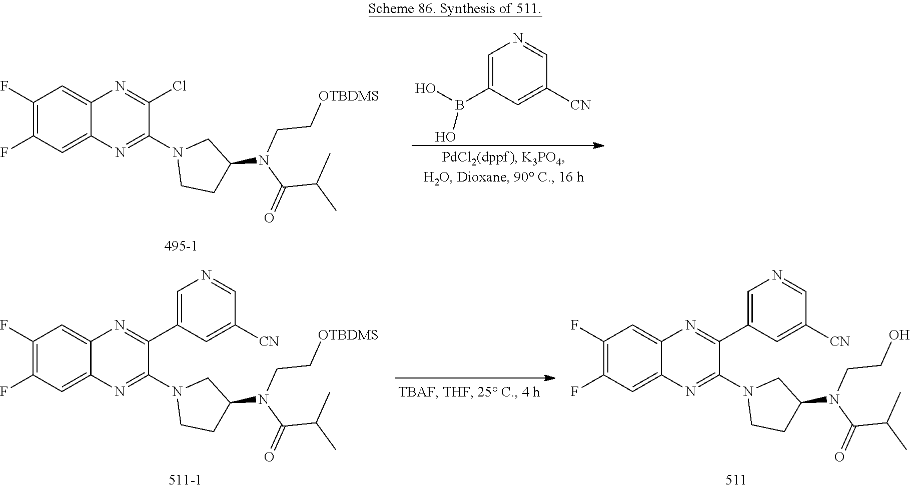 [SMILES: CC(C)C(=O)N(CCO[Si](C)(C)C(C)(C)C)[C@H]1CCN(c2nc3cc(F)c(F)cc3nc2Cl)C1 ; N#Cc1cncc(B(O)O)c1 ; CC(C)C(=O)N(CCO[Si](C)(C)C(C)(C)C)[C@H]1CCN(c2nc3cc(F)c(F)cc3nc2-c2cncc(C#N)c2)C1 ; CC(C)C(=O)N(CCO)[C@H]1CCN(c2nc3cc(F)c(F)cc3nc2-c2cncc(C#N)c2)C1]

### Example 14: Synthesis of Analogs 403 and 259

Compound 403 according to the present disclosure was prepared as shown in Scheme 87 and described below.

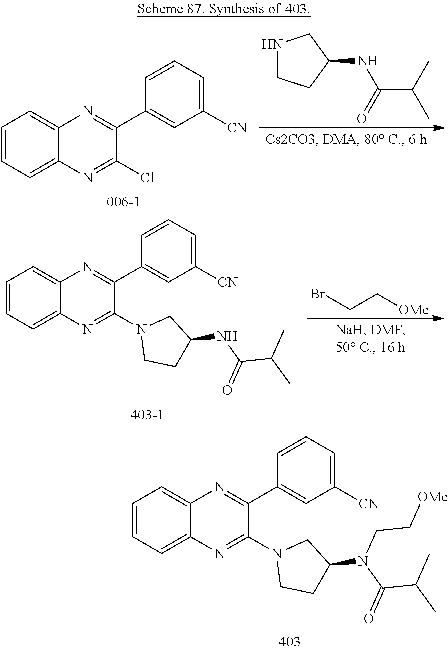 [SMILES: N#Cc1cccc(-c2nc3ccccc3nc2Cl)c1 ; CC(C)C(=O)N[C@H]1CCNC1 ; CC(C)C(=O)N[C@H]1CCN(c2nc3ccccc3nc2-c2cccc(C#N)c2)C1 ; COCCBr ; COCCN(C(=O)C(C)C)[C@H]1CCN(c2nc3ccccc3nc2-c2cccc(C#N)c2)C1]

### Example 14A: Preparation of Compound 403-1

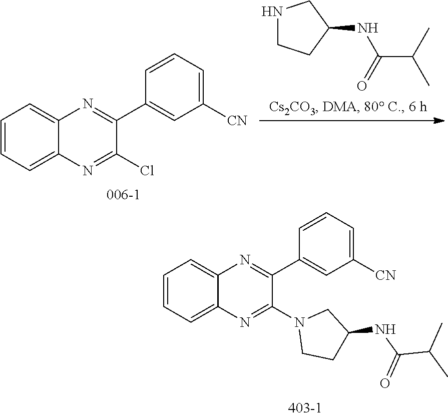 [SMILES: N#Cc1cccc(-c2nc3ccccc3nc2Cl)c1 ; CC(C)C(=O)N[C@H]1CCNC1 ; CC(C)C(=O)N[C@H]1CCN(c2nc3ccccc3nc2-c2cccc(C#N)c2)C1]

A solution of 3-(3-chloroquinoxalin-2-yl)benzonitrile 006-1 (100 mg, 0.38 mmol) and Cs₂CO₃ (613 mg, 1.88 mmol) in DMA (6 mL) was stirred under a nitrogen atmosphere at 25° C. 2-Methyl-N-[(3S)-pyrrolidin-3-yl]propenamide (71 mg, 0.45 mmol) was added. The reaction mixture was stirred at 80° C. for 6 h. The reaction was quenched with ice-water (10 mL) and extracted with EtOAc (50 mL). The organic phase was washed with brine (50 mL), dried over Na₂SO₄, concentrated under reduced pressure and purified by chromatography on silica gel, eluting with 70% EtOAc in petroleum ether to afford N-[(3S)-1-[3-(3-cyanophenyl)quinoxalin-2-yl]pyrrolidin-3-yl]-2-methylpropanamide 403-1 (90 mg, 61.4% yield) as a yellow solid. LC MS of 403-1: Ret. Time: 1.280 min, MS (ESI) m/z: 386.2, [M+H]⁺.

### Example 14B: Preparation of Compound 403

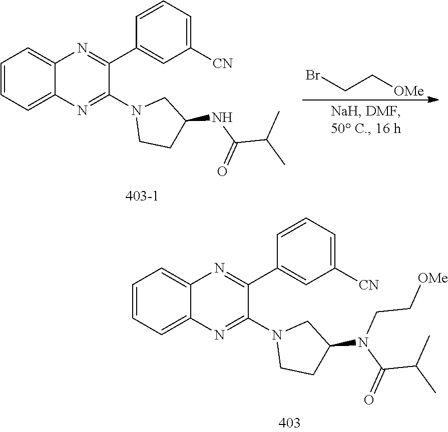 [SMILES: CC(C)C(=O)N[C@H]1CCN(c2nc3ccccc3nc2-c2cccc(C#N)c2)C1 ; COCCBr ; COCCN(C(=O)C(C)C)[C@H]1CCN(c2nc3ccccc3nc2-c2cccc(C#N)c2)C1]

A solution of (S)—N-(1-(3-(3-cyanophenyl)quinoxalin-2-yl)pyrrolidin-3-yl)isobutyramide 403-1 (250 mg, 0.645 mmol) in DMF (8 mL) was stirred under a nitrogen atmosphere at 0° C. NaH (23 mg, 0.67 mmol) was added. The reaction mixture was stirred at room temperature for 1 h. 1-Bromo-2-methoxyethane (361 mg, 2.59 mmol) was added. The reaction mixture was stirred at 50° C. for 16 h. The reaction was quenched with ice-water (30 mL) and extracted with EtOAc (100 mL). The organic phase was washed with brine (100 mL), dried over Na₂SO₄, concentrated under reduced pressure and purified by chromatography on silica gel, eluting with 25% EtOAc in petroleum ether to afford (S)—N-(1-(3-(3-cyanophenyl)quinoxalin-2-yl)pyrrolidin-3-yl)-N-(2-methoxyethyl)isobutyramide 403 (33.2 mg, 11.5% yield) as a yellow solid. LC MS of 403: Ret. Time: 1.368 min, MS (ESI) m/z: 444.2, [M+H]⁺. ¹H NMR of 403: ¹H NMR (400 MHz, CDCl₃) δ 8.26-8.00 (m, 2H), 7.99-7.81 (m, 2H), 7.75 (d, J=7.4 Hz, 1H), 7.65 (dt, J=14.8, 7.4 Hz, 2H), 7.50 (dt, J=8.2, 2.2 Hz, 1H), 4.70-4.45 (m, 1H), 3.35-3.70 (m, 8H), 3.29 (d, J=26.0 Hz 3H), 2.87-2.73 (m, 1H), 2.21-2.00 (m, 2H), 1.18-0.98 (m, 6H).

### Example 14C: Preparation of Analogous Compound 259

Table 12, below, depicts exemplary compounds synthesized according to an analogous procedure to the one described for 403.

TABLE 12
Compounds synthesized analogously to 403.

| | Compound | | |
|---|---|---|---|
| Compound Structure | No. | Yield | Characterization |
| 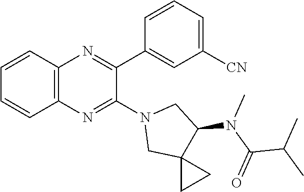 [SMILES: CC(C)C(=O)N(C)[C@@H]1CN(c2nc3ccccc3nc2-c2cccc(C#N)c2)CC12CC2] | 259 | 11.7% yield | LC MS of 259: Ret. Time: 1.302 min, MS (ESI) m/z: 426.1, M + H+. ¹H NMR of 259: ¹H NMR (400 MHz, DMSO) δ 8.21 (d, J = 14.4 Hz, 1H), 8.05 (d, J = 8.0 Hz, 1H), 7.95 (dd, J = 20.4, 8.0 Hz, 2H), 7.84-7.62 (m, 3H), 7.51 (t, J = 7.6 Hz, 1H), 4.81-4.23 (m, 1H), 4.06-3.76 (m, 1H), 3.47 (d, J = 10.8 Hz, 1H), 3.10-2.91 (m, 3H), 2.80 (dd, J = 28.4, 9.6 Hz, 2H), 2.67 (s, 1H), 1.09-0.85 (m, 6H), 0.70-0.60 (m, 3H), 0.48 (d, J = 10.4 Hz, 1H). |

Compound 259 according to the present disclosure was prepared as shown in Scheme 88 by an analogous procedure to the one described in Examples 14A-14B.

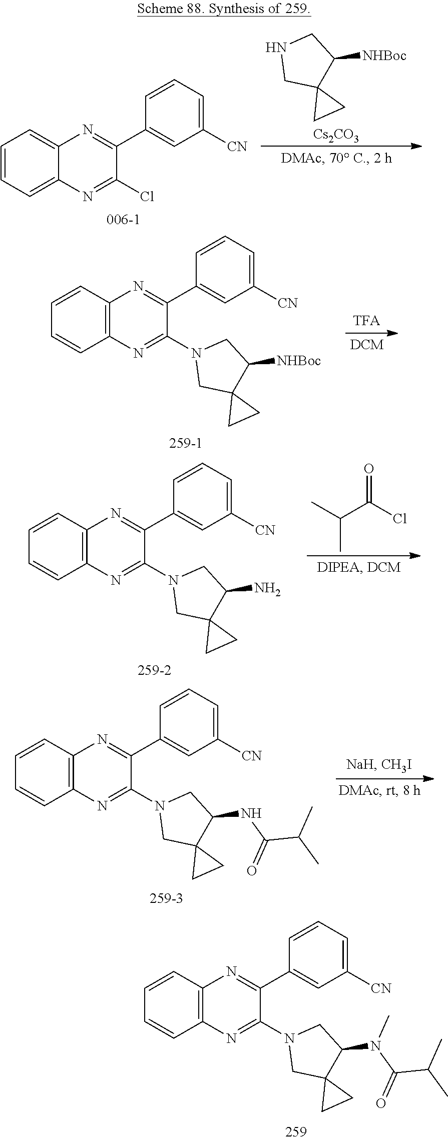

### Example 15: Synthesis of Analog 407

Compound 407 according to the present disclosure was prepared as shown in Scheme 89 and described below.

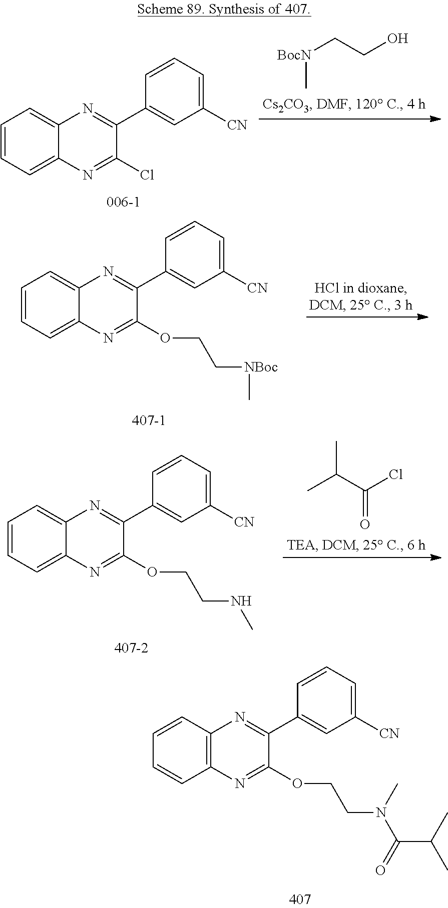

### Example 15A: Preparation of Compound 407-1

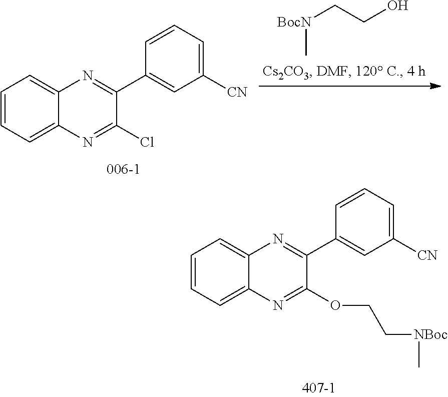 [SMILES: N#Cc1cccc(-c2nc3ccccc3nc2Cl)c1 ; CN(CCO)C(=O)OC(C)(C)C ; CN(CCOc1nc2ccccc2nc1-c1cccc(C#N)c1)C(=O)OC(C)(C)C]

A solution of 3-(3-chloroquinoxalin-2-yl)benzonitrile 006-1 (550 mg, 2.07 mmol) and Cs₂CO₃ (2023 mg, 6.21 mmol) in DMF (20 mL) was stirred under a nitrogen atmosphere at 25° C. tert-Butyl N-(2-hydroxyethyl)-N-methylcarbamate (544 mg, 3.10 mmol) was added. The reaction mixture was stirred at 120° C. for 6 h. The reaction was quenched with ice-water (30 mL) and extracted with EtOAc (100 mL). The organic phase was washed with brine (100 mL), dried over Na₂SO₄, concentrated under reduced pressure and purified by chromatography on silica gel, eluting with 20% EtOAc in petroleum ether to afford tert-butyl (2-((3-(3-cyanophenyl)quinoxalin-2-yl)oxy)ethyl)(methyl)carbamate 407-1 (600 mg, 70.9% yield) as a colorless oil. LC MS of 407-1: Ret. Time: 1.510 min, MS (ESI) m/z: 405.2, [M+H]⁺.

### Example 15B: Preparation of Compound 407-2

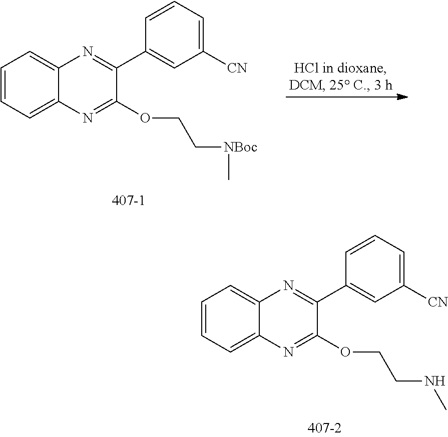 [SMILES: CN(CCOc1nc2ccccc2nc1-c1cccc(C#N)c1)C(=O)OC(C)(C)C ; CNCCOc1nc2ccccc2nc1-c1cccc(C#N)c1]

A solution of tert-butyl (2-((3-(3-cyanophenyl)quinoxalin-2-yl)oxy)ethyl)(methyl)-carbamate 407-1 (600 mg, 1.48 mmol) in DCM (10 mL) was stirred at 25° C. HCl in dioxane (3 mL) was added. The reaction mixture was stirred at 25° C. for 3 h. The reaction was concentrated under reduced pressure and purified by preparative HPLC to afford 3-(3-(2-(methylamino)ethoxy)quinoxalin-2-yl)benzonitrile 407-2 (400 mg, 87.7% yield) as yellow oil. LC MS of 407-2: Ret. Time: 1.014 min, MS (ESI) m/z: 305.1, [M+H]⁺.

### Example 15C: Preparation of Compound 407

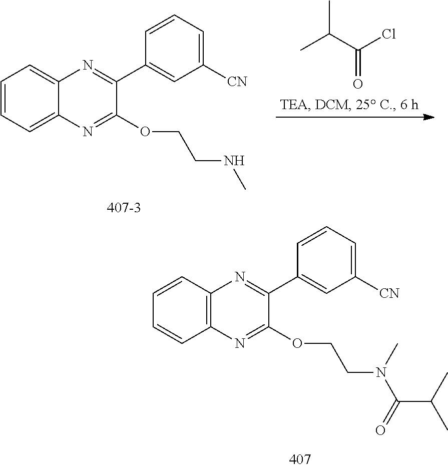

A solution of 3-(3-(2-(methylamino)ethoxy)quinoxalin-2-yl)benzonitrile 407-2 (200 mg, 0.66 mmol) and 2-methylpropanoyl chloride (105 mg, 0.99 mmol) in DCM (8 mL) was stirred under a nitrogen atmosphere at 25° C. TEA (399 mg, 3.94 mmol) was added. The reaction mixture was stirred at room temperature for 6 h. The reaction was quenched with ice-water (10 mL) and extracted with DCM (30 mL). The organic phase was washed with brine (30 mL), dried with Na₂SO₄, concentrated under reduced pressure and purified by chromatography on silica gel, eluting with 30% EtOAc in petroleum ether to afford N-(2-((3-(3-cyanophenyl)quinoxalin-2-yl)oxy)ethyl)-N-methylisobutyramide 407 (222.1 mg, 90.2% yield) as colorless oil. LC MS of 407: Ret. Time: 1.363 min, MS (ESI) m/z: 375.2, [M+H]⁻. ¹H NMR of 407: ¹H NMR (400 MHz, CDCl₃) δ 8.50-8.31 (m, 2H), 8.12-8.04 (m, 1H), 7.86 (dd, J=8.2, 1.0 Hz, 1H), 7.82-7.59 (m, 4H), 4.77 (t, J=5.6 Hz, 2H), 3.95-3.79 (m, 2H), 3.11 (s, 2H), 3.02 (s, 1H), 2.76 (dt, J=13.6, 6.8 Hz, 1H), 1.06 (t, J=7.0 Hz, 6H).

### Example 16: Synthesis of Analogs 408 and 409

Compound 408 according to the present disclosure was prepared as shown in Scheme 90 and described below.

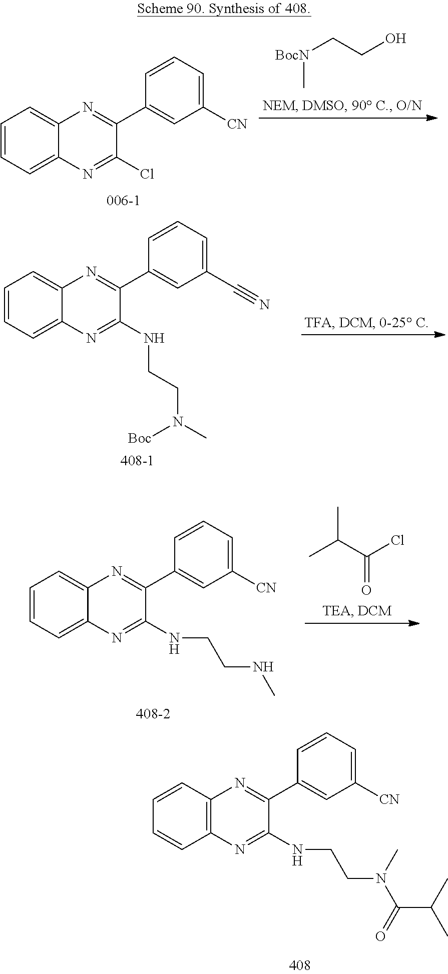

### Example 16A: Preparation of Compound 408-1

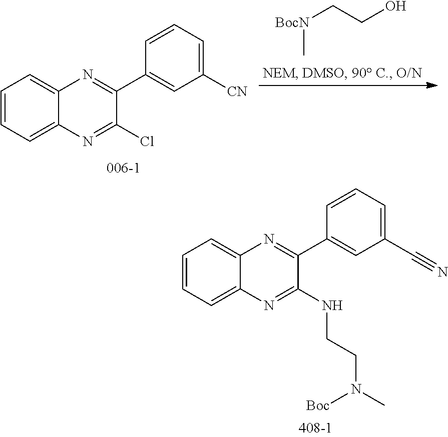 [SMILES: N#Cc1cccc(-c2nc3ccccc3nc2Cl)c1 ; CN(CCO)C(=O)OC(C)(C)C ; CN(CCNc1nc2ccccc2nc1-c1cccc(C#N)c1)C(=O)OC(C)(C)C]

To a solution of 3-(3-chloroquinoxalin-2-yl)benzonitrile 006-1 (500 mg, 1.88 mmol, 1.0 eq) and tert-butyl (2-aminoethyl)(methyl)carbamate (820 mg, 4.70 mmol, 2.5 eq) in DMSO (20 mL) stirred under nitrogen at 25° C. was added NEM (759 mg, 3.5 mmol, 3.5 eq). The reaction mixture was stirred at 90° C. for 16 h. After completion, the mixture was concentrated under reduced pressure at 60° C. The residue was purified by flash chromatography eluting with 1:1 petroleum ether/EtOAC to afford tert-butyl (2-((3-(3-cyanophenyl)quinoxalin-2-yl)amino)ethyl)(methyl)carbamate 408-1 (250 mg, yield: 51.2%) as a yellow oil. LC MS of 408-1: Ret. Time: 1.46 min, MS (ESI) m/z: 404.0, [M+H]⁺.

### Example 16B: Preparation of Compound 408-2

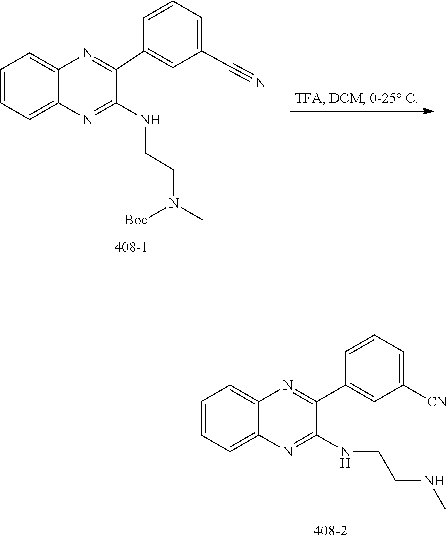 [SMILES: CN(CCNc1nc2ccccc2nc1-c1cccc(C#N)c1)C(=O)OC(C)(C)C ; CNCCNc1nc2ccccc2nc1-c1cccc(C#N)c1]

To a solution of tert-butyl (2-((3-(3-cyanophenyl)quinoxalin-2-yl)amino)ethyl)(methyl)carbamate 408-1 (250 mg, 0.37 mmol, 1.0 eq) in DCM (10 mL) stirred under nitrogen at 0° C. was added TFA (2 mL). The reaction mixture was stirred at 25° C. for 2 h.

After completion, the mixture was concentrated under reduced pressure at 40° C. The mixture was adjusted to pH 8. The residue was purified by flash chromatography eluting with 2:1 petroleum ether/EtOAC to afford 3-(3-((2-(methylamino)ethyl)amino)quinoxalin-2-yl)benzonitrile 408-2 (37 mg, yield: 13.9%) as a yellow oil. LC MS of 408-2: Ret. Time: 0.96 min, MS (ESI) m/z: 304.1, [M+H]⁺.

### Example 16C: Preparation of Compound 408

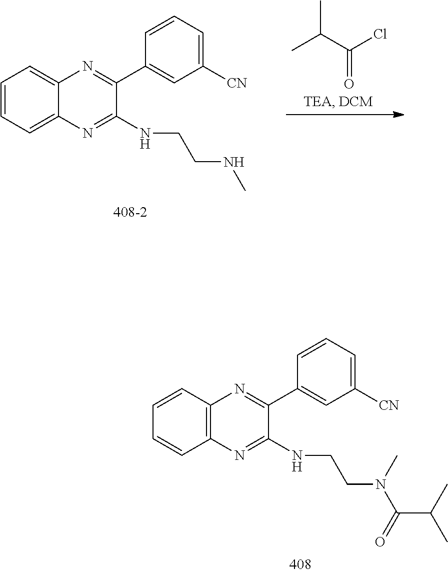

To a solution of 3-(3-((2-(methylamino)ethyl)amino)quinoxalin-2-yl)benzonitrile 408-2 (370 mg, 1.22 mmol, 1.0 eq) and TEA (740 mg, 7.32 mmol, 6 eq) in DCM (10 mL) stirred under nitrogen at 0° C. was added isobutyryl chloride (130 mg, 1.22 mmol, 1.0 eq). The reaction mixture was stirred at 25° C. for 2 h. After completion, the mixture was concentrated under reduced pressure at 40° C. The residue was purified by a preparative HPLC to afford N-(2-((3-(3-cyanophenyl)quinoxalin-2-yl)amino)ethyl)-N-methylisobutyramide 408 (85.6 mg, 18.8% yield) as a yellow solid. LC MS of 408: Ret. Time: 1.33 min, MS (ESI) m/z: 374.1, [M+H]⁺. ¹H NMR of 408: ¹H NMR (400 MHz, CDCl₃) δ 7.99-7.92 (m, 2H), 7.88 (dd, J=8.2, 1.2 Hz, 1H), 7.83-7.75 (m, 2H), 7.68 (t, J=7.8 Hz, 1H), 7.60 (dd, J=11.2, 4.2 Hz, 1H), 7.40 (t, J=7.8 Hz, 1H), 3.73 (s, 2H), 3.66 (dd, J=6.8, 3.6 Hz, 2H), 3.08 (s, 3H), 2.73-2.62 (m, 1H), 0.94 (d, J=6.8 Hz, 6H).

### Example 16D: Preparation of Analogous Compound 409

Table 13, below, depicts exemplary compounds synthesized according to an analogous procedure to the one described for 408.

TABLE 13
Compounds synthesized analogously to 408.

| | Compound | | |
|---|---|---|---|
| Compound Structure | No. | Yield | Characterization |
| 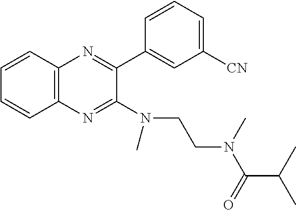 [SMILES: CC(C)C(=O)N(C)CCN(C)c1nc2ccccc2nc1-c1cccc(C#N)c1] | 409 | 21.2% yield | LC MS of 409: Ret. Time: 1.38 min, MS (ESI) m/z: 388.1, [M + H]+. ¹H NMR of 409: ¹H NMR (400 MHz, CDCl₃) δ 8.23-8.18 (m, 1H), 8.12-8.08 (m, 1H), 8.00-7.90 (m, 1H), 7.78-7.70 (m, 2H), 7.64- 7.59 (m, 2H), 7.57-7.45 (m, 1H), 3.68- 3.50 (m, 4H), 2.97 (s, 3H), 2.85 (s, 3H), 2.65- 2.53 (m, 1H), 1.20-0.90 (m, 6H). |

Compound 409 according to the present disclosure was prepared as shown in Scheme 91 by an analogous procedure to the one described in Examples 16A-16C.

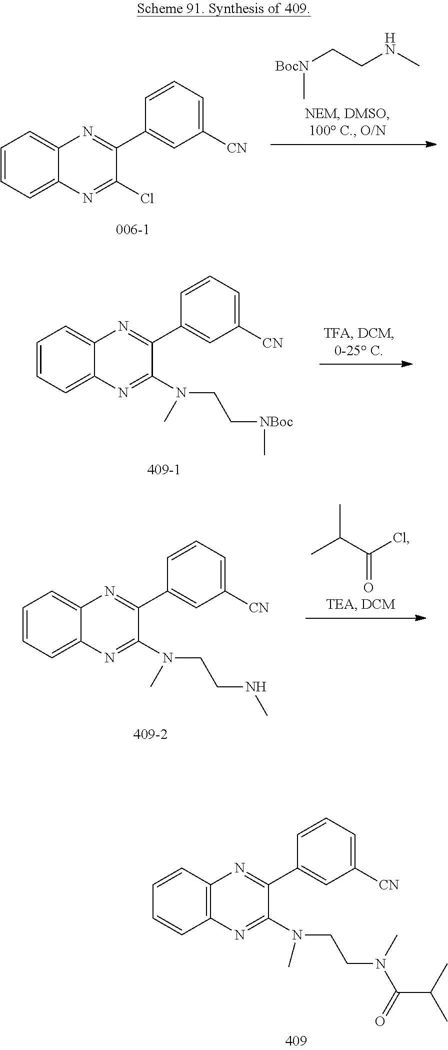

### Example 17: Synthesis of Analogs 410 and 200, 238, 411, 412, 414, 415, 416, 417, 418, 419, 420, 421, 422, 423, 424, 426, 427, 452, 466, 480, 481, 483, 497, 498, 499, 517

Compound 410 according to the present disclosure was prepared as shown in Scheme 92 and described below.

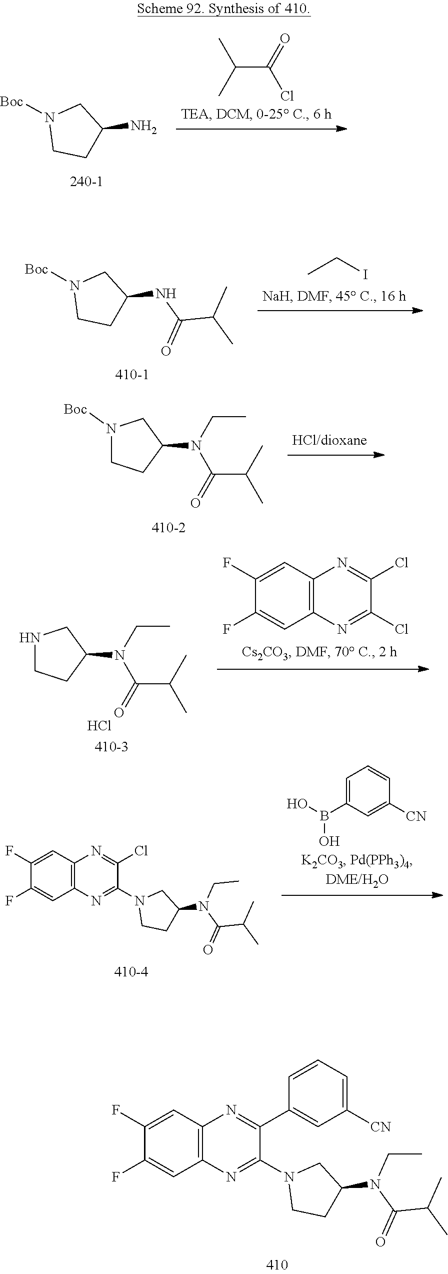

### Example 17A: Preparation of Compound 410-1

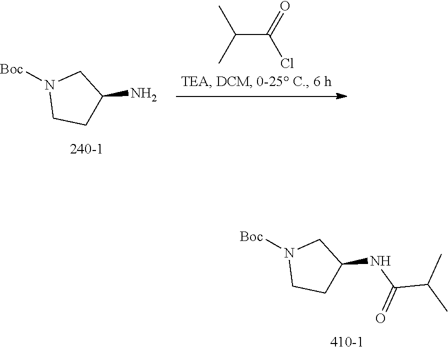

A solution of tert-butyl (S)-3-aminopyrrolidine-1-carboxylate 240-1 (14.5 g, 77.4 mmol, 1.0 eq) and triethylamine (19.58 g, 193.5 mmol, 2.5 eq) in DCM (200 mL) was stirred under nitrogen at 0° C. 2-Methylpropanoyl chloride (9.48 mg, 89 mmol, 1.15 eq) in DCM (50 mL) was slowly added dropwise to the reaction system at 0° C. The reaction mixture was stirred at 25° C. for 6 h. Saturated brine (200 mL) was added. The residue was extracted with DCM (3×100 mL). The combined organic layers were concentrated under reduced pressure at 30° C. The residue was purified by flash chromatography eluting with 1:1 petroleum ether/EtOAc to afford tert-butyl (S)-3-isobutyramidopyrrolidine-1-carboxylate 410-1 (18 g, yield: 85.8%) as a yellow oil. LC MS of 410-1: Ret. Time: 1.16 min, MS (ESI) m/z: 201.2, [M−55]⁺.

### Example 17B: Preparation of Compound 410-2

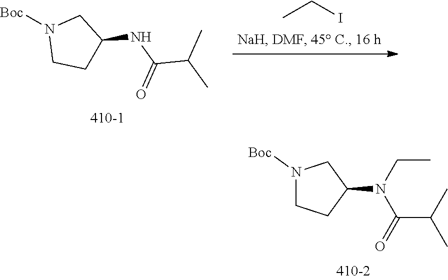

To anhydrous DMF (120 mL) was added sodium hydride (3.12 g, 2.0 eq, 60% dispersion in mineral oil) and the mixture was allowed to stir for 5 min. A solution of tert-butyl (3S)-3-(2-methylpropanamido)-pyrrolidine-1-carboxylate 410-1 (10 g, 39 mmol) in anhydrous DMF (30 mL) was added dropwise over a minute and allowed to stir for 30 min. Iodoethane (30.41 g, 195 mmol) was added and the reaction was allowed to stir at 45° C. for 12 h. Water (100 mL) was added. The residue was extracted with ethyl acetate (3×200 mL). The combined organic phases were washed with saturated brine (100 mL). The organic phase was concentrated under reduced pressure at 45° C. The residue was purified via flash chromatography eluting with petroleum ether/EtOAc to give the product 410-2 (10 g, yield: 85.6%) as a yellow oil. LC MS of 410-2: Ret. Time: 1.253 min, MS (ESI) m/z: 229.2, [M−55]⁻.

### Example 17C: Preparation of Compound 410-3

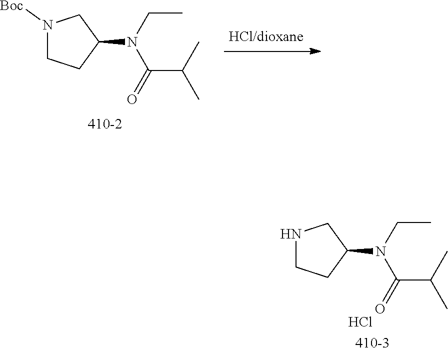 [SMILES: CCN(C(=O)C(C)C)[C@H]1CCN(C(=O)OC(C)(C)C)C1 ; CCN(C(=O)C(C)C)[C@H]1CCNC1 ; Cl]

To a solution of tert-butyl (3S)-3-(N-ethyl-2-methylpropanamido)pyrrolidine-1-carboxylate 410-2 (5.0 g, 17.58 mmol) in dioxane (50 mL) at 25° C., HCl in dioxane (30 mL, 4M, 120 mmol) was added. This reaction mixture was stirred at 25° C. for 3 h. The reaction mixture was concentrated under reduced pressure at 40° C. to give (S)—N-ethyl-N-(pyrrolidin-3-yl)isobutyramide hydrochloride 410-3 (3.7 g, yield: 95%) as a yellow oil which was used directly in the next step without further purification. LC MS of 410-3: Ret. Time: 0.452 min, MS (ESI) m/z: 185.2, [M+H]⁺.

### Example 17D: Preparation of Compound 410-4

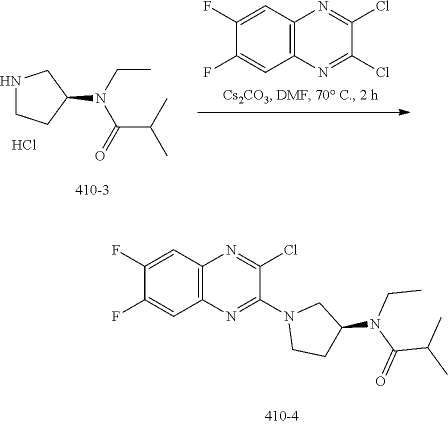 [SMILES: CCN(C(=O)C(C)C)[C@H]1CCNC1 ; Cl ; Fc1cc2nc(Cl)c(Cl)nc2cc1F ; CCN(C(=O)C(C)C)[C@H]1CCN(c2nc3cc(F)c(F)cc3nc2Cl)C1]

A round-bottom flask containing a mixture of 2,3-dichloro-6,7-difluoroquinoxaline (12.78 g, 54.3 mmol), N-ethyl-2-methyl-N-[(3S)-pyrrolidin-3-yl]propanamide hydrochloride 410-3 (10 g, 45.3 mmol) and cesium carbonate (73.8 g, 226.5 mmol) in DMF (250 mL) was placed in an oil bath heated to 70° C. The mixture was continuously stirred at 70° C. for 2 h. The reaction was cooled to 25° C. The mixture was diluted with water (200 mL). The residue was extracted with ethyl acetate (200 mL×3). The combined organic phases were washed with saturated brine (100 mL×5). The organic layer was concentrated under reduced pressure at 40° C. The residue was purified via flash chromatography eluting with petroleum ether/EtOAc to give the product (S)—N-(1-(3-chloro-6,7-difluoroquinoxalin-2-yl)pyrrolidin-3-yl)-N-ethylisobutyramide 410-4 (15 g, 79% yield) as a yellow solid. LC MS of 410-4: Ret. Time: 1.450 min, MS (ESI) m/z: 383.1, [M+1]⁺.

### Example 17E: Preparation of Compound 410

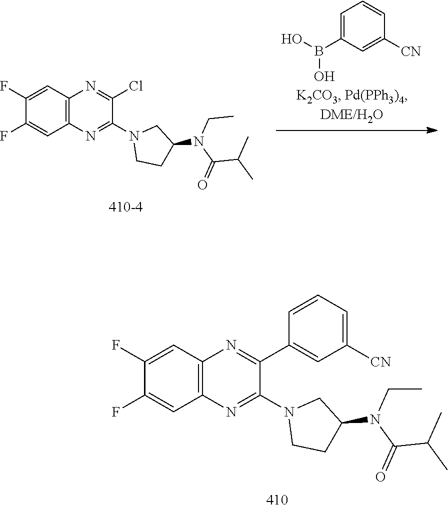 [SMILES: N#Cc1cccc(B(O)O)c1 ; CCN(C(=O)C(C)C)[C@H]1CCN(c2nc3cc(F)c(F)cc3nc2Cl)C1 ; CCN(C(=O)C(C)C)[C@H]1CCN(c2nc3cc(F)c(F)cc3nc2-c2cccc(C#N)c2)C1]

To a solution of (S)—N-(1-(3-chloro-6,7-difluoroquinoxalin-2-yl)pyrrolidin-3-yl)-N-ethylisobutyramide 410-4 (150 mg, 0.39 mmol, 1.0 eq), (3-cyanophenyl)boronic acid (86 mg, 0.59 mmol, 1.5 eq) and K₂CO₃ (162 mg, 1.18 mmol, 3.0 eq) in a 5:1 DME/H₂O solution (6 mL) stirred under nitrogen at 25° C. was added Pd(PPh₃)₄ (45 mg, 0.039 mmol, 0.1 eq). The reaction mixture was stirred at 100° C. for 16 h. After completion, the mixture was cooled to room temperature, quenched with H₂O (50 mL) and extracted with EtOAc (3×50 mL). The combined organic layers were washed with brine for three times and dried over sodium sulphate, filtered and concentrated. The residue was purified by a preparative HPLC to afford (S)—N-(1-(3-(3-cyanophenyl)-6,7-difluoroquinoxalin-2-yl)pyrrolidin-3-yl)-N-ethylisobutyramide 410 (76 mg, 43.2% yield) as a yellow solid. LC MS of 410: Ret. Time: 1.44 min, MS (ESI) m/z: 450.1, [M+H]⁺. ¹H NMR of 410: ¹H NMR (400 MHz, CDCl₃) δ 8.03 (s, 1H), 8.01-7.95 (m, 1H), 7.68 (m, 4H), 4.93-4.43 (m, 1H), 3.49-3.12 (m, 6H), 2.85-2.64 (m, 1H), 2.14-1.98 (m, 2H), 1.22-1.05 (m, 9H).

### Example 17F: Preparation of Analogous Compounds 200, 238, 411, 412, 414, 415, 416, 417, 418, 419, 420, 421, 422, 423, 424, 426, 427, 452, 466, 480, 481, 483, 497, 498, 499, and 517

Table 14, below, depicts exemplary compounds synthesized according to an analogous procedure to the one described for 410.

TABLE 14
Compounds synthesized analogously of 410.

| | Compound | | |
|---|---|---|---|
| Compound Structure | No. | Yield | Characterization |
| 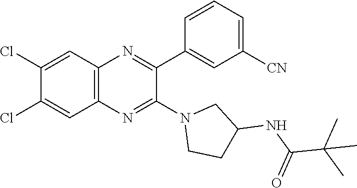 [SMILES: CC(C)(C)C(=O)NC1CCN(c2nc3cc(Cl)c(Cl)cc3nc2-c2cccc(C#N)c2)C1] | 200 | 13.7% yield | LC MS of 200: Ret. Time: 1.458 min, MS (ESI) m/z: 468.2, M + H⁺. ¹H NMR of 200: ¹H NMR (400 MHz, DMSO) δ 8.13 (d, J = 13.6 Hz, 2H), 7.99 (dd, J = 13.2, 9.2 Hz, 3H), 7.74 (d, J = 7.6 Hz, 1H), 7.43 (d, J = 6.4 Hz, 1H), 4.21 (d, J = 5.6 Hz, 1H), 3.46-3.43 (dd, J = 10.8, 6.4 Hz, 1H), 3.20 (d, J = 15.2 Hz, 3H), 1.94 (dd, J = 12.0, 5.8 Hz, 1H), 1.84-1.81 (dd, J = 12.4, 6.4 Hz, 1H), 1.06 (s, 9H). |
| 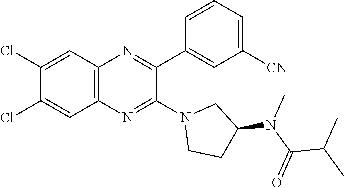 [SMILES: CC(C)C(=O)N(C)[C@H]1CCN(c2nc3cc(Cl)c(Cl)cc3nc2-c2cccc(C#N)c2)C1] | 238 | 5.8% yield | LC MS of 238: Ret. Time: 1.480 min, MS (ESI) m/z: 468.3, M + H⁺. ¹H NMR of 238: ¹H NMR (400 MHz, DMSO) δ 8.15 (d, J = 6.0 Hz, 2H), 8.05- 7.91 (m, 3H), 7.74 (t, J = 7.6 Hz, 1H), 5.05- 4.66 (m, 1H), 3.40 (d, J = 9.2 Hz, 1H), 3.28- 3.16 (m, 3H), 3.01-2.74 (m, 3H), 2.67 (s, 1H), 2.09-1.84 (m, 2H), 0.97 (d, J = 6.4 Hz, 6H). |
| 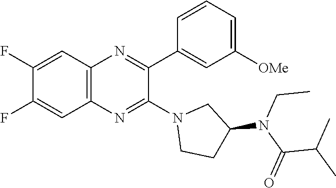 [SMILES: CCN(C(=O)C(C)C)[C@H]1CCN(c2nc3cc(F)c(F)cc3nc2-c2cccc(OC)c2)C1] | 411 | 37.6% yield | LC MS of 411: Ret. Time: 1.48 min, MS (ESI) m/z: 455.0, [M + H]⁺. ¹H NMR of 411: ¹H NMR (400 MHz, CDCl₃) δ 7.75-7.65 (m, 1H), 7.61-7.49 (m, 1H), 7.38 (t, J = 8.2 Hz, 1H), 7.21 (s, 2H), 7.03-6.95 (m, 1H), 5.02-4.38 (m, 1H), 3.88 (s, 3H), 3.50-3.19 (m, 6H), 2.82- 2.62 (m, 1H), 2.15-1.85 (m, 2H), 1.20- 1.03 (m, 9H). |
| 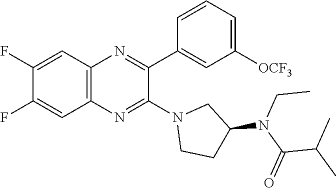 [SMILES: CCN(C(=O)C(C)C)[C@H]1CCN(c2nc3cc(F)c(F)cc3nc2-c2cccc(OC(F)(F)F)c2)C1] | 412 | 54.5% yield | LC MS of 412: Ret. Time: 1.55 min, MS (ESI) m/z: 509.1, [M + H]⁺. ¹H NMR of 412: ¹H NMR (400 MHz, CDCl₃) δ 7.73-7.63 (m, 2H), 7.59-7.50 (m, 3H), 7.36-7.24 (m, 1H), 5.02-4.38 (m, 1H), 3.50-3.11 (m, 6H), 2.82-2.62 (m, 1H), 2.23-1.89 (m, 2H), 1.23-1.01 (m, 9H). |
| 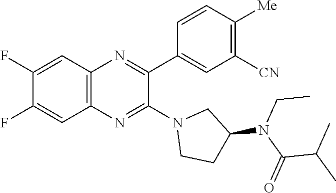 [SMILES: CCN(C(=O)C(C)C)[C@H]1CCN(c2nc3cc(F)c(F)cc3nc2-c2ccc(C)c(C#N)c2)C1] | 414 | 71.3% yield | LC MS of 414: Ret. Time: 1.469 min, MS (ESI) m/z: 464.2, [M + H]⁺. ¹H NMR of 414: ¹H NMR (400 MHz, CDCl₃) δ 7.97 (s, 1H), 7.90-7.72 (m, 2H), 7.72-7.63 (m, 1H), 7.46 (d, J = 7.4 Hz, 1H), 4.81 (s, 1H), 3.66-3.16 (m, 6H), 2.87- 2.67 (m, 1H), 2.63 (s, 3H), 2.15-1.95 (m, 2H), 1.16 (m, 9H). |
| 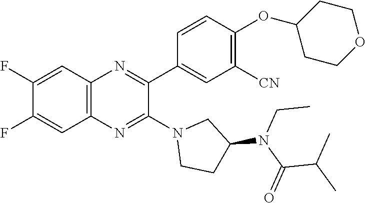 [SMILES: CCN(C(=O)C(C)C)[C@H]1CCN(c2nc3cc(F)c(F)cc3nc2-c2ccc(OC3CCOCC3)c(C#N)c2)C1] | 415 | 51.6% yield | LC MS of 415: Ret. Time: 1.433 min, MS (ESI) m/z: 550.3, [M + H]⁺. ¹H NMR of 415: ¹H NMR (400 MHz, CDCl₃) δ 8.05-7.80 (m, 3H), 7.74-7.63 (m, 1H), 7.12 (s, 1H), 4.76 (s, 2H), 4.04 (s, 2H), 3.79-3.12 (m, 8H), 2.87-2.68 (m, 1H), 2.06 (s 4H), 1.94 (s, 2H), 1.40-0.96 (m, 9H). |
| 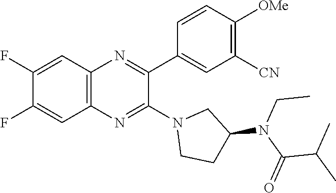 [SMILES: CCN(C(=O)C(C)C)[C@H]1CCN(c2nc3cc(F)c(F)cc3nc2-c2ccc(OC)c(C#N)c2)C1] | 416 | 42.3% yield | LC MS of 416: Ret. Time: 1.424 min, MS (ESI) m/z: 480.2, [M + H]⁺. ¹H NMR of 416: ¹H NMR (400 MHz, CDCl₃) δ 8.00-7.90 (m, 2H), 7.73-7.73- 7.50 (m, 2H), 7.10 (d, J = 7.8 Hz, 1H), 4.84 (s, 1H), 4.02 (s, 3H), 3.57-3.11 (m, 6H), 2.85-2.67 (m, 1H), 2.07 (s, 2H), 1.25- 0.99 (m, 9H). |
| 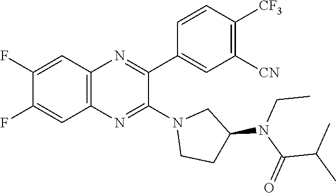 [SMILES: CCN(C(=O)C(C)C)[C@H]1CCN(c2nc3cc(F)c(F)cc3nc2-c2ccc(C(F)(F)F)c(C#N)c2)C1] | 417 | 42.8% yield | LC MS of 417: Ret. Time: 1.488 min, MS (ESI) m/z: 518.2, [M + H]⁺. ¹H NMR of 417: ¹H NMR (400 MHz, CDCl₃) δ 8.26 (s, 1H), 8.09 (d, J = 7.6 Hz, 1H), 7.93 (d, J = 8.0 Hz, 1H), 7.84-7.58 (m, 2H), 4.80-4.50 (s, 1H), 3.57-3.10 (m, 6H), 2.85-2.65 (m, 1H), 2.17-2.07 (m, 2H), 1.44-0.88 (m, 9H). |
| 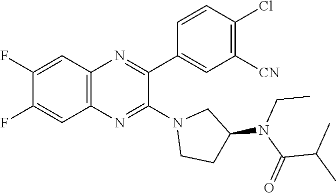 [SMILES: CCN(C(=O)C(C)C)[C@H]1CCN(c2nc3cc(F)c(F)cc3nc2-c2ccc(Cl)c(C#N)c2)C1] | 418 | 17.6% yield | LC MS of 418: Ret. Time: 1.484 min, MS (ESI) m/z: 484.2, [M + H]⁺. ¹H NMR of 418: ¹H NMR (400 MHz, CDCl₃) δ 8.09 (s, 1H), 7.92 (d, J = 8.6 Hz, 1H), 7.83-7.74 (m, 1H), 7.73-7.59 (m, 2H), 4.74 (s, 1H), 3.62-3.15 (m, 6H), 2.82- 2.66 (m, 1H), 2.11 (s, 2H), 1.22 (dd, J = 16.0, 9.2 Hz, 3H), 1.12 (dd, J = 6.6, 2.2 Hz, 6H). |
| 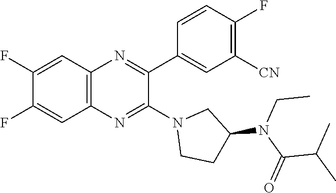 [SMILES: CCN(C(=O)C(C)C)[C@H]1CCN(c2nc3cc(F)c(F)cc3nc2-c2ccc(F)c(C#N)c2)C1] | 419 | 38.4% yield | LC MS of 419: Ret. Time: 1.445 min, MS (ESI) m/z: 468.2, [M + H]⁺. ¹H NMR of 419: ¹H NMR (400 MHz, CDCl₃) δ 8.09-7.93 (m, 2H), 7.75-7.52 (m, 2H), 7.36 (t, J = 8.3 Hz, 1H), 4.90-4.45 (m, 1H), 3.60-3.16 (m, 6H), 2.90-2.65 (m, 1H), 2.20-2.00 (m, 2H), 1.26-1.06 (m, 9H). |
| 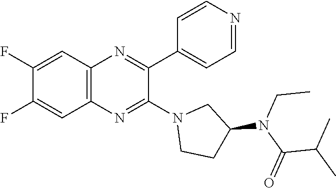 [SMILES: CCN(C(=O)C(C)C)[C@H]1CCN(c2nc3cc(F)c(F)cc3nc2-c2ccncc2)C1] | 420 | 51.0% yield | LC MS of 420: Ret. Time: 1.272 min, MS (ESI) m/z: 426.2, [M + H]⁺. ¹H NMR of 420: ¹H NMR (400 MHz, CDCl₃) δ 8.76 (d, J = 4.6 Hz, 2H), 7.78- 7.63 (m, 3H), 7.53 (dd, J = 11.0, 8.0 Hz, 1H), 4.94-4.75 (m, 1H), 3.51-3.18 (m, 6H), 2.87-2.68 (m, 1H), 2.12-1.99 (m, 2H), 1.23-1.05 (m, 9H). |
| 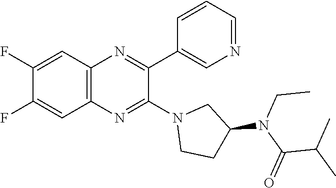 [SMILES: CCN(C(=O)C(C)C)[C@H]1CCN(c2nc3cc(F)c(F)cc3nc2-c2cccnc2)C1] | 421 | 54.0% yield | LC MS of 421: Ret. Time: 1.318 min, MS (ESI) m/z: 426.1, [M + H]⁺. ¹H NMR of 421: ¹H NMR (400 MHz, CDCl₃) δ 8.98 (s, 1H), 8.70 (s, 1H), 8.25- 8.10 (m, 1H), 7.85-7.60 (m, 1H), 7.60- 7.45 (m, 2H), 4.85 (s, 1H), 3.60-3.06 (m, 6H), 2.85-2.65 (m, 1H), 2.14-1.98 (m, 2H), 1.25-1.00 (m, 9H). |
| 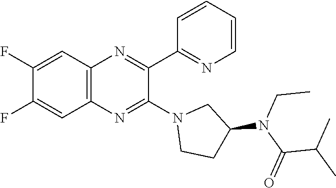 [SMILES: CCN(C(=O)C(C)C)[C@H]1CCN(c2nc3cc(F)c(F)cc3nc2-c2ccccn2)C1] | 422 | 99.0% yield | LC MS of 422: Ret. Time: 1.346 min, MS (ESI) m/z: 426.3, [M + H]⁺. ¹H NMR of 422: ¹H NMR (400 MHz, CDCl₃) δ 8.66 (d, J = 4.7 Hz, 1H), 8.01 (t, J = 7.7 Hz, 1H), 7.92 (dd, J = 18.1, 6.7 Hz, 2H), 7.70 (ddd, J = 12.1, 8.2, 4.3 Hz, 1H), 7.51 (t, J = 5.3 Hz, 1H), 4.79-4.37 (m, 1H), 3.42-3.34 (m, 1H), 3.33-3.00 (m, 5H), 2.97-2.59 (m, 1H), 2.14-1.80 (m, 2H), 1.13-0.71 (m, 9H). |
| 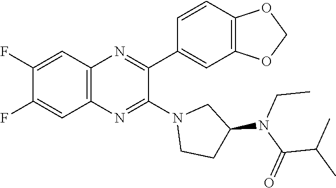 [SMILES: CCN(C(=O)C(C)C)[C@H]1CCN(c2nc3cc(F)c(F)cc3nc2-c2ccc3c(c2)OCO3)C1] | 423 | 73.1% yield | LC MS of 423: Ret. Time: 1.46 min, MS (ESI) m/z: 469.1, [M + H]⁺. ¹H NMR of 423: ¹H NMR (400 MHz, CDCl₃) δ 7.71-7.48 (m, 2H), 7.20-7.08 (m, 2H), 6.91 (d, J = 7.8 Hz, 1H), 6.05 (s, 2H), 5.02-4.38 (m, 1H), 3.55-3.15 (m, 6H), 2.83-2.67 (m, 1H), 2.15-1.85 (m, 2H), 1.21-1.03 (m, 9H). |
| 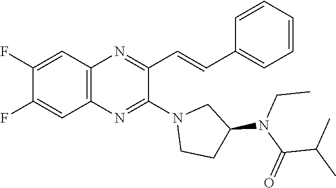 [SMILES: CCN(C(=O)C(C)C)[C@H]1CCN(c2nc3cc(F)c(F)cc3nc2/C=C/c2ccccc2)C1] | 424 | 50.6% yield | LC MS of 424: Ret. Time: 1.561 min, MS (ESI) m/z: 451.2, M + H⁺. ¹H NMR of 424: ¹H NMR (400 MHz, DMSO-d₆) δ 7.93-7.83 (m, 1H), 7.79- 7.71 (m, 2H), 7.71-7.63 (m, 2H), 7.63- 7.51 (m, 1H), 7.43 (t, J = 7.5 Hz, 2H), 7.37 (t, J = 7.2 Hz, 1H), 4.86-4.56 (m, 1H), 3.98-3.67 (m, 4H), 3.45-3.35 (m, 1H), 3.33-3.22 (m, 1H), 3.05-2.75 (m, 1H), 2.23-2.03 (m, 2H), 1.18-0.91 (m, 9H). |
| 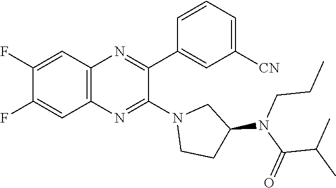 [SMILES: CCCN(C(=O)C(C)C)[C@H]1CCN(c2nc3cc(F)c(F)cc3nc2-c2cccc(C#N)c2)C1] | 426 | 20.0% yield | LC MS of 426: Ret. Time: 1.476 min, MS (ESI) m/z: 464.1, [M + H]⁺. ¹H NMR of 426: ¹H NMR (400 MHz, CDCl₃) δ 7.98-7.85 (m, 2H), 7.80-7.55 (m, 3H), 7.55-7.45 (m, 1H), 4.85-4.40 (m, 1H), 3.36-3.21 (m, 3H), 3.16-2.92 (m, 3H), 2.83-2.60 (m, 1H), 2.06-1.89 (m, 2H), 1.52-1.38 (m, 2H), 1.09-0.98 (m, 6H), 0.88-0.77 (m, 3H). |
| 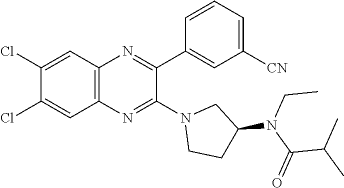 [SMILES: CCN(C(=O)C(C)C)[C@H]1CCN(c2nc3cc(Cl)c(Cl)cc3nc2-c2cccc(C#N)c2)C1] | 427 | 51.8% yield | LC MS of 427: Ret. Time: 1.543 min, MS (ESI) m/z: 482.0, [M + H]⁺. ¹H NMR of 427: ¹NMR (400 MHz, CDCl₃) δ 8.11 (s, 1H), 8.03 (d, J = 3.4 Hz, 2H), 7.95 (d, J = 7.6 Hz, 2H), 7.76 (d, J = 7.6 Hz, 2H), 7.63 (t, J = 9.8 Hz, 2H), 4.87-4.45 (m, 1H), 3.65-3.10 (m, 6H), 2.85-2.66 (m, 1H), 2.15-2.00 (m, 2H), 1.44-0.95 (m, 9H). |
| 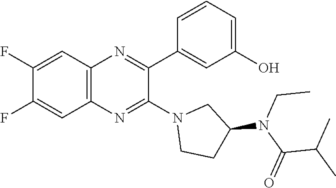 [SMILES: CCN(C(=O)C(C)C)[C@H]1CCN(c2nc3cc(F)c(F)cc3nc2-c2cccc(O)c2)C1] | 452 | 94.7% yield | LC MS of 452: Ret. Time: 1.357 min, MS (ESI) m/z: 441.2, [M + H]⁺. ¹H NMR of 452: ¹H NMR (400 MHz, CDCl₃) δ 7.76-7.62 (m, 1H), 7.56-7.46 (m, 1H), 7.30-7.20 (m, 1H), 7.20-7.06 (m, 2H), 6.90 (d, J = 8.4 Hz, 1H), 4.83- 4.40 (m, 1H), 3.56-3.05 (m, 6H), 2.81- 2.67 (m, 1H), 2.12-1.98 (m, 2H), 1.26- 0.86 (m, 9H). |
| 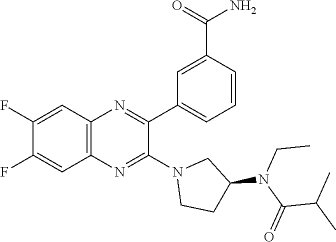 [SMILES: CCN(C(=O)C(C)C)[C@H]1CCN(c2nc3cc(F)c(F)cc3nc2-c2cccc(C(N)=O)c2)C1] | 466 | 42.7% yield | LC MS of 466: Ret Time: 1.276 min, MS (ESI) m/z: 468.2, [M + H]⁺. ¹H NMR of 466: ¹H NMR (400 MHz, DMSO-d₆) δ 8.14 (t, J = 1.7 Hz, 1H), 7.97 (dt, J = 5.2, 3.9 Hz, 1H), 7.86 (dd, J = 11.2, 8.7 Hz, 1H), 7.82-7.78 (m, 1H), 7.65 (dd, J = 11.9, 8.3 Hz, 1H), 7.58 (t, J = 7.7 Hz, 1H), 4.67-4.47 (m, 1H), 3.50-3.30 (m, 2H), 3.30-3.10 (m, 4H), 2.85-2.71 (m, 1H), 2.06-1.94 (m, 2H), 1.03-0.90 (m, 9H). |
| 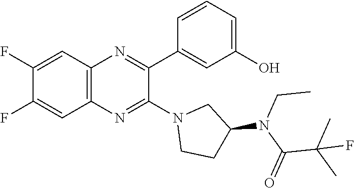 | 480 | 44.8% yield | LC MS of 480: Ret. Time: 1.461 min, MS (ESI) m/z: 468.2, [M + H]⁺. ¹H NMR of 480: ¹H NMR (400 MHz, DMSO-d₆) δ 8.10 (s, 1H), 8.00 (d, J = 7.9 Hz, 1H), 7.94-7.90 (m, 1H), 7.87 (dd, J = 11.1, 8.7 Hz, 1H), 7.76-7.69 (m, 1H), 7.67 (dd, J = 11.9, 8.3 Hz, 1H), 4.80-4.50 (m, 1H), 3.57-3.16 (m, 6H), 2.17-1.90 (m, 2H), 1.52 (dd, J = 21.7, 5.9 Hz, 6H), 1.03 (t, J = 6.9 Hz, 3H). |
| 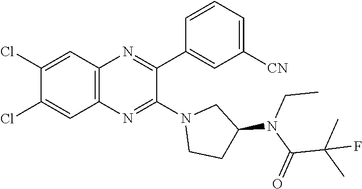 [SMILES: CCN(C(=O)C(C)(C)F)[C@H]1CCN(c2nc3cc(Cl)c(Cl)cc3nc2-c2cccc(C#N)c2)C1] | 481 | 38.7% yield | LC MS of 481: Ret. Time: 1.581 min, MS (ESI) m/z: 500.0, [M + H]⁺. ¹H NMR of 481: ¹H NMR (400 MHz, DMSO-d₆) δ 8.10 (s, 2H), 8.02-7.97 (m, 1H), 7.95-7.90 (m, 2H), 7.72 (t, J = 7.8 Hz, 1H), 4.78-4.45 (m, 1H), 3.55-3.35 (m, 2H), 3.35-3.21 (m, 4H), 2.16-1.95 (m, 2H), 1.52 (dd, J = 21.7, 5.6 Hz, 6H), 1.03 (t, J = 6.9 Hz, 3H). |
| 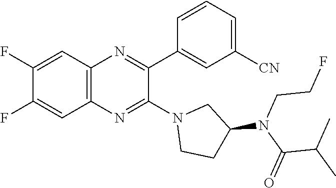 [SMILES: CC(C)C(=O)N(CCF)[C@H]1CCN(c2nc3cc(F)c(F)cc3nc2-c2cccc(C#N)c2)C1] | 483 | 31.7% yield | LC MS of 483: Ret. Time: 1.386 min, MS (ESI) m/z: 468.1, [M + H]⁺. ¹H NMR of 483: ¹H NMR (400 MHz, DMSO-d₆) δ 8.08 (t, J = 1.7 Hz, 1H), 8.01- 7.96 (m, 1H), 7.93-7.89 (m, 1H), 7.89- 7.83 (m, 1H), 7.70 (t, J = 6.7 Hz, 1H), 7.66 (dd, J = 10.7, 7.1 Hz, 1H), 4.61-4.51 (m, 1H), 4.50-4.42 (m, 1H), 4.39-4.31 (m, 1H), 3.53 (d, J = 19.1 Hz, 2H), 3.45-3.33 (m, 2H), 3.33-3.24 (m, 1H), 3.24-3.13 (m, 1H), 2.95-2.80 (m, 1H), 2.10-1.98 (m, 2H), 0.98 (dd, J = 8.9, 6.7 Hz, 6H). |
| 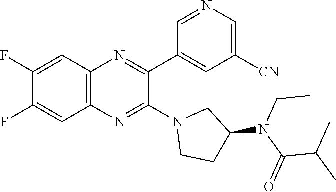 [SMILES: CCN(C(=O)C(C)C)[C@H]1CCN(c2nc3cc(F)c(F)cc3nc2-c2cncc(C#N)c2)C1] | 497 | 52.4% yield | LC MS of 497: Ret. Time: 1.369 min, MS (ESI) m/z: 451.2, [M + H]⁺. ¹H NMR of 497: ¹H NMR (400 MHz, CDCl₃) δ 9.16 (s, 1H), 8.94 (s, 1H), 8.42 (s, 1H), 7.78-7.66 (m, 2H), 4.86-4.48 (m, 1H), 3.59-3.11 (m, 6H), 2.86-2.65 (m, 1H), 2.20-2.04 (m, 2H), 1.34-1.02 (m, 9H). |
| 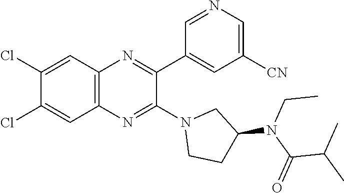 [SMILES: CCN(C(=O)C(C)C)[C@H]1CCN(c2nc3cc(Cl)c(Cl)cc3nc2-c2cncc(C#N)c2)C1] | 498 | 18.5% yield | LC MS of 498: Ret. Time: 1.479 min, MS (ESI) m/z: 483.1, [M + H]⁺. ¹H NMR of 498: ¹H NMR (400 MHz, CDCl₃) δ 9.15 (s, 1H), 8.94 (s, 1H), 8.39 (s, 1H), 8.08-7.99 (m, 2H), 4.83-4.68 (m, 1H), 3.55-3.25 (m, 6H), 2.77-2.64 (m, 1H), 2.17-2.06 (m, 2H), 1.20-1.09 (m, 9H). |
| 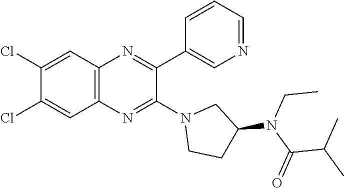 [SMILES: CCN(C(=O)C(C)C)[C@H]1CCN(c2nc3cc(Cl)c(Cl)cc3nc2-c2cccnc2)C1] | 499 | 30.2% yield | LC MS of 499: Ret. Time: 1.424 min, MS (ESI) m/z: 458.1, [M + H]⁺. ¹H NMR of 499: ¹H NMR (400 MHz, CDCl₃) δ 8.96 (s, 1H), 8.71 (s, 1H), 8.25- 8.09 (m, 1H), 8.04 (s, 1H), 7.90 (s, 1H), 7.59- 7.50 (m, 1H), 4.87-4.78 (m, 1H), 3.50- 3.10 (m, 6H), 2.82-2.65 (m, 1H), 2.12- 2.00 (m, 2H), 1.20-1.03 (m, 9H). |
| 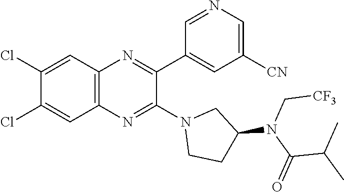 [SMILES: CC(C)C(=O)N(CC(F)(F)F)[C@H]1CCN(c2nc3cc(Cl)c(Cl)cc3nc2-c2cncc(C#N)c2)C1] | 517 | 51.1% yield | LC MS of 517: Ret. Time: 1.502 min, MS (ESI) m/z: 537.1, [M + H]⁺. ¹H NMR of 517: ¹H NMR (400 MHz, DMSO-d₆) δ 9.13 (d, J = 2.1 Hz, 1H), 9.08 (d, J = 2.0 Hz, 1H), 8.52 (t, J = 2.1 Hz, 1H), 8.13 (s, 1H), 7.94 (s, 1H), 4.52 (s, 1H), 4.39- 3.94 (m, 2H), 3.53-3.27 (m, 3H), 3.20 (t, J = 9.8 Hz, 1H), 3.00-2.85 (m, 1H), 2.18- 2.00 (m, 2H), 1.00 (dd, J = 11.0, 6.6 Hz, 6H). |

Compound 200 according to the present disclosure was prepared as shown in Scheme 93 by an analogous procedure to the one described in Examples 17A-17E.

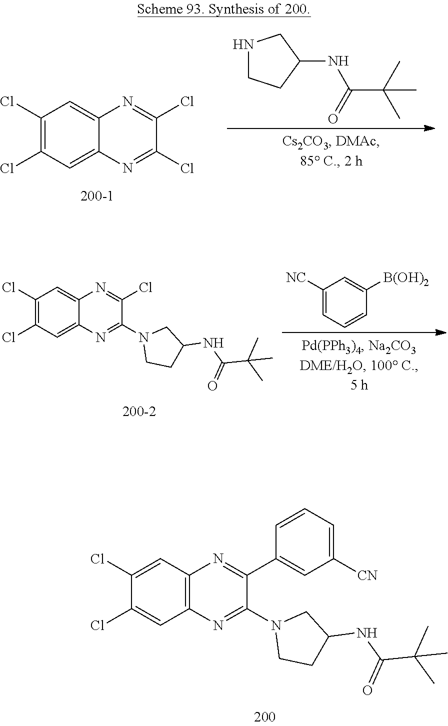 [SMILES: Clc1cc2nc(Cl)c(Cl)nc2cc1Cl ; CC(C)(C)C(=O)NC1CCNC1 ; CC(C)(C)C(=O)NC1CCN(c2nc3cc(Cl)c(Cl)cc3nc2Cl)C1 ; N#Cc1cccc(B(O)O)c1 ; CC(C)(C)C(=O)NC1CCN(c2nc3cc(Cl)c(Cl)cc3nc2-c2cccc(C#N)c2)C1]

Compound 238 according to the present disclosure was prepared as shown in Scheme 94 by an analogous procedure to the one described in Examples 17A-17E.

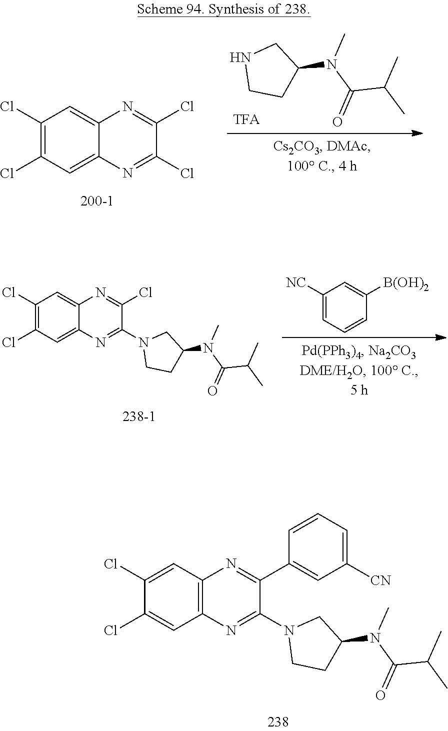 [SMILES: Clc1cc2nc(Cl)c(Cl)nc2cc1Cl ; CC(C)C(=O)N(C)[C@H]1CCNC1 ; CC(C)C(=O)N(C)[C@H]1CCN(c2nc3cc(Cl)c(Cl)cc3nc2Cl)C1 ; N#Cc1cccc(B(O)O)c1 ; CC(C)C(=O)N(C)[C@H]1CCN(c2nc3cc(Cl)c(Cl)cc3nc2-c2cccc(C#N)c2)C1]

Compound 411 according to the present disclosure was prepared as shown in Scheme 95 by an analogous procedure to the one described in Examples 17A-17E.

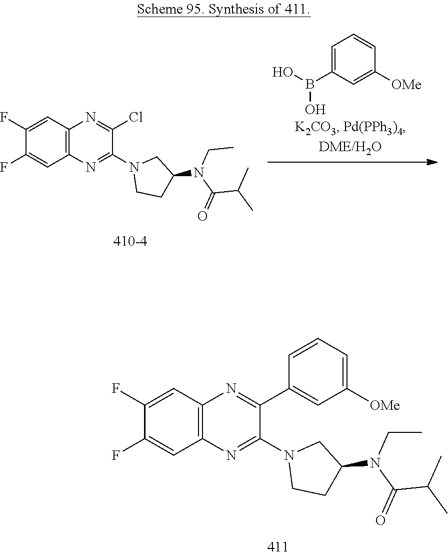 [SMILES: COc1cccc(B(O)O)c1 ; CCN(C(=O)C(C)C)[C@H]1CCN(c2nc3cc(F)c(F)cc3nc2Cl)C1 ; CCN(C(=O)C(C)C)[C@H]1CCN(c2nc3cc(F)c(F)cc3nc2-c2cccc(OC)c2)C1]

Compound 412 according to the present disclosure was prepared as shown in Scheme 96 by an analogous procedure to the one described in Examples 17A-17E.

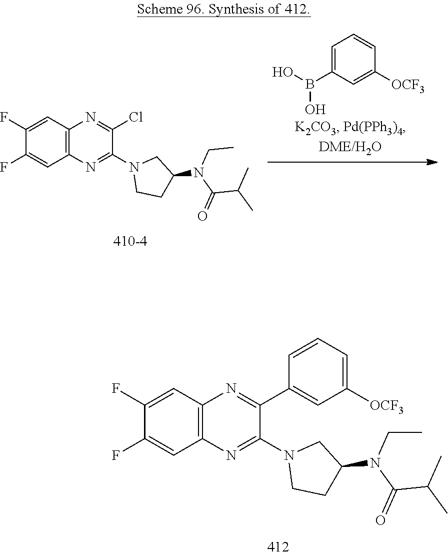 [SMILES: OB(O)c1cccc(OC(F)(F)F)c1 ; CCN(C(=O)C(C)C)[C@H]1CCN(c2nc3cc(F)c(F)cc3nc2Cl)C1 ; CCN(C(=O)C(C)C)[C@H]1CCN(c2nc3cc(F)c(F)cc3nc2-c2cccc(OC(F)(F)F)c2)C1]

Compound 414 according to the present disclosure was prepared as shown in Scheme 97 by an analogous procedure to the one described in Examples 17A-17E.

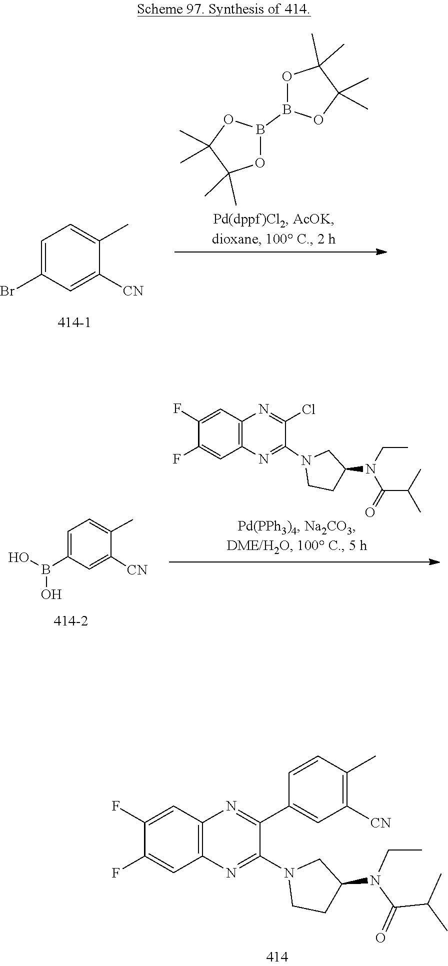

Compound 415 according to the present disclosure was prepared as shown in Scheme 98 by an analogous procedure to the one described in Examples 17A-17E.

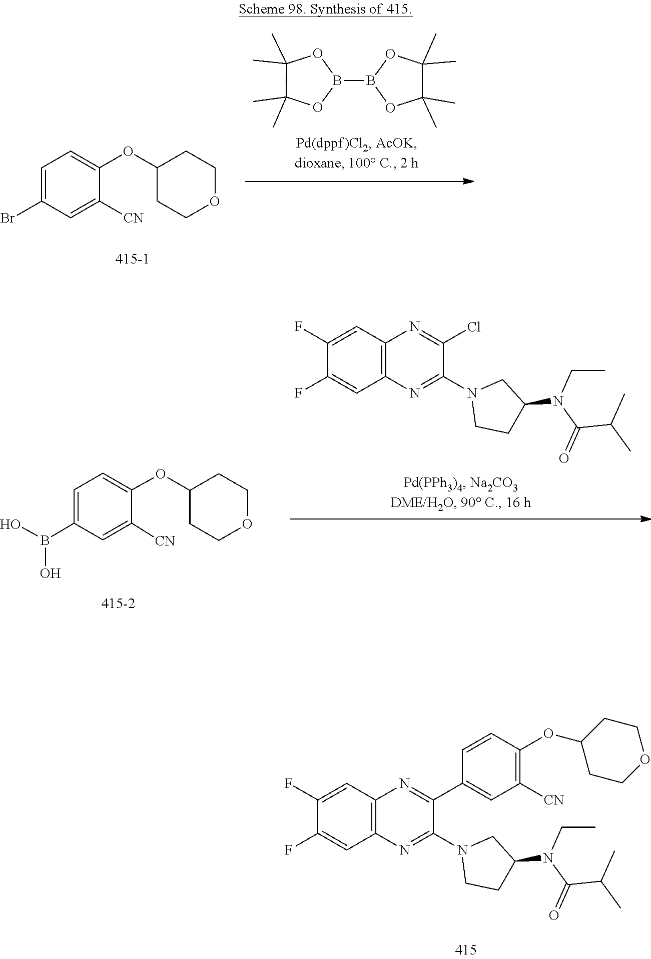

Compound 416 according to the present disclosure was prepared as shown in Scheme 99 by an analogous procedure to the one described in Examples 17A-17E.

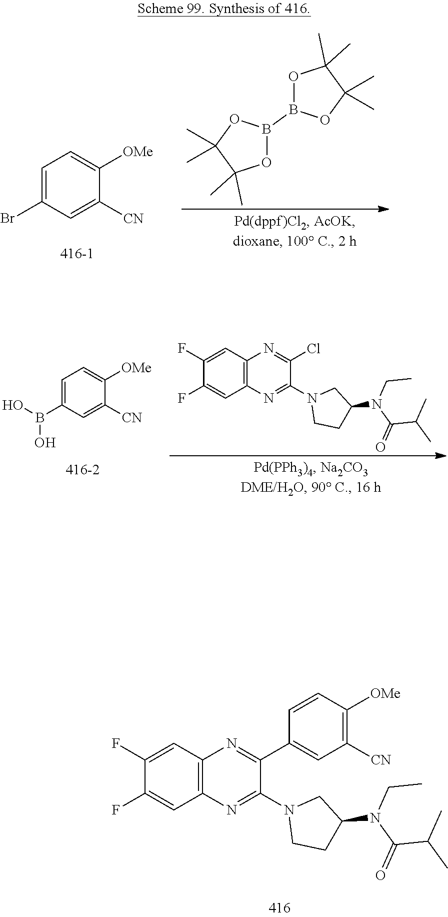

Compound 417 according to the present disclosure was prepared as shown in Scheme 100 by an analogous procedure to the one described in Examples 17A-17E.

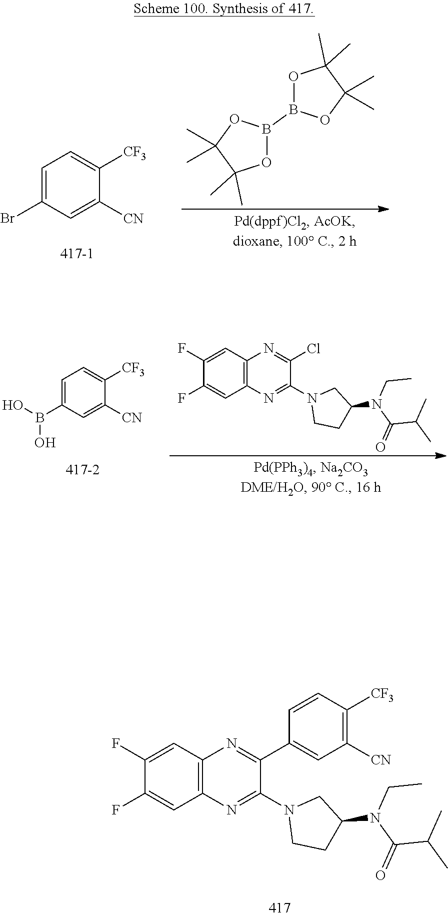

Compound 418 according to the present disclosure was prepared as shown in Scheme 101 by an analogous procedure to the one described in Examples 17A-17E.

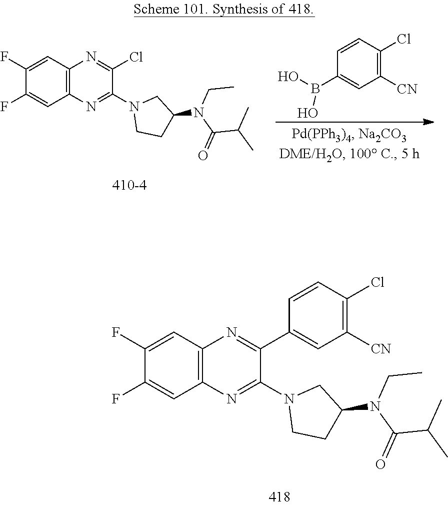 [SMILES: CCN(C(=O)C(C)C)[C@H]1CCN(c2nc3cc(F)c(F)cc3nc2Cl)C1 ; N#Cc1cc(B(O)O)ccc1Cl ; CCN(C(=O)C(C)C)[C@H]1CCN(c2nc3cc(F)c(F)cc3nc2-c2ccc(Cl)c(C#N)c2)C1]

Compound 419 according to the present disclosure was prepared as shown in Scheme 102 by an analogous procedure to the one described in Examples 17A-17E.

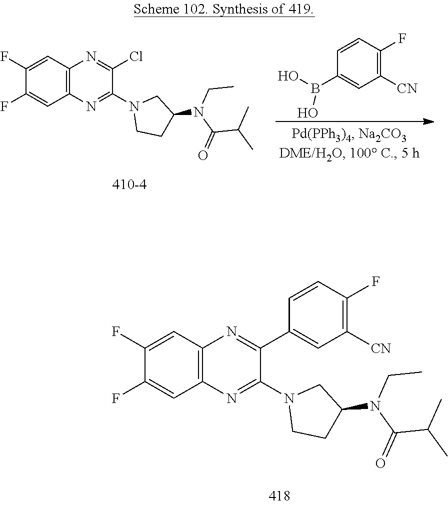 [SMILES: CCN(C(=O)C(C)C)[C@H]1CCN(c2nc3cc(F)c(F)cc3nc2Cl)C1 ; N#Cc1cc(B(O)O)ccc1F ; CCN(C(=O)C(C)C)[C@H]1CCN(c2nc3cc(F)c(F)cc3nc2-c2ccc(F)c(C#N)c2)C1]

Compound 420 according to the present disclosure was prepared as shown in Scheme 103 by an analogous procedure to the one described in Examples 17A-17E.

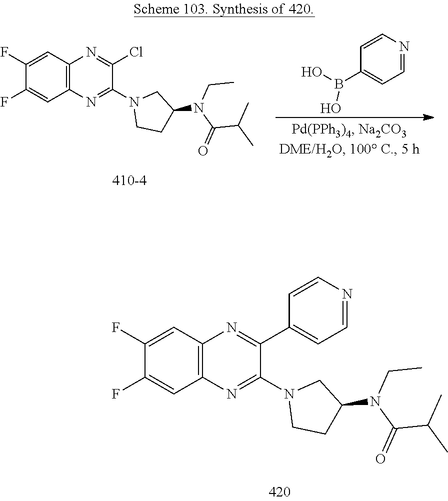 [SMILES: CCN(C(=O)C(C)C)[C@H]1CCN(c2nc3cc(F)c(F)cc3nc2Cl)C1 ; OB(O)c1ccncc1 ; CCN(C(=O)C(C)C)[C@H]1CCN(c2nc3cc(F)c(F)cc3nc2-c2ccncc2)C1]

Compound 421 according to the present disclosure was prepared as shown in Scheme 104 by an analogous procedure to the one described in Examples 17A-17E.

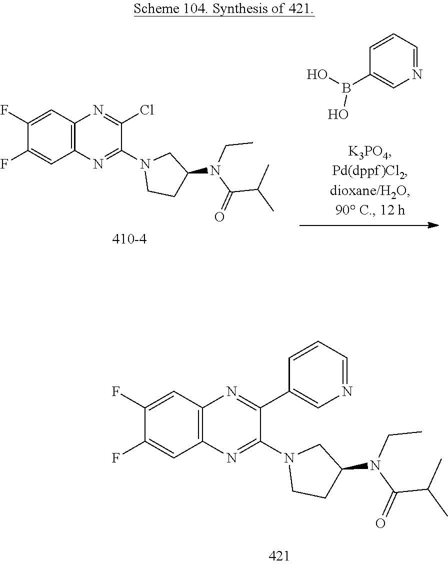 [SMILES: CCN(C(=O)C(C)C)[C@H]1CCN(c2nc3cc(F)c(F)cc3nc2Cl)C1 ; OB(O)c1cccnc1 ; CCN(C(=O)C(C)C)[C@H]1CCN(c2nc3cc(F)c(F)cc3nc2-c2cccnc2)C1]

Compound 422 according to the present disclosure was prepared as shown in Scheme 105 by an analogous procedure to the one described in Examples 17A-17E.

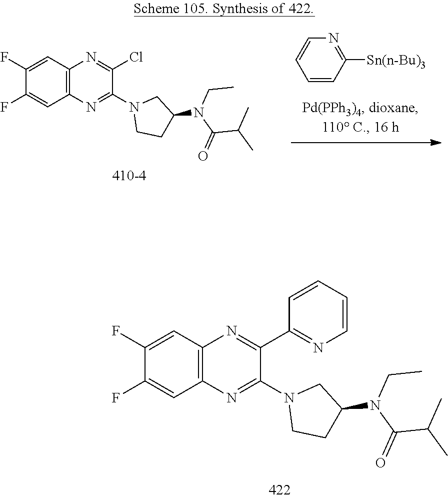

Compound 423 according to the present disclosure was prepared as shown in Scheme 106 by an analogous procedure to the one described in Examples 17A-17E.

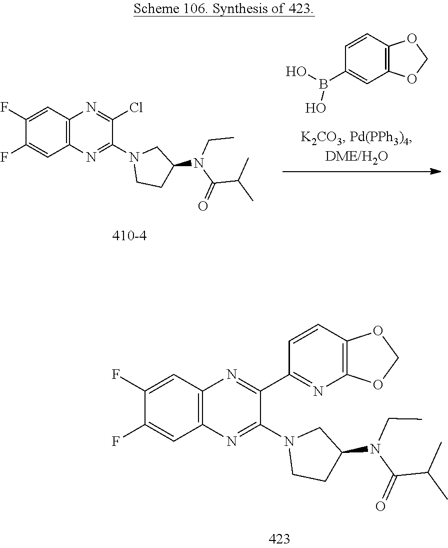 [SMILES: CCN(C(=O)C(C)C)[C@H]1CCN(c2nc3cc(F)c(F)cc3nc2Cl)C1 ; OB(O)c1ccc2c(c1)OCO2 ; CCN(C(=O)C(C)C)[C@H]1CCN(c2nc3cc(F)c(F)cc3nc2-c2ccc3c(n2)OCO3)C1]

Compound 424 according to the present disclosure was prepared as shown in Scheme 107 by an analogous procedure to the one described in Examples 17A-17E.

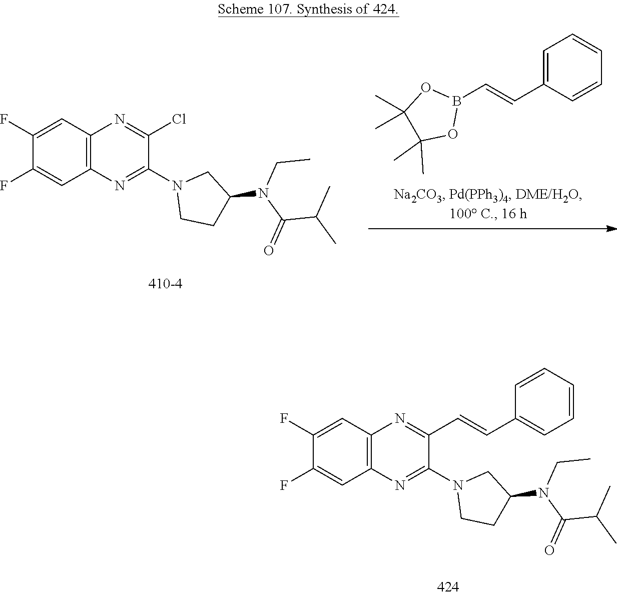 [SMILES: CCN(C(=O)C(C)C)[C@H]1CCN(c2nc3cc(F)c(F)cc3nc2Cl)C1 ; CC1(C)OB(/C=C/c2ccccc2)OC1(C)C ; CCN(C(=O)C(C)C)[C@H]1CCN(c2nc3cc(F)c(F)cc3nc2/C=C/c2ccccc2)C1]

Compound 426 according to the present disclosure was prepared as shown in Scheme 108 by an analogous procedure to the one described in Examples 17A-17E.

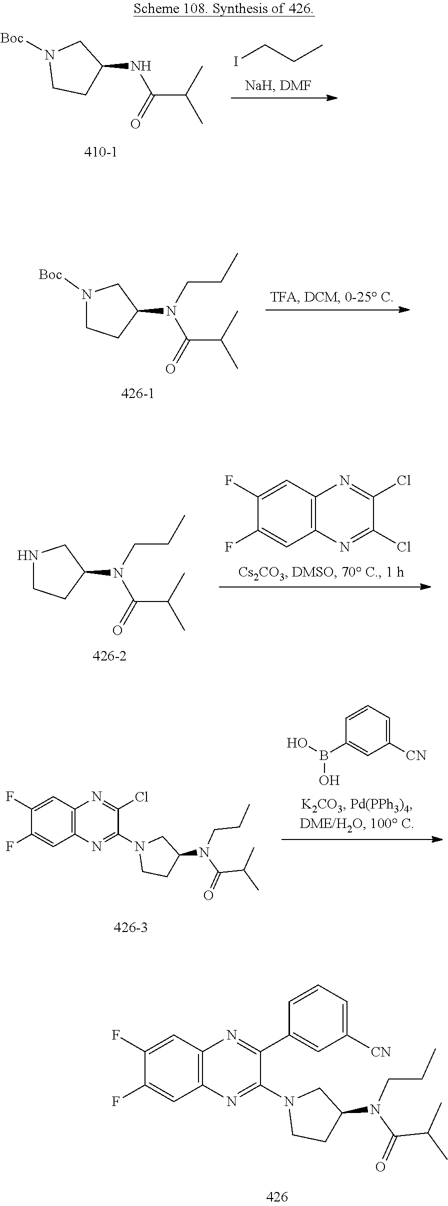

Compound 427 according to the present disclosure was prepared as shown in Scheme 109 by an analogous procedure to the one described in Examples 17A-17E.

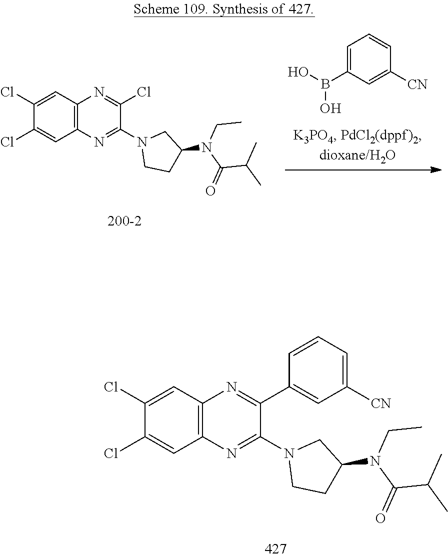 [SMILES: CCN(C(=O)C(C)C)[C@H]1CCN(c2nc3cc(Cl)c(Cl)cc3nc2Cl)C1 ; N#Cc1cccc(B(O)O)c1 ; CCN(C(=O)C(C)C)[C@H]1CCN(c2nc3cc(Cl)c(Cl)cc3nc2-c2cccc(C#N)c2)C1]

Compound 452 according to the present disclosure was prepared as shown in Scheme 110 by an analogous procedure to the one described in Examples 17A-17E.

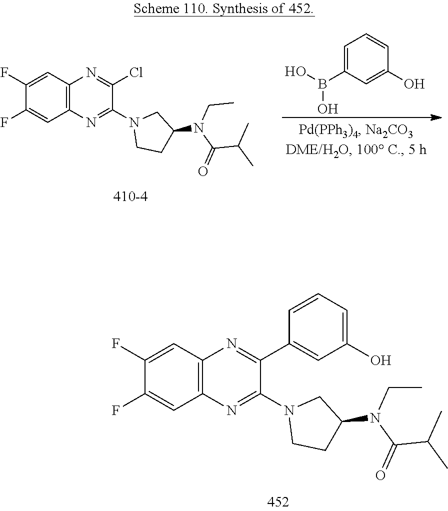 [SMILES: CCN(C(=O)C(C)C)[C@H]1CCN(c2nc3cc(F)c(F)cc3nc2Cl)C1 ; OB(O)c1cccc(O)c1 ; CCN(C(=O)C(C)C)[C@H]1CCN(c2nc3cc(F)c(F)cc3nc2-c2cccc(O)c2)C1]

Compound 466 according to the present disclosure was prepared as shown in Scheme 111 by an analogous procedure to the one described in Examples 17A-17E.

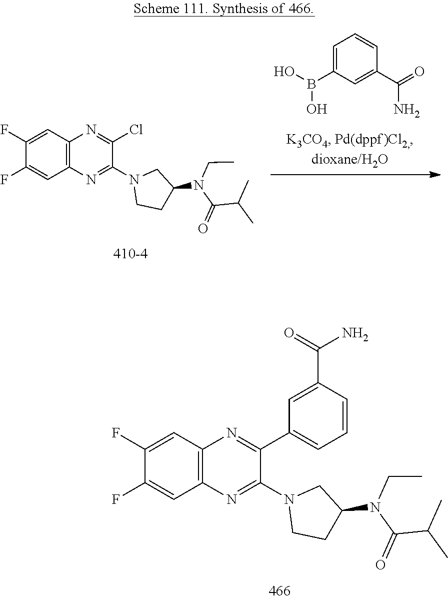 [SMILES: NC(=O)c1cccc(B(O)O)c1 ; CCN(C(=O)C(C)C)[C@H]1CCN(c2nc3cc(F)c(F)cc3nc2Cl)C1 ; CCN(C(=O)C(C)C)[C@H]1CCN(c2nc3cc(F)c(F)cc3nc2-c2cccc(C(N)=O)c2)C1]

Compound 480 according to the present disclosure was prepared as shown in Scheme 112 by an analogous procedure to the one described in Examples 17A-17E.

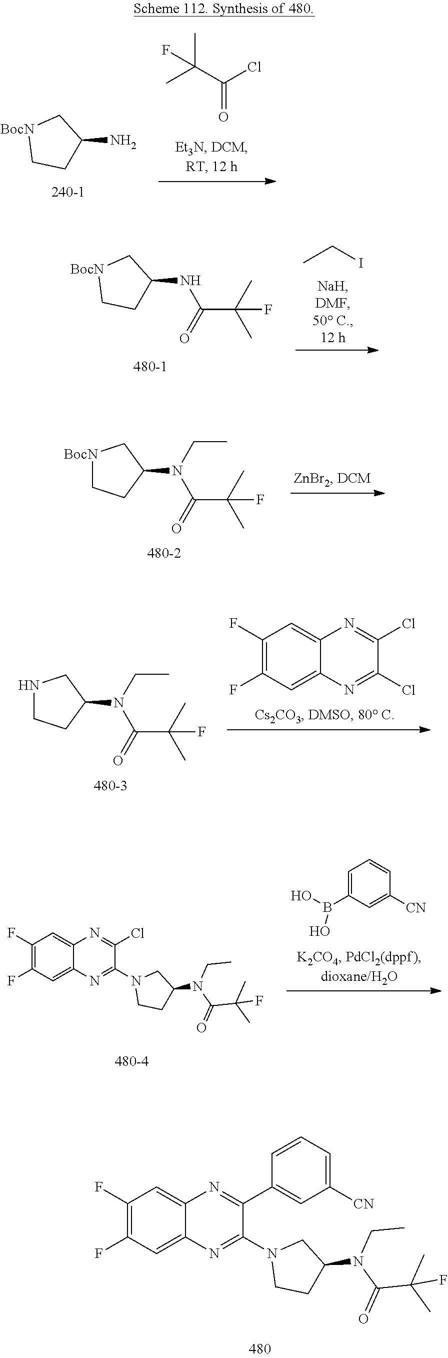

Compound 481 according to the present disclosure was prepared as shown in Scheme 113 by an analogous procedure to the one described in Examples 17A-17E.

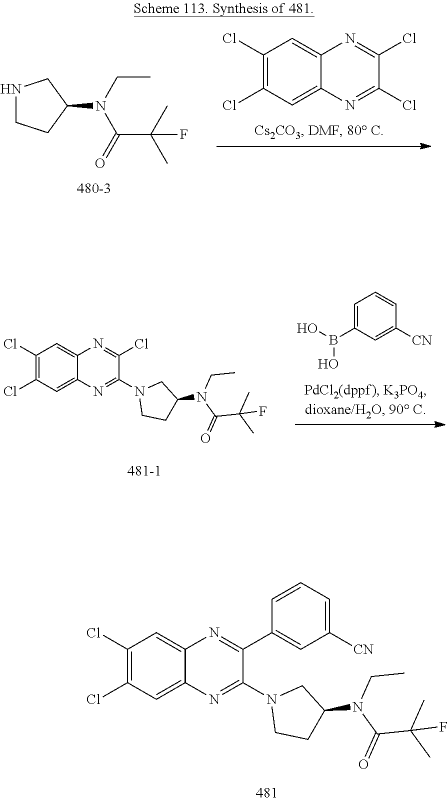 [SMILES: CCN(C(=O)C(C)(C)F)[C@H]1CCNC1 ; Clc1cc2nc(Cl)c(Cl)nc2cc1Cl ; CCN(C(=O)C(C)(C)F)[C@H]1CCN(c2nc3cc(Cl)c(Cl)cc3nc2Cl)C1 ; N#Cc1cccc(B(O)O)c1 ; CCN(C(=O)C(C)(C)F)[C@H]1CCN(c2nc3cc(Cl)c(Cl)cc3nc2-c2cccc(C#N)c2)C1]

Compound 483 according to the present disclosure was prepared as shown in Scheme 114 by an analogous procedure to the one described in Examples 17A-17E.

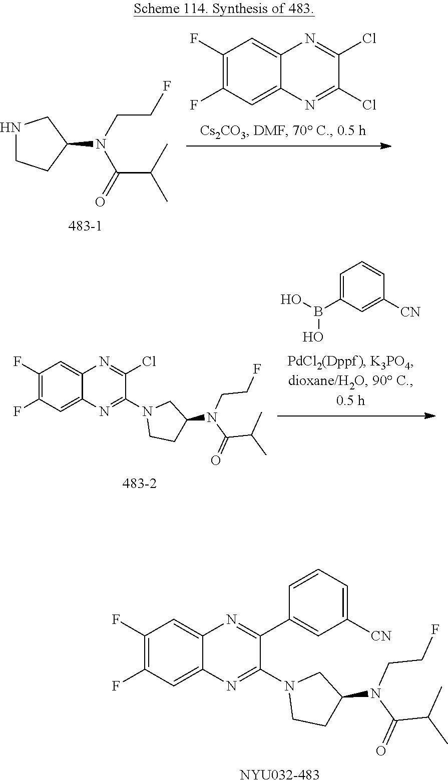

Compound 497 according to the present disclosure was prepared as shown in Scheme 115 by an analogous procedure to the one described in Examples 17A-17E.

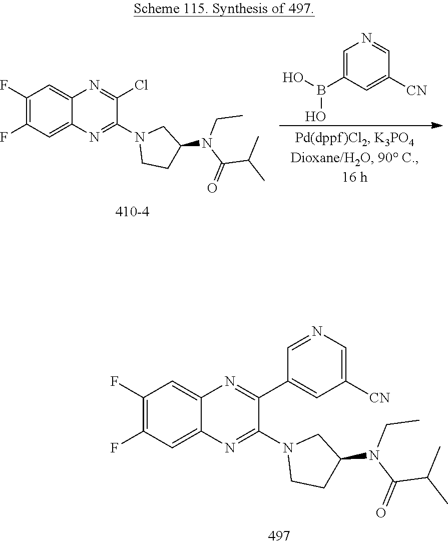 [SMILES: CCN(C(=O)C(C)C)[C@H]1CCN(c2nc3cc(F)c(F)cc3nc2Cl)C1 ; N#Cc1cncc(B(O)O)c1 ; CCN(C(=O)C(C)C)[C@H]1CCN(c2nc3cc(F)c(F)cc3nc2-c2cncc(C#N)c2)C1]

Compound 498 according to the present disclosure was prepared as shown in Scheme 116 by an analogous procedure to the one described in Examples 17A-17E.

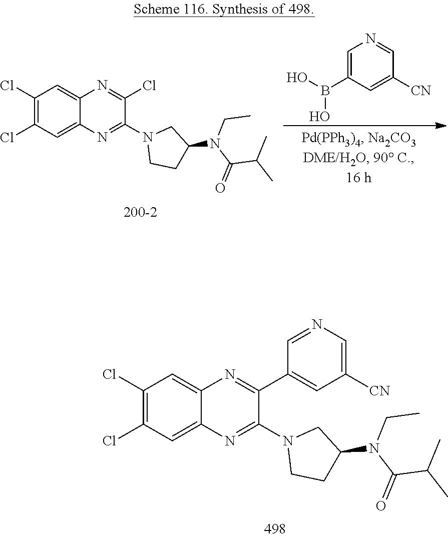 [SMILES: CCN(C(=O)C(C)C)[C@H]1CCN(c2nc3cc(Cl)c(Cl)cc3nc2Cl)C1 ; N#Cc1cncc(B(O)O)c1 ; CCN(C(=O)C(C)C)[C@H]1CCN(c2nc3cc(Cl)c(Cl)cc3nc2-c2cncc(C#N)c2)C1]

Compound 499 according to the present disclosure was prepared as shown in Scheme 117 by an analogous procedure to the one described in Examples 17A-17E.

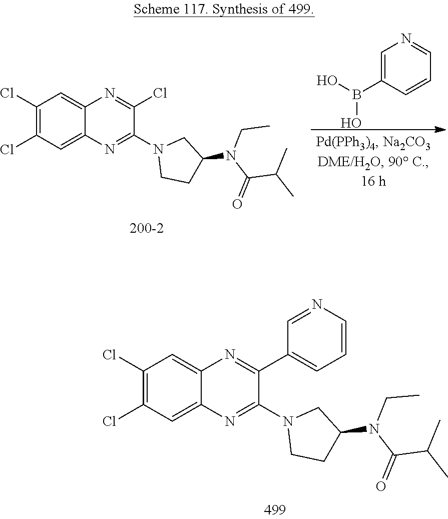 [SMILES: CCN(C(=O)C(C)C)[C@H]1CCN(c2nc3cc(Cl)c(Cl)cc3nc2Cl)C1 ; OB(O)c1cccnc1 ; CCN(C(=O)C(C)C)[C@H]1CCN(c2nc3cc(Cl)c(Cl)cc3nc2-c2cccnc2)C1]

Compound 517 according to the present disclosure was prepared as shown in Scheme 118 by an analogous procedure to the one described in Examples 17A-17E.

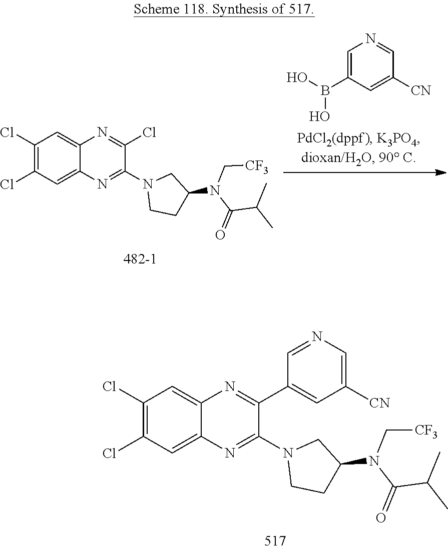 [SMILES: N#Cc1cncc(B(O)O)c1 ; CC(C)C(=O)N(CC(F)(F)F)[C@H]1CCN(c2nc3cc(Cl)c(Cl)cc3nc2Cl)C1 ; CC(C)C(=O)N(CC(F)(F)F)[C@H]1CCN(c2nc3cc(Cl)c(Cl)cc3nc2-c2cncc(C#N)c2)C1]

### Example 18: Synthesis of Analog 413

Compound 413 according to the present disclosure was prepared as shown in Scheme 119 and described below.

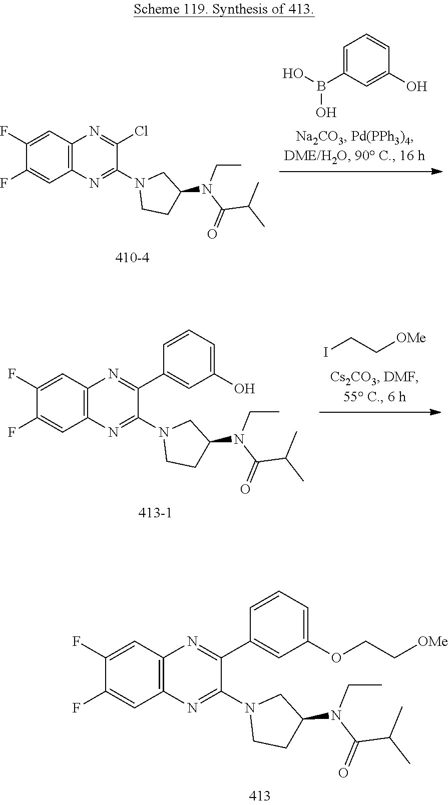 [SMILES: OB(O)c1cccc(O)c1 ; CCN(C(=O)C(C)C)[C@H]1CCN(c2nc3cc(F)c(F)cc3nc2Cl)C1 ; CCN(C(=O)C(C)C)[C@H]1CCN(c2nc3cc(F)c(F)cc3nc2-c2cccc(O)c2)C1 ; COCCI ; CCN(C(=O)C(C)C)[C@H]1CCN(c2nc3cc(F)c(F)cc3nc2-c2cccc(OCCOC)c2)C1]

### Example 18A: Preparation of Compound 413-1

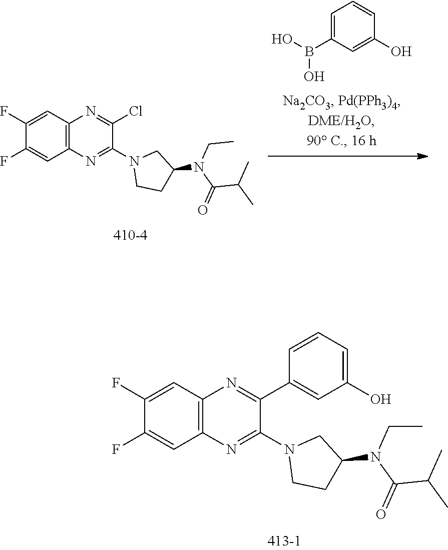 [SMILES: OB(O)c1cccc(O)c1 ; CCN(C(=O)C(C)C)[C@H]1CCN(c2nc3cc(F)c(F)cc3nc2Cl)C1 ; CCN(C(=O)C(C)C)[C@H]1CCN(c2nc3cc(F)c(F)cc3nc2-c2cccc(O)c2)C1]

To a solution of N-[(3S)-1-(3-chloro-6,7-difluoroquinoxalin-2-yl)pyrrolidin-3-yl]-N-ethyl-2-methylpropanamide 410-4 (500 mg, 1.306 mmol), (3-hydroxyphenyl)boranediol (270 mg, 1.959 mmol) and K₂CO₃ (542 mg, 3.918 mmol) in DME (20 mL) and water (4 mL) stirred under nitrogen at 25° C., Pd(PPh₃)₄ (151 mg, 0.1306 mmol) was added. The reaction mixture was stirred at 90° C. for 16 h. The reaction mixture was evaporated under reduced pressure to give a black oil. The residue was purified by chromatography on silica gel eluting with 50% EtOAc in petroleum ether to afford (S)—N-(1-(6,7-difluoro-3-(3-hydroxyphenyl)quinoxalin-2-yl)pyrrolidin-3-yl)-N-ethylisobutyramide 413-1 (550 mg, 94.65% yield) as a yellow oil. LC MS of 413-1: Ret. Time: 1.357 min, MS (ESI) m/z: 441.2, M+H⁺.

### Example 18B: Preparation of Compound 413

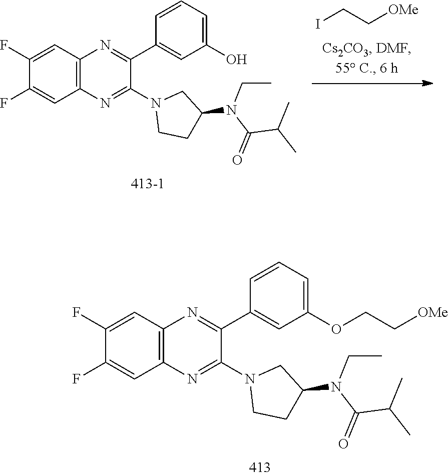 [SMILES: CCN(C(=O)C(C)C)[C@H]1CCN(c2nc3cc(F)c(F)cc3nc2-c2cccc(O)c2)C1 ; COCCI ; CCN(C(=O)C(C)C)[C@H]1CCN(c2nc3cc(F)c(F)cc3nc2-c2cccc(OCCOC)c2)C1]

A solution of (S)—N-(1-(6,7-difluoro-3-(3-hydroxyphenyl)quinoxalin-2-yl)pyrrolidin-3-yl)-N-ethylisobutyramide 413-1 (150 mg, 0.34 mmol) and Cs₂CO₃ (333 mg, 1.02 mmol) in DMF (8 mL) was stirred under a nitrogen atmosphere at 25° C. 1-Iodo-2-methoxyethane (190 mg, 1.02 mmol) was added. The reaction mixture was stirred at 55° C. for 6 h. The reaction was quenched with ice-water (10 mL) and extracted with EtOAc (50 mL). The organic phase was washed with brine (50 mL), dried over Na₂SO₄, concentrated under reduced pressure and purified by chromatography on silica gel eluting with 25% EtOAc in petroleum ether to afford (S)—N-(1-(6,7-difluoro-3-(3-(2-methoxyethoxy)phenyl)quinoxalin-2-yl)pyrrolidin-3-yl)-N-ethylisobutyramide 413 (74.7 mg, 43.9% yield) as a yellow solid. LC MS of 413: Ret. Time: 1.460 min, MS (ESI) m/z: 499.2, [M+H]⁺. ¹H NMR of 413: ¹H NMR (400 MHz, CDCl₃) δ 7.78-7.49 (m, 2H), 7.38 (t, J=7.8 Hz, 1H), 7.26-7.14 (m, 2H), 7.02 (dt, J=8.4, 4.2 Hz, 1H), 4.95-4.80 (m, 1H), 4.27-4.10 (m, 2H), 3.83-3.70 (m, 2H), 3.59-3.09 (m, 9H), 2.84-2.66 (m, 1H), 2.13-1.89 (m, 2H), 1.24-0.90 (m, 9H).

### Example 19: Synthesis of Analog 425

Compound 425 according to the present disclosure was prepared as shown in Scheme 120 and described below.

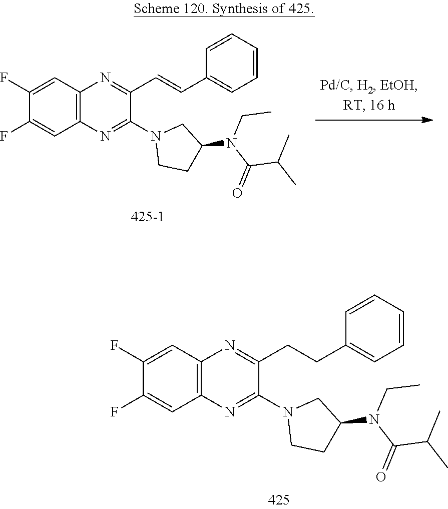 [SMILES: CCN(C(=O)C(C)C)[C@H]1CCN(c2nc3cc(F)c(F)cc3nc2/C=C/c2ccccc2)C1 ; CCN(C(=O)C(C)C)[C@H]1CCN(c2nc3cc(F)c(F)cc3nc2CCc2ccccc2)C1]

### Example 19A: Preparation of Compound 425

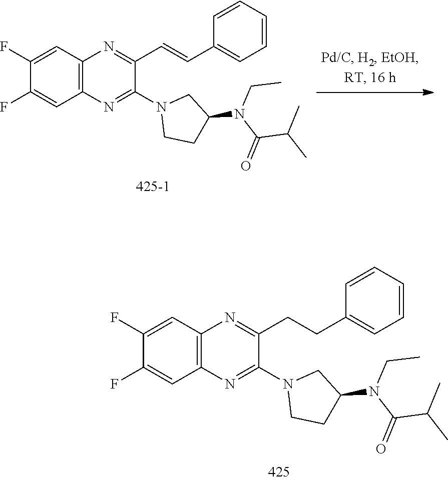 [SMILES: CCN(C(=O)C(C)C)[C@H]1CCN(c2nc3cc(F)c(F)cc3nc2/C=C/c2ccccc2)C1 ; CCN(C(=O)C(C)C)[C@H]1CCN(c2nc3cc(F)c(F)cc3nc2CCc2ccccc2)C1]

To a solution of (S,E)-N-(1-(6,7-difluoro-3-styrylquinoxalin-2-yl)pyrrolidin-3-yl)-N-ethylisobutyramide 425-1 (200 mg, 0.44 mmol) in MeOH (20 mL) at 25° C., palladium on carbon (105 mg, 0.44 mmol) was added. This reaction mixture was stirred at 25° C. The flask is equipped with a magnetic stirring bar and a three-way stopcock attached to a balloon filled with H₂. The mixture is stirred for 12 h at 25° C. The solution was filtered and the filtrate was concentrated under reduced pressure at 40° C. The residue was purified via preparative HPLC to give the product (S)—N-(1-(6,7-difluoro-3-phenethylquinoxalin-2-yl)pyrrolidin-3-yl)-N-ethylisobutyramide 425 (85 mg, yield: 42%) as a yellow gum. LC MS of 425: Ret. Time: 1.457 min, MS (ESI) m/z: 453.2, [M+H]⁺. ¹H NMR of 425: ¹H NMR (400 MHz, DMSO-d₆) δ 7.99-7.71 (m, 1H), 7.72-7.49 (m, 1H), 7.42-7.21 (m, 4H), 7.21-7.12 (m, 1H), 4.82-4.58 (m, 1H), 3.88-3.68 (m, 3H), 3.48-3.38 (m, 1H), 3.35-3.00 (m, 4H), 3.00-2.75 (m, 1H), 2.21-1.99 (m, 2H), 1.25-0.90 (m, 9H).

### Example 20: Synthesis of Analogs 429 and 428, 430, 431, 432, 434, 435, 436, 437, 453, 455, 456, 457, 459, 461, 462, 463, 484, 485, 486, 487, 500, 501, 502, 504, 505

Compound 429 according to the present disclosure was prepared as shown in Scheme 121 and described below.

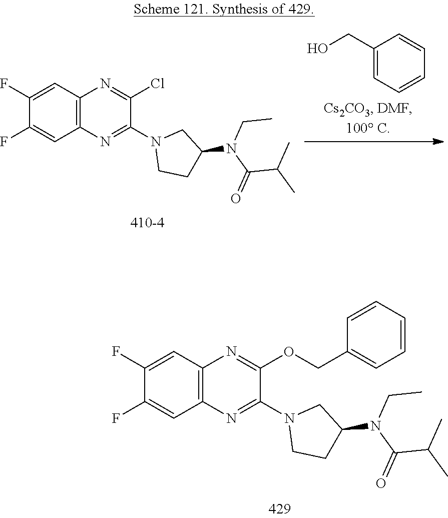 [SMILES: CCN(C(=O)C(C)C)[C@H]1CCN(c2nc3cc(F)c(F)cc3nc2Cl)C1 ; OCc1ccccc1 ; CCN(C(=O)C(C)C)[C@H]1CCN(c2nc3cc(F)c(F)cc3nc2OCc2ccccc2)C1]

### Example 20A: Preparation of Compound 429

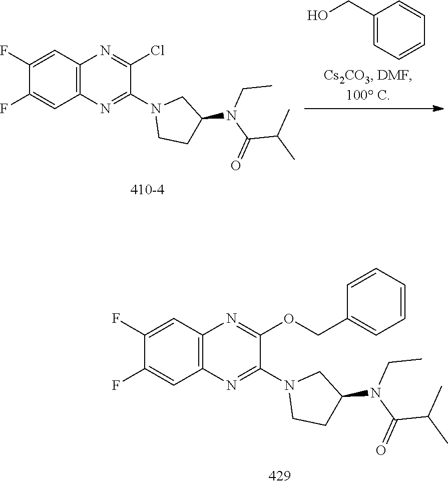 [SMILES: CCN(C(=O)C(C)C)[C@H]1CCN(c2nc3cc(F)c(F)cc3nc2Cl)C1 ; OCc1ccccc1 ; CCN(C(=O)C(C)C)[C@H]1CCN(c2nc3cc(F)c(F)cc3nc2OCc2ccccc2)C1]

To a solution of (S)—N-(1-(3-chloro-6,7-difluoroquinoxalin-2-yl)pyrrolidin-3-yl)-N-ethylisobutyramide 410-4 (150 mg, 0.39 mmol, 1.0 eq) and Cs₂CO₃ (511 mg, 1.57 mmol, 4.0 eq) in DMF (20 mL) stirred under nitrogen at 25° C. was added phenylmethanol (127 mg, 1.1754 mmol, 3 eq). The reaction mixture was stirred at 100° C. for 16 h. After completion, the mixture was quenched with H₂O (100 mL) and extracted with EtOAc (3×50 mL). The combined organic layers were washed with brine for three times and dried with sodium sulphate, filtered and concentrated. The residue was purified by a preparative HPLC to afford (S)—N-(1-(3-(benzyloxy)-6,7-difluoroquinoxalin-2-yl)pyrrolidin-3-yl)-N-ethylisobutyramide 429 (50.1 mg, 28.2% yield) as a white solid. LC MS of 429: Ret. Time: 1.56 min, MS (ESI) m/z: 455.2, [M+H]⁺. ¹H NMR of 429: ¹H NMR (400 MHz, CDCl₃) δ 7.45 (d, J=6.8 Hz, 2H), 7.43-7.30 (m, 5H), 5.46 (s, 2H), 5.10-4.45 (m, 1H), 4.13-3.93 (m, 2H), 3.85-3.55 (m, 2H), 3.43-3.20 (m, 2H), 2.83-2.67 (m, 1H), 2.21-1.93 (m, 2H), 1.22-1.03 (m, 9H).

### Example 20B: Preparation of Analogous Compounds 428, 430, 431, 432, 434, 435, 436, 437, 453, 455, 456, 457, 459, 461, 462, 463, 484, 485, 486, 487, 500, 501, 502, 504, and 505

Table 15, below, depicts exemplary compounds synthesized according to an analogous procedure to the one described for 429.

TABLE 15
Compounds synthesized analogously to 429.

| | Compound | | |
|---|---|---|---|
| Compound Structure | No. | Yield | Characterization |
| 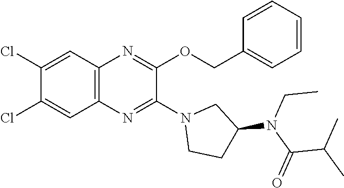 [SMILES: CCN(C(=O)C(C)C)[C@H]1CCN(c2nc3cc(Cl)c(Cl)cc3nc2OCc2ccccc2)C1] | 428 | 44.9% yield | LC MS of 428: Ret. Time: 1.747 min, MS (ESI) m/z: 487.2, [M + H]⁺. ¹H NMR of 428: ¹H NMR (400 MHz, CDCl₃) δ 7.95 (s, 1H), 7.81-7.68 (m, 1H), 7.46 (d, J = 6.6 Hz, 2H), 7.42-7.31 (m, 3H), 5.49 (s, 2H), 5.00-4.45 (m, 1H), 4.30-4.00 (m, 2H), 3.93-3.62 (m, 2H), 3.43-3.17 (m, 3H), 2.89-2.68 (m, 1H), 2.25-2.00 (m, 2H), 1.35-0.91 (m, 9H). |
| 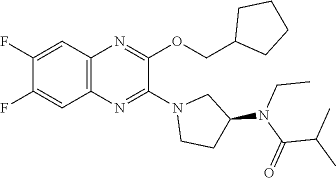 [SMILES: CCN(C(=O)C(C)C)[C@H]1CCN(c2nc3cc(F)c(F)cc3nc2OCC2CCCC2)C1] | 430 | 56.2% yield | LC MS of 430: Ret. Time: 1.698 min, MS (ESI) m/z: 447.3, [M + H]⁺. ¹H NMR of 430: ¹H NMR (400 MHz, CDCl₃) δ 7.56-7.29 (m, 2H), 5.02 (s, 1H), 4.36-4.24 (m, 2H), 4.07 (dd, J = 18.8, 10.2 Hz, 2H), 3.89-3.62 (m, 2H), 3.53-3.17 (m, 2H), 2.94-2.73 (m, 1H), 2.40 (dt, J = 15.0, 7.6 Hz, 1H), 2.23-2.00 (m, 2H), 1.83 (s, 2H), 1.74-1.46 (m, 4H), 1.46-1.30 (m, 2H), 1.30-1.06 (m, 9H). |
| 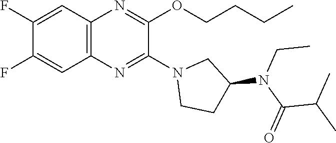 [SMILES: CCCCOc1nc2cc(F)c(F)cc2nc1N1CC[C@H](N(CC)C(=O)C(C)C)C1] | 431 | 38.6% yield | LC MS of 431: Ret. Time: 1.67 min, MS (ESI) m/z: 421.1, [M + H]⁺. ¹H NMR of 431: ¹H NMR (400 MHz, CDCl₃) δ 7.42-7.30 (m, 2H), 5.15-4.45 (m, 1H), 4.48-4.36 (m, 2H), 4.12-3.99 (m, 2H), 3.86-3.59 (m, 2H), 3.50-3.24 (m, 2H), 2.95-2.73 (m, 1H), 2.26-1.98 (m, 2H), 1.87-1.76 (m, 2H), 1.55-1.44 (m, 2H), 1.26 (t, J = 7.0 Hz, 3H), 1.17 (d, J = 6.4 Hz, 6H), 0.99 (t, J = 7.2 Hz, 3H). |
| 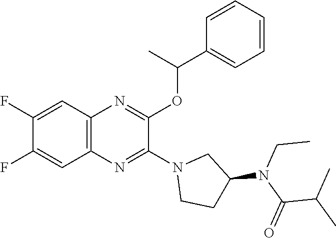 [SMILES: CCN(C(=O)C(C)C)[C@H]1CCN(c2nc3cc(F)c(F)cc3nc2OC(C)c2ccccc2)C1] | 432 | 35.9% yield | LC MS of 432: Ret. Time: 1.59 min, MS (ESI) m/z: 469.1, [M + H]⁺. ¹H NMR of 432: ¹H NMR (400 MHz, CDCl₃) δ 7.44 (t, J = 7.2 Hz, 2H), 7.37-7.26 (m, 5H), 6.51-6.31 (m, 1H), 5.12-4.40 (m, 1H), 4.18-3.96 (m, 2H), 3.92-3.60 (m, 2H), 3.50-3.20 (m, 2H), 2.91-2.68 (m, 1H), 2.23-1.96 (m, 2H), 1.76-1.66 (m, 3H), 1.25-1.14 (m, 9H). |
| 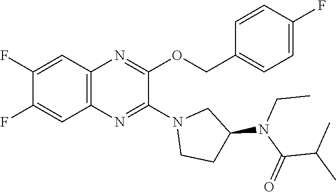 [SMILES: CCN(C(=O)C(C)C)[C@H]1CCN(c2nc3cc(F)c(F)cc3nc2OCc2ccc(F)cc2)C1] | 434 | 15.3% yield | LC MS of 434: Ret. Time: 1.543 min, MS (ESI) m/z: 473.2, [M + H]⁺. ¹H NMR of 434: ¹H NMR (400 MHz, DMSO) δ 7.68-7.56 (m, 3H), 7.56-7.45 (m, 1H), 7.22 (t, J = 8.8 Hz, 2H), 5.45 (d, J = 4.3 Hz, 2H), 4.82-4.47 (m, 1H), 4.14-3.78 (m, 2H), 3.84-3.43 (m, 2H), 3.32-3.12 (m, 2H), 3.06-2.67 (m, 1H), 2.27-1.78 (m, 2H), 1.15-0.63 (m, 9H). |
| 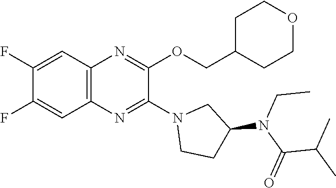 [SMILES: CCN(C(=O)C(C)C)[C@H]1CCN(c2nc3cc(F)c(F)cc3nc2OCC2CCOCC2)C1] | 435 | 34.5% yield | LC MS of 435: Ret. Time: 1.475 min, MS (ESI) m/z: 463.2, [M + H]⁺. ¹H NMR of 435: ¹H NMR (400 MHz, DMSO-d₆) δ 7.68-7.30 (m, 2H), 4.80- 4.60 (m, 1H), 4.38-4.13 (m, 2H), 4.10- 3.83 (m, 3H), 3.80-3.53 (m, 2H), 3.49- 3.34 (m, 2H), 3.33-3.19 (m, 2H), 3.05- 2.75 (m, 1H), 2.23-1.92 (m, 2H), 1.67 (d, J = 11.0 Hz, 2H), 1.39 (td, J = 11.8, 6.5 Hz, 2H), 1.23-0.90 (m, 9H). |
| 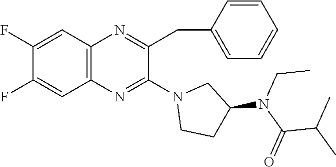 [SMILES: CCN(C(=O)C(C)C)[C@H]1CCN(c2nc3cc(F)c(F)cc3nc2Cc2ccccc2)C1] | 436 | 48.3% yield | LC MS of 436: Ret. Time: 1.502 min, MS (ESI) m/z: 441.2, [M + H]⁺. ¹H NMR of 436: ¹H NMR (400 MHz, CDCl₃) δ 7.63-7.37 (m, 3H), 7.33-7.26 (m, 2H), 7.21 (dd, J = 6.8, 5.8 Hz, 2H), 5.25- 4.91 (m, 1H), 4.28-4.08 (m, 2H), 3.94 (s, 1H), 3.85-3.76 (m, 1H), 3.54-3.19 (m, 2H), 2.96-2.63 (m, 1H), 2.33-2.09 (m, 2H), 1.52-0.67 (m, 9H). |
| 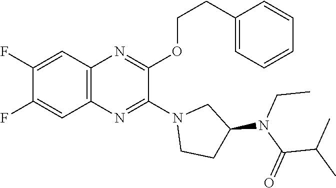 [SMILES: CCN(C(=O)C(C)C)[C@H]1CCN(c2nc3cc(F)c(F)cc3nc2OCCc2ccccc2)C1] | 437 | 34.6% yield | LC MS of 437: Ret. Time: 1.593 min, MS (ESI) m/z: 469.2, [M + H]⁺. ¹H NMR of 437: ¹H NMR (400 MHz, CDCl₃) δ 7.42-7.28 (m, 5H), 7.26-7.19 (m, 2H), 4.92 (s, 1H), 4.80-4.65 (m, 2H), 4.15-3.85 (m, 2H), 3.85-3.41 (m, 3H), 3.32 (td, J = 15.8, 8.0 Hz, 1H), 3.15 (t, J = 6.8 Hz, 2H), 2.85-2.72 (m, 1H), 2.19-1.95 (m, 2H), 1.30-1.08 (m, 9H). |
| 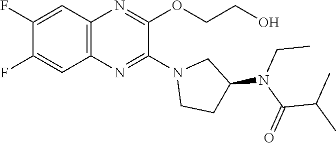 [SMILES: CCN(C(=O)C(C)C)[C@H]1CCN(c2nc3cc(F)c(F)cc3nc2OCCO)C1] | 453 | 89.1% yield | LC MS of 453: Ret. Time: 1.32 min, MS (ESI) m/z: 409.1, [M + H]⁺. ¹H NMR of 453: ¹H NMR (400 MHz, CDCl₃) δ 7.50-7.30 (m, 2H), 5.02-4.80 (m, 1H), 4.65-4.56 (m, 2H), 4.21-3.96 (m, 4H), 3.86-3.61 (m, 2H), 3.52-3.24 (m, 2H), 2.91-2.72 (m, 1H), 2.26-2.03 (m, 2H), 1.33-1.07 (m, 9H). |
| 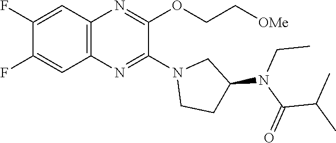 [SMILES: CCN(C(=O)C(C)C)[C@H]1CCN(c2nc3cc(F)c(F)cc3nc2OCCOC)C1] | 455 | 63.6% yield | LC MS of 455: Ret. Time: 1.45 min, MS (ESI) m/z: 423.1, [M + H]⁺. ¹H NMR of 455: ¹H NMR (400 MHz, CDCl₃) δ 7.44-7.30 (m, 2H), 5.14-4.98 (s, 1H), 4.64-4.53 (m, 2H), 4.15-4.02 (m, 2H), 3.90-3.60 (m, 4H), 3.47-3.25 (m, 5H), 2.92-2.72 (m, 1H), 2.25-2.00 (s, 2H), 1.29-1.15 (m, 9H). |
| 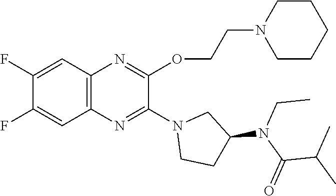 [SMILES: CCN(C(=O)C(C)C)[C@H]1CCN(c2nc3cc(F)c(F)cc3nc2OCCN2CCCCC2)C1] | 456 | 31.3% yield | LC MS of 456: Ret. Time: 1.13 min, MS (ESI) m/z: 476.1, [M + H]⁺. ¹H NMR of 456: ¹H NMR (400 MHz, CDCl₃) δ 7.40-7.28 (m, 2H), 5.05-4.89 (m, 1H), 4.80-4.68 (m, 2H), 4.12-3.98 (m, 2H), 3.86-3.57 (m, 2H), 3.53-3.20 (m, 2H), 3.20-3.02 (m, 2H), 2.94-2.72 (m, 5H), 2.26-2.03 (m, 2H), 1.86-1.68 (m, 4H), 1.55 (s, 2H), 1.29-1.12 (m, 9H). |
| 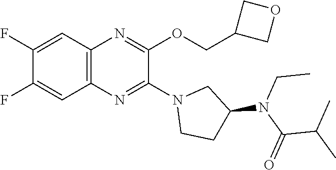 [SMILES: CCN(C(=O)C(C)C)[C@H]1CCN(c2nc3cc(F)c(F)cc3nc2OCC2COC2)C1] | 457 | 17.2% yield | LC MS of 457: Ret. Time: 1.40 min, MS (ESI) m/z: 435.1, [M + H]⁺. ¹H NMR of 457: ¹H NMR (400 MHz, CDCl₃) δ 7.48-7.31 (m, 2H), 4.94-4.85 (m, 3H), 4.75-4.55 (m, 4H), 4.20-3.95 (m, 2H), 3.98-3.60 (m, 2H), 3.54-3.25 (m, 3H), 2.91-2.72 (m, 1H), 2.26-2.04 (m, 2H), 1.29-1.13 (m, 9H). |
| 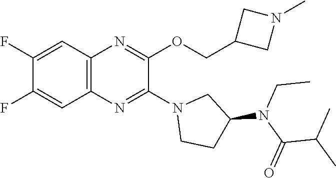 [SMILES: CCN(C(=O)C(C)C)[C@H]1CCN(c2nc3cc(F)c(F)cc3nc2OCC2CN(C)C2)C1] | 459 | 39.4% yield | LC MS of 459: Ret. Time: 1.14 min, MS (ESI) m/z: 448.1, [M + H]⁺. ¹H NMR of 459: ¹H NMR (400 MHz, DMSO-d₆) δ 7.61-7.44 (m, 2H), 4.79-4.59 (m, 1H), 4.57-4.44 (m, 2H), 4.10-3.85 (m, 2H), 3.79-3.52 (m, 3H), 3.40-3.26 (m, 2H), 3.19 (s, 2H), 3.03-2.74 (m, 3H), 2.30 (s, 3H), 2.15-2.01 (m, 2H), 1.21-1.03 (m, 9H). |
| 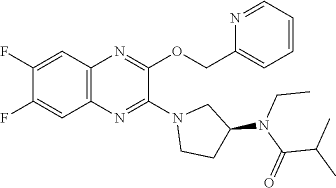 [SMILES: CCN(C(=O)C(C)C)[C@H]1CCN(c2nc3cc(F)c(F)cc3nc2OCc2ccccn2)C1] | 461 | 50.7% yield | LC MS of 461: Ret. Time: 1.41 min, MS (ESI) m/z: 456.1, [M + H]⁺. ¹H NMR of 461: ¹H NMR (400 MHz, CDCl₃) δ 8.66 (d, J = 4.4 Hz, 1H), 7.95-7.80 (m, 1H), 7.63-7.48 (m, 1H), 7.47-7.31 (m, 3H), 5.71 (s, 2H), 5.05-4.50 (m, 1H), 4.20- 4.11 (m, 2H), 3.89-3.77 (s, 1H), 3.73 (t, J = 9.6 Hz, 1H), 3.44-3.22 (m, 2H), 2.89-2.71 (m, 1H), 2.20-2.05 (m, 2H), 1.26-1.11 (m, 9H). |
| 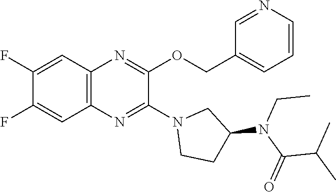 [SMILES: CCN(C(=O)C(C)C)[C@H]1CCN(c2nc3cc(F)c(F)cc3nc2OCc2cccnc2)C1] | 462 | 34.2% yield | LC MS of 462: Ret. Time: 1.34 min, MS (ESI) m/z: 456.1, [M + H]⁺. ¹H NMR of 462: ¹H NMR (400 MHz, DMSO-d₆) δ 8.72 (s, 1H), 8.52 (d, J = 4.0 Hz, 1H), 7.91 (d, J = 7.8 Hz, 1H), 7.52 (dd, J = 11.6, 8.4 Hz, 1H), 7.57-7.34 (m, 2H), 5.51 (s, 2H), 4.61 (dd, J = 16.4, 8.4 Hz, 1H), 3.95 (dt, J = 10.8, 6.8 Hz, 2H), 3.74-3.52 (m, 2H), 3.26 (d, J = 6.6 Hz, 2H), 2.82 (s, 1H), 2.07 (dd, J = 14.2, 8.1 Hz, 2H), 1.08-0.92 (m, 9H). |
| 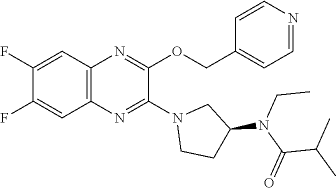 [SMILES: CCN(C(=O)C(C)C)[C@H]1CCN(c2nc3cc(F)c(F)cc3nc2OCc2ccncc2)C1] | 463 | 17.5% yield | LC MS of 463: Ret. Time: 1.28 min, MS (ESI) m/z: 456.0, [M + H]⁺. ¹H NMR of 463: ¹H NMR (400 MHz, DMSO) δ 8.64 (d, J = 4.8, 2H), 7.65 (d, J = 6.0 Hz, 2H), 7.50-7.40 (m, 2H), 5.60 (s, 2H), 4.64 (p, 8.4 Hz, 1H), 4.06-3.95 (m, 2H), 3.78-3.71 (m, 1H), 3.64 (dd, J = 11.2, 8.4 Hz, 1H), 3.36-3.23 (m, 2H), 2.84 (s, 1H), 2.11 (q, J = 8.4 Hz, 2H), 1.11-0.98 (m, 9H). |
| 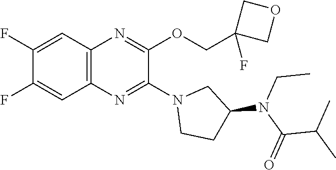 | 484 | 17.5% yield | LC MS of 484: Ret. Time: 1.28 min, MS (ESI) m/z: 456.0, [M + H]⁺. ¹H NMR of 484: ¹H NMR (400 MHz, DMSO) δ 8.64 (d, J = 4.8, 2H), 7.65 (d, J = 6.0 Hz, 2H), 7.50-7.40 (m, 2H), 5.60 (s, 2H), 4.64 (p, 8.4 Hz, 1H), 4.06-3.95 (m, 2H), 3.78-3.71 (m, 1H), 3.64 (dd, J = 11.2, 8.4 Hz, 1H), 3.36-3.23 (m, 2H), 2.84 (s, 1H), 2.11 (q, J = 8.4 Hz, 2H), 1.11-0.98 (m, 9H). |
| 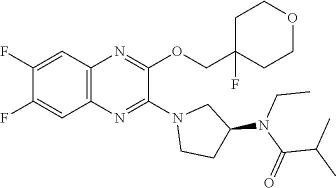 [SMILES: CCN(C(=O)C(C)C)[C@H]1CCN(c2nc3cc(F)c(F)cc3nc2OCC2(F)CCOCC2)C1] | 485 | 8.3% yield | LC MS of 485: Ret. Time: 1.43 min, MS (ESI) m/z: 481.2, [M + H]⁺. ¹H NMR of 485: ¹H NMR (400 MHz, CDCl₃) δ 7.67-7.48 (m, 1H), 7.45-7.32 (m, 1H), 5.03-4.55 (m, 1H), 4.49 (d, J = 20.0 Hz, 2H), 4.25-4.00 (m, 8.6 Hz, 2H), 3.94-3.83 (m, 3H), 3.83-3.68 (m, 3H), 3.50-3.25 (m, 2H), 2.93-2.71 (m, 1H), 2.27-2.07 (m, 2H), 1.97-1.78 (m, 4H), 1.28-1.13 (m, 9H). |
| 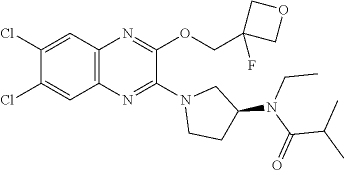 [SMILES: CCN(C(=O)C(C)C)[C@H]1CCN(c2nc3cc(Cl)c(Cl)cc3nc2OCC2(F)COC2)C1] | 486 | 14.4% yield | LC MS of 486: Ret. Time: 1.50 min, MS (ESI) m/z: 485.1, [M + H]⁺. ¹H NMR of 486: ¹H NMR (400 MHz, CDCl₃) δ 8.05 (s, 1H), 7.69 (s, 1H), 4.95- 4.78 (m, 4H), 4.71 (dd, J = 17.6, 8.0 Hz, 2H), 4.32-3.66 (m, 5H), 3.44-3.25 (m, 2H), 2.90-2.70 (m, 1H), 2.29-2.08 (m, 2H), 1.30-1.11 (m, 9H). |
| 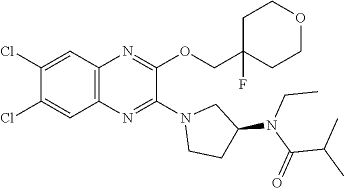 [SMILES: CCN(C(=O)C(C)C)[C@H]1CCN(c2nc3cc(Cl)c(Cl)cc3nc2OCC2(F)CCOCC2)C1] | 487 | 26.5% yield | LC MS of 487: Ret. Time: 1.56 min, MS (ESI) m/z: 513.2, [M + H]⁺. ¹H NMR of 487: ¹H NMR (400 MHz, CDCl₃) δ 7.85-7.64 (m, 2H), 5.03-4.55 (m, 1H), 7.85-7.64 (m, 2H), 5.03-4.55 (m, 1H), 4.50 (d, J = 20.2, 2H), 4.23- 4.03 (m, 2H), 3.93-3.67 (m, 6H), 3.46-3.26 (m, 2H), 2.90-2.71 (m, 1H), 2.25-2.06 (m, 2H), 1.99-1.78 (m, 4H), 1.28-1.11 (m, 9H). |
| 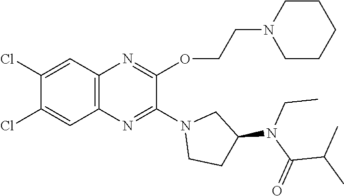 [SMILES: CCN(C(=O)C(C)C)[C@H]1CCN(c2nc3cc(Cl)c(Cl)cc3nc2OCCN2CCCCC2)C1] | 500 | 26.5% yield | LC MS of 500: Ret. Time: 1.17 min, MS (ESI) m/z: 508.2, [M + H]⁺. ¹H NMR of 500: ¹H NMR (400 MHz, CDCl₃) δ 7.71-7.63 (m, 2H), 5.06-4.92 (m, 1H), 4.72-4.50 (m, 2H), 4.17-4.02 (m, 2H), 3.87-3.57 (m, 2H), 3.52-3.20 (m, 2H), 2.96-2.72 (m, 3H), 2.72-2.41 (m, 4H), 2.24- 2.01 (m, 2H), 1.73-1.54 (m, 4H), 1.46 (s, 2H), 1.29-1.18 (t, J = 7.2 Hz, 3H), 1.21- 1.03 (d, J = 6.8 Hz, 6H). |
| 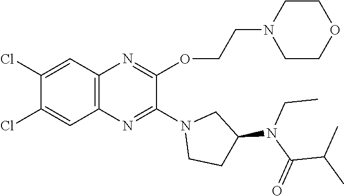 [SMILES: CCN(C(=O)C(C)C)[C@H]1CCN(c2nc3cc(Cl)c(Cl)cc3nc2OCCN2CCOCC2)C1] | 501 | 42.7% yield | LC MS of 501: Ret. Time: 1.22 min, MS (ESI) m/z: 510.0, [M + H]⁺. ¹H NMR of 501: ¹H NMR (400 MHz, CDCl₃) δ 7.71-7.63 (m, 2H), 5.06-4.97 (s, 1H), 4.80-4.45 (m, 2H), 4.15-4.00 (m 2H), 3.90-3.55 (m, 6H), 3.51-3.20 (m, 2H), 3.05-2.83 (m, 2H), 2.83-2.74 (m, 1H), 2.77-2.26 (m, 4H), 2.25-2.03 (m, 2H), 1.29-1.13 (m, 9H). |
| 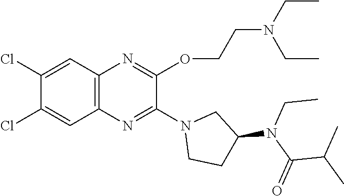 [SMILES: CCN(CC)CCOc1nc2cc(Cl)c(Cl)cc2nc1N1CC[C@H](N(CC)C(=O)C(C)C)C1] | 502 | 15.2% yield | LC MS of 502: Ret. Time: 1.16 min, MS (ESI) m/z: 496.0, [M + H]⁺. ¹H NMR of 502: ¹H NMR (400 MHz, CDCl₃) δ 7.70-7.62 (m, 2H), 5.10-4.93 (m, 1H), 4.70-4.48 (m, 2H), 4.16-4.01 (m, 2H), 3.85-3.58 (m, 2H), 3.53-3.18 (m, 2H), 3.05-2.91 (m, 2H), 2.91-2.76 (m, 1H), 2.76-2.60 (m, 4H), 2.25-2.02 (m, 2H), 1.30-1.01 (m, 15H). |
| 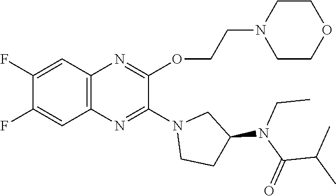 [SMILES: CCN(C(=O)C(C)C)[C@H]1CCN(c2nc3cc(F)c(F)cc3nc2OCCN2CCOCC2)C1] | 504 | 32.4% yield | LC MS of 504: Ret. Time: 1.14 min, MS (ESI) m/z: 478.1, [M + H]⁺. ¹H NMR of 504: ¹H NMR (400 MHz, CDCl₃) δ 7.40-7.29 (m, 2H), 5.05-4.94 (m, 1H), 4.69-4.52 (m, 2H), 4.08 (dd, J = 19.2, 11.2 Hz, 2H), 3.84-3.70 (m, 5H), 3.63 (dd, J = 11.2, 8.8 Hz, 1H), 3.49-3.23 (m, 2H), 2.97-2.72 (m, 3H), 2.73-2.55 (m, 4H), 2.26-2.02 (m, 2H), 1.31-1.10 (m, 9H). |
| 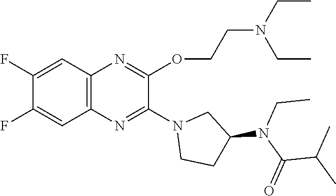 [SMILES: CCN(CC)CCOc1nc2cc(F)c(F)cc2nc1N1CC[C@H](N(CC)C(=O)C(C)C)C1] | 505 | 29.5% yield | LC MS of 505: Ret. Time: 1.13 min, MS (ESI) m/z: 464.1, [M + H]⁺. ¹H NMR of 505: ¹H NMR (400 MHz, CDCl₃) δ 7.39-7.28 (m, 2H), 4.98 (dt, J = 16.4, 8.2 Hz, 1H), 4.72-4.50 (m, 2H), 4.12- 3.98 (m, 2H), 3.84-3.59 (m, 2H), 3.54-3.12 (m, 2H), 3.11 (s, 2H), 2.93-2.72 (m, 5H), 2.24-2.02 (m 2H), 1.37-1.08 (m, 15H). |

Compound 428 according to the present disclosure was prepared as shown in Scheme 122 by an analogous procedure to the one described in Example 20A.

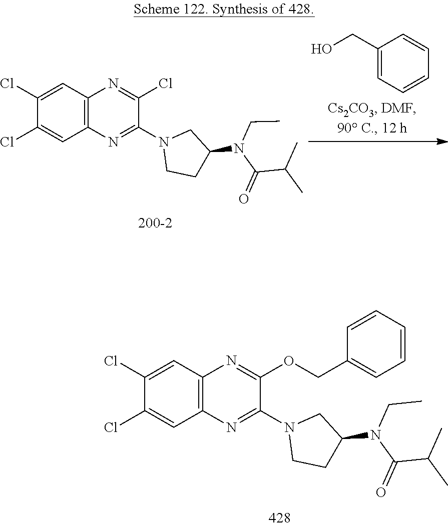 [SMILES: CCN(C(=O)C(C)C)[C@H]1CCN(c2nc3cc(Cl)c(Cl)cc3nc2Cl)C1 ; OCc1ccccc1 ; CCN(C(=O)C(C)C)[C@H]1CCN(c2nc3cc(Cl)c(Cl)cc3nc2OCc2ccccc2)C1]

Compound 430 according to the present disclosure was prepared as shown in Scheme 123 by an analogous procedure to the one described in Example 20A.

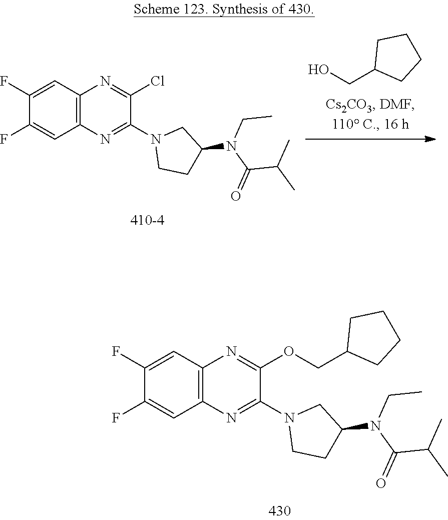 [SMILES: CCN(C(=O)C(C)C)[C@H]1CCN(c2nc3cc(F)c(F)cc3nc2Cl)C1 ; OCC1CCCC1 ; CCN(C(=O)C(C)C)[C@H]1CCN(c2nc3cc(F)c(F)cc3nc2OCC2CCCC2)C1]

Compound 431 according to the present disclosure was prepared as shown in Scheme 124 by an analogous procedure to the one described in Example 20A.

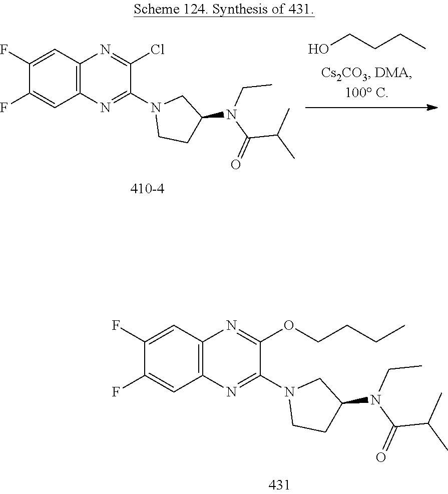

Compound 432 according to the present disclosure was prepared as shown in Scheme 125 by an analogous procedure to the one described in Example 20A.

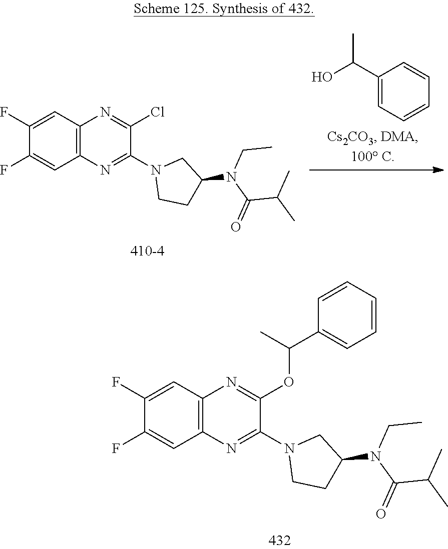

Compound 434 according to the present disclosure was prepared as shown in Scheme 126 by an analogous procedure to the one described in Example 20A.

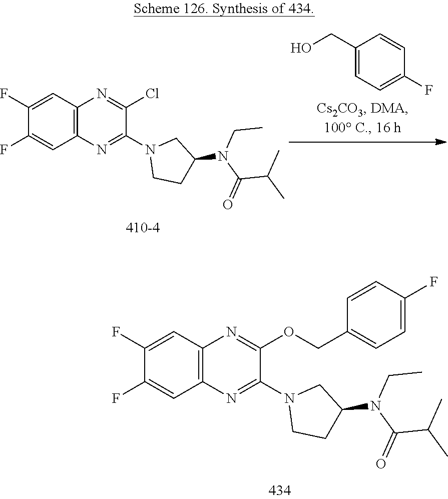 [SMILES: OCc1ccc(F)cc1 ; CCN(C(=O)C(C)C)[C@H]1CCN(c2nc3cc(F)c(F)cc3nc2Cl)C1 ; CCN(C(=O)C(C)C)[C@H]1CCN(c2nc3cc(F)c(F)cc3nc2OCc2ccc(F)cc2)C1]

Compound 435 according to the present disclosure was prepared as shown in Scheme 127 by an analogous procedure to the one described in Example 20A.

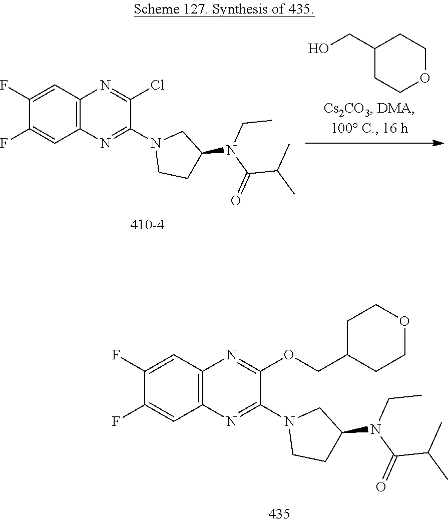 [SMILES: CCN(C(=O)C(C)C)[C@H]1CCN(c2nc3cc(F)c(F)cc3nc2Cl)C1 ; OCC1CCOCC1 ; CCN(C(=O)C(C)C)[C@H]1CCN(c2nc3cc(F)c(F)cc3nc2OCC2CCOCC2)C1]

Compound 436 according to the present disclosure was prepared as shown in Scheme 128 by an analogous procedure to the one described in Example 20A.

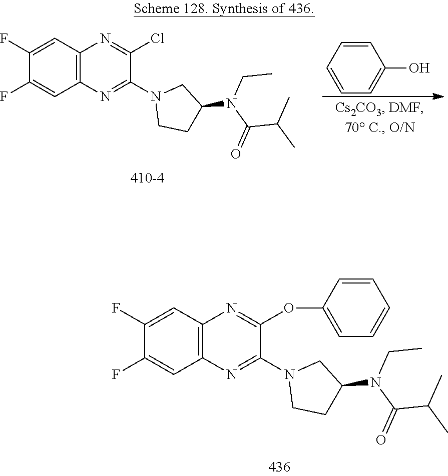 [SMILES: CCN(C(=O)C(C)C)[C@H]1CCN(c2nc3cc(F)c(F)cc3nc2Cl)C1 ; Oc1ccccc1 ; CCN(C(=O)C(C)C)[C@H]1CCN(c2nc3cc(F)c(F)cc3nc2Oc2ccccc2)C1]

Compound 437 according to the present disclosure was prepared as shown in Scheme 129 by an analogous procedure to the one described in Example 20A.

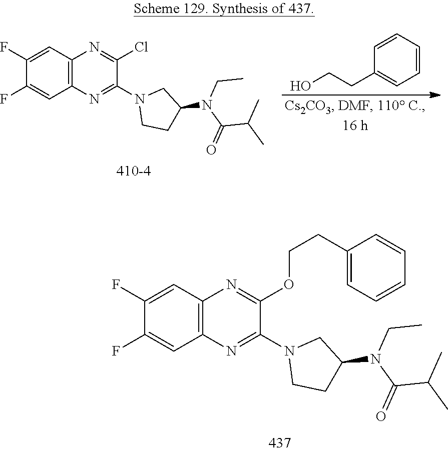 [SMILES: CCN(C(=O)C(C)C)[C@H]1CCN(c2nc3cc(F)c(F)cc3nc2Cl)C1 ; OCCc1ccccc1 ; CCN(C(=O)C(C)C)[C@H]1CCN(c2nc3cc(F)c(F)cc3nc2OCCc2ccccc2)C1]

Compound 453 according to the present disclosure was prepared as shown in Scheme 130 by an analogous procedure to the one described in Example 20A.

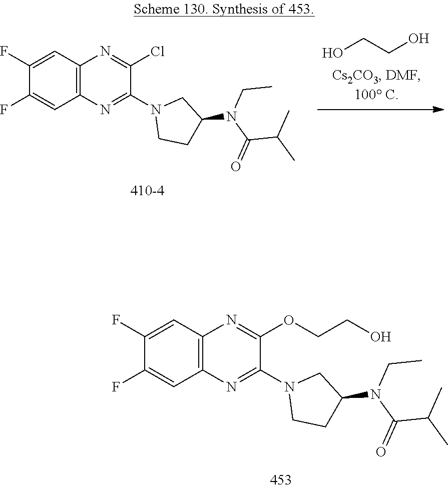

Compound 455 according to the present disclosure was prepared as shown in Scheme 131 by an analogous procedure to the one described in Example 20A.

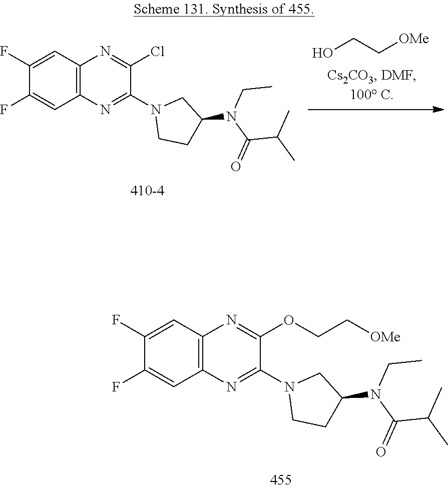 [SMILES: CCN(C(=O)C(C)C)[C@H]1CCN(c2nc3cc(F)c(F)cc3nc2Cl)C1 ; COCCO ; CCN(C(=O)C(C)C)[C@H]1CCN(c2nc3cc(F)c(F)cc3nc2OCCOC)C1]

Compound 456 according to the present disclosure was prepared as shown in Scheme 132 by an analogous procedure to the one described in Example 20A.

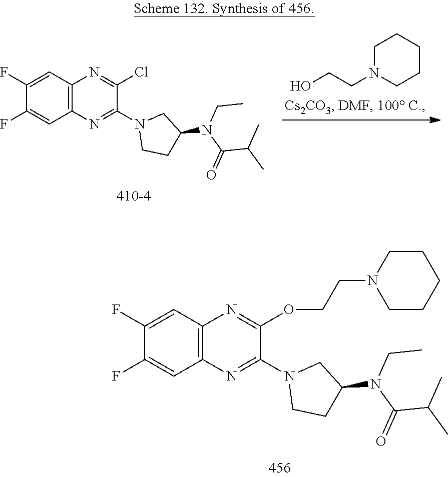 [SMILES: CCN(C(=O)C(C)C)[C@H]1CCN(c2nc3cc(F)c(F)cc3nc2Cl)C1 ; OCCN1CCCCC1 ; CCN(C(=O)C(C)C)[C@H]1CCN(c2nc3cc(F)c(F)cc3nc2OCCN2CCCCC2)C1]

Compound 457 according to the present disclosure was prepared as shown in Scheme 133 by an analogous procedure to the one described in Example 20A.

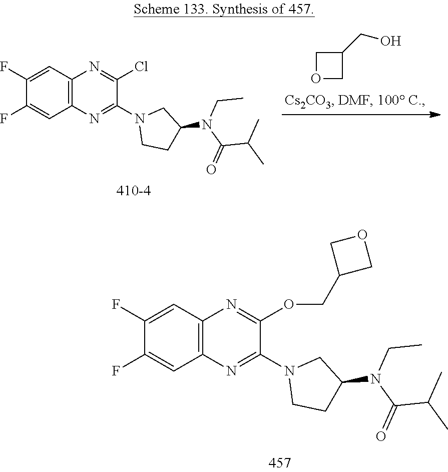 [SMILES: CCN(C(=O)C(C)C)[C@H]1CCN(c2nc3cc(F)c(F)cc3nc2Cl)C1 ; OCC1COC1 ; CCN(C(=O)C(C)C)[C@H]1CCN(c2nc3cc(F)c(F)cc3nc2OCC2COC2)C1]

Compound 459 according to the present disclosure was prepared as shown in Scheme 134 by an analogous procedure to the one described in Example 20A.

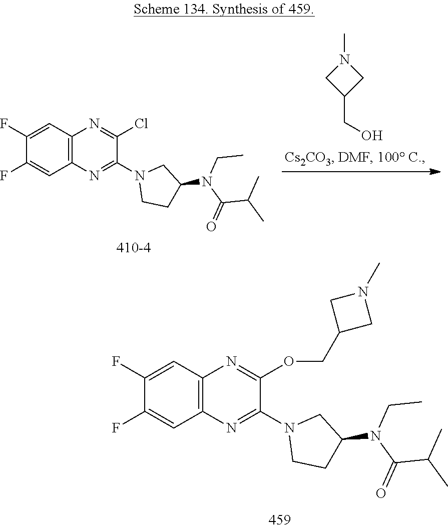

Compound 461 according to the present disclosure was prepared as shown in Scheme 135 by an analogous procedure to the one described in Example 20A.

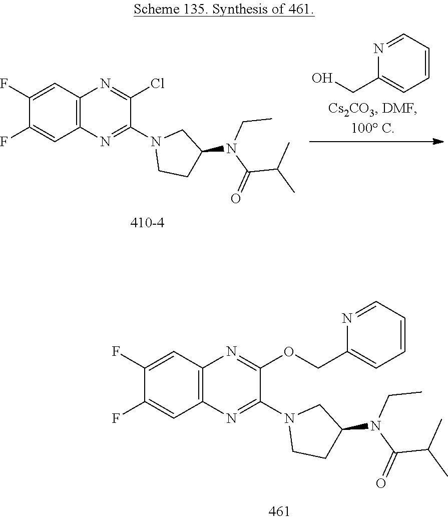 [SMILES: CCN(C(=O)C(C)C)[C@H]1CCN(c2nc3cc(F)c(F)cc3nc2Cl)C1 ; OCc1ccccn1 ; CCN(C(=O)C(C)C)[C@H]1CCN(c2nc3cc(F)c(F)cc3nc2OCc2ccccn2)C1]

Compound 462 according to the present disclosure was prepared as shown in Scheme 136 by an analogous procedure to the one described in Example 20A.

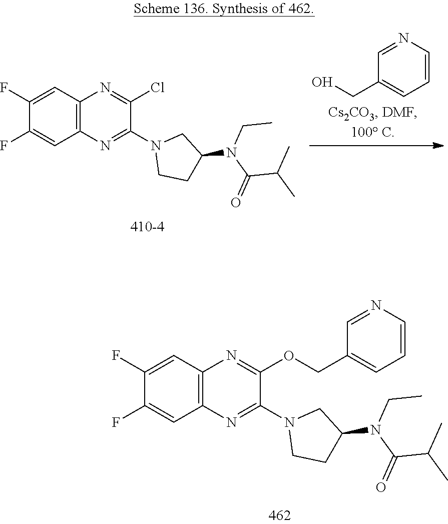 [SMILES: CCN(C(=O)C(C)C)[C@H]1CCN(c2nc3cc(F)c(F)cc3nc2Cl)C1 ; OCc1cccnc1 ; CCN(C(=O)C(C)C)[C@H]1CCN(c2nc3cc(F)c(F)cc3nc2OCc2cccnc2)C1]

Compound 463 according to the present disclosure was prepared as shown in Scheme 137 by an analogous procedure to the one described in Example 20A.

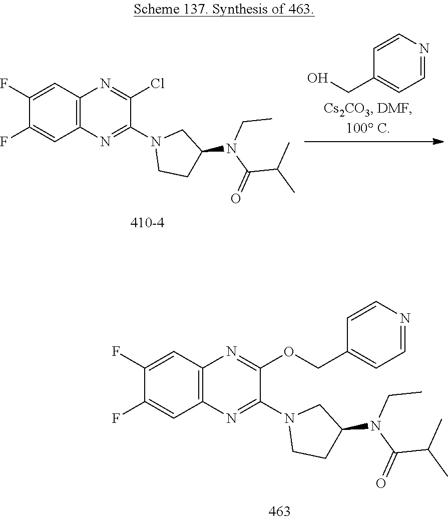 [SMILES: CCN(C(=O)C(C)C)[C@H]1CCN(c2nc3cc(F)c(F)cc3nc2Cl)C1 ; OCc1ccncc1 ; CCN(C(=O)C(C)C)[C@H]1CCN(c2nc3cc(F)c(F)cc3nc2OCc2ccncc2)C1]

Compound 484 according to the present disclosure was prepared as shown in Scheme 138 by an analogous procedure to the one described in Example 20A.

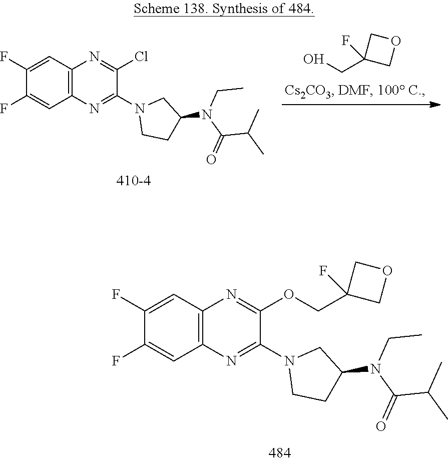 [SMILES: CCN(C(=O)C(C)C)[C@H]1CCN(c2nc3cc(F)c(F)cc3nc2Cl)C1 ; OCC1(F)COC1 ; CCN(C(=O)C(C)C)[C@H]1CCN(c2nc3cc(F)c(F)cc3nc2OCC2(F)COC2)C1]

Compound 485 according to the present disclosure was prepared as shown in Scheme 139 by an analogous procedure to the one described in Example 20A.

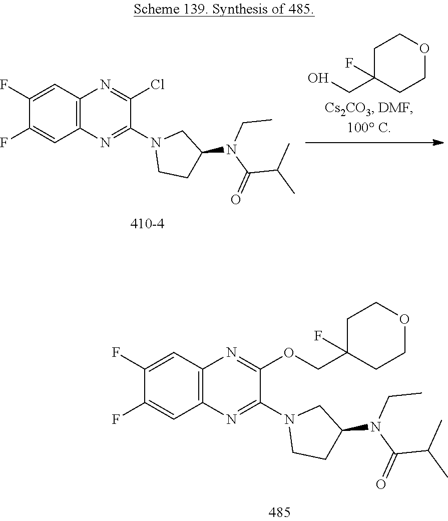 [SMILES: CCN(C(=O)C(C)C)[C@H]1CCN(c2nc3cc(F)c(F)cc3nc2Cl)C1 ; OCC1(F)CCOCC1 ; CCN(C(=O)C(C)C)[C@H]1CCN(c2nc3cc(F)c(F)cc3nc2OCC2(F)CCOCC2)C1]

Compound 486 according to the present disclosure was prepared as shown in Scheme 140 by an analogous procedure to the one described in Example 20A.

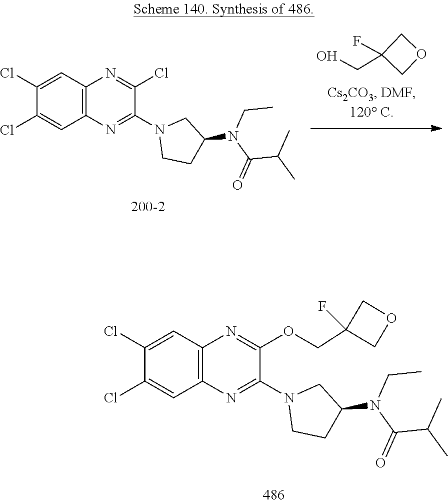 [SMILES: CCN(C(=O)C(C)C)[C@H]1CCN(c2nc3cc(Cl)c(Cl)cc3nc2Cl)C1 ; OCC1(F)COC1 ; CCN(C(=O)C(C)C)[C@H]1CCN(c2nc3cc(Cl)c(Cl)cc3nc2OCC2(F)COC2)C1]

Compound 487 according to the present disclosure was prepared as shown in Scheme 141 by an analogous procedure to the one described in Example 20A.

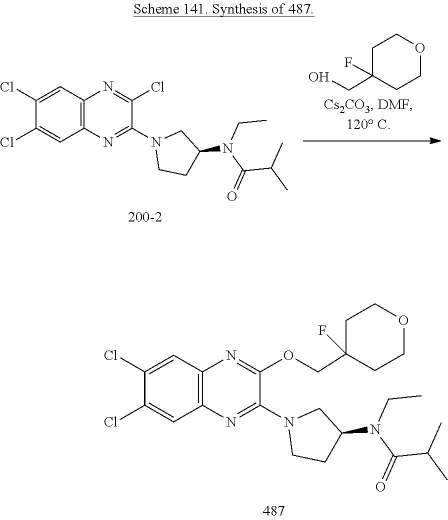 [SMILES: CCN(C(=O)C(C)C)[C@H]1CCN(c2nc3cc(Cl)c(Cl)cc3nc2Cl)C1 ; OCC1(F)CCOCC1 ; CCN(C(=O)C(C)C)[C@H]1CCN(c2nc3cc(Cl)c(Cl)cc3nc2OCC2(F)CCOCC2)C1]

Compound 500 according to the present disclosure was prepared as shown in Scheme 142 by an analogous procedure to the one described in Example 20A.

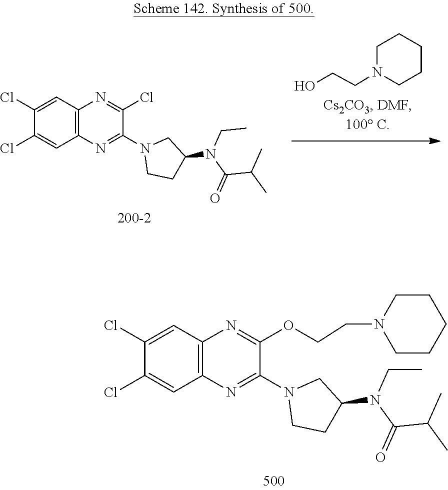 [SMILES: OCCN1CCCCC1 ; CCN(C(=O)C(C)C)[C@H]1CCN(c2nc3cc(Cl)c(Cl)cc3nc2Cl)C1 ; CCN(C(=O)C(C)C)[C@H]1CCN(c2nc3cc(Cl)c(Cl)cc3nc2OCCN2CCCCC2)C1]

Compound 501 according to the present disclosure was prepared as shown in Scheme 143 by an analogous procedure to the one described in Example 20A.

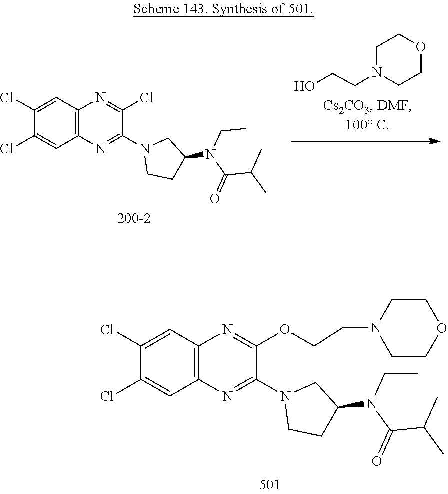 [SMILES: OCCN1CCOCC1 ; CCN(C(=O)C(C)C)[C@H]1CCN(c2nc3cc(Cl)c(Cl)cc3nc2Cl)C1 ; CCN(C(=O)C(C)C)[C@H]1CCN(c2nc3cc(Cl)c(Cl)cc3nc2OCCN2CCOCC2)C1]

Compound 502 according to the present disclosure was prepared as shown in Scheme 144 by an analogous procedure to the one described in Example 20A.

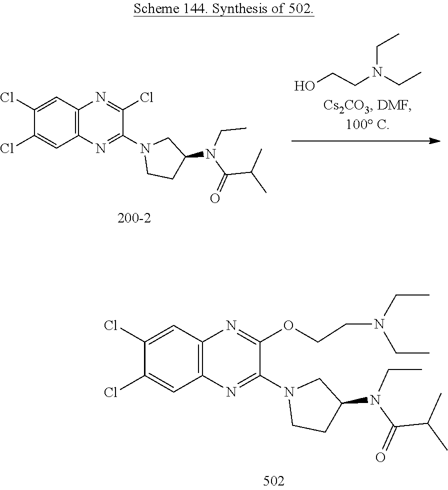

Compound 504 according to the present disclosure was prepared as shown in Scheme 145 by an analogous procedure to the one described in Example 20A.

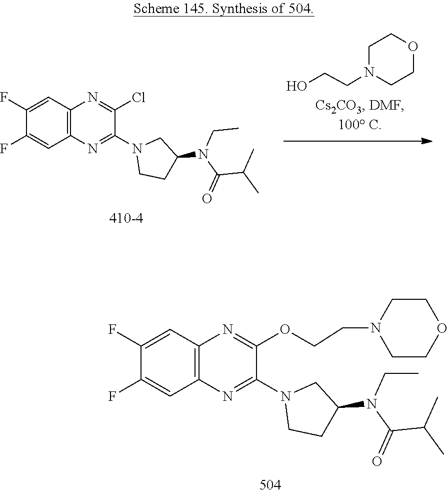 [SMILES: OCCN1CCOCC1 ; CCN(C(=O)C(C)C)[C@H]1CCN(c2nc3cc(F)c(F)cc3nc2Cl)C1 ; CCN(C(=O)C(C)C)[C@H]1CCN(c2nc3cc(F)c(F)cc3nc2OCCN2CCOCC2)C1]

Compound 505 according to the present disclosure was prepared as shown in Scheme 146 by an analogous procedure to the one described in Example 20A.

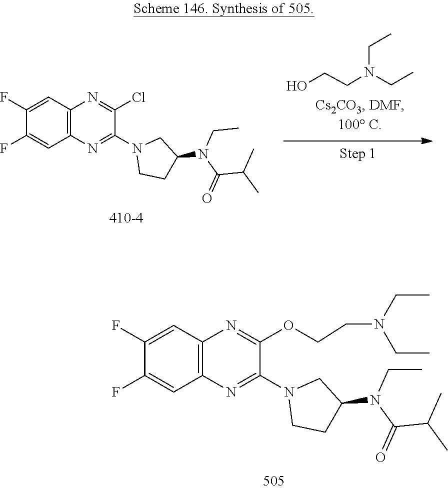

### Example 21: Synthesis of Analogs 438, 439, and 460

Compound 438 according to the present disclosure was prepared as shown in Scheme 147 and described below.

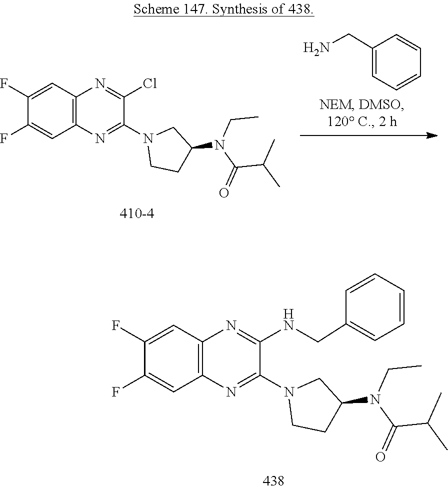 [SMILES: CCN(C(=O)C(C)C)[C@H]1CCN(c2nc3cc(F)c(F)cc3nc2Cl)C1 ; NCc1ccccc1 ; CCN(C(=O)C(C)C)[C@H]1CCN(c2nc3cc(F)c(F)cc3nc2NCc2ccccc2)C1]

### Example 21A: Preparation of Compound 438

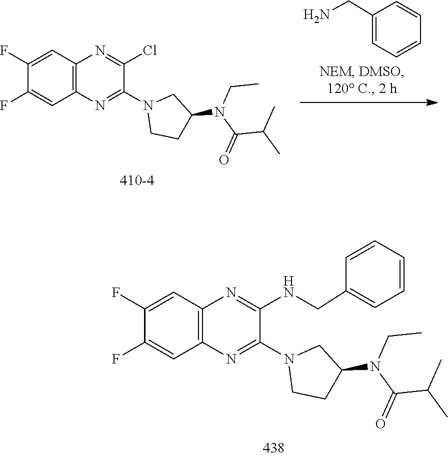 [SMILES: CCN(C(=O)C(C)C)[C@H]1CCN(c2nc3cc(F)c(F)cc3nc2Cl)C1 ; NCc1ccccc1 ; CCN(C(=O)C(C)C)[C@H]1CCN(c2nc3cc(F)c(F)cc3nc2NCc2ccccc2)C1]

The solution of (S)—N-(1-(3-chloro-6,7-difluoroquinoxalin-2-yl)pyrrolidin-3-yl)-N-ethylisobutyramide 410-1 (150 mg, 0.3918 mmol), phenylmethanamine (210 mg, 1.96 mmol) and N-ethylmorpholine (135 mg, 1.18 mmol) in DMSO (10 mL) was warmed to 120° C. in an oil bath. The flask is equipped with a magnetic stirring bar and a three-way stopcock attached to a balloon filled with nitrogen. The mixture was stirred for 2 h at 120° C. The reaction was cooled to 25° C. The residue was extracted with ethyl acetate (3×50 mL) and the combined organic layers were concentrated under reduced pressure at 40° C. The residue was purified via flash chromatography eluting with petroleum ether/EtOAc to provide the crude product. The crude product was purified via preparative HPLC to give (S)—N-(1-(3-(benzyl-amino)-6,7-difluoroquinoxalin-2-yl)pyrrolidin-3-yl)nethylisobutyramide 438 (60.9 mg, yield: 34.2%) as a pale white solid. LC MS of 438: Ret. Time: 1.502 min, MS (ESI) m/z: 454.2, [M+H]⁺. ¹H NMR of 438: ¹H NMR (400 MHz, CDCl₃) δ 7.60-7.21 (m, 7H), 5.73 (s, 1H), 5.29-4.37 (m, 3H), 3.87-3.51 (m, 3H), 3.53-3.12 (m, 3H), 2.92-2.62 (m, 1H), 2.33-1.99 (m, 2H), 1.33-1.02 (m, 9H).

### Example 21B: Preparation of Analogous Compounds 439 and 460

Table 16, below, depicts exemplary compounds synthesized according to an analogous procedure to the one described for 438.

TABLE 16
Compounds synthesized analogously to 438.

| | Compound | | |
|---|---|---|---|
| Compound Structure | No. | Yield | Characterization |
| 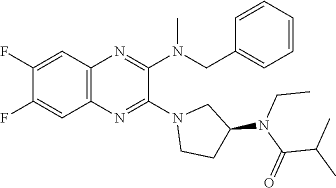 [SMILES: CCN(C(=O)C(C)C)[C@H]1CCN(c2nc3cc(F)c(F)cc3nc2N(C)Cc2ccccc2)C1] | 439 | 36.4% yield | LC MS of 439: Ret. Time: 1.568 min, MS (ESI) m/z: 468.3, [M + H]⁺. ¹H NMR of 439: ¹H NMR (400 MHz, CDCl₃) δ 7.53-7.35 (m, 2H), 7.35-7.07 (m, 5H), 5.13-4.86 (m, 1H), 4.76 (dd, J = 14.1, 7.3 Hz, 1H), 4.36 (d, J = 14.2 Hz, 1H), 3.96- 3.72 (m, 2H), 3.72-3.60 (m, 1H), 3.60-3.18 (m, 3H), 2.95-2.66 (m, 4H), 2.36-1.94 (m, 2H), 1.38-1.03 (m, 9H). |
| 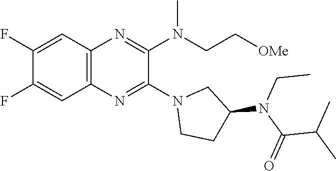 [SMILES: CCN(C(=O)C(C)C)[C@H]1CCN(c2nc3cc(F)c(F)cc3nc2N(C)CCOC)C1] | 460 | 56.6% yield | LC MS of 460: Ret. Time: 1.45 min, MS (ESI) m/z: 436.1, [M + H]⁺. ¹H NMR of 460: ¹H NMR (400 MHz, CDCl₃) δ 7.65-7.35 (m, 2H), 5.05-4.50 (m, 1H), 3.95-3.72 (m, 3H), 3.72-3.57 (m, 1H), 3.57- 3.27 (m, 6H), 3.22 (s, 3H), 2.97 (s, 3H), 2.86-2.71 (m, 1H), 2.27-2.02 (m, 2H), 1.29- 1.13 (m, 9H). |

Compound 439 according to the present disclosure was prepared as shown in Scheme 148 by an analogous procedure to the one described in Example 21A.

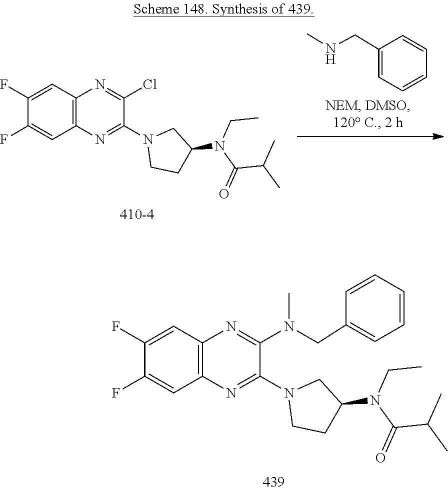

Compound 460 according to the present disclosure was prepared as shown in Scheme 149 by an analogous procedure to the one described in Example 21A.

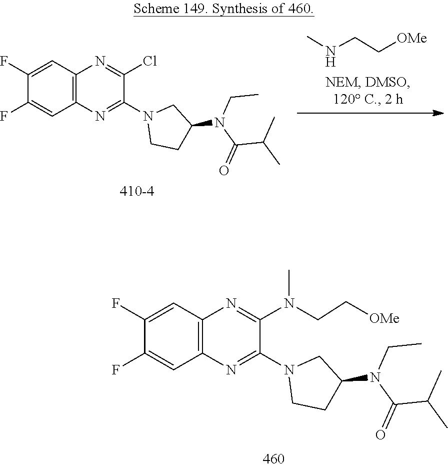

### Example 22: Synthesis of Analog 440

Compound 440 according to the present disclosure was prepared as shown in Scheme 150 and described below.

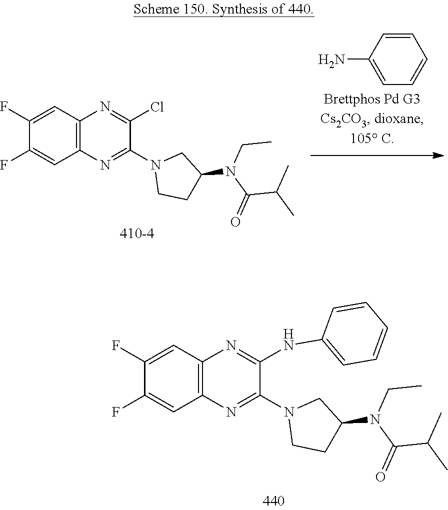 [SMILES: Nc1ccccc1 ; CCN(C(=O)C(C)C)[C@H]1CCN(c2nc3cc(F)c(F)cc3nc2Cl)C1 ; CCN(C(=O)C(C)C)[C@H]1CCN(c2nc3cc(F)c(F)cc3nc2Nc2ccccc2)C1]

### Example 22A: Preparation of Compound 440

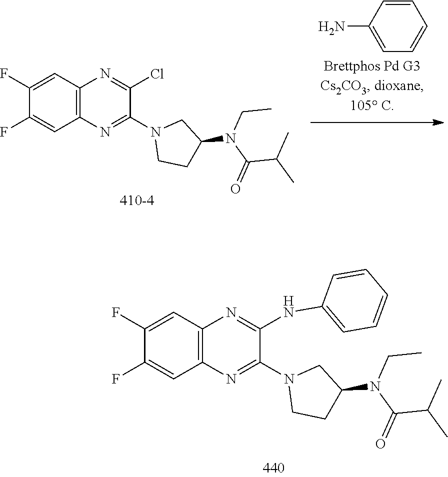 [SMILES: Nc1ccccc1 ; CCN(C(=O)C(C)C)[C@H]1CCN(c2nc3cc(F)c(F)cc3nc2Cl)C1 ; CCN(C(=O)C(C)C)[C@H]1CCN(c2nc3cc(F)c(F)cc3nc2Nc2ccccc2)C1]

To a solution of (S)—N-(1-(3-chloro-6,7-difluoroquinoxalin-2-yl)pyrrolidin-3-yl)-N-ethylisobutyramide 410-4 (150 mg, 0.39 mmol, 1.0 eq), aniline (182 mg, 1.96 mmol, 5.0 eq) and Cs₂CO₃ (383 mg, 1.18 mmol, 3.0 eq) in dioxane (20 mL) stirred under nitrogen at 25° C. was added BrettPhos Pd G3 (71 mg, 0.078 mmol, 0.2 eq). The reaction mixture was stirred at 105° C. for 16 h. After completion, the mixture was quenched with H₂O (100 mL) and extracted with EtOAc (3×50 mL). The combined organic layers were washed with brine for three times and dried over sodium sulphate, filtered and concentrated. The residue was purified by a preparative HPLC to afford (S)—N-(1-(6,7-difluoro-3-(phenylamino)quinoxalin-2-yl)pyrrolidin-3-yl)-N-ethylisobutyramide 440 (55.1 mg, 32.0% yield) as a yellow solid. LC MS of 440: Ret. Time: 1.55 min, MS (ESI) m/z: 440.0, [M+H]⁺. ¹H NMR of 440: 1H NMR (400 MHz, CDCl₃) δ 8.36 (s, 1H), 8.01 (d, J=8.0 Hz, 2H), 7.53-7.44 (m, 2H), 7.37 (t, J=7.8 Hz, 2H), 7.06 (t, J=7.3 Hz, 1H), 4.21-4.01 (m, 2H), 3.76-3.49 (m, 2H), 3.43 (q, J=6.9 Hz, 2H), 3.09 (t, J=8.5 Hz, 1H), 2.78 (dt, J=13.2, 6.8 Hz, 1H), 2.37-2.11 (m, 2H), 1.30-1.14 (m, 9H).

### Example 23: Synthesis of Analog 441

Compound 441 according to the present disclosure was prepared as shown in Scheme 151 and described below.

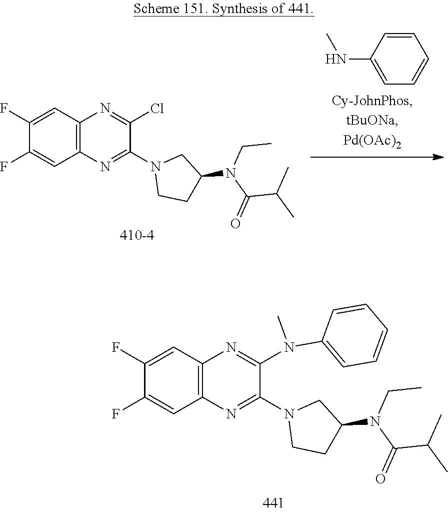

### Example 23A: Preparation of Compound 441

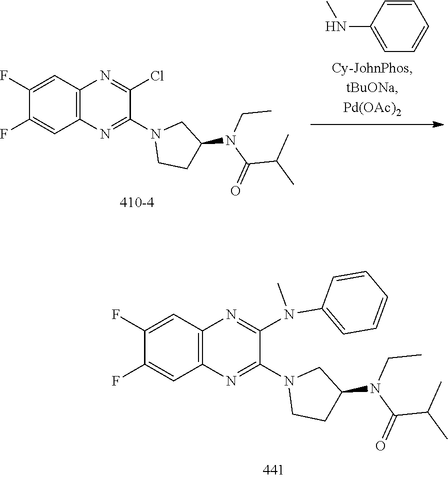

To a solution of (S)—N-(1-(3-chloro-6,7-difluoroquinoxalin-2-yl)pyrrolidin-3-yl)-N-ethylisobutyramide 410-4 (150 mg, 0.39 mmol, 1.0 eq), N-methylaniline (210 mg, 1.96 mmol, 5.0 eq), Cy-JohnPhos (14 mg, 0.039 mmol, 0.1 eq) and t-BuONa (113 mg, 1.18 mmol, 3.0 eq) in Tol (20 mL) stirred under nitrogen at 25° C. was added Pd(OAc)₂ (9 mg, 0.039 mmol, 0.1 eq). The reaction mixture was stirred at 150° C. for 3 h in a microwave. After completion, the mixture was concentrated under reduced pressure at 50° C. The residue was purified by a preparative HPLC to afford (S)—N-(1-(6,7-difluoro-3-(methyl(phenyl)amino)quinoxalin-2-yl)pyrrolidin-3-yl)-N-ethylisobutyramide 441 (67.8 mg, 38.3% yield) as a yellow solid. LC MS of 441: Ret. Time: 1.54 min, MS (ESI) m/z: 454.1, [M+H]⁺. ¹H NMR of 441: ¹H NMR (400 MHz, CDCl₃) δ 7.70-7.44 (m, 2H), 7.24 (t, J=6.2 Hz, 2H), 7.06-6.98 (m, 1H), 6.80 (t, J=8.2 Hz, 2H), 4.95-4.20 (s, 1H), 3.78-3.58 (m, 2H), 3.56-3.48 (m, 3H), 3.45-3.30 (m, 1H), 3.25-3.10 (m, 1H), 3.05-2.86 (m, 2H), 2.72-2.62 (m, 1H), 2.00-1.90 (m, 1H), 1.75-1.65 (m, 1H), 1.13-0.96 (m, 9H).

### Example 24: Synthesis of Analogs 444 and 445

Compounds 444 and 445 according to the present disclosure were prepared as shown in Scheme 152 and described below.

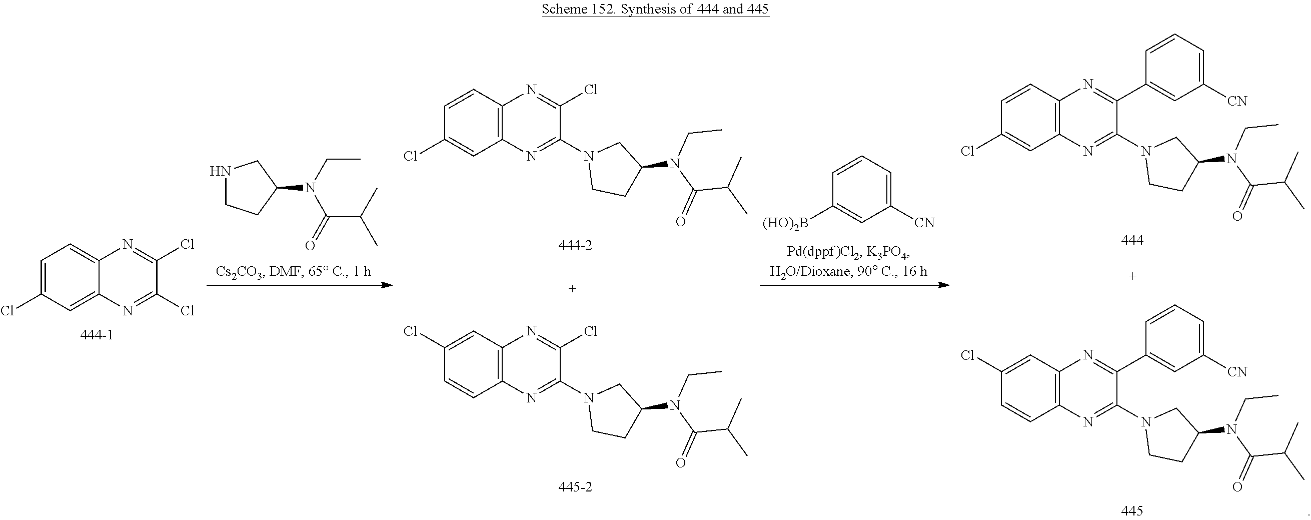

### Example 24A: Preparation of Compound 444-2 and 445-2

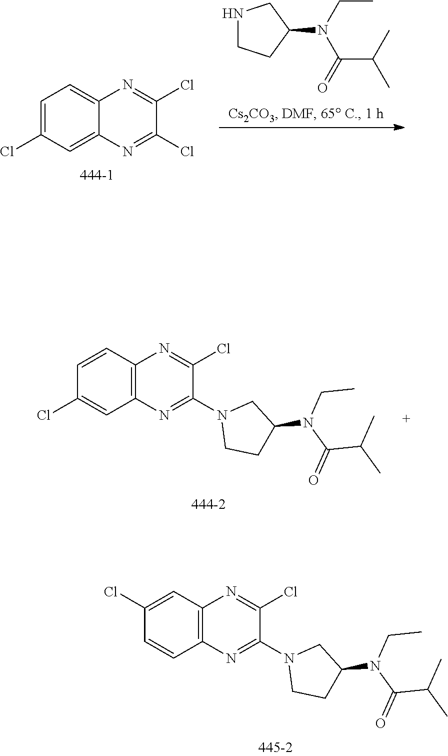 [SMILES: Clc1ccc2nc(Cl)c(Cl)nc2c1 ; CCN(C(=O)C(C)C)[C@H]1CCNC1 ; CCN(C(=O)C(C)C)[C@H]1CCN(c2nc3cc(Cl)ccc3nc2Cl)C1 ; CCN(C(=O)C(C)C)[C@H]1CCN(c2nc3ccc(Cl)cc3nc2Cl)C1]

A mixture of 2,3,6-trichloroquinoxaline 444-1 (400 mg, 1.71 mmol) and Cs₂CO₃ (1675 mg, 5.14 mmol) in DMF (10 mL) was stirred under nitrogen atmosphere at 25° C. N-Ethyl-2-methyl-N-[(3S)-pyrrolidin-3-yl]propenamide (316 mg, 1.71 mmol) was added. The reaction mixture was stirred at 65° C. for 1 h. The reaction was quenched with ice-water (30 mL) and extracted with EtOAc (100 mL). The organic phase was washed with brine (100 mL), dried over Na₂SO₄, concentrated under reduced pressure and purified by chromatography on silica gel, eluting with 10% MeOH in DCM to afford a mixture of (S)—N-(1-(3,7-dichloroquinoxalin-2-yl)pyrrolidin-3-yl)-N-ethylisobutyramide 444-2 and (S)—N-(1-(3,6-dichloroquinoxalin-2-yl)pyrrolidin-3-yl)-N-ethylisobutyramide 445-2 (250 mg, 38.3% yield) as a yellow oil. LC MS of 444-2 and 445-2: Ret. Time: 1.458 min, MS (ESI) m/z: 381.1, [M+H]⁺.

### Example 24B: Preparation of Compound 444 and 445

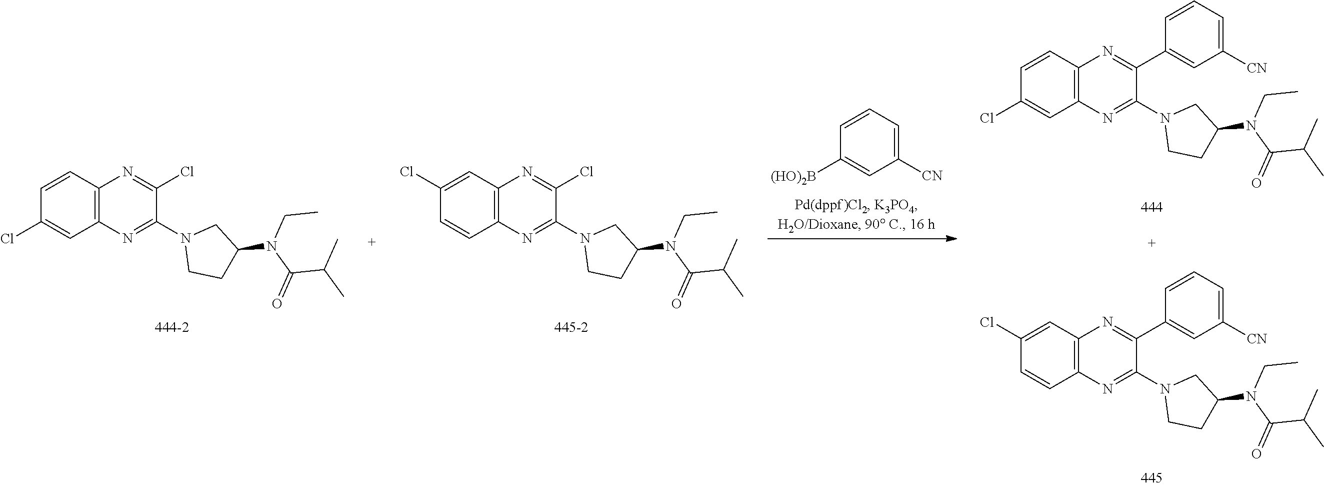 [SMILES: CCN(C(=O)C(C)C)[C@H]1CCN(c2nc3cc(Cl)ccc3nc2Cl)C1 ; CCN(C(=O)C(C)C)[C@H]1CCN(c2nc3ccc(Cl)cc3nc2Cl)C1 ; N#Cc1cccc(B(O)O)c1 ; CCN(C(=O)C(C)C)[C@H]1CCN(c2nc3cc(Cl)ccc3nc2-c2cccc(C#N)c2)C1 ; CCN(C(=O)C(C)C)[C@H]1CCN(c2nc3ccc(Cl)cc3nc2-c2cccc(C#N)c2)C1]

To a solution of (S)—N-(1-(3,7-dichloroquinoxalin-2-yl)pyrrolidin-3-yl)-N-ethylisobutyramide 444-2, (S)—N-(1-(3,6-dichloroquinoxalin-2-yl)pyrrolidin-3-yl)-N-ethylisobutyramide 445-2 (250 mg, 0.66 mmol), 3-(dihydroxyboranyl)benzonitrile (116 mg, 0.79 mmol) and K₃PO₄ (418 mg, 1.97 mmol) in 1,4-dioxane (10 mL) and water (1 mL) stirred under nitrogen at 25° C., Pd(dppf)Cl₂ (96 mg, 0.13 mmol) was added. The reaction mixture was stirred at 90° C. for 16 h. The reaction mixture was evaporated under reduced pressure to give a black oil.

The residue was purified by chromatography on silica gel, eluting with 25% EtOAc in petroleum ether to afford a mixture of (S)—N-(1-(7-chloro-3-(3-cyanophenyl)quinoxalin-2-yl)pyrrolidin-3-yl)-N-ethylisobutyramide 444 and (S)—N-(1-(6-chloro-3-(3-cyanophenyl)quinoxalin-2-yl)pyrrolidin-3-yl)-N-ethylisobutyramide 445 (160 mg, 53.9% yield) as a yellow oil. The mixture of 444 and 445 was separated by chiral preparative HPLC to afford (S)—N-(1-(7-chloro-3-(3-cyanophenyl)quinoxalin-2-yl)pyrrolidin-3-yl)-N-ethylisobutyramide (444, 27.8 mg) and (S)—N-(1-(6-chloro-3-(3-cyanophenyl)quinoxalin-2-yl)pyrrolidin-3-yl)-N-ethylisobutyramide (445, 15.3 mg), each as a yellow solid.

### Example 25: Synthesis of Analogs 446 and 447, 448, 449

Compound 446 according to the present disclosure was prepared as shown in Scheme 153 and described below.

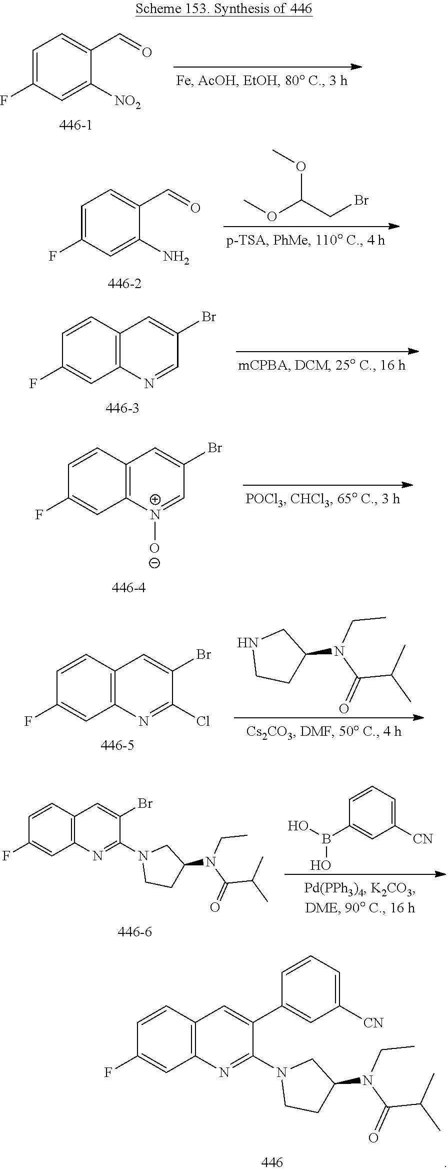

### Example 25A: Preparation of Compound 446-2

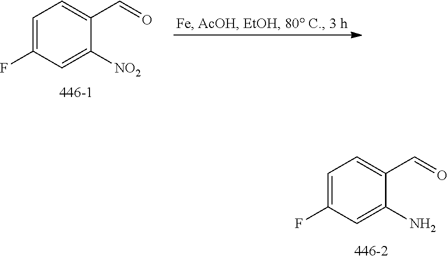 [SMILES: O=Cc1ccc(F)cc1[N+](=O)[O-] ; Nc1cc(F)ccc1C=O]

A solution of 4-fluoro-2-nitrobenzaldehyde 446-1 (6 g, 0.036 mol) in AcOH (24 mL) and EtOH (48 mL) was stirred at 25° C. Iron powder (5.95 g, 0.11 mol) was added. The reaction mixture was stirred at 80° C. for 3 h. The reaction was filtered and the filtrate was concentrated under reduced pressure. The resulting residue was purified by chromatography on silica gel, eluting with 25% EtOAc in petroleum ether to afford 2-amino-4-fluorobenzaldehyde 446-2 (0.62 g, 12.4% yield) as a yellow oil. LC MS of 446-2: Ret. Time: 1.105 min, MS (ESI) m/z: 140.0, [M+H]⁺.

### Example 25B: Preparation of Compound 446-3

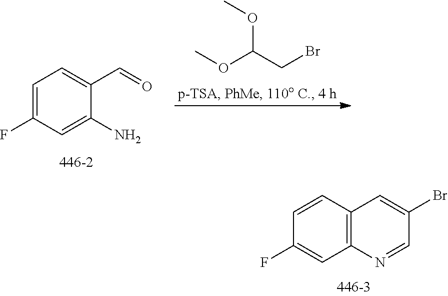

A solution of 2-amino-4-fluorobenzaldehyde 446-2 (1.52 g, 10.9 mmol) and 2-bromo-1,1-dimethoxyethane (2.21 g, 13 mmol) in PhMe (30 mL) was stirred under a nitrogen atmosphere. PTSA (1.88 g, 10.9 mmol) was added in portions. The reaction mixture was stirred at 110° C. for 4 h. The reaction was quenched with aqueous NaHCO₃ (30 mL) and extracted with EtOAc (100 mL). The organic phase was washed with brine (100 mL), dried over Na₂SO₄, concentrated under reduced pressure and purified by chromatography on silica gel, eluting with 20% EtOAc in DCM to afford 3-bromo-7-fluoroquinoline 446-3 (0.58 g, 22.9% yield) as a yellow solid. LC MS of 446-3: Ret. Time: 1.324 min, MS (ESI) m/z: 226.0, [M+H]⁺.

### Example 25C: Preparation of Compound 446-4

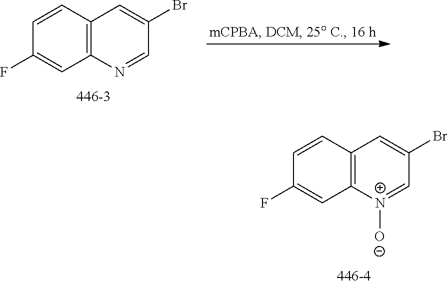 [SMILES: Fc1ccc2cc(Br)cnc2c1 ; [O-][n+]1cc(Br)cc2ccc(F)cc21]

A solution of 3-bromo-7-fluoroquinoline 446-3 (620 mg, 2.74 mmol) in DCM (10 mL) was stirred under a nitrogen atmosphere at 0° C. m-CPBA (805 mg, 4.66 mmol) was added in portions. The reaction mixture was stirred at room temperature for 16 h. The reaction was quenched with aqueous NaHCO₃ (30 mL) and extracted with DCM (100 mL). The organic phase was washed with brine (100 mL), dried over Na₂SO₄, concentrated under reduced pressure and purified by chromatography on silica gel, eluting with 25% EtOAc in petroleum ether to afford 3-bromo-7-fluoroquinolin-1-ium-1-olate 446-4 (530 mg, 79.0% yield) as a white solid. LC MS of 446-4: Ret. Time: 1.056 min, MS (ESI) m/z: 242.0, [M+H]⁺.

### Example 25D: Preparation of Compound 446-5

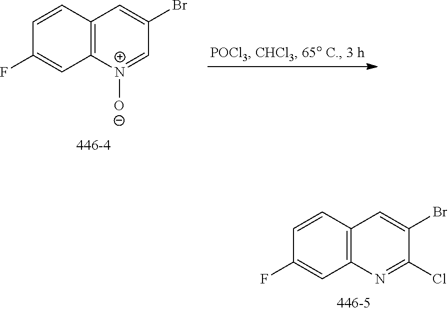 [SMILES: [O-][n+]1cc(Br)cc2ccc(F)cc21 ; Fc1ccc2cc(Br)c(Cl)nc2c1]

A solution of 3-bromo-7-fluoroquinolin-1-ium-1-olate 446-4 (530 mg, 2.19 mmol) was stirred under a nitrogen atmosphere in CHCl₃ (10 mL) and phosphoryl trichloride (3357 mg, 21.90 mmol) was added. The reaction mixture was heated slowly to 65° C. and maintained at that temperature for 3 h. The reaction was concentrated under reduced pressure and quenched with sodium bicarbonate, adjusted to pH 7 and extracted with EtOAc (20 mL). The organic phase was dried with Na₂SO₄, concentrated under reduced pressure and purified by chromatography on silica gel, eluting with 25% EtOAc in petroleum ether to afford 3-bromo-2-chloro-7-fluoroquinoline 446-5 (300 mg, 52.1% yield) as yellow oil. LC MS of 446-5: Ret. Time: 1.418 min, MS (ESI) m/z: 259.9, [M+H]⁺.

### Example 25E: Preparation of Compound 446-6

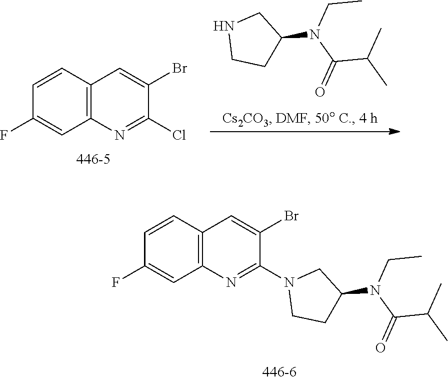 [SMILES: Fc1ccc2cc(Br)c(Cl)nc2c1 ; CCN(C(=O)C(C)C)[C@H]1CCNC1 ; CCN(C(=O)C(C)C)[C@H]1CCN(c2nc3cc(F)ccc3cc2Br)C1]

A solution of 3-bromo-2-chloro-7-fluoroquinoline 446-5 (300 mg, 1.15 mmol), N-ethyl-2-methyl-N-[(3S)-pyrrolidin-3-yl]propenamide (255 mg, 1.38 mmol) in DMF (10 mL) was stirred under a nitrogen atmosphere at 25° C. Cs₂CO₃ (1126 mg, 3.46 mmol) was added. The reaction mixture was stirred at 50° C. for 4 h. The reaction was quenched with water (30 mL) and extracted with EtOAc (100 mL). The organic phase was washed with brine (100 mL), dried with Na₂SO₄, concentrated under reduced pressure and purified by chromatography on silica gel, eluting with 20% EtOAc in petroleum ether to afford N-[(3S)-1-(3-bromo-7-fluoroquinolin-2-yl)pyrrolidin-3-yl]-N-ethyl-2-methyl-propenamide 446-6 (390 mg, 82.1% yield) as a yellow oil. LC MS of 446-6: Ret. Time: 1.483 min, MS (ESI) m/z: 408.1, [M+H]⁺.

### Example 25F. Preparation of Compound 446

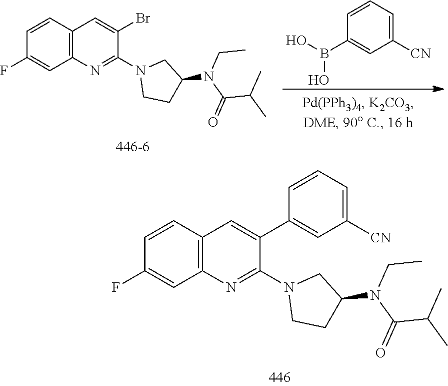 [SMILES: CCN(C(=O)C(C)C)[C@H]1CCN(c2nc3cc(F)ccc3cc2Br)C1 ; N#Cc1cccc(B(O)O)c1 ; CCN(C(=O)C(C)C)[C@H]1CCN(c2nc3cc(F)ccc3cc2-c2cccc(C#N)c2)C1]

To a solution of N-[(3S)-1-(3-bromo-7-fluoroquinolin-2-yl)pyrrolidin-3-yl]-N-ethyl-2-methylpropanamide 446-6 (130 mg, 0.32 mmol), 3-(dihydroxyboranyl)benzonitrile (70 mg, 0.48 mmol) and K₂CO₃ (132 mg, 0.96 mmol) in DME (10 mL) and water (2 mL) stirred under nitrogen at 25° C., Pd(PPh₃)₄ (74 mg, 0.064 mmol) was added. The reaction mixture was stirred at 90° C. for 16 h. The reaction mixture was evaporated under reduced pressure to give a black oil. The residue was purified by chromatography on silica gel, eluting with 33% EtOAc in petroleum ether to afford (S)—N-(1-(3-(3-cyanophenyl)-7-fluoroquinolin-2-yl)pyrrolidin-3-yl)-N-ethylisobutyramide 446 (61.5 mg, 44.9% yield) as a white solid. LC MS of 446: Ret. Time: 1.282 min, MS (ESI) m/z: 431.2, [M+H]⁺. ¹H NMR of 446: ¹H NMR (400 MHz, CDCl₃), δ 7.90-7.32 (m, 7H), 7.17-7.01 (m, 1H), 4.95 (s, 1H), 3.63-3.00 (m, 6H), 2.82-2.67 (m, 1H), 2.15-1.89 (m, 2H), 1.21-0.93 (m, 9H).

### Example 25G: Preparation of Analogous Compounds 447, 448, and 449

Table 17, below, depicts exemplary compounds synthesized according to an analogous procedure to the one described for 446.

TABLE 17
Compounds synthesized analogously to 446.

| | Compound | | |
|---|---|---|---|
| Compound Structure | No. | Yield | Characterization |
| 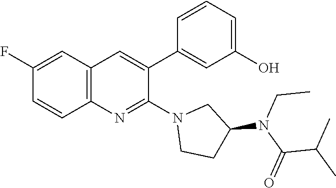 | 447 | 42.4% yield | LC MS of 447: Ret. Time: 1.255 min, MS (ESI) m/z: 431.2, [M + H]⁺. ¹H NMR of 447: ¹H NMR (400 MHz, CDCl₃) δ 8.04-7.53 (m, 6H), 7.45-7.28 (m, 2H), 5.10-4.40 (m, 1H), 3.60-3.05 (m, 6H), 2.88-2.65 (m, 1H), 2.15-1.85 (m, 2H), 1.32-0.84 (m, 9H). |
| 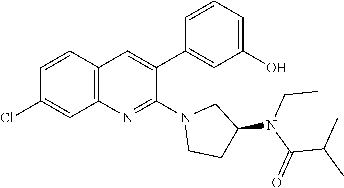 | 448 | 48.6% yield | LC MS of 448: Ret. Time: 1.375 min, MS (ESI) m/z: 447.2, [M + H]⁺. ¹H NMR of 448: ¹H NMR (400 MHz, CDCl₃) δ 7.90-7.65 (m, 5H), 7.65-7.53 (m, 2H), 7.27 (s, 1H), 5.00-4.44 (m, 1H), 3.68- 3.05 (m, 6H), 2.85-2.62 (m, 1H), 2.16-1.84 (m, 2H), 1.22-0.96 (m, 9H). |
| 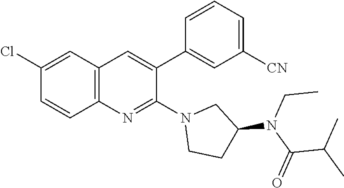 [SMILES: CCN(C(=O)C(C)C)[C@H]1CCN(c2nc3ccc(Cl)cc3cc2-c2cccc(C#N)c2)C1] | 449 | 58.5% yield | LC MS of 449: Ret. Time: 1.358 min, MS (ESI) m/z: 447.2, [M + H]⁺. ¹H NMR of 449: ¹H NMR (400 MHz, CDCl₃) δ 7.83-7.65 (m, 5H), 7.64-7.50 (m, 3H), 4.94-4.34 (m, 1H), 3.59-3.06 (m, 6H), 2.78-2.65 (m, 1H), 2.20-1.85 (m, 2H), 1.17-1.06 (m, 9H). |

Compound 447 according to the present disclosure was prepared as shown in Scheme 154 by an analogous procedure to the one described in Examples 25A-25F.

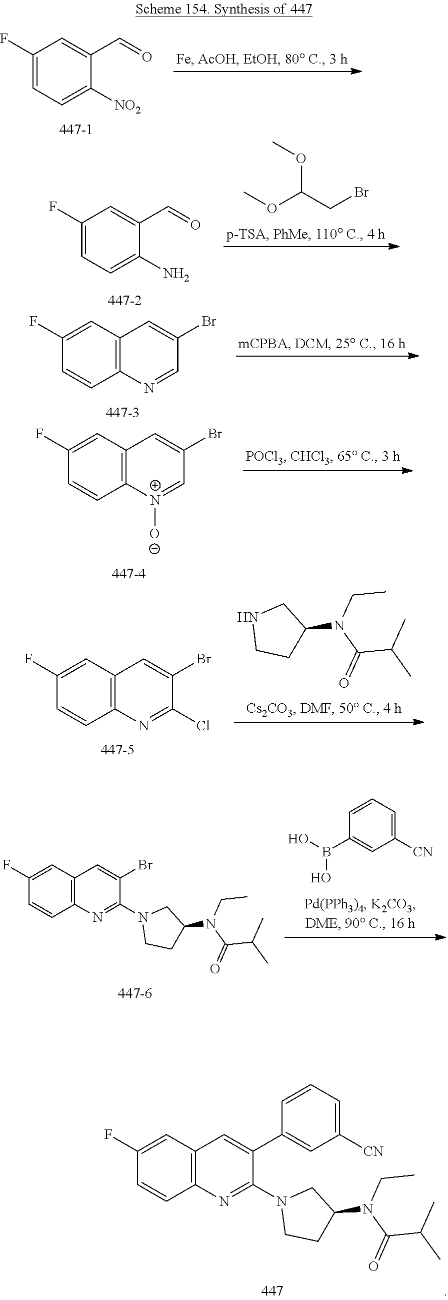

Compound 448 according to the present disclosure was prepared as shown in Scheme 155 by an analogous procedure to the one described in Examples 25A-25F.

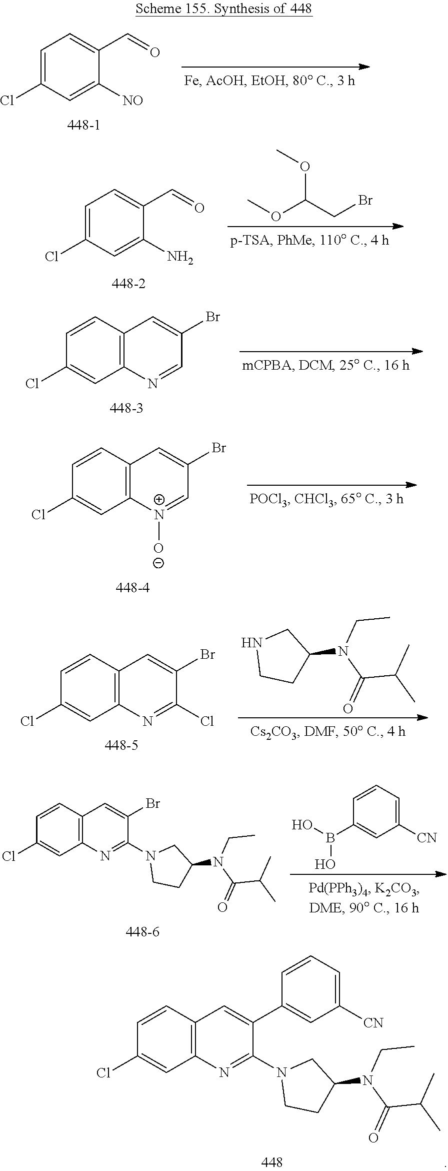

Compound 449 according to the present disclosure was prepared as shown in Scheme 156 by an analogous procedure to the one described in Examples 25A-25F.

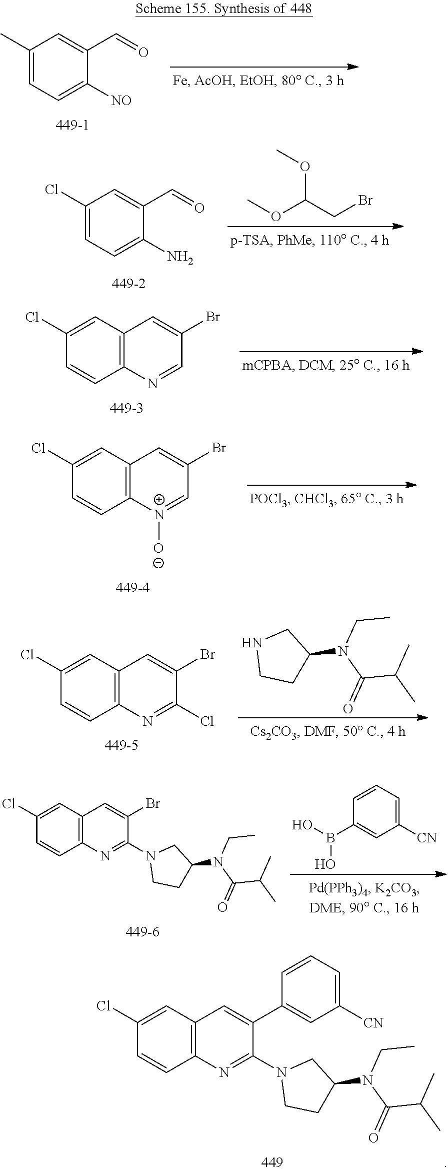

### Example 26: Synthesis of Analogs 454, 458, and 503

Compound 454 according to the present disclosure was prepared as shown in Scheme 157 and described below.

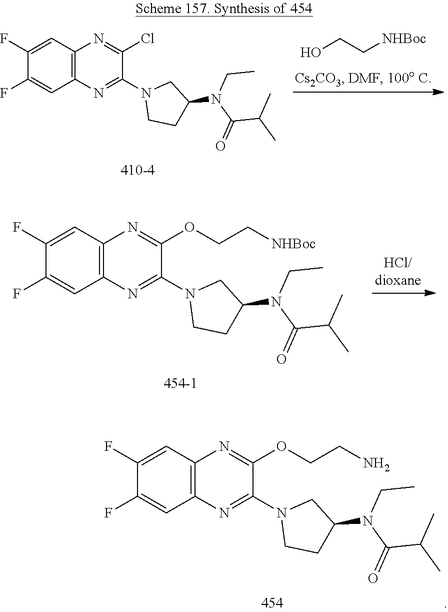 [SMILES: CCN(C(=O)C(C)C)[C@H]1CCN(c2nc3cc(F)c(F)cc3nc2Cl)C1 ; CC(C)(C)OC(=O)NCCO ; CCN(C(=O)C(C)C)[C@H]1CCN(c2nc3cc(F)c(F)cc3nc2OCCNC(=O)OC(C)(C)C)C1 ; CCN(C(=O)C(C)C)[C@H]1CCN(c2nc3cc(F)c(F)cc3nc2OCCN)C1]

### Example 26A: Preparation of Compound 454-1

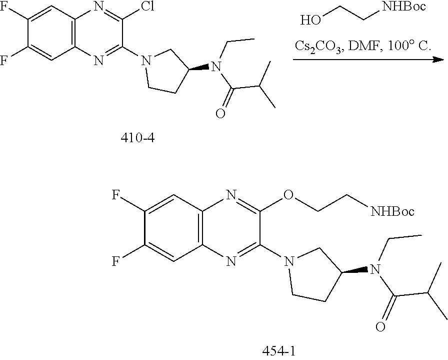 [SMILES: CCN(C(=O)C(C)C)[C@H]1CCN(c2nc3cc(F)c(F)cc3nc2Cl)C1 ; CC(C)(C)OC(=O)NCCO ; CCN(C(=O)C(C)C)[C@H]1CCN(c2nc3cc(F)c(F)cc3nc2OCCNC(=O)OC(C)(C)C)C1]

To a solution of (S)—N-(1-(3-chloro-6,7-difluoroquinoxalin-2-yl)pyrrolidin-3-yl)-N-ethylisobutyramide 410-4 (200 mg, 0.52 mmol, 1.0 eq) and Cs₂CO₃ (511 mg, 1.57 mmol, 3.0 eq) in DMF (20 mL) stirred under nitrogen at 25° C. was added tert-butyl (2-hydroxyethyl)carbamate (253 mg, 1.57 mmol, 3.0 eq). The reaction mixture was stirred at 100° C. for 16 h. After completion, the mixture was concentrated under reduced pressure at 60° C. The residue was purified by flash chromatography eluting with 2:1 petroleum ether/EtOAc to afford tert-butyl (S)-(2-((3-(3-(N-ethylisobutyramido)pyrrolidin-1-yl)-6,7-difluoro-quinoxalin-2-yl)oxy)ethyl)carbamate 454-1 (120 mg, yield: 56.47%) as a yellow oil. LC MS of 454-1: Ret. Time: 1.478 min, MS (ESI) m/z: 508.1, [M+H]⁺.

### Example 26B: Preparation of Compound 454

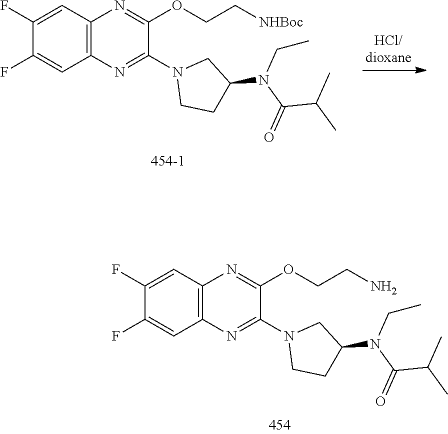 [SMILES: CCN(C(=O)C(C)C)[C@H]1CCN(c2nc3cc(F)c(F)cc3nc2OCCNC(=O)OC(C)(C)C)C1 ; CCN(C(=O)C(C)C)[C@H]1CCN(c2nc3cc(F)c(F)cc3nc2OCCN)C1]

To tert-butyl (S)-(2-((3-(3-(N-ethylisobutyramido)pyrrolidin-1-yl)-6,7-difluoroquinoxalin-2-yl)oxy)ethyl)carbamate 454-1 (120 mg, 0.52 mmol, 1.0 eq) stirred under nitrogen at 25° C. was added HCl/dioxane (15 mL).The reaction mixture was stirred at 25° C. for 2 h. After completion, the mixture was concentrated under reduced pressure at 40° C. The mixture was adjusted to pH 8. The residue was purified by a preparative HPLC to afford (S)—N-(1-(3-(2-aminoethoxy)-6,7-difluoroquinoxalin-2-yl)pyrrolidin-3-yl)-N-ethylisobutyramide 454 (35.1 mg, 35.7% yield) as a white solid. LC MS of 454: Ret. Time: 1.11 min, MS (ESI) m/z: 408.1, [M+H]⁺. ¹H NMR of 454: ¹H NMR (400 MHz, CDCl₃) δ 8.35 (s, 1H), 7.38-7.27 (m, 2H), 4.93-4.79 (m, 1H), 4.80-4.50 (m, 2H), 4.17-3.93 (m, 2H), 3.85-3.63 (m, 2H), 3.67-3.09 (m, 4H), 2.81-2.71 (m, 1H), 2.15-1.85 (m, 2H), 1.30-1.02 (m, 9H).

### Example 26C: Preparation of Analogous Compounds 458 and 503

Table 18, below, depicts exemplary compounds synthesized according to an analogous procedure to the one described for 454.

TABLE 18
Compounds synthesized analogously to 454.

| | Compound | | |
|---|---|---|---|
| Compound Structure | No. | Yield | Characterization |
| 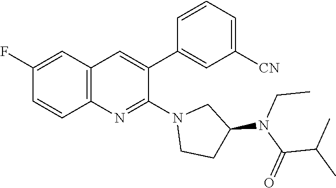 | 458 | 55.7% yield | LC MS of 458: Ret. Time: 1.14 min, MS (ESI) m/z: 434.1, [M + H]⁺. ¹H NMR of 458: ¹H NMR (400 MHz, CDCl3) δ 7.42-7.30 (m, 2H), 4.77-4.47 (m, 4H), 4.25-4.16 (m, 2H), 4.15-3.95 (m, 3H), 3.95-3.72 (m, 1H), 3.65-3.51 (m, 2H), 3.48-3.32 (m, 2H), 2.83-2.67 (m, 1H), 2.15-1.85 (m, 2H), 1.29-1.09 (m, 9H). |
| 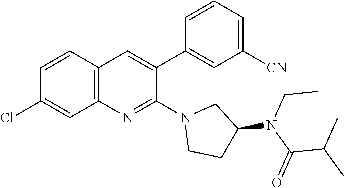 | 503 | 42.3% yield | LC MS of 503: Ret. Time: 1.14 min, MS (ESI) m/z: 436.1, [M + H]⁺. ¹H NMR of 503: ¹H NMR (400 MHz, CDCl₃) δ 8.41 (s, 1H), 7.33-7.23 (m, 2H), 5.00-4.87 (m, 1H), 4.70 (s, 2H), 4.09-3.93 (m, 2H), 3.79-3.53 (m, 2H), 3.53-3.20 (m, 4H), 3.00 (q, J = 7.0 Hz, 2H), 2.94-2.72 (m, 1H), 2.24-2.02 (m, 2H), 1.34-1.12 (m, 12H). |

Compound 458 according to the present disclosure was prepared as shown in Scheme 158 by an analogous procedure to the one described in Examples 26A-26B.

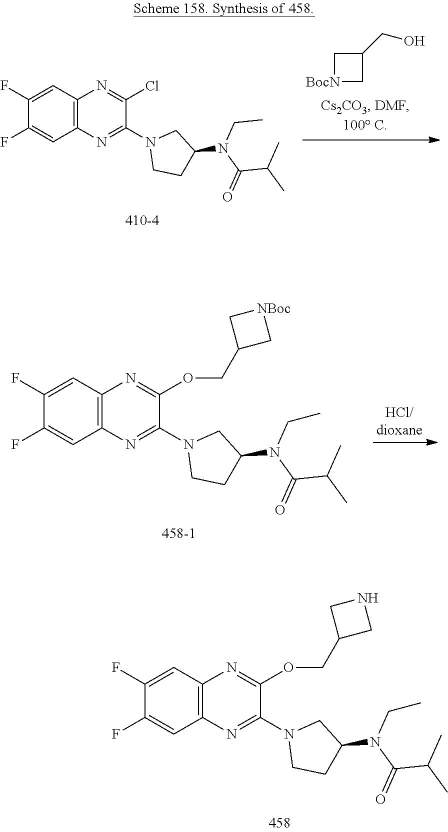 [SMILES: CC(C)(C)OC(=O)N1CC(CO)C1 ; CCN(C(=O)C(C)C)[C@H]1CCN(c2nc3cc(F)c(F)cc3nc2Cl)C1 ; CCN(C(=O)C(C)C)[C@H]1CCN(c2nc3cc(F)c(F)cc3nc2OCC2CN(C(=O)OC(C)(C)C)C2)C1 ; CCN(C(=O)C(C)C)[C@H]1CCN(c2nc3cc(F)c(F)cc3nc2OCC2CNC2)C1]

Compound 503 according to the present disclosure was prepared as shown in Scheme 159 by an analogous procedure to the one described in Examples 26A-26B.

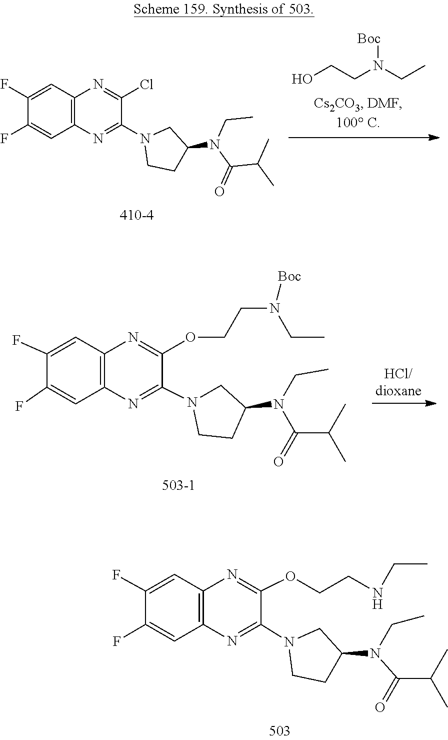 [SMILES: CCN(CCO)C(=O)OC(C)(C)C ; CCN(C(=O)C(C)C)[C@H]1CCN(c2nc3cc(F)c(F)cc3nc2Cl)C1 ; CCN(CCOc1nc2cc(F)c(F)cc2nc1N1CC[C@H](N(CC)C(=O)C(C)C)C1)C(=O)OC(C)(C)C ; CCNCCOc1nc2cc(F)c(F)cc2nc1N1CC[C@H](N(CC)C(=O)C(C)C)C1]

### Example 27: Synthesis of Analogs 508, 509, and 529

Compound 508 according to the present disclosure was prepared as shown in Scheme 160 and described below.

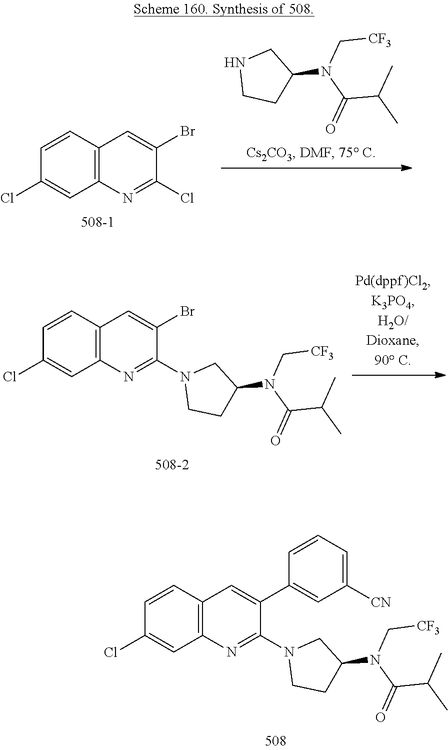 [SMILES: CC(C)C(=O)N(CC(F)(F)F)[C@H]1CCNC1 ; Clc1ccc2cc(Br)c(Cl)nc2c1 ; CC(C)C(=O)N(CC(F)(F)F)[C@H]1CCN(c2nc3cc(Cl)ccc3cc2Br)C1 ; CC(C)C(=O)N(CC(F)(F)F)[C@H]1CCN(c2nc3cc(Cl)ccc3cc2-c2cccc(C#N)c2)C1]

### Example 27A.: Preparation of Compound 508-2

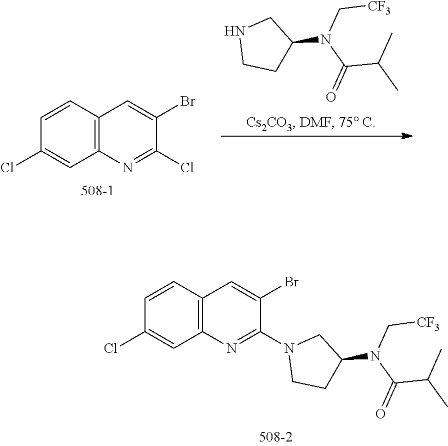 [SMILES: CC(C)C(=O)N(CC(F)(F)F)[C@H]1CCNC1 ; Clc1ccc2cc(Br)c(Cl)nc2c1 ; CC(C)C(=O)N(CC(F)(F)F)[C@H]1CCN(c2nc3cc(Cl)ccc3cc2Br)C1]

A round-bottom flask containing a mixture of (S)—N-(pyrrolidin-3-yl)-N-(2,2,2-trifluoroethyl)isobutyramide (465 mg, 1.95 mmol), 3-bromo-2,7-dichloroquinoline 508-1 (450 mg, 1.62 mmol) and cesium carbonate (1.59 g, 4.87 mmol) in DMF (20 mL) was placed in an oil bath heated to 70° C. The mixture was continuously stirred at 70° C. for 1 h. The reaction was cooled to 25° C. Saturated brine (100 mL) was added. The residue was extracted with EtOAc (100 mL×3). The organic phase was washed with saturated brine (50 mL×5) and concentrated under reduced pressure at 40° C. The residue was purified via flash chromatography eluting with 80:20 petroleum ether/EtOAc to give the product (S)—N-(1-(3-bromo-7-chloroquinolin-2-yl)pyrrolidin-3-yl)-N-(2,2,2-trifluoroethyl)isobutyramide 508-2 (360 mg, yield: 45.5%) as a yellow solid. LC MS of 508-2: Ret. Time: 1.508 min, MS (ESI) m/z: 478.0, [M+H]⁺; 480.0, [M+3]⁺.

### Example 27B: Preparation of Compound 508

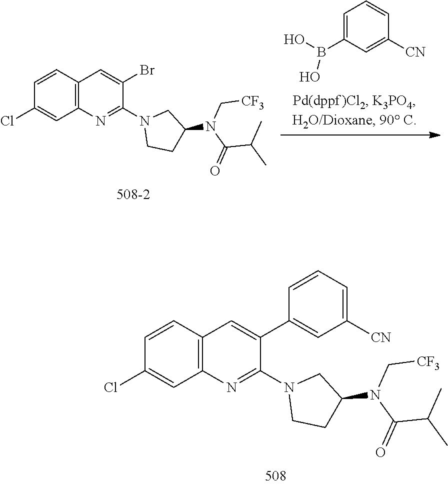 [SMILES: CC(C)C(=O)N(CC(F)(F)F)[C@H]1CCN(c2nc3cc(Cl)ccc3cc2Br)C1 ; N#Cc1cccc(B(O)O)c1 ; CC(C)C(=O)N(CC(F)(F)F)[C@H]1CCN(c2nc3cc(Cl)ccc3cc2-c2cccc(C#N)c2)C1]

The solution of (S)—N-(1-(3-bromo-7-chloroquinolin-2-yl)pyrrolidin-3-yl)-N-(2,2,2-trifluoroethyl) isobutyramide 508-2 (120 mg, 0.25 mmol), 3-cyanophenylboronic acid (44 mg, 0.30 mmol), Pd(dppf)Cl₂ (18.3 mg, 0.025 mmol) and K₃PO₄ (159 mg, 0.75 mmol) in dioxane (10 mL) was warmed to 90° C. in an oil bath. The flask was equipped with a magnetic stirring bar and a three-way stopcock attached to a balloon filled with nitrogen. The mixture is stirred for 2 h at 90° C. The reaction was cooled to 25° C. The reaction mixture was concentrated under reduced pressure at 45° C. The residue was purified via flash chromatography eluting with hexane/EtOAc to give the impure product. The impure product was further purified via preparative HPLC to give the product (S)—N-(1-(7-chloro-3-(3-cyanophenyl)quinolin-2-yl)pyrrolidin-3-yl)-N-(2,2,2-trifluoroethyl)isobutyramide 508 (57.8 mg, yield: 47.9%) as a white solid. LC MS of 508: Ret. Time: 1.442 min, MS (ESI) m/z: 501.2, [M+H]⁺. ¹H NMR of 508: ¹H NMR (400 MHz, DMSO-d₆) δ 8.01 (s, 1H), 7.90 (s, 1H), 7.85-7.75 (m, 3H), 7.65 (dt, J=10.6, 5.4 Hz, 2H), 7.26 (dd, J=8.5, 2.1 Hz, 1H), 4.48 (s, 1H), 4.31-3.92 (m, 2H), 3.50-3.20 (m, 3H), 3.19-3.05 (m, 1H), 2.97-2.82 (m, 1H), 2.11-1.89 (m, 2H), 0.98 (dd, J=11.1, 6.6 Hz, 6H).

### Example 27C: Preparation of Analogous Compounds 509 and 529

Table 19, below, depicts exemplary compounds synthesized according to an analogous procedure to the one described for 508.

TABLE 19
Compounds synthesized analogously to 508.

| | Compound | | |
|---|---|---|---|
| Compound Structure | No. | Yield | Characterization |
| 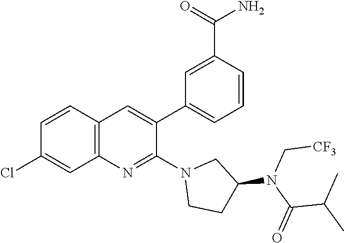 [SMILES: CC(C)C(=O)N(CC(F)(F)F)[C@H]1CCN(c2nc3cc(Cl)ccc3cc2-c2cccc(C(N)=O)c2)C1] | 509 | 26.3% yield | LC MS of 509: Ret. Time: 1.223 min, MS (ESI) m/z: 519.2, [M + H]⁺. ¹H NMR of 509: ¹H NMR (400 MHz, DMSO-d₆) δ 7.99-7.93 (m, 2H), 7.91-7.85 (m, 1H), 7.78 (d, J = 8.5 Hz, 1H), 7.65 (d, J = 2.1 Hz, 1H), 7.59 (dt, J = 7.6, 1.4 Hz, 1H), 7.51 (t, J = 7.7 Hz, 1H), 7.25 (dd, J = 8.5, 2.1 Hz, 1H), 4.56-4.40 (m, 1H), 4.25-4.00 (m, 2H), 3.45 (dd, J = 11.0, 7.7 Hz, 1H), 3.37- 3.29 (m, 1H), 3.28-3.13 (m, 2H), 2.96-2.85 (m, 1H), 2.06-1.94 (m, 2H), 0.97 (dd, J = 12.2, 6.6 Hz, 6H). |
| 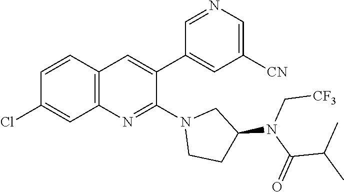 [SMILES: CC(C)C(=O)N(CC(F)(F)F)[C@H]1CCN(c2nc3cc(Cl)ccc3cc2-c2cncc(C#N)c2)C1] | 529 | 47.6% yield | LC MS of 529: Ret. Time: 1.409 min, MS (ESI) m/z: 502.2, [M + H]⁺. ¹H NMR of 529: ¹H NMR (400 MHz, DMSO-d₆) δ 8.99 (d, J = 2.0 Hz, 1H), 8.96 (d, J = 2.2 Hz, 1H), 8.38 (t, J = 2.0 Hz, 1H), 8.10 (s, 1H), 7.79 (d, J = 8.6 Hz, 1H), 7.67 (d, J = 2.1 Hz, 1H), 7.28 (dd, J = 8.5, 2.1 Hz, 1H), 4.60-4.40 (m, 1H), 4.33-3.96 (m, 2H), 3.41 (dd, J = 10.7, 7.7 Hz, 1H), 3.36- 3.20 (m, 2H), 3.20-3.09 (m, 1H), 3.00-2.85 (m, 1H), 2.10-1.95 (m, 2H), 0.99 (dd, J = 9.2, 6.6 Hz, 6H). |

Compound 509 according to the present disclosure was prepared as shown in Scheme 161 by an analogous procedure to the one described in Examples 27A-27B.

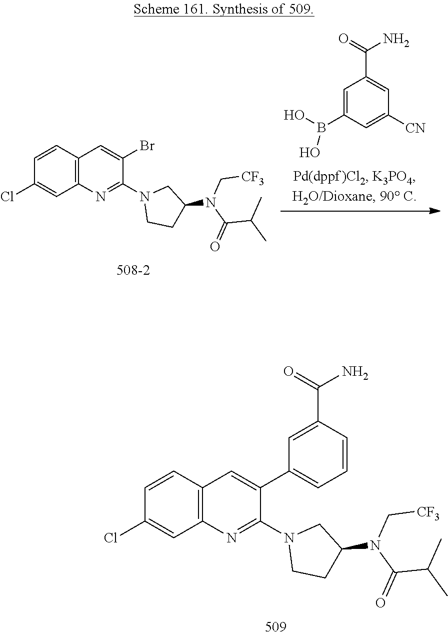 [SMILES: CC(C)C(=O)N(CC(F)(F)F)[C@H]1CCN(c2nc3cc(Cl)ccc3cc2Br)C1 ; N#Cc1cc(B(O)O)cc(C(N)=O)c1 ; CC(C)C(=O)N(CC(F)(F)F)[C@H]1CCN(c2nc3cc(Cl)ccc3cc2-c2cccc(C(N)=O)c2)C1]

Compound 529 according to the present disclosure was prepared as shown in Scheme 162 by an analogous procedure to the one described in Examples 27A-27B.

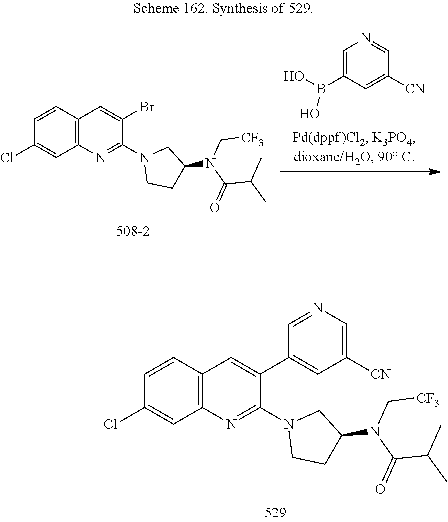 [SMILES: CC(C)C(=O)N(CC(F)(F)F)[C@H]1CCN(c2nc3cc(Cl)ccc3cc2Br)C1 ; N#Cc1cncc(B(O)O)c1 ; CC(C)C(=O)N(CC(F)(F)F)[C@H]1CCN(c2nc3cc(Cl)ccc3cc2-c2cncc(C#N)c2)C1]

### Example 28: Full Synthesis of Analog 510

Compound 510 according to the present disclosure was prepared as shown in Scheme 163 and described below.

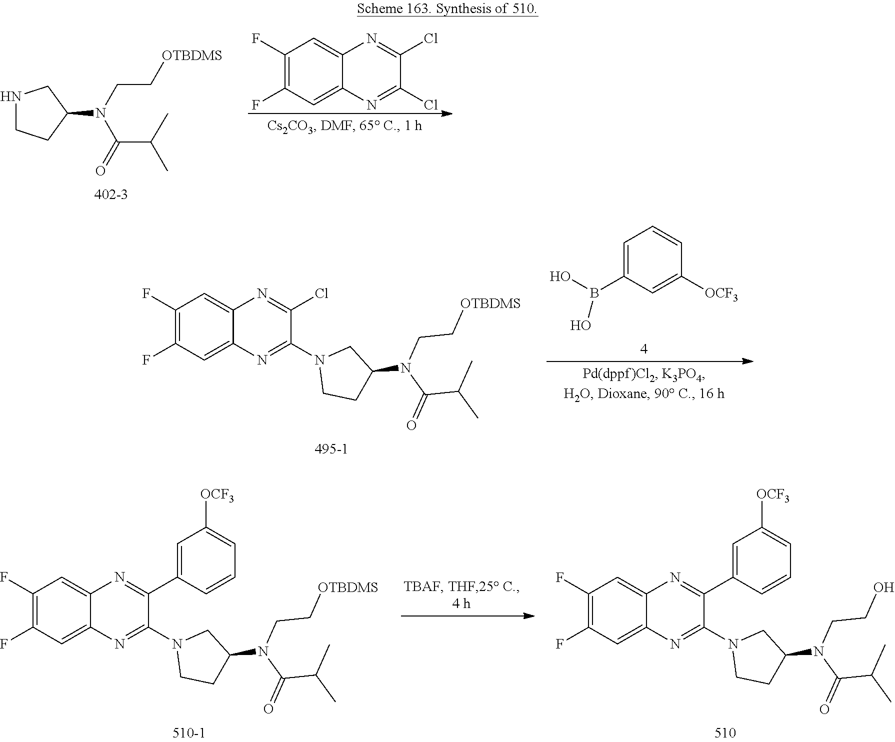 [SMILES: CC(C)C(=O)N(CCO[Si](C)(C)C(C)(C)C)[C@H]1CCNC1 ; Fc1cc2nc(Cl)c(Cl)nc2cc1F ; CC(C)C(=O)N(CCO[Si](C)(C)C(C)(C)C)[C@H]1CCN(c2nc3cc(F)c(F)cc3nc2Cl)C1 ; OB(O)c1cccc(OC(F)(F)F)c1 ; CC(C)C(=O)N(CCO[Si](C)(C)C(C)(C)C)[C@H]1CCN(c2nc3cc(F)c(F)cc3nc2-c2cccc(OC(F)(F)F)c2)C1 ; CC(C)C(=O)N(CCO)[C@H]1CCN(c2nc3cc(F)c(F)cc3nc2-c2cccc(OC(F)(F)F)c2)C1]

### Example 28A: Preparation of Compound 495-1

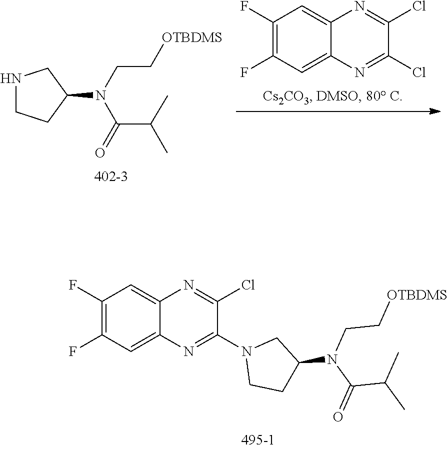 [SMILES: CC(C)C(=O)N(CCO[Si](C)(C)C(C)(C)C)[C@H]1CCNC1 ; Fc1cc2nc(Cl)c(Cl)nc2cc1F ; CC(C)C(=O)N(CCO[Si](C)(C)C(C)(C)C)[C@H]1CCN(c2nc3cc(F)c(F)cc3nc2Cl)C1]

A solution of N-{2-[(tert-butyldimethylsilyl)oxy]ethyl}-2-methyl-N-[(3S)-pyrrolidin-3-yl]propenamide (1.8 g, 5.70 mmol), Cs₂CO₃ (7.43 g, 22.80 mmol) in DMF (30 mL) was stirred under nitrogen atmosphere at 25° C. 2,3-dichloro-6,7-difluoroquinoxaline (1.74 g, 7.40 mol) was added. The reaction mixture was stirred at 65° C. for 1 h. The reaction was quenched with ice-water (30 mL) and extracted with EtOAc (100 mL). The organic phase was washed with brine (100 ml), dried with Na₂SO₄, concentrated under pressure and purified by chromatography on silica gel, eluting 10% MeOH in DCM to afford N-{2-[(tert-butyldimethylsilyl)oxy]ethyl}-N-[(3S)-1-(3-chloro-6,7-difluoroquinoxalin-2-yl)pyrrolidin-3-yl]-2-methylpropanamide (1.0 g, 33.3% yield) as a yellow oil. LC-MS of 495-1: Ret. Time: 1.787 min, MS (ESI) m/z: 513.2, [M+H]⁺.

### Example 28B: Preparation of Compound 510-1

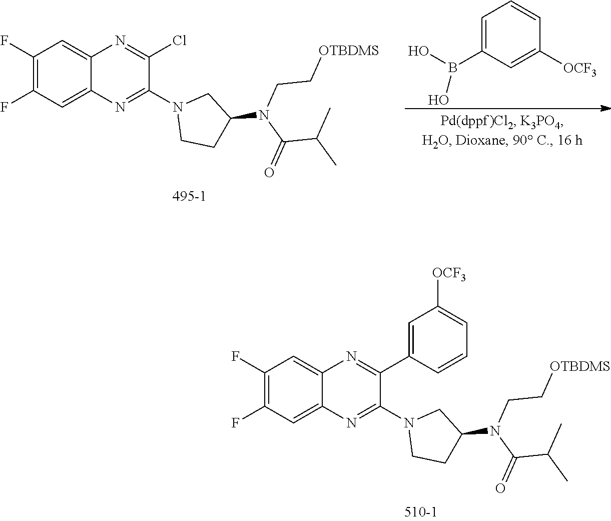 [SMILES: CC(C)C(=O)N(CCO[Si](C)(C)C(C)(C)C)[C@H]1CCN(c2nc3cc(F)c(F)cc3nc2Cl)C1 ; OB(O)c1cccc(OC(F)(F)F)c1 ; CC(C)C(=O)N(CCO[Si](C)(C)C(C)(C)C)[C@H]1CCN(c2nc3cc(F)c(F)cc3nc2-c2cccc(OC(F)(F)F)c2)C1]

To a solution of N-{2-[(tert-butyldimethylsilyl)oxy]ethyl}-N-[(3S)-1-(3-chloro-6,7-difluoroquinoxalin-2-yl)pyrrolidin-3-yl]-2-methylpropanamide (150 mg, 0.29 mmol), [3-(trifluoromethoxy)phenyl]boranediol (66 mg, 0.32 mmol) and K₃PO₄ (186 mg, 0.88 mmol) in 1,4-dioxane (10 mL) and water (1 mL) stirred under nitrogen at 25° C., Pd(dppf)Cl₂ (43 mg, 0.058 mmol) was added. The reaction mixture was stirred at 90° C. for 16 h. The reaction mixture was evaporated under reduced pressure giving a black oil. The residue was purified by chromatography on silica gel, eluting 25% EtOAc in PE to afford the N-{2-[(tert-butyldimethylsilyl)oxy]ethyl}-N-[(3S)-1-{6,7-difluoro-3-[3-(trifluoromethoxy)phenyl]quinoxalin-2-yl}pyrrolidin-3-yl]-2-methylpropanamide (140 mg, 75.0% yield) as a yellow oil. LC MS of 510-1: Ret. Time: 1.893 min, MS (ESI) m/z: 639.3, [M+H]⁺.

### Example 28C: Preparation of Compound 510

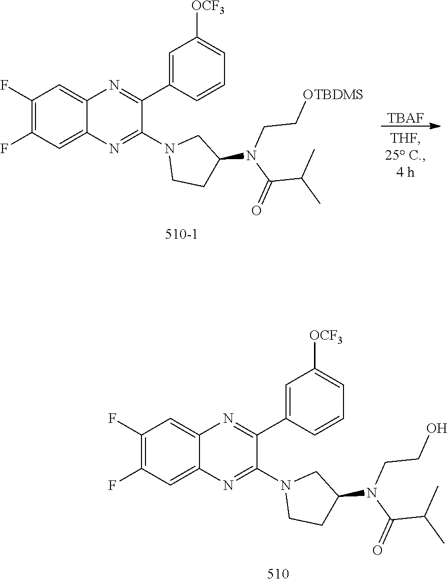 [SMILES: CC(C)C(=O)N(CCO[Si](C)(C)C(C)(C)C)[C@H]1CCN(c2nc3cc(F)c(F)cc3nc2-c2cccc(OC(F)(F)F)c2)C1 ; "TBAF" ; CC(C)C(=O)N(CCO)[C@H]1CCN(c2nc3cc(F)c(F)cc3nc2-c2cccc(OC(F)(F)F)c2)C1]

A solution of N-{2-[(tert-butyldimethylsilyl)oxy]ethyl}-N-[(3S)-1-{6,7-difluoro-3-[3-(trifluoromethoxy)phenyl]quinoxalin-2-yl}pyrrolidin-3-yl]-2-methylpropanamide (140 mg, 0.22 mmol) in THF (8 mL) was stirred under nitrogen atmosphere at 25° C. TBAF (172 mg, 0.66 mmol) was added. The reaction mixture was stirred at 25° C. for 4 h. The reaction was quenched with ice-water (30 mL), extracted with EtOAc (100 mL). The organic phase was washed with brine (100 mL), dried with Na₂SO₄, concentrated under pressure and purified by chromatography on silica gel, eluting 90% EtOAc in petroleum ether to afford (S)—N-(1-(6,7-difluoro-3-(3-(trifluoromethoxy)-phenyl)quinoxalin-2-yl)pyrrolidin-3-yl)-N-(2-hydroxyethyl)isobutyramide (68.9 mg, 60.0% yield) as white solid. LCMS of NYU032-510: Ret. Time: 1.429 min, MS (ESI) m/z: 525.2, [M+H]⁺. ¹H NMR of NYU032-510: ¹H NMR (400 MHz, CDCl₃) δ 8.43 (s, 1H), 7.79-7.48 (m, 4H), 7.44-7.31 (m, 1H), 4.53 (s, 1H), 4.11-3.22 (m, 8H), 2.83 (td, J=12.8, 6.4 Hz, 1H), 2.15 (s, 2H), 1.37-1.02 (m, 6H).

### Example 29: hSPNS2 Inhibition Assay: Assay to Measure Transport of S1P Out of Cells

A cell-based assay to measure transport of sphingosine-1-phosphate (S1P) out of cells was used. CHO+hSPNS2 clone 1B6 expressing the SPNS2 gene was cultivated with Ham's F12 (Gibco, #21765-029) including 10% FBS (Gibco, #10270-106), 2 mM L-glutamine, 1% Penicillin/Streptomycin (Biosera, #XC-A4122) and 400 μg/ml Hygromycin B (ABCONE, #H12894) at 37° C. and 5% CO₂ atmosphere. For sub-cultivation, media was removed and the cell layer rinsed with PBS. Cells were detached through addition of pre-warmed 0.25% Trypsin-EDTA (Invitrogen, #25200) and incubation for 3-5 min at 37° C. Cells to be seeded for assays were centrifuged for 10 min at 1000 rpm and the cell pellet was resuspended in the assay media (Ham's F12) and the cell concentration determined. For the assay, Cells were dispensed into 384-well plates (20,000 cells/30 μl/well) using the Thermo Fisher Multidrop 384.

Cells were incubated overnight at 37° C. (5% CO₂) to allow the cells to attach. Prior to the assay, 150 nl of various test compounds was transferred form 10 mM compound stock solution into 384-well intermediate plates (ECHO550). Next 45 μl/well of the freshly prepared 2× releasing medium was dispensed to the intermediate plate using a BioTek EL406. 2× releasing media was Ham's F-12 medium with 2 mM L-glutamine and contained 1.54% BSA (Fatty-acid free) Sigma Aldrich #SRE0098), 11 mM Sodium Fluoride, 2.2 mM Semicarbazide Hydrochloride, 22 mM β-Glycerophosphate Disodium and 5.5 μM D-Sphingosine.

To initiate the assay, 25 μl of 2× releasing media containing the test compounds and sphingosine (2.5 μM final conc.) was transferred from intermediate plate to 384-well plate containing the cells. The plates were incubated at 37° C. (5% CO₂) for 2 h. To stop the reaction, 30 μl/well of the supernatant was transferred to a 384-well readout plate and 90 μl/well of methanol was added to readout plate. The read-out plate was spun down at 15000 rpm for 1 min at room temperature, sealed and stored at −80° C. until ready for Mass Spec analysis. Usually, plates were stored over night before measurement. Samples from the read-out plate were injected into a Rapid Fire Mass Spectrometer to measure S1P vs a standard curve in 75% methanol.

For RapidFire tandem-mass spectrometer (RF-MS/MS), a chromatographic separation was performed on an Agilent 1200 infinity HPLC system, and injected onto an Agilent RapidFire High-throughput Mass Spectrometry System with cartridge HILIC type H6. The samples were aspirated for 500 ms in Buffer A (0.1% formic acid, water) at a flow rate of 1.5 mL/minute, and then loaded/washed for 3000 ms in Buffer B (0.1% formic acid, acetonitrile) at a flow rate of 1 mL/minute, eluted for 4500 ms in Buffer B at a flow rate of 0.75 mL/minute, finally re-equilibrated for 1000 ms in Buffer A at a flow rate of 1.5 mL/minute. Tandem MS was performed by positive ion mode ESI on an API 4000+ mass spectrometer (AB) with a source temperature of 600° C., a curtain gas of 30 psi, a gas1 of 60 psi, a gas2 of 70 psi, a cad gas of 12 psi, an ion spy voltage of 5500 V. Collision energies were optimized to 22.27 V for S1p; 14.72 V for Sph. Multiple reaction monitoring (MRM) mass transitions were S1p 380.3→264.3 m/z; Sph 300.3→282.4. Results of the assay are shown in Table 19 below.

TABLE 19
Results of hSPNS2 Inhibition Assay

| Compound ID | hSPNS2 IC₅₀ (mM) |
|---|---|
| 001A | 0.33 |
| 001B | 25 |
| 002A | 0.51 |
| 002B | >30 |
| 003 | 4.1 |
| 004 | 4.5 |
| 005 | >30 |
| 006 | 1.1 |
| 008 | 0.32 |
| 009 | >30 |
| 010 | >30 |
| 011 | 9.2 |
| 012 | 4.1 |
| 014 | 1.5 |
| 015 | 4.8 |
| 016 | 17 |
| 017 | 3.5 |
| 018 | >30 |
| 103 | >30 |
| 104 | 13 |
| 107 | 14 |
| 110 | 0.6 |
| 111 | 13 |
| 113 | 1.1 |
| 126 | 0.74 |
| 127 | 19 |
| 200 | 0.43 |
| 201 | 1.5 |
| 202 | >30 |
| 203 | 15 |
| 204 | >30 |
| 205 | 0.36 |
| 206 | 0.21 |
| 211 | 0.065 |
| 212 | 0.18 |
| 213 | 0.54 |
| 214 | >30 |
| 216 | >30 |
| 217 | >30 |
| 218 | >30 |
| 219 | >30 |
| 220 | >30 |
| 221 | >30 |
| 222 | >30 |
| 223 | 1.5 |
| 224 | 2.1 |
| 225 | 0.3 |
| 226 | 0.69 |
| 227 | 0.55 |
| 229 | 0.88 |
| 238 | 0.18 |
| 239 | 0.076 |
| 240 | 0.58 |
| 241 | 0.3 |
| 248 | 0.049 |
| 249 | 0.02 |
| 250 | 0.02 |
| 259 | 21 |
| 260 | 0.25 |
| 261 | 0.11 |
| 262 | 0.17 |
| 263 | 0.12 |
| 264 | 5.6 |
| 265 | 0.21 |
| 266 | 0.056 |
| 267 | 0.34 |
| 268 | 0.074 |
| 269 | 12 |
| 270 | 0.083 |
| 271 | >30 |
| 272 | 0.05 |
| 273 | 0.031 |
| 274 | 0.92 |
| 275 | >30 |
| 276 | 0.12 |
| 277 | 0.55 |
| 278 | 4.7 |
| 279 | 0.48 |
| 280 | 0.17 |
| 281 | 1.4 |
| 298 | 0.54 |
| 299 | 1.8 |
| 300 | 0.26 |
| 304 | 0.088 |
| 400 | 0.013 |
| 401 | 0.0094 |
| 402 | 0.012 |
| 403 | 0.01 |
| 405 | 0.033 |
| 406 | 0.064 |
| 407 | 0.69 |
| 408 | 0.44 |
| 409 | >30 |
| 410 | 0.0034 |
| 411 | 0.0058 |
| 412 | 0.0031 |
| 413 | 0.029 |
| 414 | 0.0029 |
| 415 | 0.0032 |
| 416 | 0.0088 |
| 417 | 0.012 |
| 418 | 0.0087 |
| 419 | 0.013 |
| 420 | 0.089 |
| 421 | 0.045 |
| 422 | 0.21 |
| 423 | 0.0088 |
| 424 | 0.0051 |
| 425 | 0.0057 |
| 426 | 0.0041 |
| 427 | 0.0082 |
| 428 | 0.07 |
| 429 | 0.015 |
| 430 | 0.0086 |
| 431 | 0.019 |
| 432 | 0.54 |
| 434 | 0.038 |
| 435 | 0.006 |
| 436 | 0.16 |
| 437 | 0.39 |
| 438 | 0.042 |
| 439 | 0.0024 |
| 440 | 0.48 |
| 441 | 8 |
| 444 | 0.0033 |
| 445 | 0.014 |
| 446 | 0.0016 |
| 447 | 0.0023 |
| 448 | 0.0016 |
| 449 | 0.037 |
| 452 | 0.029 |
| 453 | 0.53 |
| 454 | >3.0 |
| 455 | 0.12 |
| 456 | 0.046 |
| 457 | 0.022 |
| 458 | 0.29 |
| 459 | 0.2 |
| 460 | 0.31 |
| 461 | 0.054 |
| 462 | 0.026 |
| 463 | 0.041 |
| 466 | 0.041 |
| 471 | 0.21 |
| 472 | 0.56 |
| 480 | 0.014 |
| 481 | 0.091 |
| 482 | 0.018 |
| 483 | 0.07 |
| 484 | 0.033 |
| 485 | 0.0047 |
| 486 | 0.077 |
| 487 | 0.016 |
| 495 | 0.0033 |
| 496 | 0.015 |
| 497 | 0.0053 |
| 498 | 0.0097 |
| 499 | 0.18 |
| 500 | 0.34 |
| 501 | 0.045 |
| 502 | 0.32 |
| 503 | 0.67 |
| 504 | 0.014 |
| 505 | 0.077 |
| 506 | 0.0022 |
| 507 | 0.41 |
| 508 | 0.01 |
| 509 | 0.98 |
| 510 | 0.0012 |
| 511 | 0.021 |
| 517 | 0.035 |
| 521 | 0.012 |
| 522 | 0.049 |
| 528 | 0.015 |

### Example 30: Animal Study

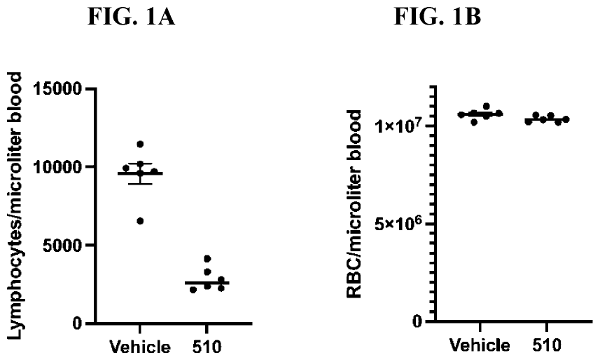

Female C57BL/6 mice 6-8 weeks old were treated by oral gavage with vehicle (PEG300) or compound 510 at 400 micromol/kg. 24 hours later, lymphocytes and red blood cells (RBC) in blood were enumerated by hematology analyzer (XT-2000i). The results are depicted in FIG. 1. FIG. 1A shows that compound 510 substantially reduced the number of lymphocytes. FIG. 1B shows that the number of red blood cells remained unchanged. Thus, treatment with compound 510 induced substantial lymphopenia without affecting RBC numbers, demonstrating that compound 510 is an effective SPNS2 inhibitor.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

1. A compound having a structure according to Formula (I): 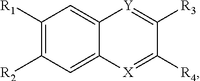
wherein:
X is —C(H)═ or —N═;
Y is —C(H)═ or —N═, wherein at least one of X and Y is not —C(H)═;
R₁ is a hydrogen, —Cl, —F, or a C₁-C₆ alkyl;
R₂ is a hydrogen, —Cl, —F, or a C₁-C₆ alkyl;
R₃ is —Cl, R₅, —OR₅, —NHR₅, or —N(R₅)₂;
R₄ is —OR₅, —NHR₅, —N(R₅)₂, 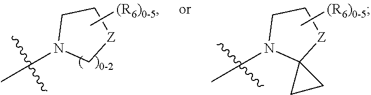
R₅ is independently at each occurrence an optionally substituted C₁-C₁₂ alkyl, an optionally substituted C₁-C₁₂ alkenyl, an optionally substituted C₁-C₁₂ alkoxy, an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 5-6 membered heteroaryl having 1-4 heteroatoms independently selected from N, O, and S, a fused bicyclic ring system having 1-4 heteroatoms independently selected from N, O, and S, or an optionally substituted combination of any two of a C₁-C₁₂ alkyl, a C₁-C₁₂ alkenyl and a ring;
Z is —C(H)—, —CH₂—, or —O—;
R₆ is independently at each occurrence an optionally substituted C₁-C₁₂ alkyl; an optionally substituted C₁-C₁₂ alkenyl; an optionally substituted phenyl, —N(R*)₂, —OR*, —N(R*)C(O)R*, —NH—SO₂—R*, —C(O)N(R*)₂, or 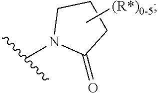
R* is independently at each occurrence hydrogen, an optionally substituted C₁-C₁₂ alkyl, an optionally substituted C₁-C₁₂ polyfluoroalkyl; an optionally substituted phenyl, an optionally substituted 3-7 membered saturated or partially unsaturated carbocyclyl; a 4-7 membered saturated or partially unsaturated heterocyclyl having 1-2 heteroatoms independently selected from N, O, and S,
or a pharmaceutically acceptable salt thereof,
with a proviso that wherein (i) R₁ and R₂ are both hydrogens, X and Y are both —N═, and R₃ is a 6 membered saturated heterocyclyl having 2 nitrogen atoms, R₄ is not 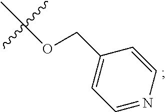 [SMILES: *OCc1ccncc1]
(ii) R₁ and R₂ are both hydrogens, X and Y are both —N═, and R₃ is phenyl substituted with —OCH₃ or —F,
R₄ is not 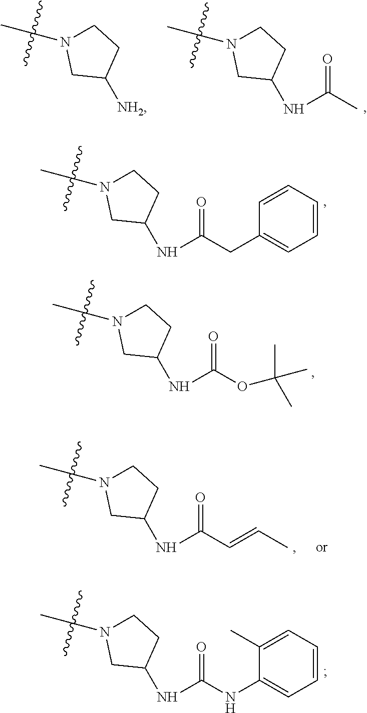
or
(iii) R₁ is a hydrogen, —Cl, —F, or a C₁-C₆ alkyl; R₂ is a hydrogen, —Cl, —F, or a C₁-C₆ alkyl; X is —N═; Y is —C(H)═ or —N═; and R₃ is —CH₃ or a combination of C₁-C₄ alkylene and a fused bicyclic ring system consisting of two fused six-membered aromatic rings containing at least one nitrogen atom, R₄ is not

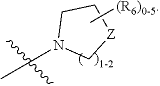

2.-3. (canceled)

4. The compound of claim 1, wherein at least one of X and Y is —N═.

5.-6. (canceled)

7. The compound of claim 1, wherein R₁ and R₂ are independently selected from the group consisting of hydrogen, —Cl, —F, methyl, or C₂-C₆ alkyl.

8.-19. (canceled)

20. The compound of claim 1, wherein R₃ is selected from the group consisting of —Cl, R₅, —OR₅, —NHR₅, and —N(R₅)₂.

21. (canceled)

22. The compound of claim 20, wherein R₅ is an optionally substituted phenyl, optionally substituted with one or more of —CN, —F, —Cl, —CH₃, —CH₂CH₃, —CF₃, —OH, —OCH₃, —OCH₂CH₃, —OCF₃, —CH₂OCH₃, —O—CH₂CH₂OCH₃, CONH₂, —CO₂CH₃,

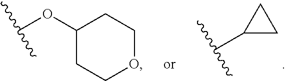

23.-24. (canceled)

25. The compound of claim 20, wherein —OR₅ is —O—(CH₂)₂OH, —O—(CH₂)₃CH₃, —O—CH₂—CH(CH₃)₂, —O—(CH₂)₂NH₂, —O(CH₂)₂OCH₃, —O—(CH₂)₂—N(CH₂CH₃)₂, —O—(CH₂)₂—NH—CH₂—CH₃, —O—CH₂CH₂—N(CH₃)—C(O)—CH(CH₃)₂, or selected from the group consisting of:

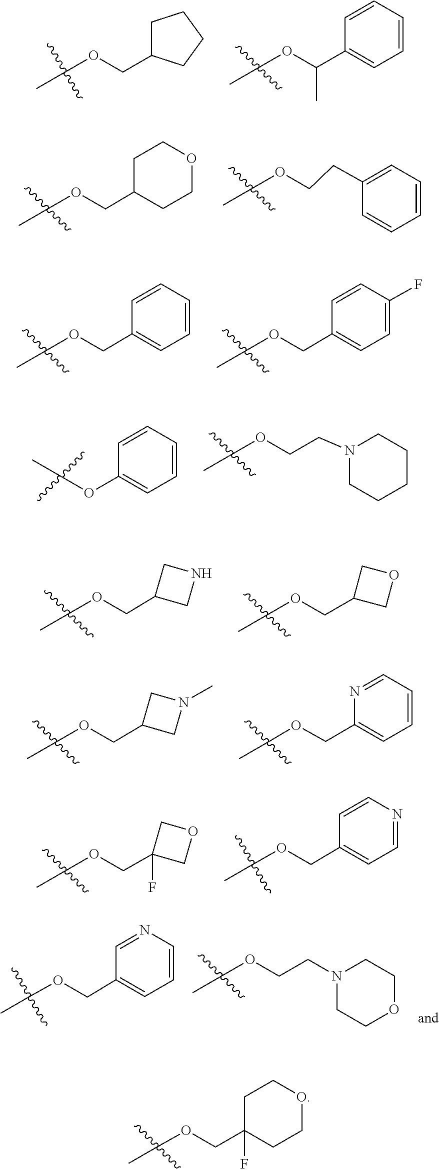

26. (canceled)

27. The compound of claim 20, wherein —NHR₅ is

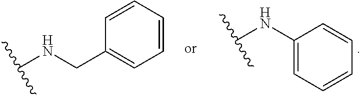 [SMILES: *NCc1ccccc1 ; *Nc1ccccc1]

28. (canceled)

29. The compound of claim 20, wherein —N(R₅)₂ is

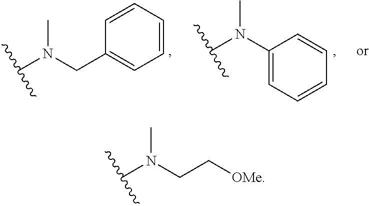

30. The compound of claim 1 having the structure according to Formula (II): 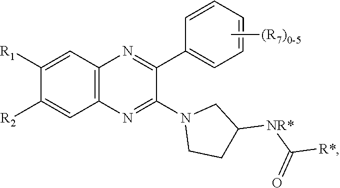
wherein
R₁ is a hydrogen, —Cl, —F, or a C₁-C₆ alkyl;
R₂ is a hydrogen, —Cl, —F, or a C₁-C₆ alkyl;
R₇ is independently at each occurrence —CN, —F, —Cl, —CH₃, —CH₂CH₃, —CF₃, —OH, —OCH₃, —OCH₂CH₃, —OCF₃, —CH₂OCH₃, —O—CH₂CH₂OCH₃, CONH₂, —CO₂CH₃, 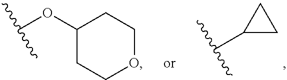
and
R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S,
or a pharmaceutically acceptable salt thereof.

31. (canceled)

32. The compound of claim 1 having the structure according to Formula (III), Formula (IV), or Formula (VI): 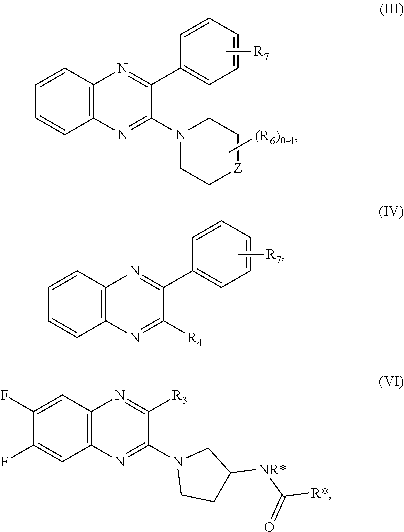
wherein
R₃ is 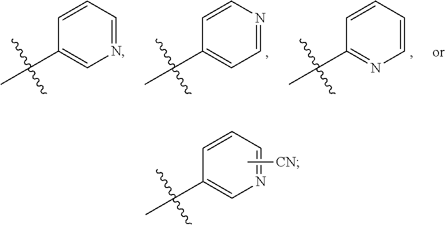
R₄ is —OR₅, —NHR₅, or —N(R₅)₂;
R₅ is independently at each occurrence —CH₃ or —(CH₂)₂—N(CH₃)—C(O)—CH(CH₃)₂;
R₆ is —CH₃, —NH—C(O)—CH₃, —NH—C(O)—CH(CH₃)₂, or —NH—C(O)—C(CH₃)₃;
R₇ is —CN;
R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S; and
Z is —CH₂— or —O—,
or a pharmaceutically acceptable salt thereof.

33. (canceled)

34. The compound of claim 1 having the structure according to Formula (V) or Formula (VII): 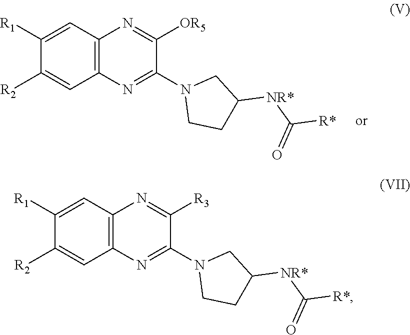
wherein
R₁ is a hydrogen, —Cl, or —F;
R₂ is a hydrogen, —Cl, or —F;
R₃ is —NHR₅, or —N(R₅)₂, wherein R₅ is —CH₃, —CH₂CH₂OCH₃, -phenyl, or —CH₂-phenyl;
—OR₅ is —O—CH₂CH₂—N(CH₃)—C(O)—CH(CH₃)₂, —O—(CH₂)₃—CH₃, —O—CH₂—CH(CH₃)₂, —O—(CH₂)₂OH, —O—(CH₂)₂NH₂, —O(CH₂)₂OCH₃, —O—(CH₂)₂—N(CH₂CH₃)₂, —O—(CH₂)₂—NH—CH₂—CH₃, or selected from the group consisting of: 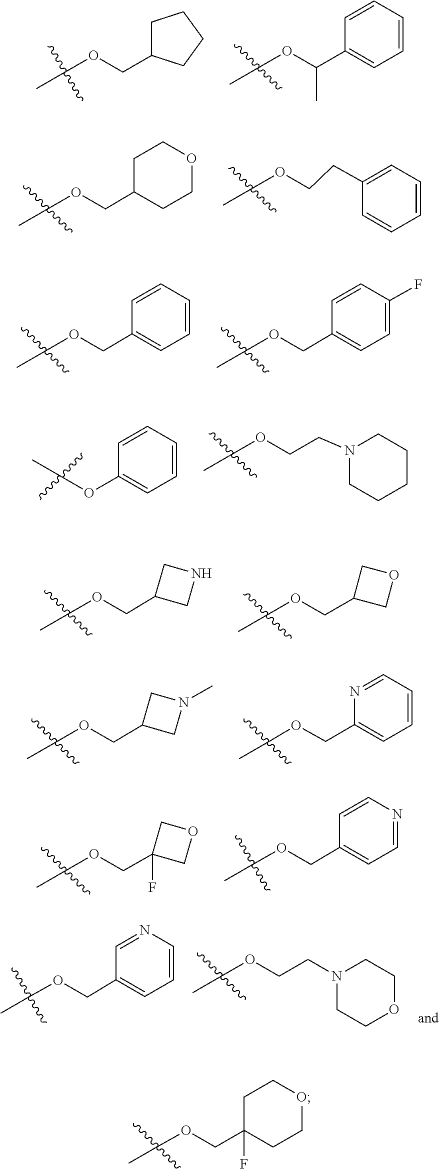
and
R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S;
or a pharmaceutically acceptable salt thereof.

35.-40. (canceled)

41. The compound of claim 1 having the structure according to Formula (VIII) or Formula (IX): 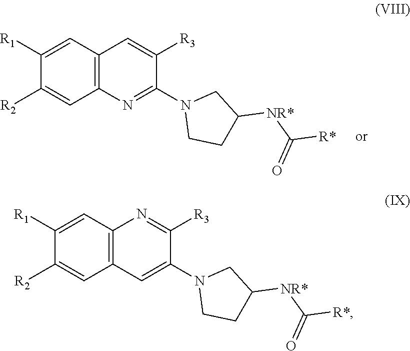
wherein
R₁ is a hydrogen, —Cl, or —F;
R₂ is a hydrogen, —Cl, or —F;
R₃ is 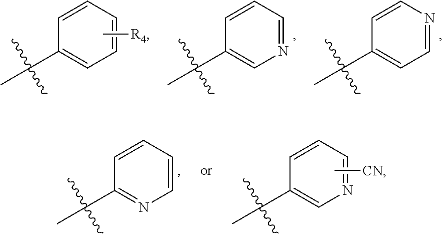
wherein
R₄ is a hydrogen, —C(O)NH₂, or —CN;
R* is independently at each occurrence hydrogen, an optionally substituted C₁-C₁₂ alkyl, an optionally substituted C₁-C₁₂ polyfluoroalkyl, an optionally substituted phenyl, an optionally substituted 3-7 membered saturated or partially unsaturated carbocyclyl, a 4-7 membered saturated or partially unsaturated heterocyclyl having 1-2 heteroatoms independently selected from N, O, and S, or a pharmaceutically acceptable salt thereof.

42. The compound of claim 41, wherein R* is independently at each occurrence —H, —CH₃, —CH₂CH₃, —CH₂CF₃, —CH₂CH₂OH, —CH(CH₃)₂, or —C(CH₃)₃.

43.-44. (canceled)

45. The compound of claim 1, having the structure selected from the group consisting of:

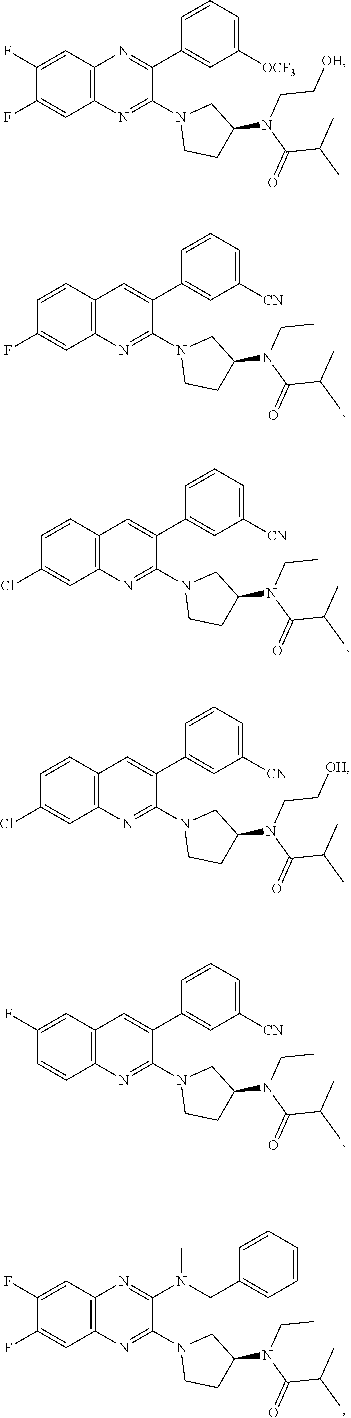 [SMILES: CC(C)C(=O)N(CCO)[C@H]1CCN(c2nc3cc(F)c(F)cc3nc2-c2cccc(OC(F)(F)F)c2)C1 ; CCN(C(=O)C(C)C)[C@H]1CCN(c2nc3cc(F)ccc3cc2-c2cccc(C#N)c2)C1 ; CCN(C(=O)C(C)C)[C@H]1CCN(c2nc3cc(Cl)ccc3cc2-c2cccc(C#N)c2)C1 ; CC(C)C(=O)N(CCO)[C@H]1CCN(c2nc3cc(Cl)ccc3cc2-c2cccc(C#N)c2)C1 ; CCN(C(=O)C(C)C)[C@H]1CCN(c2nc3ccc(F)cc3cc2-c2cccc(C#N)c2)C1 ; CCN(C(=O)C(C)C)[C@H]1CCN(c2nc3cc(F)c(F)cc3nc2N(C)Cc2ccccc2)C1]

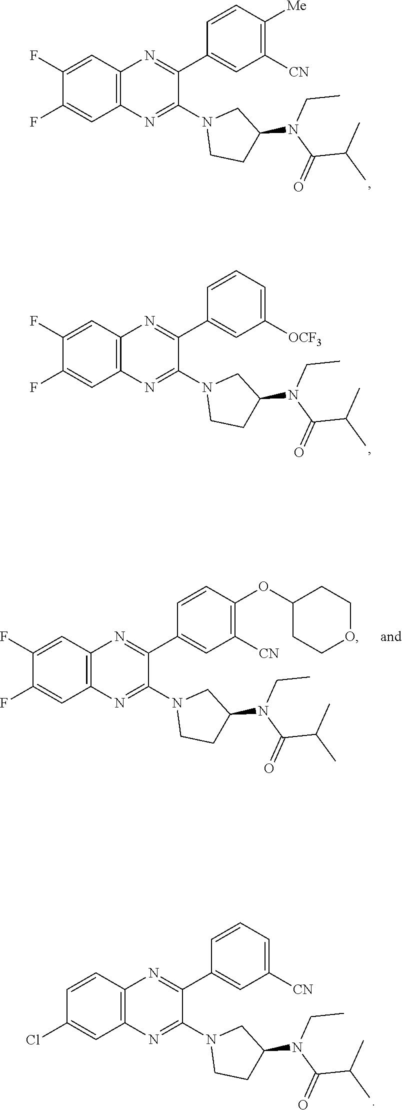 [SMILES: CCN(C(=O)C(C)C)[C@H]1CCN(c2nc3cc(F)c(F)cc3nc2-c2ccc(C)c(C#N)c2)C1 ; CCN(C(=O)C(C)C)[C@H]1CCN(c2nc3cc(F)c(F)cc3nc2-c2cccc(OC(F)(F)F)c2)C1 ; CCN(C(=O)C(C)C)[C@H]1CCN(c2nc3cc(F)c(F)cc3nc2-c2ccc(OC3CCOCC3)c(C#N)c2)C1 ; CCN(C(=O)C(C)C)[C@H]1CCN(c2nc3cc(Cl)ccc3nc2-c2cccc(C#N)c2)C1]

46. A pharmaceutical composition comprising the compound of claim 1 or a pharmaceutically acceptable salt thereof and a pharmaceutically acceptable carrier.

47. A pharmaceutical dosage form comprising the compound of claim 1 or a pharmaceutically acceptable salt thereof.

48. A method of inhibiting Sphingolipid Transporter 2 (SPNS2) in a subject in need thereof comprising administering to the subject a therapeutically effective amount of the compound of claim 1 or a pharmaceutically acceptable salt thereof.

49. A method of treating a disease or condition in a subject in need thereof comprising administering to the subject a therapeutically effective amount of the compound of claim 1 or a pharmaceutically acceptable salt thereof.

50. The method of claim 49, wherein the disease or condition is an autoimmune disease.

51. The method of claim 49, wherein the disease or condition is multiple sclerosis (MS), fibrosis, muscle wasting, metastases, acute lung injury, rheumatoid arthritis, colitis, Alzheimer's disease, or inflammatory bowel disease (IBD).

52. (canceled)
